(19) United States
(12) Patent Application Publication
(10) Pub. No.: US 2026/0191896 A1
(43) Pub. Date: Jul. 9, 2026

(54) METHODS AND SYSTEMS FOR PREVENTING CARDIOMETABOLIC DISEASES

(71) Applicant: Validae Health, L.P., Short Hills, NJ (US)

(72) Inventor: Brian A. Ference, Short Hills, NJ (US)

(73) Assignee: Validae Health, L.P., Short Hills, NJ (US)

(21) Appl. No.: 19/389,988

(22) Filed: Nov. 14, 2025

Related U.S. Application Data
(60) Provisional application No. 63/721,246, filed on Nov. 15, 2024.

(51) Int. Cl. A61K 31/7105; A61B 5/00; G16H 50/30

(52) U.S. Cl. CPC A61K 31/7105; A61B 5/4866; A61B 5/7275; G16H 50/30

(57) ABSTRACT

Some embodiments provide for a method for identifying an intervention for a subject in furtherance of preventing development of cardiovascular disease in the subject, the method comprising: obtaining cardiometabolic health data for the subject; determining, using at least some of the cardiometabolic health data, a first trained machine learning (ML) model and for each of multiple time intervals, one or more measures of risk that the subject develops cardiovascular disease to obtain multiple measures of risk; determining, using the multiple measures of risk that the subject develops cardiovascular disease and at least one second trained ML model, benefit of administering to the subject one or more therapeutic interventions designed to reduce risk of cardiovascular disease by targeting one or more modifiable causes of the cardiovascular disease; and identifying, using the determined benefit of administering the therapeutic intervention(s), at least one therapeutic intervention to recommend being administered to the subject.

### CROSS-REFERENCE TO RELATED APPLICATIONS

This application claims the benefit of priority under 35 U.S.C. 119 to U.S. Provisional Application No. 63/721,246, titled “Methods and Systems for Preventing Cardiometabolic Diseases”, filed on Nov. 15, 2024, which is incorporated by reference in its entirety herein.

### FIELD

Aspects of the present disclosure relate to methods and systems for identifying one or more intervention for an individual in furtherance of preventing development of cardiometabolic disease, such as cardiovascular disease (e.g., atherosclerotic cardiovascular disease (ASCVD)) and diabetes (e.g., type 2 diabetes).

### BACKGROUND

Personalized preventive medicine focuses on tailoring preventive strategies to individual patients based on their unique risk factors, genetics, lifestyle, and environmental influences. A personalized approach to preventative medicine aims to prevent diseases before illnesses develop, often through customized interventions.

### SUMMARY

The present technology relates to computer-implemented methods and systems for personalized cardiovascular disease risk assessment and therapeutic intervention selection using machine learning models. The disclosed methods comprise obtaining comprehensive cardiometabolic health data for a subject, including clinical characteristics, physical measurements, and biochemical markers, and processing this data through trained machine learning models to generate predictive biomarker trajectories. Unlike conventional risk assessment approaches that rely on static measurements, the present technology estimates dynamic trajectories of modifiable causes of disease, such as low-density lipoprotein (LDL) cholesterol and systolic blood pressure (SBP), and other key cardiovascular risk factors across multiple time intervals throughout a subject's lifetime.

The methods further comprise determining personalized cardiovascular disease risk measures by processing the predicted biomarker trajectories through trained survival models that utilize cumulative exposure metrics as intervals of follow-up. The potential benefit of various therapeutic interventions are then evaluated by modeling their effects on the subject's predicted risk trajectory, enabling the identification of optimal treatment strategies tailored to individual risk profiles. The disclosed methods may be applied to various therapeutic modalities, including pharmacological interventions, DNA-based therapeutics, RNA-based therapeutics, and protein-based therapeutics. Additionally, the technology provides applications in insurance underwriting, where the personalized risk assessments may be utilized to determine appropriate pricing for insurance instruments based on individualized cardiovascular disease risk profiles rather than population-based actuarial models.

Some aspects of the technology provide a method for identifying an intervention for a subject in furtherance of preventing development of cardiovascular disease in the subject, the method comprising: using at least one computer hardware processor to perform: obtaining cardiometabolic health data for the subject; determining, using at least some of the cardiometabolic health data, a first trained machine learning (ML) model and for each of multiple time intervals, one or more measures of risk that the subject develops cardiovascular disease to obtain multiple measures of risk corresponding to the multiple time intervals; determining, using the multiple measures of risk that the subject develops cardiovascular disease and at least one second trained ML model, benefit of administering to the subject one or more therapeutic interventions designed to reduce risk of cardiovascular disease by targeting one or more modifiable causes of the cardiovascular disease; and identifying, using the determined benefit of administering the one or more therapeutic interventions, at least one therapeutic intervention to recommend being administered to the subject.

Other aspects of the technology provide a method for identifying an intervention for a subject in furtherance of preventing development of cardiovascular disease in the subject, the method comprising: using at least one computer hardware processor to perform: obtaining cardiometabolic health data for the subject; determining, using at least some of the cardiometabolic health data, a survival model with cumulative LDL exposure as interval of follow-up, and for each of multiple time intervals, one or more measures of risk that the subject develops cardiovascular disease to obtain multiple measures of risk corresponding to the multiple time intervals; determining, using the multiple measures of risk that the subject develops cardiovascular disease, benefit of administering to the subject one or more therapeutic interventions designed to reduce risk of cardiovascular disease by targeting one or more modifiable causes of the cardiovascular disease; and identifying, using the determined benefit of administering the one or more therapeutic interventions, at least one therapeutic intervention to recommend to be administered to the subject.

Yet other aspects of the technology provide a method for identifying an intervention for a subject in furtherance of preventing development of cardiovascular disease in the subject, the method comprising: using at least one computer hardware processor to perform: obtaining cardiometabolic health data for the subject, the cardiometabolic health data comprising clinical, physical, and biochemical measurements of the subject, the obtaining further comprising estimating a cumulative LDL exposure trajectory for the subject using a trained ML model and the physical and biochemical measurements; determining, using at least some of the cardiometabolic health data including the cumulative LDL exposure trajectory and for each of multiple time intervals, one or more measures of risk that the subject develops cardiovascular disease to obtain multiple measures of risk corresponding to the multiple time intervals; determining, using the multiple measures of risk that the subject develops cardiovascular disease, benefit of administering to the subject one or more therapeutic interventions designed to reduce risk of cardiovascular disease by targeting one or more modifiable causes of the cardiovascular disease; and identifying, using the determined benefit of administering the one or more therapeutic interventions, at least one therapeutic intervention to recommend to be administered to the subject.

Additional aspects of the technology provide a method of estimating a biomarker for a subject in furtherance of preventing development of cardiovascular disease in the subject, the method comprising: obtaining cardiometabolic health data for the subject, the cardiometabolic health data comprising subject characteristic and/or measurement data comprising: one or more values for one or more clinical characteristics of the subject, one or more values for one or more physical measurements of the subject, and/or one or more values for one or biochemical measurements of the subject; estimating, using a trained machine learning (ML) model and the subject characteristic and/or measurement data, an LDL level trajectory for the subject, wherein the LDL level trajectory for the subject comprises an estimated LDL level for the subject for each of multiple prior ages of the subject and multiple future ages of the subject; and determining a cumulative LDL exposure trajectory for the subject as the biomarker for use in identifying a therapeutic intervention for the subject in furtherance of preventing development of cardiovascular disease in the subject.

Further aspects of the technology provide a method of estimating a biomarker for a subject in furtherance of preventing development of cardiovascular disease in the subject, the method comprising: obtaining cardiometabolic health data for the subject, the cardiometabolic health data comprising subject characteristic and/or measurement data comprising: one or more values for one or more clinical characteristics of the subject, one or more values for one or more physical measurements of the subject, and/or one or more values for one or biochemical measurements of the subject; estimating, using a trained machine learning (ML) model and the subject characteristic and/or measurement data, an SBP level trajectory for the subject, wherein the SBP level trajectory for the subject comprises an estimated SBP level for the subject for each of multiple prior ages of the subject and multiple future ages of the subject; and determining a cumulative SBP exposure trajectory for the subject as the biomarker for use in identifying a therapeutic intervention for the subject in furtherance of preventing development of cardiovascular disease in the subject.

Some aspects of the technology provide a method of estimating a biomarker for a subject in furtherance of preventing development of cardiovascular disease in the subject, the method comprising: obtaining cardiometabolic health data for the subject, the cardiometabolic health data comprising subject characteristic and/or measurement data comprising: one or more values for one or more clinical characteristics of the subject, one or more values for one or more physical measurements of the subject, and/or one or more values for one or biochemical measurements of the subject; estimating, using a trained machine learning (ML) model and the subject characteristic and/or measurement data, an Lp(a) level trajectory for the subject, wherein the Lp(a) level trajectory for the subject comprises an estimated Lp(a) level for the subject for each of multiple prior ages of the subject and multiple future ages of the subject; and determining a cumulative Lp(a) exposure trajectory for the subject as the biomarker for use in identifying a therapeutic intervention for the subject in furtherance of preventing development of cardiovascular disease in the subject.

Other aspects of the technology provide a method of determining one or more measures of risk that a subject develops cardiovascular disease, the method comprising, for each of multiple time intervals: using at least one computer hardware processor to perform: (a) estimating, using a trained cardiovascular risk prediction machine learning model and cardiometabolic health data for the subject including a cumulative LDL exposure trajectory for the subject, values indicative of log hazard ratios for risk of the subject having a cardiovascular event at respective levels of cumulative LDL exposure, wherein the cardiovascular risk prediction machine learning model has been trained using training data comprising, for each of a plurality of participants enrolled in one or more prospective studies, multiple LDL measurements along with a recorded age or date at which a first cardiovascular event occurred, optionally wherein the first cardiovascular event is a first episode of a fatal or non-fatal myocardial infarction (MI), fatal or non-fatal ischemic stroke, or coronary revascularization; and (b) estimating, using the values indicative of the log hazard ratios, (i) absolute instantaneous hazard rates of the subject having a cardiovascular event at the respective levels of cumulative LDL exposure, and (ii) cumulative lifetime risks of the subject having a cardiovascular event at the respective levels of cumulative LDL exposure.

Yet other aspects of the technology provide a method of determining an expected proportional reduction and/or absolute reduction in risk of cardiovascular events for a subject in response to a particular therapeutic intervention sequence, the particular therapeutic sequence indicating magnitude, duration, and timing of one or more interventions associated with a reduction of LDL level and/or SBP level over an interval of follow up for the subject, the method comprising: using at least one computer hardware processor to perform: determining multiple measures of risk of cardiovascular events for the subject comprising absolute instantaneous hazard rates, cumulative hazard rates, and cumulative event rates of the subject having a cardiovascular event at respective ones of multiple time intervals, wherein the cumulative hazard rates and the cumulative event rates are not adjusted for the particular therapeutic intervention sequence, and determining the expected proportional reduction and/or the absolute reduction in the risk of cardiovascular events for the subject in response to the particular therapeutic intervention sequence using a method that comprises: for each particular interval of follow-up for the subject from the subject's current age to an upper threshold age, (i) determining, using the multiple measures of risk, a predicted instantaneous hazard rate of the subject having a cardiovascular event at the particular interval of follow up; (ii) determining, using a benefit prediction machine learning model, a time-averaged instantaneous log hazard ratio for a one unit lower LDL or SBP corresponding to duration of treatment at the particular interval of follow-up for the subject; (iii) determining an intervention-adjusted instantaneous hazard for the particular interval of follow-up by multiplying the instantaneous hazard rate of the subject determined at (a)(i) with the time-averaged instantaneous log hazard ratio determined at (a)(ii), thereby obtaining multiple intervention-adjusted instantaneous hazards for intervals of follow-up evaluated at (a); determining, using the multiple intervention-adjusted instantaneous hazards, intervention-adjusted cumulative hazard rates and cumulative event rates of cardiovascular events for intervals of follow-up for the subject from the subject's current age to the upper threshold age; and determining predicted proportional reductions in the risk of experiencing a cardiovascular event as ratios of the intervention-adjusted cumulated hazard rates and the cumulative hazard rates that are not adjusted for the particular therapeutic intervention sequence.

Additional aspects of the technology provide a method for pricing an insurance instrument for a subject based, the method comprising: using at least one computer hardware processor to perform: obtaining cardiometabolic health data for the subject; determining, using at least some of the cardiometabolic health data and for each of multiple time intervals, one or more measures of risk that the subject develops a disease to obtain multiple measures of risk corresponding to the multiple time intervals; and pricing the insurance instrument for the subject based on the multiple measures of risk corresponding to the multiple time intervals.

Further aspects of the technology provide a system, comprising: at least one computer hardware processor; and at least one non-transitory computer readable storage medium storing software comprising: a cardiometabolic health data module comprising processor-executable instructions that, when executed by at least one computer hardware processor, cause at least one computer hardware processor to perform obtaining cardiometabolic health data for a subject; a risk assessment module comprising processor-executable instructions that, when executed by at least one computer hardware processor, cause at least one computer hardware processor to perform determining, using at least some of the cardiometabolic health data and for each of multiple time intervals, one or more measures of risk that the subject develops disease to obtain multiple measures of risk corresponding to the multiple time intervals; a therapeutic intervention benefit assessment module comprising processor executable instructions that, when executed by at least one computer hardware processor, cause at least one computer hardware processor to perform determining, using the multiple measures of risk that the subject develops the disease, benefit of administering to the subject one or more therapeutic interventions designed to reduce risk of the disease by targeting one or more modifiable causes of the disease; and a therapeutic intervention selection module comprising processor executable instructions that, when executed by at least one computer hardware processor, cause at least one computer hardware processor to perform identifying, using the determined benefit of administering the one or more therapeutic interventions, at least one therapeutic intervention to recommend to be administered to the subject.

Some aspects of the technology provide a system, comprising: at least one computer hardware processor; and at least one non-transitory computer readable storage medium storing processor-executable instructions that, when executed by at least one processor, cause at least one computer hardware processor to perform any one of the methods described herein.

Other aspects of the technology provide at least one non-transitory computer readable storage medium storing processor-executable instructions that, when executed by at least one processor, cause at least one processor to perform any one of the methods described herein.

Yet other aspects of the technology provide a method for identifying an intervention for a subject in furtherance of preventing development of cardiovascular disease in the subject, the method comprising: using at least one computer hardware processor to perform: obtaining cardiometabolic health data for the subject; determining, using at least some of the cardiometabolic health data, a first trained machine learning (ML) model and for each of multiple time intervals, one or more measures of risk that the subject develops cardiovascular disease to obtain multiple measures of risk corresponding to the multiple time intervals; determining, using the multiple measures of risk that the subject develops cardiovascular disease and at least one second ML model, benefit of administering to the subject one or more therapeutic interventions designed to reduce risk of cardiovascular disease by targeting one or more modifiable causes of the cardiovascular disease; and identifying, using the determined benefit of administering the one or more therapeutic interventions, at least one therapeutic intervention to recommend being administered to the subject.

Additional aspects of the technology provide a system, comprising: at least one computer hardware processor; and at least one non-transitory computer-readable storage medium storing processor executable instructions that, when executed by at least one computer hardware processor, cause at least one computer hardware processor to perform a method for identifying an intervention for a subject in furtherance of preventing development of cardiovascular disease in the subject, the method comprising: obtaining cardiometabolic health data for the subject; determining, using at least some of the cardiometabolic health data, a first trained machine learning (ML) model and for each of multiple time intervals, one or more measures of risk that the subject develops cardiovascular disease to obtain multiple measures of risk corresponding to the multiple time intervals; determining, using the multiple measures of risk that the subject develops cardiovascular disease and at least one second ML model, benefit of administering to the subject one or more therapeutic interventions designed to reduce risk of cardiovascular disease by targeting one or more modifiable causes of the cardiovascular disease; and identifying, using the determined benefit of administering the one or more therapeutic interventions, at least one therapeutic intervention to recommend being administered to the subject.

Further aspects of the technology provide at least one non-transitory computer-readable storage medium storing processor executable instructions that, when executed by at least one computer hardware processor, cause at least one computer hardware processor to perform a method for identifying an intervention for a subject in furtherance of preventing development of cardiovascular disease in the subject, the method comprising: obtaining cardiometabolic health data for the subject; determining, using at least some of the cardiometabolic health data, a first trained machine learning (ML) model and for each of multiple time intervals, one or more measures of risk that the subject develops cardiovascular disease to obtain multiple measures of risk corresponding to the multiple time intervals; determining, using the multiple measures of risk that the subject develops cardiovascular disease and at least one second ML model, benefit of administering to the subject one or more therapeutic interventions designed to reduce risk of cardiovascular disease by targeting one or more modifiable causes of the cardiovascular disease; and identifying, using the determined benefit of administering the one or more therapeutic interventions, at least one therapeutic intervention to recommend being administered to the subject.

Some aspects of the technology provide a method for estimating a cumulative LDL exposure trajectory for a subject, the method comprising: using at least one computer hardware processor to perform: obtaining cardiometabolic health data for a subject comprising: one or more values for one or more clinical characteristics of the subject, and/or one or more values for one or more physical measurements of the subject, and/or one or more values for one or more biochemical measurements of the subject; encoding the cardiometabolic health data for the subject into a feature vector; estimating an LDL level trajectory for the subject by processing the feature vector using an LDL trajectory prediction machine learning (ML) model that has been trained to estimate an LDL level for a subject at each of multiple prior ages and each of multiple future ages using training data comprising for each of a plurality of participants, repeated longitudinal measures of LDL levels and values for the one or more clinical characteristics and/or the one or more physical measurements and/or the one or more biochemical measurements over at least 10, at least 20, at least 30, or at least 50 years of follow-up, wherein the LDL level trajectory for the subject comprises estimated LDL levels for the subject including an estimated LDL level for each of multiple prior ages of the subject and multiple future ages of the subject; estimating, using the LDL level trajectory, a cumulative LDL exposure trajectory for the subject with respect to a set of ages, wherein the cumulative LDL exposure trajectory comprises an estimated cumulative LDL exposure level for the subject at each age in the set of ages; and outputting the estimate cumulative LDL exposure trajectory for the subject.

Other aspects of the technology provide a system, comprising: at least one computer hardware processor; and at least one non-transitory computer-readable storage medium storing processor executable instructions that, when executed by at least one computer hardware processor, cause at least one computer hardware processor to perform a method for estimating a cumulative LDL exposure trajectory for a subject, the method comprising: obtaining cardiometabolic health data for a subject comprising: one or more values for one or more clinical characteristics of the subject, and/or one or more values for one or more physical measurements of the subject, and/or one or more values for one or more biochemical measurements of the subject; encoding the cardiometabolic health data for the subject into a feature vector; estimating an LDL level trajectory for the subject by processing the feature vector using an LDL trajectory prediction machine learning (ML) model that has been trained to estimate am LDL level for a subject at each of multiple prior ages and each of multiple future ages using training data comprising for each of a plurality of participants, repeated longitudinal measures of LDL levels and values for the one or more clinical characteristics and/or the one or more physical measurements and/or the one or more biochemical measurements over at least 10, at least 20, at least 30, or at least 50 years of follow-up, wherein the LDL level trajectory for the subject comprises estimated LDL levels for the subject including an estimated LDL level for each of multiple prior ages of the subject and multiple future ages of the subject; estimating, using the LDL level trajectory, a cumulative LDL exposure trajectory for the subject with respect to a set of ages, wherein the cumulative LDL exposure trajectory comprises an estimated cumulative LDL exposure level for the subject at each age in the set of ages; and outputting the estimate cumulative LDL exposure trajectory for the subject.

Yet other aspects of the technology provide at least one non-transitory computer-readable storage medium storing processor executable instructions that, when executed by at least one computer hardware processor, cause at least one computer hardware processor to perform a method for estimating a cumulative LDL exposure trajectory for a subject, the method comprising: obtaining cardiometabolic health data for a subject comprising: one or more values for one or more clinical characteristics of the subject, and/or one or more values for one or more physical measurements of the subject, and/or one or more values for one or more biochemical measurements of the subject; encoding the cardiometabolic health data for the subject into a feature vector; estimating an LDL level trajectory for the subject by processing the feature vector using an LDL trajectory prediction machine learning (ML) model that has been trained to estimate am LDL level for a subject at each of multiple prior ages and each of multiple future ages using training data comprising for each of a plurality of participants, repeated longitudinal measures of LDL levels and values for the one or more clinical characteristics and/or the one or more physical measurements and/or the one or more biochemical measurements over at least 10, at least 20, at least 30, or at least 50 years of follow-up, wherein the LDL level trajectory for the subject comprises estimated LDL levels for the subject including an estimated LDL level for each of multiple prior ages of the subject and multiple future ages of the subject; estimating, using the LDL level trajectory, a cumulative LDL exposure trajectory for the subject with respect to a set of ages, wherein the cumulative LDL exposure trajectory comprises an estimated cumulative LDL exposure level for the subject at each age in the set of ages; and outputting the estimate cumulative LDL exposure trajectory for the subject.

Additional aspects of the technology provide a computer-implemented method, comprising: obtaining cardiometabolic health data for a subject comprising: one or more values for one or more clinical characteristics of the subject, and/or one or more values for one or more physical measurements of the subject, and/or one or more values for one or more biochemical measurements of the subject; encoding the cardiometabolic health data for the subject into a first feature vector; estimating an LDL level trajectory for the subject by processing the first feature vector using an LDL trajectory prediction machine learning (ML) model that has been trained to estimate an LDL level for a subject at each of multiple prior ages and each of multiple future ages using training data comprising for each of a plurality of participants, repeated longitudinal measures of LDL levels and values for the one or more clinical characteristics and/or the one or more physical measurements and/or the one or more biochemical measurements over at least 10, at least 20, at least 30, or at least 50 years of follow-up, wherein the LDL level trajectory for the subject comprises an estimated LDL level for the subject for each of multiple prior ages of the subject and multiple future ages of the subject.

Further aspects of the technology provide a computer-implemented method of determining one or more measures of risk that a subject develops cardiovascular disease, the method comprising, for each of multiple time intervals: (a) estimating, using a trained cardiovascular risk prediction machine learning model and cardiometabolic health data for the subject including a cumulative LDL exposure trajectory for the subject, values indicative of log hazard ratios for risk of the subject having a cardiovascular event at respective levels of cumulative LDL exposure, wherein the cardiovascular risk prediction machine learning model has been trained using training data comprising, for each of a plurality of participants enrolled in one or more prospective studies, multiple LDL measurements along with a recorded age or date at which a first cardiovascular event occurred, optionally wherein the first cardiovascular event is a first episode of a fatal or non-fatal myocardial infarction (MI), fatal or non-fatal ischemic stroke, or coronary revascularization; and (b) estimating, using the values indicative of the log hazard ratios, (i) absolute instantaneous hazard rates of the subject having a cardiovascular event at the respective levels of cumulative LDL exposure, and (ii) cumulative lifetime risks of the subject having a cardiovascular event at the respective levels of cumulative LDL exposure.

Some aspects of the technology provide a computer-implemented method of determining an expected proportional reduction and/or absolute reduction in risk of cardiovascular events for a subject in response to a particular therapeutic intervention sequence, the particular therapeutic sequence indicating magnitude, duration, and timing of one or more interventions associated with a reduction of LDL level and/or SBP level over an interval of follow up for the subject, the method comprising: determining multiple measures of risk of cardiovascular events for the subject comprising absolute instantaneous hazard rates, cumulative hazard rates, and cumulative event rates of the subject having a cardiovascular event at respective ones of multiple time intervals, wherein the cumulative hazard rates and the cumulative event rates are not adjusted for the particular therapeutic intervention sequence, and determining the expected proportional reduction and/or the absolute reduction in the risk of cardiovascular events for the subject in response to the particular therapeutic intervention sequence using a method that comprises: (a) for each particular interval of follow-up for the subject from the subject's current age to an upper threshold age, (i) determining, using the multiple measures of risk, a predicted instantaneous hazard rate of the subject having a cardiovascular event at the particular interval of follow up; (ii) determining, using a benefit prediction machine learning (ML) model, a time-averaged instantaneous log hazard ratio for a one unit lower LDL or SBP corresponding to duration of treatment at the particular interval of follow-up for the subject, wherein the benefit prediction machine learning model has been trained using training data from randomized trials of LDL lowering therapies and/or randomized trials of SBP therapies and Mendelian randomization studies evaluating genetic variants associated with lower LDL and/or lower SBP, said training data comprising for each of a plurality of participants in said trials, at least one LDL or SBP measurement along with a recorded age or date at which a first cardiovascular event occurred; (iii) determining an intervention-adjusted instantaneous hazard for the particular interval of follow-up by multiplying the instantaneous hazard rate of the subject determined at (a)(i) with the time-averaged instantaneous log hazard ratio determined at (a)(ii), thereby obtaining multiple intervention-adjusted instantaneous hazards for intervals of follow-up evaluated at (a); (b) determining, using the multiple intervention-adjusted instantaneous hazards, intervention-adjusted cumulative hazard rates and cumulative event rates of cardiovascular events for intervals of follow-up for the subject from the subject's current age to the upper threshold age; and (c) determining predicted proportional reductions in the risk of experiencing a cardiovascular event as ratios of the intervention-adjusted cumulated hazard rates and the cumulative hazard rates that are not adjusted for the particular therapeutic intervention sequence.

Other aspects of the technology provide a computer-implemented method for pricing an insurance instrument for a subject, the method comprising: obtaining cardiometabolic health data for the subject including a cumulative LDL exposure trajectory for the subject; determining multiple measures of risk that the subject develops cardiovascular disease using a method comprising: (a) estimating, using a trained cardiovascular risk prediction machine learning model and the cardiometabolic health data for the subject, values indicative of log hazard ratios for risk of the subject having a cardiovascular event at respective levels of cumulative LDL exposure, wherein the cardiovascular risk prediction machine learning model has been trained using training data comprising, for each of a plurality of participants enrolled in one or more prospective studies, at least one LDL along with a recorded age or date at which a first cardiovascular event occurred, optionally wherein the first cardiovascular event is a first episode of a fatal or non-fatal myocardial infarction (MI), fatal or non-fatal ischemic stroke, or coronary revascularization occurred; (b) estimating, using the values indicative of the log hazard ratios, (i) absolute instantaneous hazard rates of the subject having a cardiovascular event at the respective levels of cumulative LDL exposure, and (ii) cumulative lifetime risks of the subject having a cardiovascular event at the respective levels of cumulative LDL exposure; and (c) estimating, using the cumulative LDL exposure trajectory for the subject, multiple measures of risk to include: (i) absolute instantaneous hazard rates of the subject having a cardiovascular event at respective ones of multiple time intervals, and (ii) cumulative lifetime hazard and event rates of the subject having a cardiovascular event at the respective ones of the multiple time intervals; and pricing the insurance instrument for the subject based on the multiple measures of risk corresponding to the multiple time intervals.

Yet other aspects of the technology provide a computer-implemented method for pricing an insurance instrument for a subject, the method comprising: obtaining cardiometabolic health data for a subject comprising: one or more values for one or more clinical characteristics of the subject, and/or one or more values for one or more physical measurements of the subject, and/or one or more values for one or more biochemical measurements of the subject; generating a first feature vector representing the subject from the cardiometabolic health data for the subject, and/or a second feature vector representing the subject from the cardiometabolic health data for the subject; and performing one or both of: estimating an LDL level trajectory for the subject by processing the first feature vector using a LDL trajectory prediction machine learning model that has been trained to estimate an LDL level for a subject at each of multiple prior ages and each of multiple future ages using training data comprising for each of a plurality of participants, repeated longitudinal measures of LDL levels and values for the one or more clinical characteristics and/or the one or more physical measurements and/or the one or more biochemical measurements over at least 10, at least 20, at least 30, or at least 50 years of follow-up, wherein the LDL level trajectory for the subject comprises an estimated LDL level for the subject for each of multiple prior ages of the subject and multiple future ages of the subject; and estimating an SBP level trajectory for the subject by processing the second feature vector using an SBP trajectory prediction machine learning model that has been trained to estimate an SBP level for a subject at each of multiple prior ages and each of multiple future ages using training data comprising, for each of a plurality of participants, repeated longitudinal measures of SBP levels and values for the one or more clinical characteristics and/or the one or more physical measurements and/or the one or more biochemical measurements over at least 10, at least 20, at least 30, or at least 50 years of follow-up, wherein the SBP level trajectory for the subject comprises an estimated SBP level for the subject for each of multiple prior ages of the subject and multiple future ages of the subject; and pricing the insurance instrument for the subject based on the estimated LDL level trajectory and/or the estimated SBP level trajectory.

Additional aspects of the technology provide a computer-implemented method for pricing an insurance instrument for a subject, the method comprising: determining an expected risk of cardiovascular events for a subject in response to a therapeutic intervention associated with a reduction of LDL level and/or SBP level over an interval of follow up for the subject using a method comprising: (a) determining multiple measures of risk of cardiovascular events for the subject comprising absolute instantaneous hazard rates, cumulative hazard rates, and cumulative event rates of the subject having a cardiovascular event at respective ones of multiple time intervals, wherein the cumulative hazard rates and the cumulative event rates are not adjusted for the therapeutic intervention; (b) for each particular interval of follow-up for the subject from the subject's current age to an upper threshold age, (i) determining, using the multiple measures of risk, a predicted instantaneous hazard rate of the subject having a cardiovascular event at the particular interval of follow up; (ii) determining, using a benefit prediction machine learning model, a time-averaged instantaneous log hazard ratio for a one unit lower LDL or SBP corresponding to duration of treatment at the particular interval of follow-up for the subject, wherein the benefit prediction machine learning model has been trained using training data from randomized trials of LDL lowering therapies and/or randomized trials of SBP therapies, and/or Mendelian randomization studies evaluating genetic variants associated with lower LDL and/or lower SBP, said training data comprising for each of a plurality of participants in said trials, at least one LDL or SBP measurement along with a recorded age or date at which a first cardiovascular event occurred; (iii) determining an intervention-adjusted instantaneous hazard for the particular interval of follow-up by multiplying the instantaneous hazard rate of the subject determined at (a)(i) with the time-averaged instantaneous log hazard ratio determined at (b)(ii), thereby obtaining multiple intervention-adjusted instantaneous hazards for intervals of follow-up evaluated at (b); (c) determining, using the multiple intervention-adjusted instantaneous hazards, intervention-adjusted cumulative hazard rates and cumulative event rates of cardiovascular events for intervals of follow-up for the subject from the subject's current age to the upper threshold age; and (d) pricing the insurance instrument for the subject using the intervention-adjusted cumulative hazard rates and cumulative event rates of cardiovascular events obtained at (c).

Further aspects of the technology provide a computer-implemented method for pricing an insurance instrument for a subject, the method comprising: determining the value of one or more cardiometabolic health metrics associated with the subject using any one of the methods described herein; and pricing the insurance instrument for the subject based on the results of said determining.

Some aspects of the technology provide a system, comprising: at least one computer hardware processor; and at least one non-transitory computer readable storage medium storing processor-executable instructions that, when executed by at least one processor, cause at least one processor to perform any one of the methods described herein.

Other aspects of the technology provide at least one non-transitory computer readable storage medium storing processor-executable instructions that, when executed by at least one processor, cause at least one processor to perform any one of the methods described herein.

Some aspects of the technology provide a computer-implemented method, comprising: obtaining cardiometabolic health data for a subject comprising: one or more values for one or more clinical characteristics of the subject, and/or one or more values for one or more physical measurements of the subject, and/or one or more values for one or more biochemical measurements of the subject; encoding the cardiometabolic health data for the subject into a first feature vector; estimating an SBP level trajectory for the subject by processing the first feature vector using an SBP trajectory prediction ML model that has been trained to estimate an SBP level for a subject at each of multiple prior ages and each of multiple future ages using training data comprising, for each of a plurality of participants, repeated longitudinal measures of SBP levels and values for the one or more clinical characteristics and/or the one or more physical measurements and/or the one or more biochemical measurements over at least 10, at least 20, at least 30, or at least 50 years of follow-up, wherein the SBP level trajectory for the subject comprises an estimated SBP level for the subject for each of multiple prior ages of the subject and multiple future ages of the subject.

Yet other aspects of the technology provide a computer-implemented method of determining one or more measures of risk that a subject develops cardiovascular disease, the method comprising, for each of multiple time intervals: (a) estimating, using a trained cardiovascular risk prediction machine learning model and cardiometabolic health data for the subject including a cumulative LDL exposure trajectory for the subject, values indicative of log hazard ratios for risk of the subject having a cardiovascular event at respective levels of cumulative LDL exposure, wherein the cardiovascular risk prediction machine learning model has been trained using training data comprising, for each of a plurality of participants enrolled in one or more prospective studies, multiple LDL measurements along with a recorded age or date at which a first cardiovascular event occurred, optionally wherein the first cardiovascular event is a first episode of a fatal or non-fatal myocardial infarction (MI), fatal or non-fatal ischemic stroke, or coronary revascularization; and (b) estimating, using the values indicative of the log hazard ratios, (i) absolute instantaneous hazard rates of the subject having a cardiovascular event at the respective levels of cumulative LDL exposure, and (ii) cumulative lifetime risks of the subject having a cardiovascular event at the respective levels of cumulative LDL exposure; and (c) estimating, using the cumulative LDL exposure trajectory for the subject, the multiple measures of risk to include: (i) absolute instantaneous hazard rates of the subject having a cardiovascular event at respective ones of the multiple time intervals, and (ii) cumulative lifetime hazard and event rates of the subject having a cardiovascular event at the respective ones of the multiple time intervals.

Additional aspects of the technology provide a computer-implemented method of determining an expected proportional reduction and/or absolute reduction in risk of cardiovascular events for a subject in response to a particular therapeutic intervention sequence, the particular therapeutic sequence indicating magnitude, duration, and timing of one or more interventions associated with a reduction of LDL level and/or SBP level over an interval of follow up for the subject, the method comprising: determining multiple measures of risk of cardiovascular events for the subject comprising absolute instantaneous hazard rates, cumulative hazard rates, and cumulative event rates of the subject having a cardiovascular event at respective ones of multiple time intervals, wherein the cumulative hazard rates and the cumulative event rates are not adjusted for the particular therapeutic intervention sequence, and determining the expected proportional reduction and/or the absolute reduction in the risk of cardiovascular events for the subject in response to the particular therapeutic intervention sequence using a method that comprises: (a) for each particular interval of follow-up for the subject from the subject's current age to an upper threshold age, (i) determining, using the multiple measures of risk, a predicted instantaneous hazard rate of the subject having a cardiovascular event at the particular interval of follow up; (ii) determining, using a benefit prediction machine learning model, a time-averaged instantaneous log hazard ratio for a one unit lower LDL or SBP corresponding to duration of treatment at the particular interval of follow-up for the subject, wherein the benefit prediction machine learning model has been trained using training data from randomized trials of LDL lowering therapies and/or randomized trials of SBP therapies and Mendelian randomization studies evaluating genetic variants associated with lower LDL and/or lower SBP, said training data comprising for each of a plurality of participants in said trials, at least one LDL or SBP measurement along with a recorded age or date at which a first cardiovascular event occurred; (iii) determining an intervention-adjusted instantaneous hazard for the particular interval of follow-up by multiplying the instantaneous hazard rate of the subject determined at (a)(i) with the time-averaged instantaneous log hazard ratio determined at (a)(ii), thereby obtaining multiple intervention-adjusted instantaneous hazards for intervals of follow-up evaluated at (a); (b) determining, using the multiple intervention-adjusted instantaneous hazards, intervention-adjusted cumulative hazard rates and cumulative event rates of cardiovascular events for intervals of follow-up for the subject from the subject's current age to the upper threshold age; (c) determining predicted proportional reductions in the risk of experiencing a cardiovascular event as ratios of the intervention-adjusted cumulated hazard rates and the cumulative hazard rates that are not adjusted for the particular therapeutic intervention sequence; and (d) determining predicted absolute reductions in the risk of experience of experiencing a cardiovascular event as absolute differences between the intervention-adjusted cumulative event rates and the cumulative event rates that are not adjusted for the particular therapeutic intervention sequence.

Further aspects of the technology provide a system, comprising: at least one computer hardware processor; and at least one non-transitory computer readable storage medium storing processor-executable instructions that, when executed by at least one processor, cause at least one processor to perform any one of the methods described herein.

Some aspects of the technology provide at least one non-transitory computer readable storage medium storing processor-executable instructions that, when executed by at least one processor, cause at least one processor to perform any one of the methods described herein.

The preceding Summary is non-limiting.

### BRIEF DESCRIPTION OF DRAWINGS

Various aspects and embodiments will be described with reference to the following figures. It should be appreciated that the figures are not necessarily drawn to scale. Items appearing in multiple figures are indicated by the same or a similar reference number in all the figures in which they appear.

FIGS. 1A-D are block diagrams illustrating aspects of an example architecture of an AI-model based reinforcement learning system, in accordance with some embodiments of the technology described herein.

FIG. 1E is a block diagram illustrating an example system 150 configured for preventing development of disease, in accordance with some embodiments of the technology described herein.

FIG. 2A is an example of an illustrative process 200 for identifying an intervention for a subject in furtherance of preventing development of cardiovascular disease in the subject, in accordance with some embodiments of the technology described herein.

FIG. 2B is an example of an illustrative process for obtaining cardiometabolic health data for a subject, in accordance with some embodiments of the technology described herein.

FIG. 2C is an example of an illustrative process for determining, using the cardiometabolic health data, a first trained ML model, and multiple time intervals, measure(s) of risk that the subject develops cardiovascular disease, in accordance with some embodiments of the technology described herein.

FIG. 2D is an example of an illustrative process for determining, using the measure(s) of risk and at least one second trained ML model, benefit of administering to the subject therapeutic intervention(s) to reduce risk of cardiovascular disease, in accordance with some embodiments of the technology described herein.

FIG. 2E is an example of an illustrative process identifying, using determined benefit of administering the therapeutic intervention(s), at least one therapeutic intervention to recommend to be administered to the subject, in accordance with some embodiments of the technology described herein.

FIG. 3A shows an illustrative example of the subject characteristic and/or measurement data, in accordance with some embodiments of the technology described herein.

FIG. 3B shows an illustrative example of standardizing values part of the subject characteristic and/or measurement data, in accordance with some embodiments of the technology described herein.

FIG. 3C shows an illustrative example of positionally encoding the standardized values of the subject characteristic and/or measurement data, in accordance with some embodiments of the technology described herein.

FIG. 3D shows an illustrative example of an LDL trajectory predicted for a biological male subject, in accordance with some embodiments of the technology described herein.

FIG. 3E shows illustrative examples of LDL trajectories predicted for biological male and female subjects, in accordance with some embodiments of the technology described herein.

FIG. 3F shows an illustrative example of a cumulative LDL exposure trajectory representing cumulative exposure to LDL as a function of age, in accordance with some embodiments of the technology described herein.

FIG. 3G shows an illustrative example of an SBP trajectory predicted for a biological male subject, in accordance with some embodiments of the technology described herein.

FIG. 3H shows illustrative examples of SBP trajectories predicted for biological male and female subjects, in accordance with some embodiments of the technology described herein.

FIG. 3I shows an illustrative example of a cumulative SBP exposure trajectory representing cumulative exposure to SBP as a function of age, in accordance with some embodiments of the technology described herein.

FIG. 3J shows an illustrative example of Lp(a) trajectories predicted for men and women in various percentiles of the population, each Lp(a) trajectory indicating Lp(a) levels as a function of age, in accordance with some embodiments of the technology described herein.

FIG. 3K shows illustrative examples of HbA1c, weight, and waist circumference trajectories predicted for a subject, in accordance with some embodiments of the technology described herein.

FIG. 4A shows cumulative lifetime risk of major adverse cardiovascular events (MACE) at each age among persons randomized by nature to higher, average, or lower LDL levels.

FIG. 4B shows cumulative lifetime risk of MACE at each level of cumulative exposure to LDL among persons randomized by nature to higher, average, or lower LDL levels.

FIG. 4C-1 shows cumulative lifetime risk of MACE at each age among persons randomized by nature to higher, average, or lower LDL levels by inherited risk (family history of cardiovascular disease).

FIG. 4C-2 shows cumulative lifetime risk of MACE at each level of cumulative exposure to LDL among persons naturally randomized to higher, average, or lower LDL levels by inherited risk (family history of cardiovascular disease).

FIG. 4D-1 shows cumulative lifetime risk of MACE at each age among persons randomized by nature to higher, average, or lower LDL levels by exposure to endogenous arterial injury (Type 2 Diabetes).

FIG. 4D-2 shows cumulative lifetime risk of MACE at each level of cumulative exposure to LDL among persons randomized by nature to higher, average, or lower LDL levels by exposure to endogenous arterial injury (Type 2 Diabetes).

FIG. 4E-1 shows cumulative lifetime risk of MACE at each age among persons randomized by nature to higher, average, or lower LDL by exposure to exogenous arterial injury (tobacco smoking).

FIG. 4E-2 shows cumulative lifetime risk of MACE at each level of cumulative exposure to LDL among persons randomized by nature to higher, average, or lower LDL levels by exposure to exogenous arterial injury (tobacco smoking).

FIG. 4F illustrates a survival DNN trained at each level of cumulative exposure to LDL and combined piecemeal to train AI to learn to biology of how atherosclerosis develops and how the risk of acute atherosclerotic cardiovascular events at each level of cumulative LDL conditional on exposure to other causes of arterial wall injury that reduce the capacity of the artery to tolerate accumulated plaque burden, in accordance with some embodiments of the technology described herein.

FIG. 4G shows remaining cumulative lifetime risk of MACE by cumulative LDL exposure among persons with and without hypertension, in accordance with some embodiments of the technology described herein.

FIG. 4H illustrates calculation of personal plaque thresholds from LDL cumulative exposure thresholds at which MACE occur among persons with and without hypertension, in accordance with some embodiments of the technology described herein.

FIG. 4I-1 shows an example of cumulative lifetime risk of MACE and corresponding LDL cumulative exposure thresholds for men in a reference population, in accordance with some embodiments of the technology described herein.

FIG. 4I-2 shows an example of cumulative lifetime risk of MACE and corresponding LDL cumulative exposure thresholds for women in a reference population, in accordance with some embodiments of the technology described herein.

FIG. 4J is another diagram illustrating cumulative exposure to LDL as an estimate of accumulated plaque burden and corresponding lifetime risk of MACE, in accordance with some embodiments of the technology described herein.

FIG. 5A illustrates randomized training data for training at least one second ML model—a causal DNN for ODE model in this example—used for estimating benefits of interventions, in accordance with some embodiments of the technology described herein.

FIG. 5B illustrates estimates of the benefit of lowering LDL or SBP by magnitude and duration generated using a causal deep neural network for ordinary differential equations, in accordance with some embodiments of the technology described herein.

FIG. 5C illustrates that a causal DNN for ODE model accurately predicts benefit of lower LDL during every year of life in time-to-event Mendelian randomization (MR) studies, in accordance with some embodiments of the technology described herein.

FIG. 5D illustrates that a causal DNN for ODE model accurately predicts benefit of lower SBP during every year of life in time-to event MR studies, in accordance with some embodiments of the technology described herein.

FIG. 5E illustrates that a causal DNN for ODE model accurately predicts benefit of lower LDL and SBP during every year of life in time-to-event MR studies, in accordance with some embodiments of the technology described herein.

FIG. 5F illustrates that a causal DNN for ODE model accurately predicts benefit of lower LDL during every month of follow-up in randomized trials of LDL lowering therapies, in accordance with some embodiments of the technology described herein.

FIG. 5G illustrates that a causal DNN for ODE model accurately predicts benefit of lower SBP during every month of follow-up in randomized trials of SBP lowering therapies, in accordance with some embodiments of the technology described herein.

FIG. 5H illustrates that a causal DNN for ODE model accurately predicts benefit of lower LDL and SBP during every month of follow-up in randomized trials of combination LDL and SBP lowering therapies, in accordance with some embodiments of the technology described herein.

FIG. 6A illustrates an example benefit of lowering LDL by 35% beginning at the age of 40, in accordance with some embodiments of the technology described herein.

FIG. 6B illustrates an example benefit of lowering LDL by 35% beginning at the age of 40 as compared to lowering LDL by 35% beginning at the age of 65, in accordance with some embodiments of the technology described herein.

FIG. 6C illustrates example benefits of lowering LDL by 35% beginning at the ages of 40, 50, or 60 years, in accordance with some embodiments of the technology described herein.

FIG. 6D illustrates an example benefit of lowering LDL by 35% beginning at the age of 40 as compared to lowering LDL by 50% beginning at the age of 55, in accordance with some embodiments of the technology described herein.

FIGS. 7A-7C show screenshots of illustrative graphical user interfaces providing users with information about risk and/or recommended actions in furtherance of preventing development of cardiovascular disease, in accordance with some embodiments of the technology described herein.

FIG. 8 illustrates impact of non-compliance on cumulative lifetime risk of experiencing an ASCVD event, in accordance with some embodiments of the technology described herein.

FIG. 9A illustrates an illustrative example of updated subject characteristic and/or measurement data, in accordance with some embodiments of the technology described herein.

FIG. 9B illustrates effect on remaining lifetime risk of ASCVD events from age 55 to age 80 from lowering LDL by 35% beginning at the age of 40, both with and without quantifying the benefit of legacy effect, in accordance with some embodiments of the technology described herein.

FIG. 10A shows an illustrative example of a deep neural network.

FIG. 10B shows an illustrative example of a deep neural network with ordinary differential equations (ODE).

FIG. 11 is a block diagram of an illustrative computing system that may be used in implementing some embodiments of the technology described herein.

### DETAILED DESCRIPTION

Atherosclerotic cardiovascular disease and hypertension are by far the leading causes of morbidity, mortality, and healthcare costs around the world. Atherosclerosis develops over several decades before the accumulated plaque burden becomes large enough to increase the risk of having a heart attack or stroke; and systolic blood pressure (SBP) begins to rise linearly with age several decades before the development of hypertension. Thus, it is possible to predict who is developing these diseases and then intervene to reduce exposure to modifiable causes of disease, such as low density lipoprotein (LDL), lipoprotein(a) (Lp(a)), and SBP early in the disease process to slow the trajectory of atherosclerosis and rising SBP enough to largely reduce or even eliminate the lifetime risk of heart attack, stroke, and hypertension, for example. This should extend the average healthy lifespan, for example, by 25 years or more.

To achieve this goal, most people will likely require modest sustained reductions in LDL and SBP over several years, even decades, to slow the trajectory of atherosclerosis and rising SBP enough to largely reduce or eliminate their lifetime risk of developing heart attack, stroke, and hypertension. However, long-term compliance with the therapies needed to produce large enough reductions in LDL and SBP to accomplish this goal is likely to be very poor, thus substantially undermining the potential clinical and economic benefits that can be achieved through early intervention to prevent cardiometabolic disease.

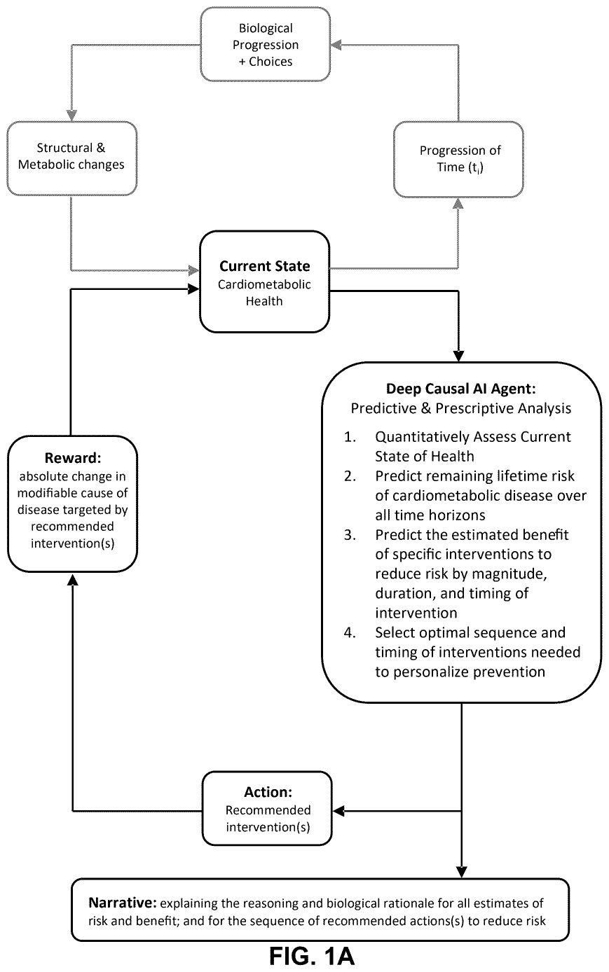

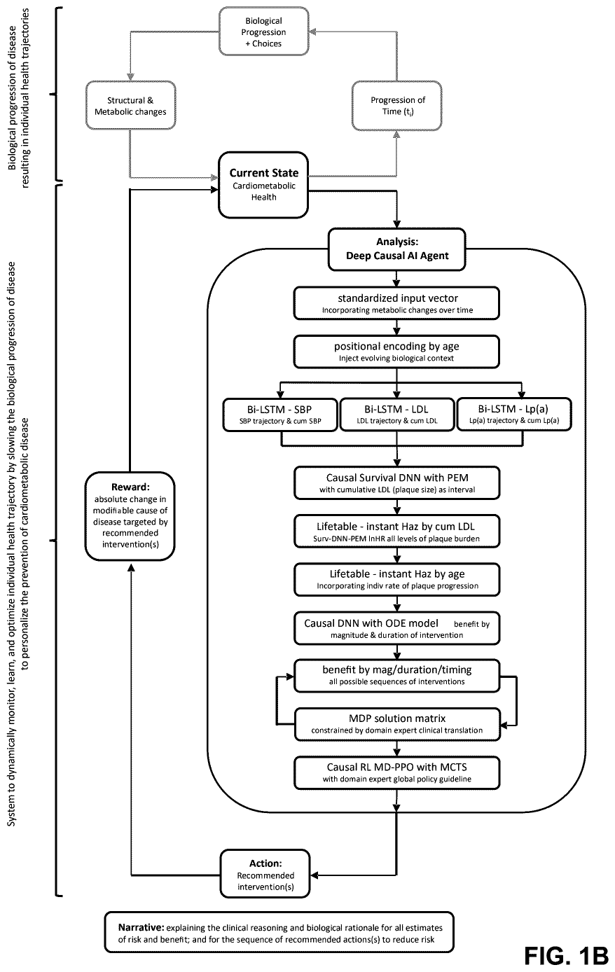

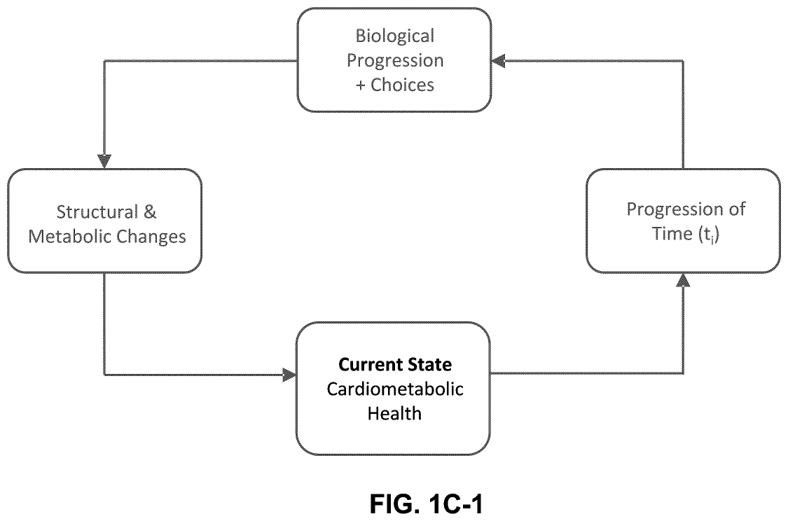

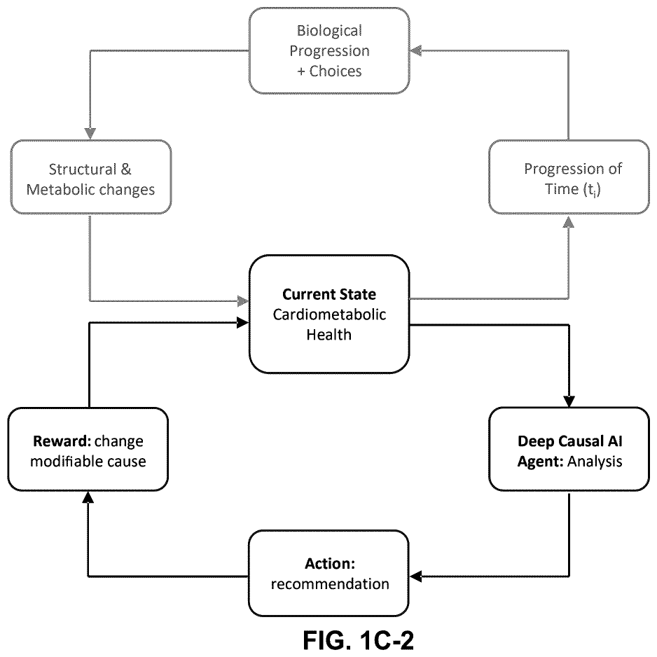

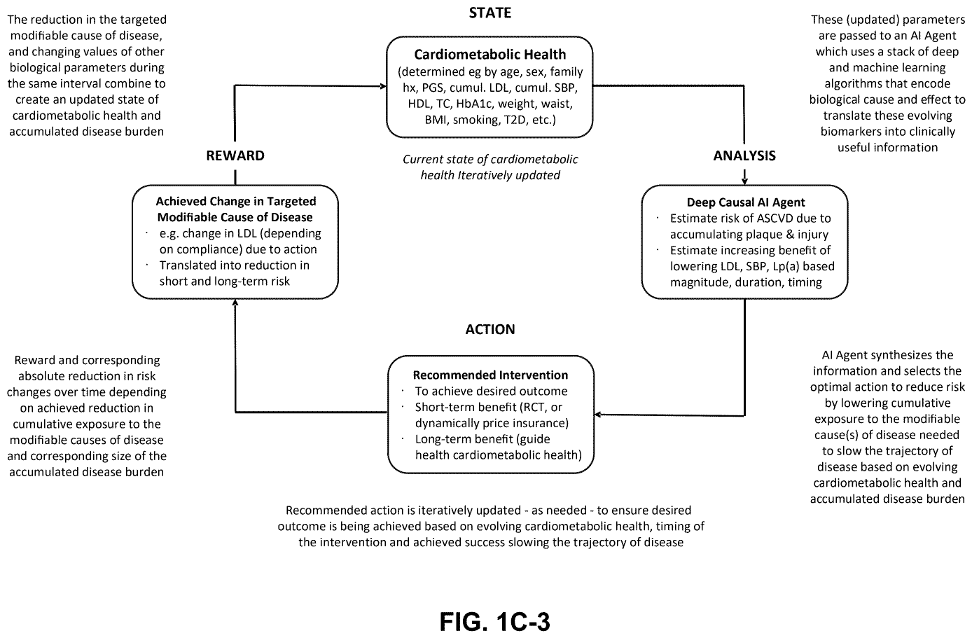

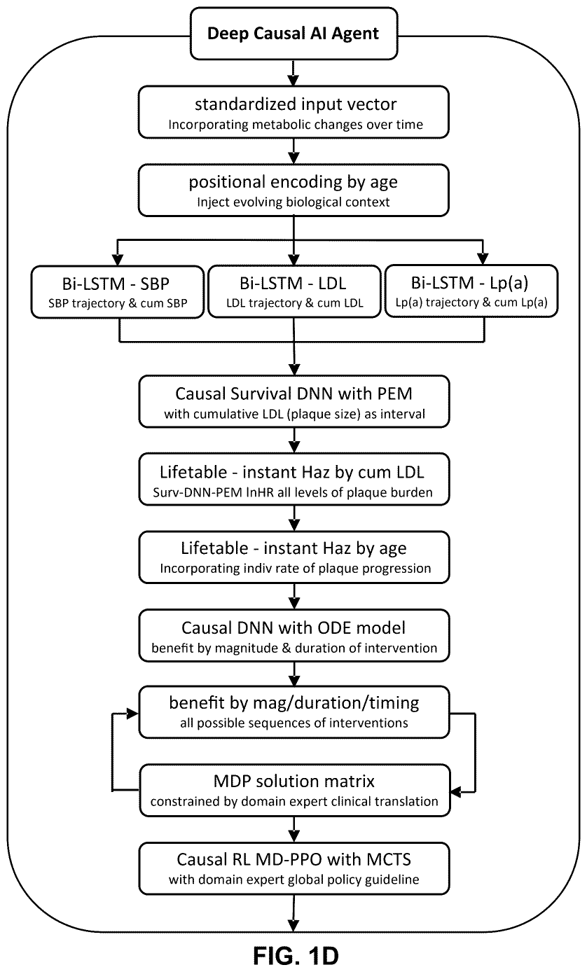

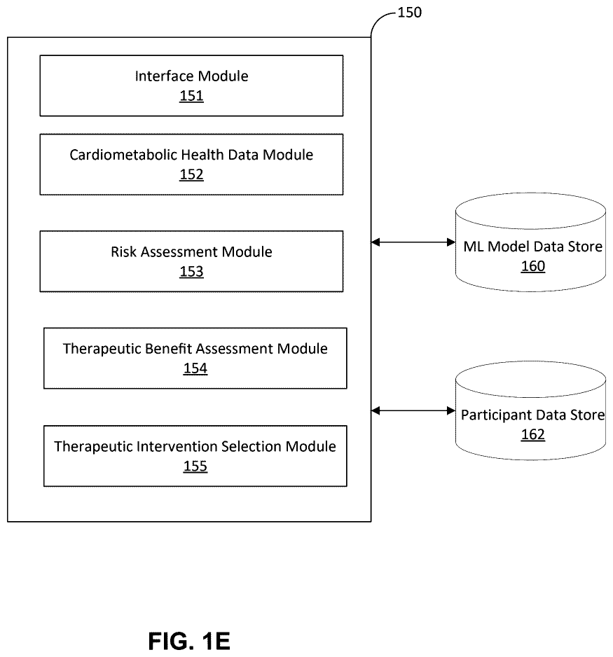

This problem can be solved, in some instances, by developing therapeutic interventions that can be administered yearly, for example, to ensure that long-term sustained reductions in LDL, Lp(a), and SBP are being achieved. Aspects of the technology described herein, including those referred to herein as Deep Causal AI (see, e.g., FIG. 1B), can be used to guide the use of these therapeutic interventions with quantitative precision, for example, by predicting each person's remaining lifetime risk of developing heart attack, stroke and hypertension; predicting the benefit that each person will receive from lowering LDL, Lp(a), and/or SBP beginning at any age and extending for any duration; and then using this information to prescribe and personalize therapeutic interventions and regimens to slow the trajectory of atherosclerosis and rising SBP enough to prevent heart attacks, stroke, and hypertension. Thus, aspects of the technology described herein provide a practical way to personalize the prevention of cardiometabolic (e.g., cardiovascular) disease.

Deep Causal AI, for example, combines AI, data analytics, and the current understanding of the biology of human diseases, with deep clinical insight to imbed randomized causal evidence into an ensemble of deep and machine learning algorithms that can predict risk and benefit. This advancement enables Deep Causal AI to both predict outcomes and prescribe actions to change those outcomes. As result, Deep Causal AI, as provided herein, can be used, for example, to enhance drug discovery and development, reimagine trial design and evidence generation, transform the delivery of therapeutic interventions, and/or define the future of Precision Cardiometabolic Health. Any one or more of the foregoing goals can be achieved using Deep Causal AI or other aspects of the technology described herein by: unlocking the unique value of therapies to extend the healthy lifespan by preventing common human diseases; generating evidence to create new markets to predict and prevent rather than diagnosis and treat disease; developing analytical tools and digital infrastructure for precision population health to deliver therapies to the right person, at the right time, and the right dose to prevent common human diseases; generating evidence to build an investment case and design innovative insurance and financial instruments to fund prevention and precision health at scale; predicting the trajectory of common human diseases over time by encoding biological cause and effect; quantifying the clinical benefit of reducing exposure to the modifiable causes of disease beginning at any age and extending for any duration; and/or using this information to prescribe specific actions that a person can take to effectively personalize the prevention of common diseases to extend a healthy lifespan.

The present technology, in some aspects, addresses the inability of existing computational systems to accurately predict personalized cardiovascular risk and identify optimal therapeutic interventions based on an individual's unique biological trajectory over time. Conventional risk assessment tools rely on static, population-based models that fail to account for the dynamic, cumulative nature of cardiovascular disease development, leading to suboptimal treatment decisions and poor patient outcomes. Moreover, conventional methods do not model the underlying biology of cardiovascular disease development.

The technology described herein, by contrast, provides a significant advancement in the state of the art of predicting cardiovascular risk of individual patients and identification of optimal therapeutic interventions for those patients. The technology described herein provides this advancement through a combination of novel components, and each such component is an advancement in its own right both in terms of the function that each such component performs (because such functions were previously not possible) and the manner in which it performs it (because the technology enabling the function is also new). The present disclosure describes these individual components and how they operate together to predict personalized cardiovascular risk and identify personalized therapeutic interventions.

One component of the technology described herein includes technology for the determination of novel biomarkers for a subject, which novel biomarkers are subsequently used in quantifying the subject's risk of experiencing major adverse cardiovascular events. The novel biomarkers include LDL, SBP, and/or Lp(a) trajectories for the subject that indicate estimated LDL, SBP, and/or Lp(a) levels for the subject at multiple ages (e.g., multiple prior and future ages, for example, from birth until 80 years old) and cumulative LDL, SBP, and/or Lp(a) exposure trajectories for the subject that indicate estimated cumulative LDL, SBP, and/or Lp(a) exposure levels for the subject at each of the multiple ages. These novel biomarkers are computationally derived using novel machine learning models and architectures developed by training on longitudinal data spanning years or decades. These biomarkers, together with conventional laboratory measurements and clinical data, create a comprehensive and precise cardiometabolic profile of an individual patient. The cardiometabolic profile of the patient may in turn be used to accurately predict the patient's cardiovascular risk and identify therapeutic interventions personalized to the patient's cardiovascular risk profile.

Another component of the technology described herein includes technology for using a patient's cardiometabolic profile to estimate the patient's cardiovascular risk. To this end, the disclosure describes a novel survival analysis enabled by new machine learning architectures. The novel survival analysis represents a fundamental departure from conventional approaches. Unlike conventional survival machine learning models, which use time (e.g., age in years) as intervals of follow-up, this disclosure introduces a family of new survival machine learning models that use cumulative exposure to LDL as intervals of follow-up. The family of new survival models includes various types of models, all using cumulative LDL exposure as interval of follow-up, including survival models with piecewise exponential modeling, deep neural networks, deep neural networks with piecewise exponential modeling, as well as other models, examples of which are provided herein. All these models are new and constitute improvements to conventional machine learning technology for survival analysis; these models did not exist prior to this disclosure and their use is not merely an application of existing models to new tasks—instead, the models described here constitute new machine learning architectures advancing the state of machine learning technology as part of survival analysis. As a result of the development of this new class of ML models, the technology described herein provides accurate estimates of an individual patient's time-varying cardiovascular risk, while accounting for how it changes over time based on the time-varying biomarker trajectories described above.

Another component of the technology described herein includes technology for predicting the expected benefit that a patient may receive in response to therapeutic interventions designed to reduce exposure to modifiable causes of cardiovascular disease (e.g., therapeutic interventions designed to lower LDL, SBP, or both LDL and SBP). One enabling part of this technology is a novel machine learning model architecture that provides, for the first time, the ability to quantify and estimate the benefit of lowering LDL and/or SBP for a particular patient (based on the patient's cardiovascular risk profile determined as above). This architecture involves a novel family of ML models, each of which estimates the benefit of administering a particular type of intervention to the patient. The novel family of ML models includes a so-called “causal” deep neural network (DNN) for ordinary differential equations (c-DNN-ODE) that has a unique architecture and is trained using a dataset comprising data from a combination of (1) randomized trials of LDL and SBP lowering therapies, respectively; and (2) Mendelian randomization studies with individual participant follow-up data evaluating genetic variants associated with lower LDL (apoB) or SBP, respectively. The disclosure sets forth how such models can be used to determine the expected proportional and/or absolute reduction in risk of cardiovascular events for the subject if the LDL and/or SBP were lowered (e.g., responsive to a particular therapeutic intervention sequence of one or more therapeutic interventions designed to target LDL and/or SBP levels). These benefits may be estimated for various types of interventions or sequences of interventions, for varying magnitude, duration, and timing of such interventions. These models are also new and provide important advantages relative to other, conventional types of ML models, as described herein. Once again, these “causal” models constitute new machine learning architectures and are not merely an application of existing models to new tasks.

Yet another component of the technology described herein includes technology for discovering the optimal timing, type, intensity, combination, and/or sequence of interventions useful for personalizing the prevention of cardiometabolic disease to a patient. This technology involves various innovations including novel machine learning (e.g., reinforcement learning (RL)) methods for efficiently exploring the solution space of intervention sequences and scoring each intervention sequence based in part on the degree to which the intervention sequence lowers a patient's risk of experience a major adverse cardiovascular event.

All these components, and others, are described herein and may be used individually or in any suitable combination. The component(s) may be used once or repeatedly, in the context of longitudinal patient monitoring and risk assessment. As the disclosure makes clear, the technology provided herein enables measurable improvements in medical practice, providing personalized treatment selection, objective insurance risk assessment, and clinical decision support with quantified benefits.

“Cardiometabolic disease” includes any metabolic, endocrine, or inflammatory disorder (herein used interchangeably with the term “disease”) or combination thereof, that increases the likelihood of adverse cardiovascular outcomes. Non-limiting examples of cardiometabolic diseases include cardiovascular diseases (e.g., atherosclerotic cardiovascular disease, stroke, arrhythmia, and heart failure), as well as hypertension, dyslipidemia, diabetes mellitus, metabolic syndrome, fatty liver disease, and obesity. Thus, cardiovascular disease represents a subset of cardiometabolic disease.

“Cardiovascular disease” includes disorders of the heart, blood vessels, or both, such as diseases of the coronary, peripheral, or cerebral vasculature, as well as structural, contractile, or electrical dysfunction of the myocardium. “Atherosclerotic cardiovascular disease” or “ASCVD” refers to cardiovascular conditions caused by the buildup of plaque (atherosclerosis) in the arterial walls. Non-limiting examples of ASCVD include coronary heart disease (also known as coronary artery disease, e.g., heart attack/myocardial infarction and angina), cerebrovascular disease (e.g., ischemic stroke and transient ischemic attack), peripheral artery disease, and aortic atherosclerotic disease (see, e.g., American Heart Association/heart.org).

“Cardiometabolic health” refers to the functional status of metabolic, endocrine, inflammatory, and/or cardiovascular systems (or any combination thereof), and exists on a continuous spectrum encompassing various states of health (e.g., optimal, intermediate, and/or poor health), as assessed by one or more cardiometabolic health data points.

“Cardiometabolic health data” refers to any qualitative or quantitative measurement indicative of cardiometabolic health, including, without limitation, one or more clinical characteristics of the subject (e.g., age, biological sex, family history of coronary heart disease, (CHD), family history of hypertension (HTN), family history of type 2 diabetes (T2D), polygenic scores, inherited predisposition or predispositions, history of tobacco use, etc.), one or more values for one or more physical measurements of the subject (systolic blood pressure (SBP), diastolic blood pressure (DBP), weight, waist circumference, height, body mass index (BMI), and waist-to-height ratio, etc.), and/or one or more values for one or biochemical measurements (e.g., lipid profile, low-density lipoprotein (LDL) level, high-density lipoprotein (HDL) level, total cholesterol level, triglyceride (TG) level, non-HDL cholesterol level, apolipoprotein (apoB) level, lipoprotein (a) (Lp(a)) level, glucose level, insulin level, glycated hemoglobin (HbA1c) level, liver function markers, inflammatory markers such as c-reactive protein (CRP) level)) of the subject.

“Major adverse cardiac event” or “MACE” refers to a composite clinical endpoint commonly used in the evaluation of cardiovascular risk, disease progression, and/or therapeutic efficacy. MACE generally encompasses serious cardiovascular outcomes that reflect clinically meaningful morbidity and mortality. As used herein, MACE refers to one of the following events: (i) non-fatal myocardial infarction (MI), (ii) fatal MI; (iii) non-fatal ischemic stroke; (iv) fatal ischemic stroke; and (v) coronary revascularization (percutaneous coronary revascularization with or without a stent, or CABG: coronary artery bypass grafting). The fatal MACE events (i.e., fatal MI and fatal ischemic stroke) can be referred to as cardiovascular death events.

Events constituting MACE can be adjudicated by predefined clinical criteria, optionally confirmed by electrocardiographic, biomarker, imaging, or procedural data. For example, myocardial infarction can be assessed by elevations in cardiac biomarkers (e.g., troponin), in some instances in combination with supporting clinical findings such as chest pain, electrocardiographic changes, or imaging evidence of new myocardial injury. As another example, stroke can include ischemic or hemorrhagic cerebrovascular events resulting in acute neurological deficit, optionally confirmed by imaging.

FIG. 1A and FIG. 1B are illustrative examples of a Deep Causal AI Model-Based Reinforcement Learning System, framing the longitudinal monitoring of cardiometabolic health and the provision of updated guidance on how to prevent cardiometabolic disease as a model-based reinforcement learning problem. This system learns, monitors, and optimizes individual health trajectory by slowing biological progression of disease to personalize the prevention of cardiometabolic diseases. This Deep Causal AI Agent includes a series of deep and machine learning algorithms that encode biological cause and effect and have been trained to learn the biology of how common cardiometabolic diseases develop over time. Aspects of this technology use a State-Analysis-Action-Reward design for reinforcement learning problems to guide precision health. As summarized in FIG. 1B, this predictive and prescriptive analysis quantitatively assesses the current state of health, predicts remaining lifetime risk of cardiometabolic disease over all time horizons, predicts the estimated benefit of specific interventions to reduce risk by magnitude, duration, and timing of intervention, and selects optimal sequences and timing of interventions needed to personalize prevention of cardiometabolic disease. This can be achieved, as follows:

### Current State

Initially, the current state of a person's cardiometabolic health is characterized. This state can be characterized by measuring the following biomarkers, for example: (1) clinical characteristics, including age, sex, family history, inherited predisposition (which can include polygenic risk), and tobacco history; (2) physical measurements, including systolic blood pressure (SBP), diastolic blood pressure (DBP), weight, waist circumference, height, and the derived measurements of body mass index (BMI), and waist-to-height ratio; and (3) biochemical measurements, including plasma low density lipoprotein (LDL) (apoB), lipoprotein(a) (Lp(a)), high density lipoprotein (HDL), triglycerides (TG), HbA1c, and others. The system is designed to be flexible and can include any bespoke set of features depending on local context and objectives.

A person's state of cardiometabolic health, however, is a dynamic process that evolves over time. FIGS. 1C-1, 1C-2, and 1C-3 schematize the evolution of cardiometabolic health over time. Additional LDL and other atherogenic apoB-containing lipoproteins become trapped within the arterial walls leading to a progressively enlarging atherosclerotic plaque burden and corresponding progressively increasing risk of having a clinical atherosclerotic cardiovascular event, including myocardial infarction (MI) and ischemic stroke. The SBP level rises over time, progressively injuring the arterial wall thus reducing the capacity of the artery to tolerate the accumulated plaque burden and increasing the risk of having an atherosclerotic cardiovascular event; while also increasing the risk of developing hypertension and other pressure related comorbidities. Weight or waist circumference may progressively increase over time due to chronic excess energy balance leading to a variable rate of rise in HbA1c, which progressively injures the arterial wall reducing its capacity to tolerate the accumulated plaque burden and thus increasing the risk of having an atherosclerotic cardiovascular event; while also increasing the risk of developing type 2 diabetes (T2D) and its related comorbidities. This understanding of the biology of how cardiometabolic diseases evolve over time, as provided herein, suggests a strategy to personalize prevention by slowing the progression of cardiometabolic disease.

First, LDL and other apoB-containing lipoproteins—including Lp(a)—are lowered to slow the progression of atherosclerosis enough to keep the size of the accumulated plaque burden below the threshold at which atherosclerotic cardiovascular events begin to occur. Next, an intervention to lower SBP is added when useful for preventing the accumulation of structural injury to the artery wall to maximize the capacity of the artery wall to tolerate the accumulated plaque burden and keep the risk of having an atherosclerotic cardiovascular event below the desired threshold; or when SBP exceeds 130 mmHg, for example, to prevent further rises in SBP and thus prevent the development of hypertension and its pressure related comorbidities. Similarly, a low-dose nutrient stimulated hormone (NuSH) or other intervention is added to prevent further weight gain caused by excess energy balance and thus prevent further rises in HbA1c, when useful for preventing the accumulation of structural injury to the artery wall caused by elevated glucose levels and thus maximizing the capacity of the artery wall to tolerate the accumulated plaque burden to keep the risk of having an atherosclerotic cardiovascular event below the desired threshold; or when HbA1c exceeds 5.7-6.0% (depending on age), for example, to prevent further rises in HbA1c and thus prevent the development of T2D and its related comorbidities. This integrated strategy is designed to personalize the prevention of myocardial infarction (MI), stroke, hypertension, and type 2 diabetes (T2D) by slowing the progression of how common cardiometabolic diseases develop. Provided herein is a system that learns biology to guide precision health so that, in some aspects, the reasoning and biological rationale for all outputs can be explained and objectively tested.

### Analysis by Deep Causal AI Agent

These measurements are passed into a Deep Causal AI Agent to perform a series of predictive and prescriptive analyses. The Deep Causal AI Agent includes a sequential stack of deep and machine learning algorithms designed to perform the following functions: (a) assess the current state of cardiometabolic health, including computing: (i) an estimate of the size of the atherosclerotic plaque burden that has accumulated, and the rate at which the plaque burden is progressing, (ii) the current SBP, with estimates of the rate at which SBP is rising over time, and how much structural injury caused by elevated SBP has accumulated, (iii) the current weight, waist circumference, and HbA1c level, with estimates of trends for average changes in weight, waist circumference and HbA1c over time; (b) predict the risk of developing cardiometabolic disease, including (i) the risk of developing an atherosclerotic cardiovascular event—including MI and stroke-based on the size of the accumulated plaque burden, and the combination of other exposures that impact the capacity of the artery to tolerate the accumulated plaque burden at any point in time (including, for example, the predicted 1-year, 2-year, 5-year, 10-year, 20-year, or remaining lifetime cumulative risk of developing an atherosclerotic cardiovascular event), (ii) the risk of developing hypertension based on the current level of SBP and the predicted rate of rise in SBP over time (including, for example, the predicted age at which hypertension is likely to occur (c) predict the expected benefit over any time period in response to actions (interventions) designed to reduce exposure to the modifiable causes of cardiometabolic disease, depending on the magnitude, duration, and timing of those interventions for all possible types, intensities, combinations, and sequences of possible interventions; and (d) discover the optimal timing, type, intensity, combination, and/or sequence of interventions useful personalizing the prevention of cardiometabolic disease by, for example, lowering LDL and other apoB-containing lipoproteins including Lp(a), SBP, weight (excess energy balance), and HbA1c by the amount needed by each person—when they need it—to prevent MI, stroke, and hypertension.

### Recommended Action

The output of the Deep Causal AI Agent is twofold: (1) a narrative explaining the reasoning and biological rationale for why a person is at risk of developing cardiometabolic disease, how they can minimize their risk, how much they will benefit from specific actions to minimize risk, and the recommended optimal sequence of actions useful preventing cardiometabolic disease; and (2) recommended immediate action (which can include observation) to personalize the prevention of cardiometabolic disease, with a description of the expected subsequent actions used and when they are likely to be used based on the person's predicted current cardiometabolic health trajectory.

### Reward

The realized reward of the recommended action is determined by the actual achieved absolute reduction in exposure to the modifiable cause of disease targeted by the recommended intervention, which determines the corresponding expected reduction in the risk of cardiometabolic disease over each subsequent time interval, including the expected reduction in the risk of cardiometabolic disease over the next 1 year, 2 years, 5 years, 10 years, 20 years, remaining lifetime, or any other interval of interest.

Because the magnitude of the reward, or expected clinical benefit, is quantified by the absolute reduction in the modifiable cause of disease achieved in response to the recommended intervention(s) or sequence of interventions, the realized reward or clinical benefit depends on compliance with the recommended intervention(s) and how well the intervention(s) are implemented. This objective quantification of the reward function as the achieved absolute reduction in the targeted modifiable cause of disease, rather than the expected absolute reduction in the modifiable cause of disease and corresponding expected proportional reduction in the risk of cardiometabolic disease that was used to inform selection of the recommended action: (a) motivates the need to develop therapies that ensure compliance with the recommended action to maximize the realized reward or clinical benefit; (b) provides an objective metric to evaluate the success of an intervention, and provides a metric to evaluate iterative attempts to improve implementation strategies; and (c) provides an objective metric to establish longitudinal reimbursement schedules for interventions designed to prevent disease based on the achieved absolute reductions in exposure to the modifiable causes of disease and the corresponding expected clinical benefit over any time horizon.

### Updated State

The updated state of cardiometabolic health can be determined by two dynamic processes: the achieved absolute reduction in the modifiable cause of disease targeted by the recommended intervention; and the absolute changes in other exposures not targeted by the recommended interventions(s) due to aging and the evolving biology of how common diseases develop during the same interval of longitudinal follow-up.

### Updated Analysis

The updated values of a person's clinical characteristics, physical biometrics, and biochemical measurements can then passed back into the Deep Causal AI Agent to provide: an updated assessment of their current state of cardiometabolic health; an updated prediction of the risk of cardiometabolic disease over all subsequent time intervals based on the updated state of cardiometabolic health and the reductions in the modifiable causes of disease achieved in response to prior interventions; an updated prediction of the expected benefit of interventions designed to reduce exposure to the modifiable causes of disease based on the updated state of cardiometabolic health and the reductions in the modifiable causes of disease achieved in response to prior interventions (including quantification of the legacy benefit from the achieved absolute reductions in the modifiable causes of disease in response to earlier interventions); and updated identification of the optimal timing, type, intensity, combination, and sequence of actions to prevent MI, stroke, and hypertension, for example.

### Recommended Next Action

The next output of the Deep Causal AI Agent includes an updated narrative explaining the reasoning and biological rationale for why a person is at risk, how their cardiometabolic health is evolving, how they can reduce their risk of cardiometabolic disease, how much they have benefited from previous actions, how much they would benefit from subsequent actions to prevent disease, and updated recommendations for the optimal sequence of actions useful preventing cardiometabolic disease; and an updated recommendation for the optimal next immediate action could include continuing the current intervention, intensifying the current intervention, and/or adding another intervention. This process can repeat iteratively at regular follow-up intervals to monitor cardiometabolic health, and adjust guidance about the optimal recommended actions and sequence of actions useful for preventing cardiometabolic disease (e.g., MI, stroke, hypertension, and/or T2D) based on the person's evolving cardiometabolic health and achieved reductions in the modifiable causes of disease in response to the recommended actions.

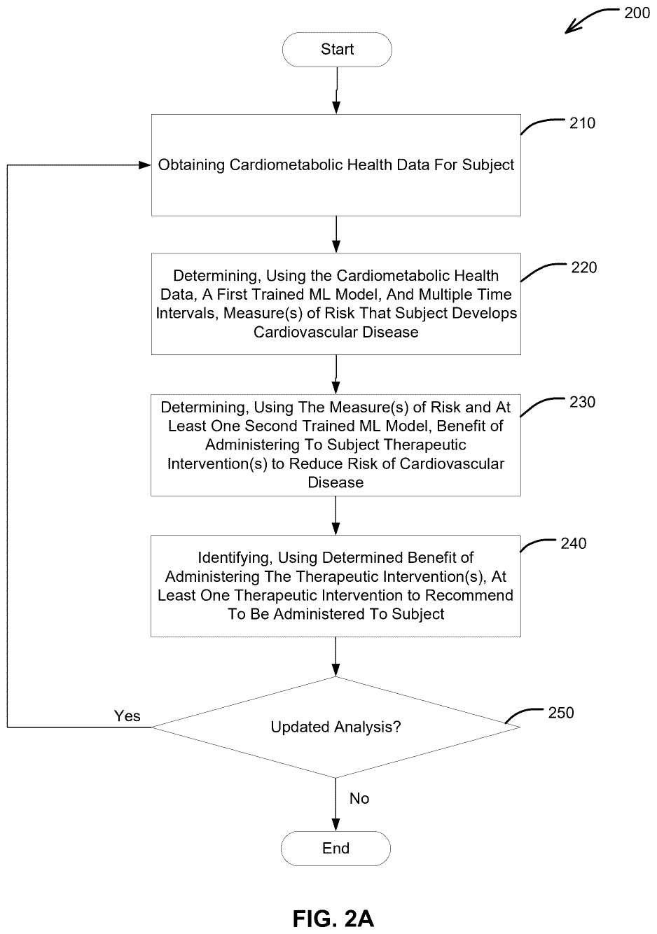

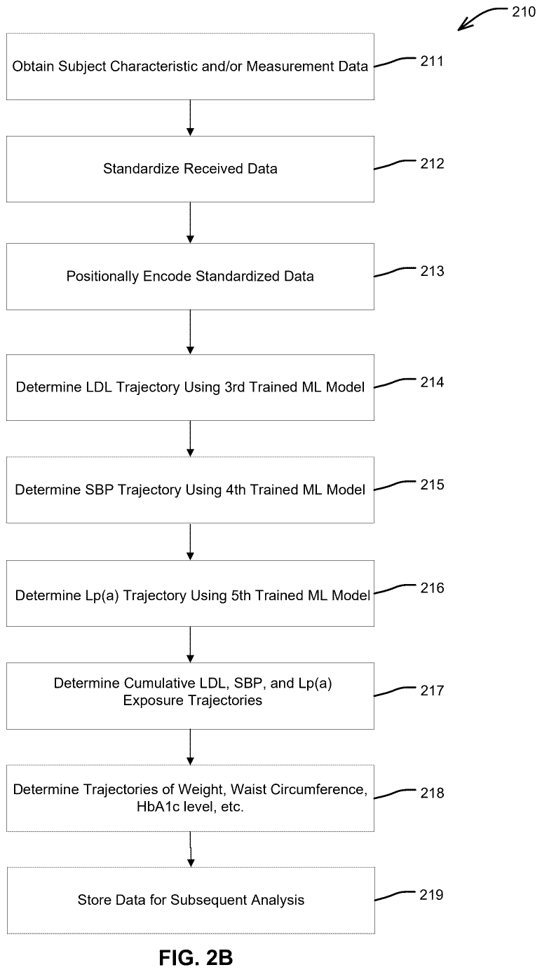

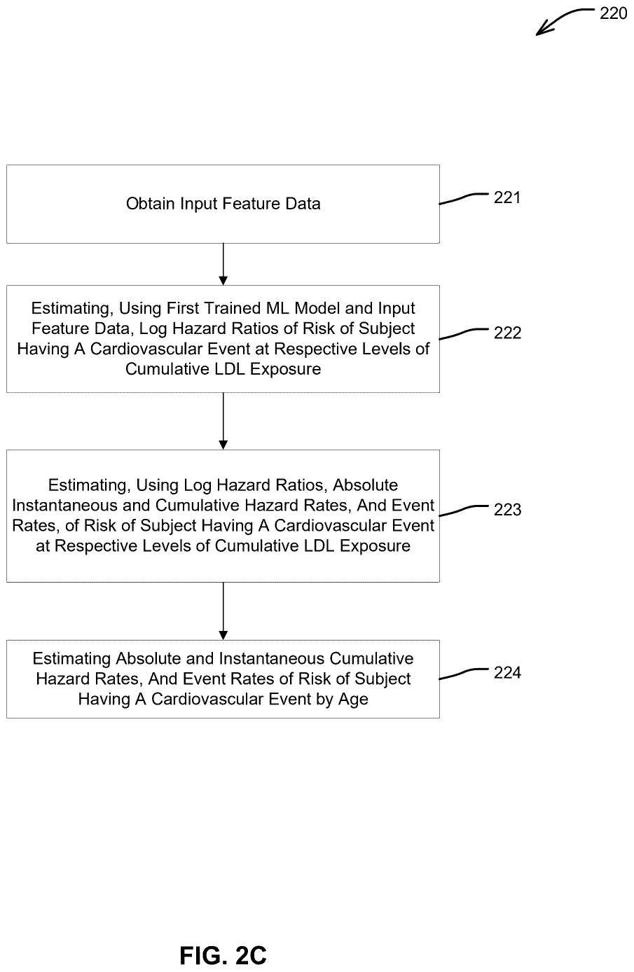

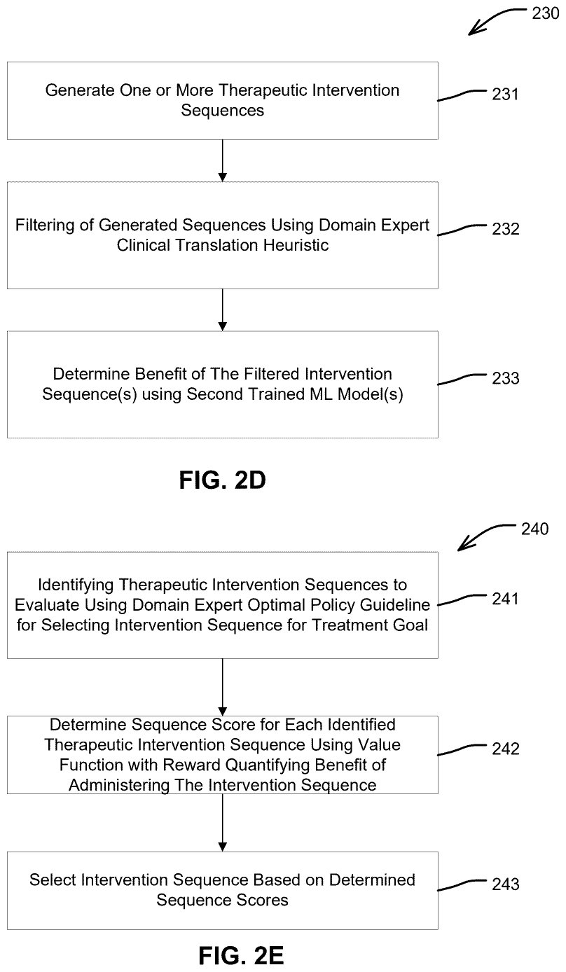

FIG. 1D is an illustration of an example embodiment of a “Deep Causal AI Agent” composed of a stack of deep and machine learning algorithms, which are described herein including with reference to FIGS. 2A-2E.

FIG. 1E is a block diagram illustrating an example system 150 configured for preventing development of disease, in accordance with some embodiments of the technology described herein. The system may include one more processors and at least one non-transitory computer-readable storage medium storing software comprising software modules. Each of the software modules may include processor-executable instructions, that when executed by the processor(s) of the system, cause the processor(s) of the system to perform the function or functions of the software module.

For example, in the illustrative embodiment of FIG. 1E, system 150 includes interface module 151, cardiometabolic health data module 152, risk assessment module 153, therapeutic benefit assessment module 154, and therapeutic intervention selection module 155. The system 150 may be configured to perform one or more (e.g., all) acts of process 200 described with reference to FIG. 2A.

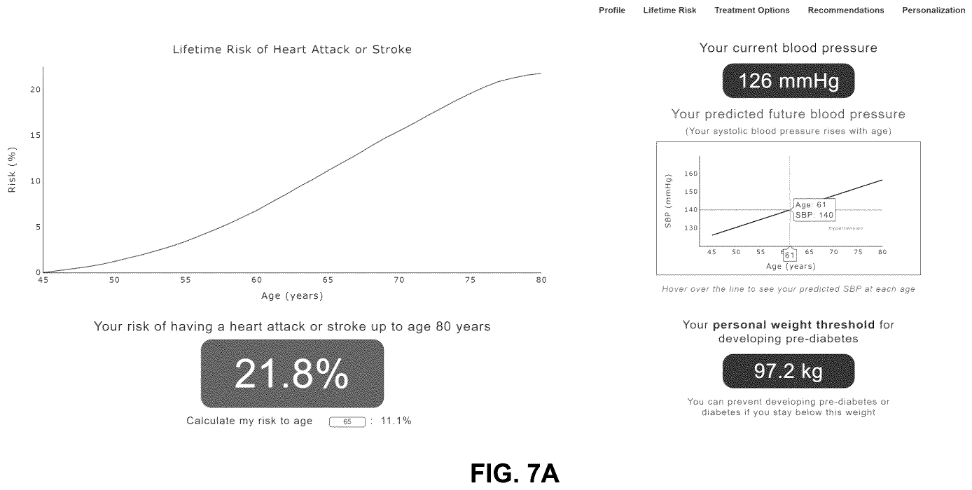

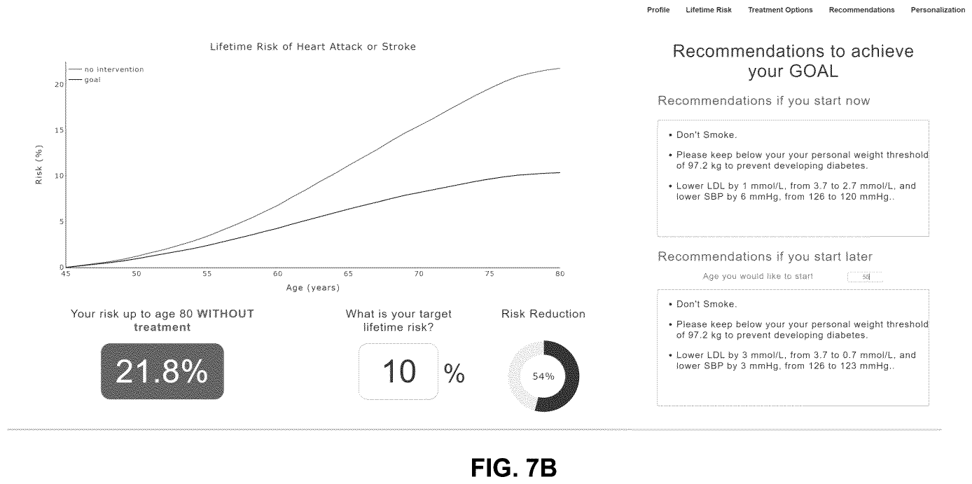

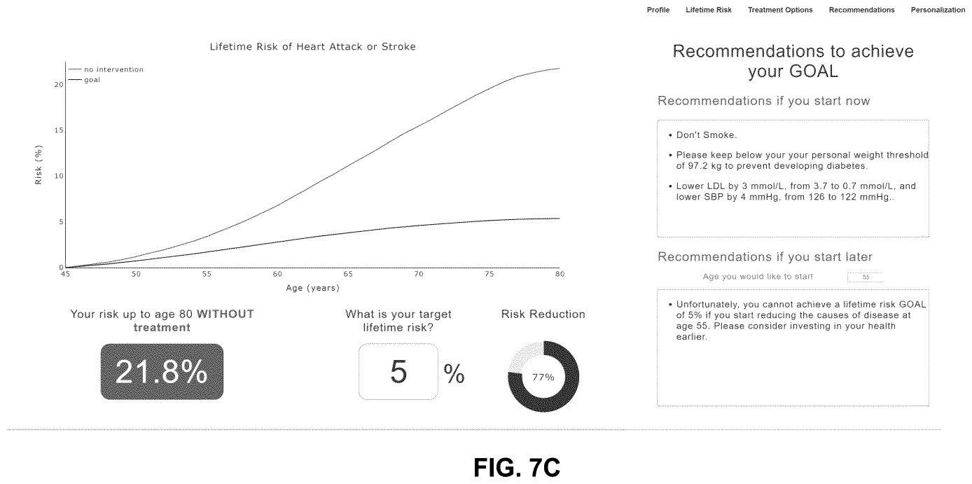

In some embodiments, the interface module 151 comprises processor-executable instructions, that when executed, provide functionality enabling interaction with the system 150. For example, interface module 151 may be configured one or more user interfaces (e.g., graphical user interfaces) that allow input of information about a subject (e.g., clinical characteristics, physical measurements, biochemical measurements) and their treatment goals (e.g., a specified target risk level in connection with cardiovascular disease, weight, etc.). The user interface(s) may additionally provide output to user(s) of the system including determined risks, benefits of treatment, recommended actions, and the like. Examples of such user interfaces are described herein including with reference to FIGS. 7A-7C.

In some embodiments, the cardiometabolic health data module 152 comprises processor-executable instructions that, when executed, cause the system 150 to perform obtaining cardiometabolic health data for the subject (e.g., as described with reference to act 210 of process 200).

In some embodiments, the risk assessment module 153 comprises processor-executable instructions that, when executed, cause the system 150 to perform determining, using at least some of the cardiometabolic health data and for each of multiple time intervals (e.g., each time interval of follow-up), one or more measures of risk that the subject develops disease to obtain multiple measures of risk corresponding to the multiple time intervals (e.g., as described with reference to act 220 of process 200).

In some embodiments, the therapeutic intervention benefit assessment module 154 comprises processor-executable instructions that, when executed, cause the system 150 to perform determining, using the multiple measures of risk that the subject develops the disease, benefit of administering to the subject one or more therapeutic interventions designed to reduce risk of the disease by targeting one or more modifiable causes of the disease (e.g., as described with reference to act 230 of process 200).

In some embodiments, the therapeutic intervention selection module comprises processor-executable instructions that, when executed, cause the system 150 to perform identifying, using the determined benefit of administering the one or more therapeutic interventions, at least one therapeutic intervention to recommend to be administered to the subject (e.g., as described with reference to act 240 of process 200).

As shown in FIG. 1E, system 150 also includes machine learning model datastore 160, which is configured to store one or more trained ML models to be used by one or more of the cardiometabolic health data module 151, the risk assessment module 152, the therapeutic intervention benefit assessment module 153, and/or the therapeutic intervention selection module 154. For example, the ML data store 160 may store parameters for a first ML model (e.g., a survival DNN-ODE model) to be used by the risk assessment module 152, parameters for at least one second ML model (e.g., one or multiple c-DNN-ODE models) to be used by the therapeutic intervention benefit assessment module 153, and/or parameters for one or more ML models (e.g., one or more bi-directional LSTM models) to be used by the cardiometabolic health data module 154 to estimate one or more trajectories of biomarker levels.

FIG. 2A is an example of an illustrative process 200 for identifying an intervention for a subject in furtherance of preventing development of cardiovascular disease in the subject, in accordance with some embodiments of the technology described herein.

As shown in FIG. 2A, process 200 comprises: (1) obtaining, at act 210, cardiometabolic health data for the subject, (2) determining, at act 220, using at least some (e.g., all) of the cardiometabolic health data, a first trained machine learning (ML) model (which may be referred to herein as a trained “cardiovascular risk prediction machine learning model” and may be, for example, a trained survival model), and for each of multiple time intervals (e.g., each time interval of follow-up, for example, in years), one or more measures of risk that the subject develops cardiovascular disease to obtain multiple measures of risk corresponding to the multiple time intervals; (3) determining, at act 230, using the multiple measures of risk that the subject develops cardiovascular disease and at least one second trained ML model (each of which may be referred to herein as a “benefit prediction machine learning model” and may be, for example, a trained deep neural network), benefit of administering to the subject one or more therapeutic interventions designed to reduce risk of cardiovascular disease by targeting one or more modifiable causes of the cardiovascular disease (e.g., LDL levels, SBP levels); and identifying, at act 240, using the determined benefit of administering the one or more therapeutic interventions, at least one therapeutic intervention (e.g., a particular sequence of interventions) to recommend to be administered to the subject. Examples of therapeutic interventions are described herein including in the Section titled “Therapeutic Interventions”. One or more of the identified therapeutic interventions may be recommended for administration to the subject. In some embodiments, the identified therapeutic intervention(s) may be administered to the subject.

In some embodiments, the subject may be monitored over time (e.g., longitudinally) and the analysis of acts 210-240 may be repeated. To this end, after act 240 is completed, process 200 may proceed to decision block 250, where it is determined that the analysis of acts 210-240 is to be repeated, for example, after some amount of time passes (e.g., a year, multiple months, a period of time between visits of the subject to their clinician, etc.). When it is determined that the analysis of acts 210-240 is not to be repeated, process 200 ends.

On the other hand, when it is determined that the analysis of acts 210-240 is to be repeated, process 200 returns to act 210, where updated cardiometabolic health data is obtained for the subject. Next process 200 involves, at act 220, determining, using at least some of the updated cardiometabolic health data, the first trained machine learning (ML) model and for each of multiple second time intervals, one or more updated measures of risk that the subject develops cardiovascular disease to obtain multiple updated measures of risk corresponding to the multiple second time intervals. Next process 200 involves, at act 230, determining, using the multiple updated measures of risk that the subject develops cardiovascular disease and the at least one second trained ML model, benefit of administering to the subject one or more second therapeutic interventions designed to reduce risk of cardiovascular disease by targeting one or more modifiable causes of the cardiovascular disease. Then, process 200 involves, at act 240, identifying, using the determined benefit of administering the one or more therapeutic interventions, at least one second therapeutic intervention to recommend to be administered to the subject. The second therapeutic intervention may involve continuing administering the initial therapeutic intervention sequence identified the first time through the process 200 (e.g., during the first time a subject's cardiometabolic data is analyzed) and/or adding a new therapeutic to the initial therapeutic intervention sequence. Other modifications to the initial therapeutic intervention sequence are also envisaged.

In this way, process 200 may be used to longitudinally monitor the health of the subject and to provide regularly updated guidance about how to personalize, to the subject, the prevention of development of cardiometabolic disease in the subject.

It should be appreciated that the process 200 is illustrative and that there are variations thereof. For example, in some embodiments, the process 200 may be performed for other diseases not just cardiovascular diseases, for example type 2 diabetes. Thus, for example, process 200 may be performed to personalize the prevention of cardiometabolic disease (e.g., cardiovascular disease, diabetes, etc.) in a subject.

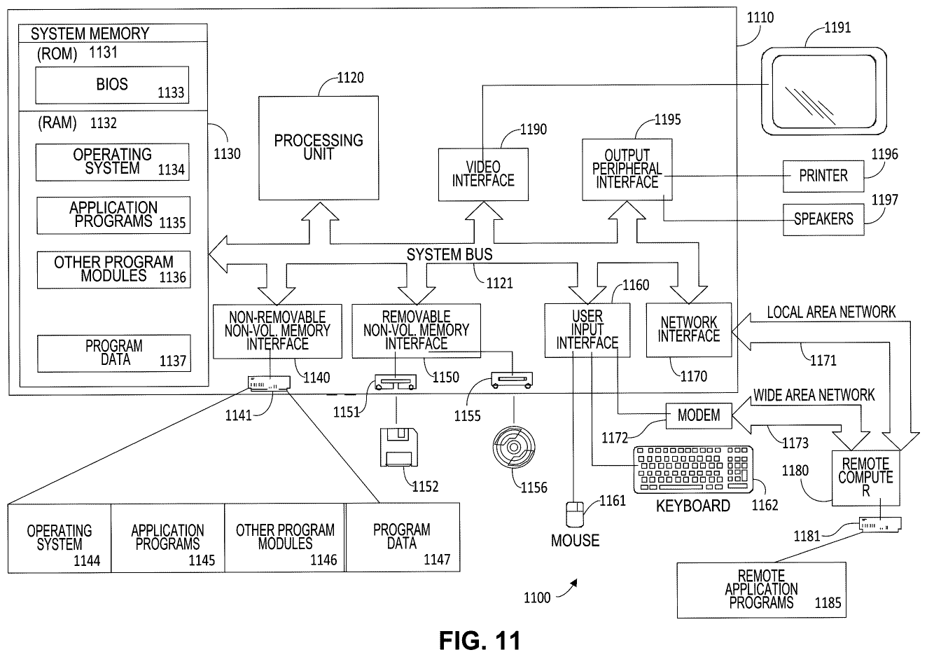

Process 200 may be performed by any suitable computing system or systems, which may be a single computing system and/or multiple computing systems (e.g., cloud computing system, distributed computing system, one or more computing devices such as laptops, desktops, smartphones, etc.), as aspects of the technology described herein are not limited in this respect. For example, process 200 may be performed using the system 150 described with reference to FIG. 1E and/or any computing device(s) described with reference to FIG. 11.

It should also be appreciated that, in some embodiments, one or more acts of process 200 may be performed by a computing system or systems while one or more other acts of process 200 may be performed, at least in part, manually. For example, in embodiments, where obtaining cardiometabolic data (at 210) involves actually making physical measurements of the subject (e.g., measuring blood pressure, weight, waist circumference, etc.) or biochemical measurements of the subject (e.g., doing a blood test with a lipid panel to determine, for example, the subject's LDL level), such actions may be performed in part by a clinician and/or laboratory technician. As another example, in embodiments in which process 200 includes further acts such as administering a therapeutic intervention or a sequence of therapeutic interventions, such acts may be performed in part by a clinician (administering a treatment to the subject) and/or the subject (e.g., a patient self-administering the treatment).

### Obtaining Cardiometabolic Health Data for Subject

As shown in FIG. 2A, process 200 includes obtaining cardiometabolic health data for the subject at act 210. In some embodiments, this may be performed in accordance with the illustrative process shown in FIG. 2B. As shown in FIG. 2B, act 210 may involve: (1) obtaining, at act 211, subject characteristic and/or measurement data; (2) standardizing, at act 212, the received subject characteristic and/or measurement data; and (3) positionally encoding, at act 213, the data standardized at act 212.

Next, in some embodiments, the standardized data obtained at 212 together with the positionally encoded data obtained at act 213 may be processed, at act 214, to determine the subject's LDL trajectory. The subject's LDL trajectory may indicate estimated LDL levels for the subject including an estimated LDL level for each of multiple prior ages of the subject (e.g., to birth) and/or multiple future ages of the subject (e.g., up until some threshold age, for example, 80, 85, or 90). The processing to determine the subject's LDL trajectory may be performed using a third trained ML model (e.g., a deep neural network such as a recurrent neural network, for example, a unidirectional or a bi-directional long short-term memory (LSTM) neural network, a neural network having a gated recurrent unit (GRU) architecture or a bi-directional GRU architecture).

Additionally, in some embodiments, the standardized data obtained at 212 together with the positionally encoded data obtained at act 213 may be processed, at act 215, to determine the subject's SBP trajectory. The subject's SBP trajectory may indicate an estimated SBP level for the subject for each of multiple prior ages of the subject (e.g., to age 20) and/or multiple future ages of the subject (e.g., up until some threshold age, for example, 80, 85, or 90). The processing to determine the SBP trajectory processing may be performed using a fourth trained ML model (e.g., a deep neural network such as a recurrent neural network, for example, a unidirectional or a bi-directional LSTM neural network, a neural network having a gated recurrent unit (GRU) architecture or a bi-directional GRU architecture) different from the third trained ML model.

Additionally, in some embodiments, the standardized data obtained at act 212 together with the positionally encoded data obtained at act 213 may be processed, at act 216, to determine the subject's Lp(a) trajectory. The subject's Lp(a) trajectory may indicate an estimated Lp(a) level for the subject for each of multiple prior ages of the subject (e.g., to birth) and/or multiple future ages of the subject (e.g., up until some threshold age, for example, 80, 85, or 90). The processing to determine the Lp(a) trajectory processing may be performed using a fifth trained ML model (e.g., a deep neural network such as a recurrent neural network, for example, a unidirectional or a bi-directional LSTM neural network, a neural network having a gated recurrent unit (GRU) architecture or a bi-directional GRU architecture) different from the third and fourth trained ML models.

In turn, at act 217, the LDL, SBP, and LP(a) trajectories (determined at acts 214-216) may be used to determine the cumulative LDL exposure trajectory (indicating cumulative exposure to LDL at each of multiple ages (e.g., each age from birth to 80)), the cumulative SBP exposure trajectory (indicating cumulative exposure to SBP at each of multiple ages (e.g., each age from 20 to 80)), and/or the cumulative Lp(a) trajectory (indicating cumulative exposure to Lp(a) at each of multiple ages (e.g., each age from birth to 80)).

Next, at act 218, one or more additional feature trajectories may be determined for the subject. For example, a weight trajectory, a waist-circumference trajectory, and/or an HbA1c trajectory may be determined for the subject (e.g., from the “average” trajectories in the reference population for a person of the same sex and age, as described herein).

Next, at act 219, the various cardiometabolic data obtained and/or determined at acts 211-218 is used for further processing. For example, the data may be stored, in memory (volatile or non-volatile), using any suitable data structure or data structures, and may be subsequently accessed when performing further processing (e.g., as part of act 220 of process 200). As another example, the data may be passed onto other software modules for processing (e.g., to software modules configured to implement the function of act 220 of process 200, for example, risk assessment module 153).

Aspects of some of the acts 211-219 are now further described.

As described herein, act 211 may involve obtaining subject characteristic and/or measurement data for the subject.

In some embodiments, obtaining the cardiometabolic health data for the subject comprises obtaining subject characteristic and/or measurement data comprising: one or more values for one or more clinical characteristics of the subject, one or more values for one or more physical measurements of the subject, and/or one or more values for one or biochemical measurements of the subject. In some cases, multiple values of each type of data (i.e., clinical characteristics, and physical measurements, and biochemical measurements) of the subject are obtained.

In some embodiments, the clinical characteristics of the subject may include demographic characteristics (e.g. age, biological sex, ethnicity, marital status, geographic location, etc.), genetic characteristics (e.g., presence of gene variants associated with cardiovascular disease, polygenic scores for coronary heart disease, etc.), family history characteristics (e.g., history of coronary heart disease), comorbidities (e.g., hypertension), and/or risk factors (e.g., history of tobacco use). For example, the clinical characteristics of the subject may include one or more values for one or more (e.g., one, some, or all) clinical characteristics of the subject selected from the group consisting of age, biological sex, family history of atherosclerotic cardiovascular disease (ASCVD) including coronary heart disease (CHD), family history of hypertension (HTN), family history of type 2 diabetes (T2D), one or more polygenic scores (e.g., polygenic score for ASCVD, polygenic score for CHD, polygenic score for HTN, polygenic score for T2D, polygenic score for body mass index (BMI), etc.), inherited predisposition or predispositions, and history of tobacco use. In some embodiments, act 211 may involve accessing clinical characteristic values (e.g., from a database or other data store, through a user interface such as a graphical user interface, from electronic health records (EHR), etc.) that were previously obtained. In other embodiments, act 211 may involve taking the clinical characteristic values in the first instance (e.g., by a clinician taking the subject's history, the subject providing such information, etc.).

As discussed above, the clinical characteristics of the subject may, in some embodiments, include a polygenic score for ASCVD. Polygenic scores (ASCVD-PGS) for ASCVD (e.g., heart attack or stroke) are crude instruments that combine a large number (n) of: (a) genetic variants associated with ASCVD; or (b) a very large number of unselected genetic variants (typically 1-10 million variants). When using a very large number of unselected variants, the ASCVD-PGS may be constructed as follows: (1) the association between each genetic variant and the outcome of interest (heart attack & stroke) is measured in a reference population; (2) the effect size of each allele (log hazard ratio or log odds ratio) is recorded for each genotype is relative to the most common genotype for that allele; (3) an Effect Size Table is constructed for each of up to 10 million variants—where rows indicate each genetic variant and the columns are the possible genotypes for that variant, and each cell in the table contains the effect size (log hazard ratio or log odds ratio) for each genotype relative to the most common genotype for that variant (note that an assigned reference log HR or log OR of 0 is equivalent to a hazard ratio or odds ratio of 1); (4) for each subject, the genotype at each variant included in the ASCVD-PGS is determined; and (5) the corresponding log HR or log OR for that genotype from the Effect Size Table is then summed for each variant included in the score for each subject.

This process creates an ASCVD-PGS (a specific polygenic score for the composite outcome of major adverse cardiovascular events (MACE), such as fatal or non-fatal MI, fatal or non-fatal ischemic stroke, and coronary revascularization) with a normal distribution by design and construction. Note that a polygenic score must correspond to the outcome being predicted to have any meaning. Alternatively, instead of or in addition to an ASCVD-PGS, one can include a variety of polygenic scores estimating the inherited risk of MI, CHD, stroke, LDL, SBP, T2D, BMI, etc. The ASCVD-PGS is then standardized to have a mean of 0 and a standard deviation of 1, and the ASCVD-PGS for each person may be adjusted to this scale. (As described below, the ASCVD-PGS may be further standardized, e.g., using min-max scaling as part of act 212).

In some implementations, an ASCVD-PGS for the subject may be calculated as part of act 211 of process 200. For example, the steps (1)-(3) may be performed prior to the start of process 200, while steps (4)-(5) may be performed for the subject as part of process 200. In other implementations, an ASCVD for the subject may have been determined prior to performance of process 200 and may be accessed (rather than calculated) as part of act 211 of process 200. Similarly, one or more other polygenic scores (e.g., for coronary heart disease, hypertension, etc.), when included, may be calculated as part of process 200 or calculated prior to the start of process 200 and instead accessed during process 200.

In some embodiments, the physical measurements of the subject may include physical measurements of quantities that are risk factors for cardiometabolic (e.g., cardiovascular) disease, and/or physiological measurements selected from blood pressure measurements (e.g., systolic and diastolic blood pressure) and measurements indicative of adiposity (e.g., weight, waist circumference, body mass index, etc.). For example, the subject characteristic and/or measurement data comprises one or more values for one or more (e.g., one, some, or all) physical measurements of the subject selected from the group consisting of systolic blood pressure (SBP), diastolic blood pressure (DBP), weight, waist circumference, height, body mass index (BMI), and waist-to-height ratio. In some embodiments, act 211 may involve accessing physical measurement values (e.g., from a database or other data store, through a user interface such as a graphical user interface, from electronic health records, etc.) that were previously obtained. In other embodiments, act 211 may involve taking the physical measurements (e.g., by a clinician).

In some embodiments, the biochemical measurements of the subject may include biochemical measurements of quantities that are risk factors for cardiometabolic (e.g., cardiovascular) disease and/or biochemical measurements selected from measurements of one or more biochemical markers, optionally a protein, lipid or lipoprotein, in a blood, serum or plasma sample from the subject. For example, the subject characteristic and/or measurement data comprises one or more values for one or more (e.g., one, some, or all) biochemical measurements of the subject selected from the group consisting of low-density lipoprotein (LDL) level, high-density lipoprotein (HDL) level, total cholesterol level, triglyceride (TG) level, non-HDL cholesterol level, apolipoprotein (apoB) level, lipoprotein (a) (Lp(a)) level, hemoglobin A1c (HbA1c) level, and c-reactive protein (CRP) level. In some embodiments, act 211 may involve accessing biochemical measurement values (e.g., from a database or other data store, through a user interface such as a graphical user interface, from electronic health records, etc.) that were previously obtained. In other embodiments, act 211 may involve obtaining the biochemical measurements (e.g., by obtaining a biological sample of the subject; and determining, by processing the biological sample, the one or more values for the one or more biochemical measurements of the subject; or determining, by processing a biological sample previously obtained from the subject the one or more values for the one or more biochemical measurements of the subject). By way of example and not limitation, HbA1c may be derived from whole blood samples (e.g., by determining as a percentage of total hemoglobin within red blood cells that are glycated), glucose may be measured from a plasma sample, lipids (e.g., LDL and apolipoproteins including apoB and Lp(a)) but also may be measured from a serum sample, but can also be measured in heparinized plasma depending on the laboratory reference being used.

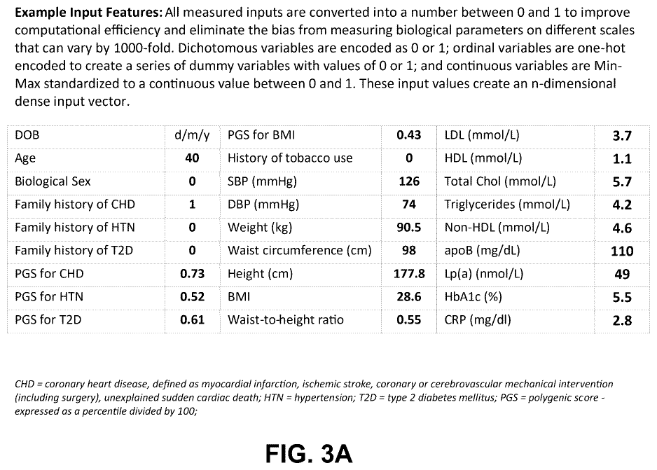

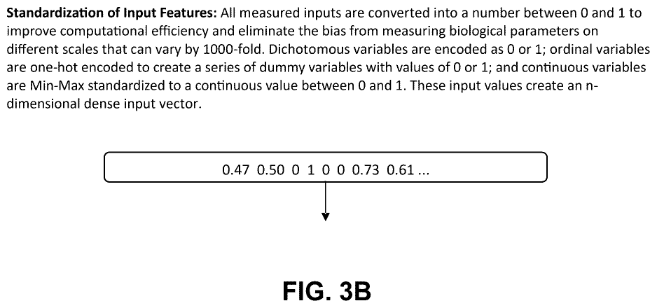

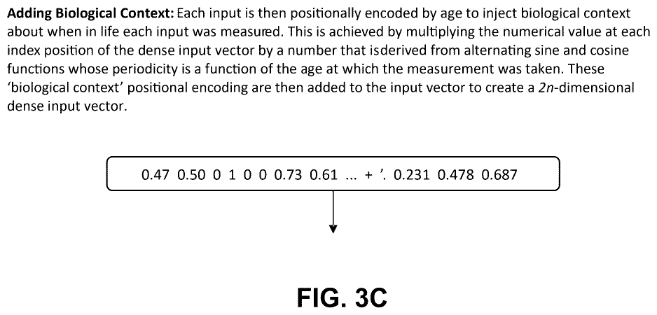

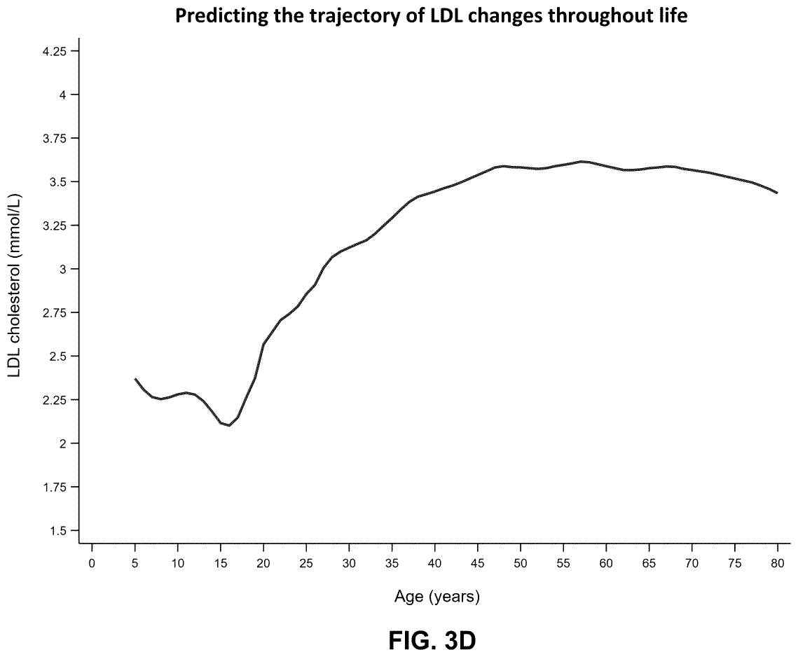

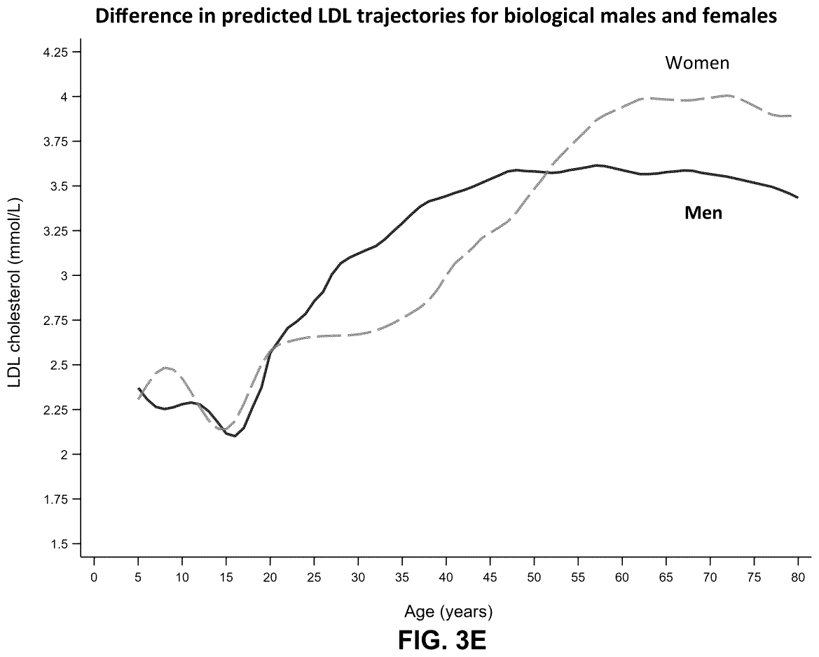

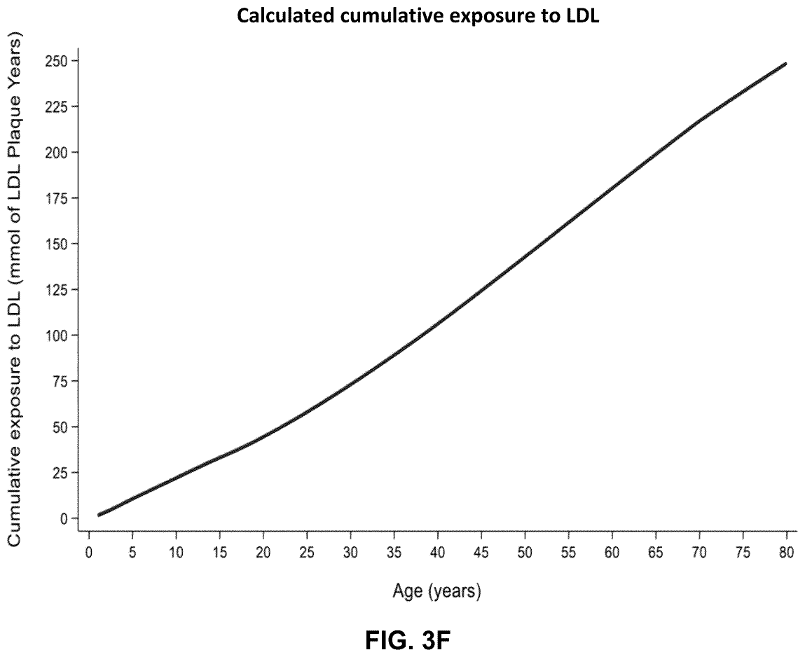

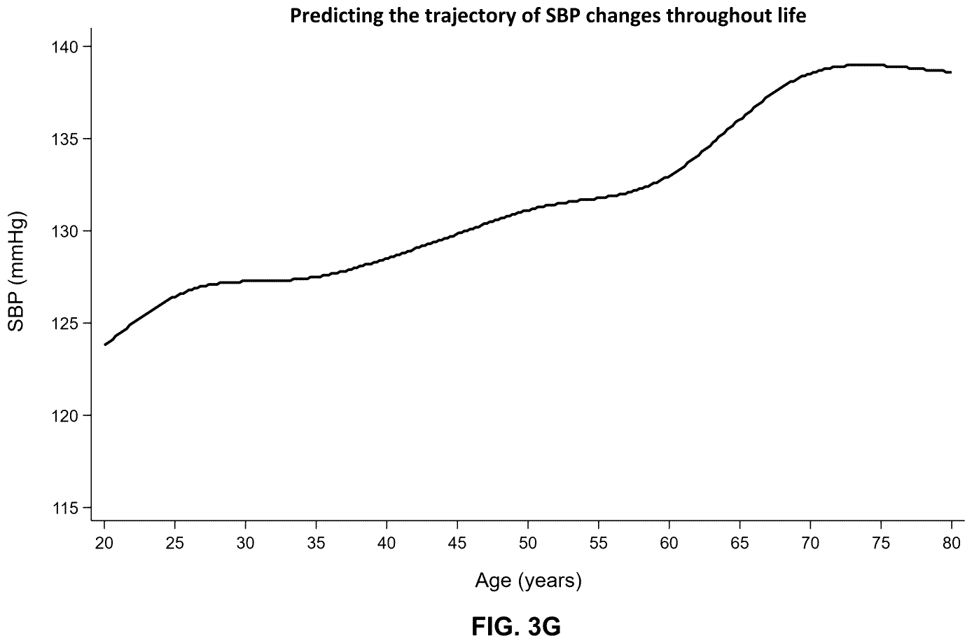

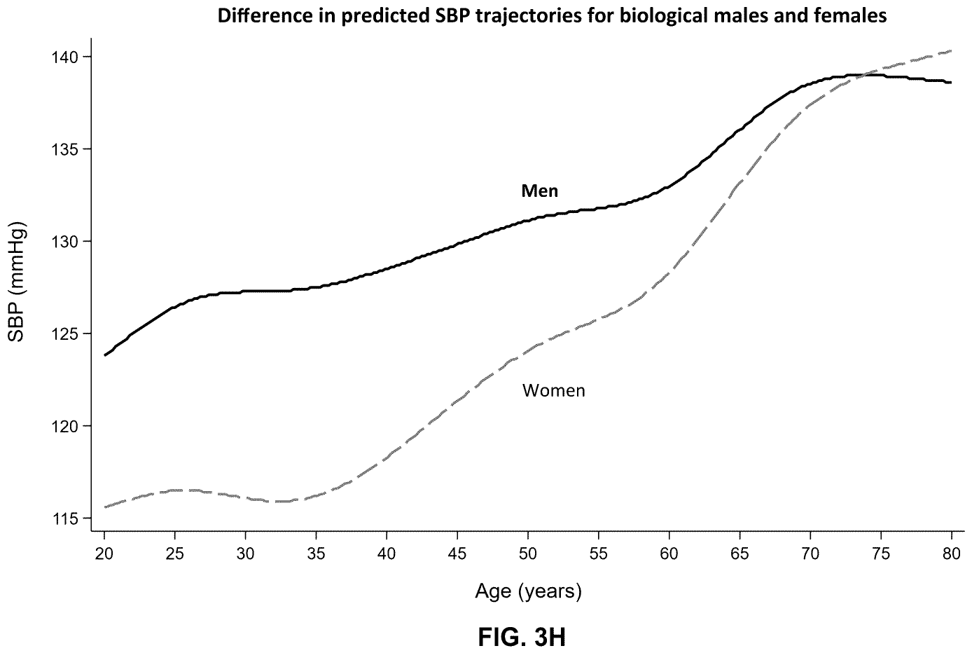

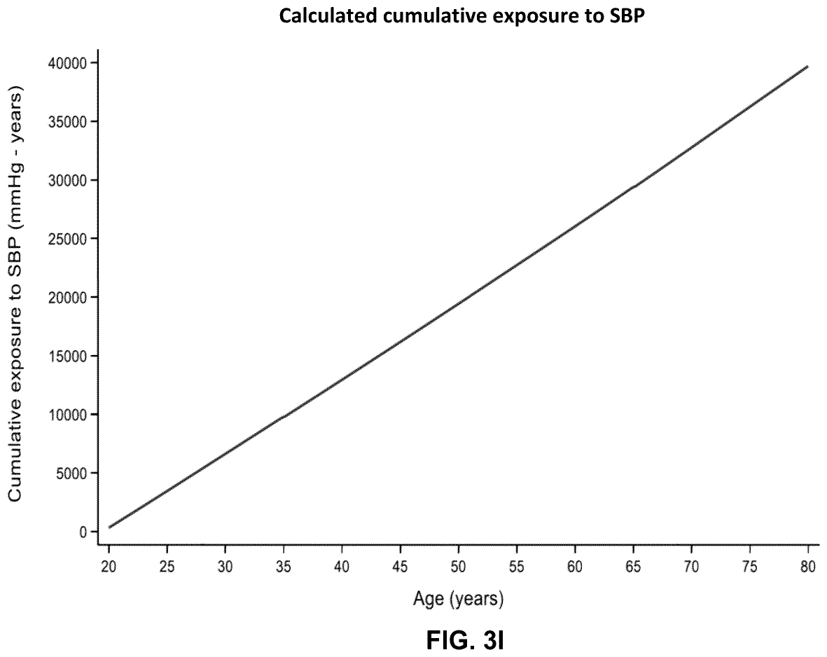

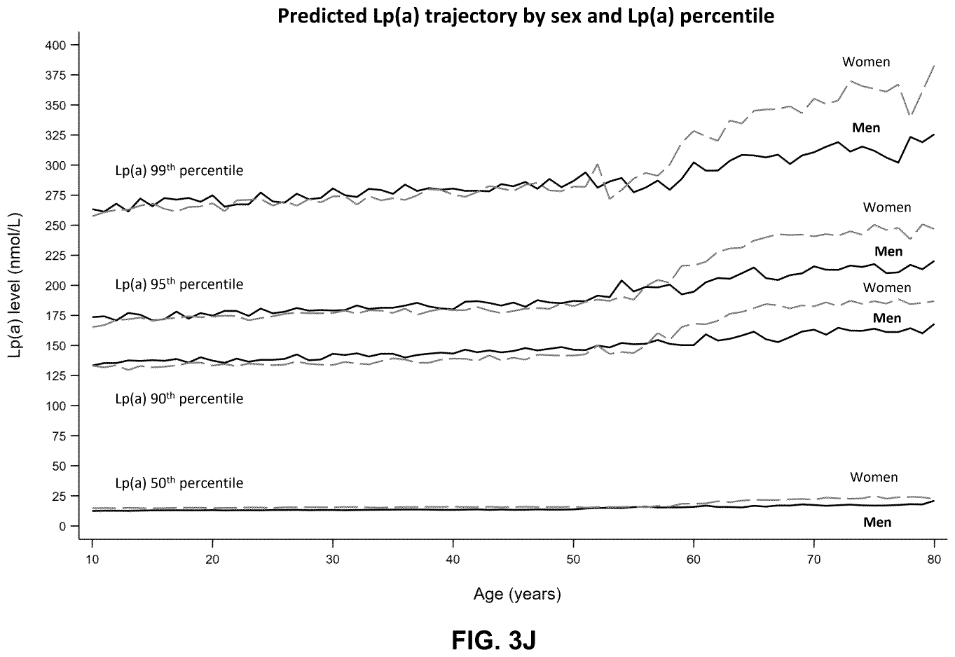

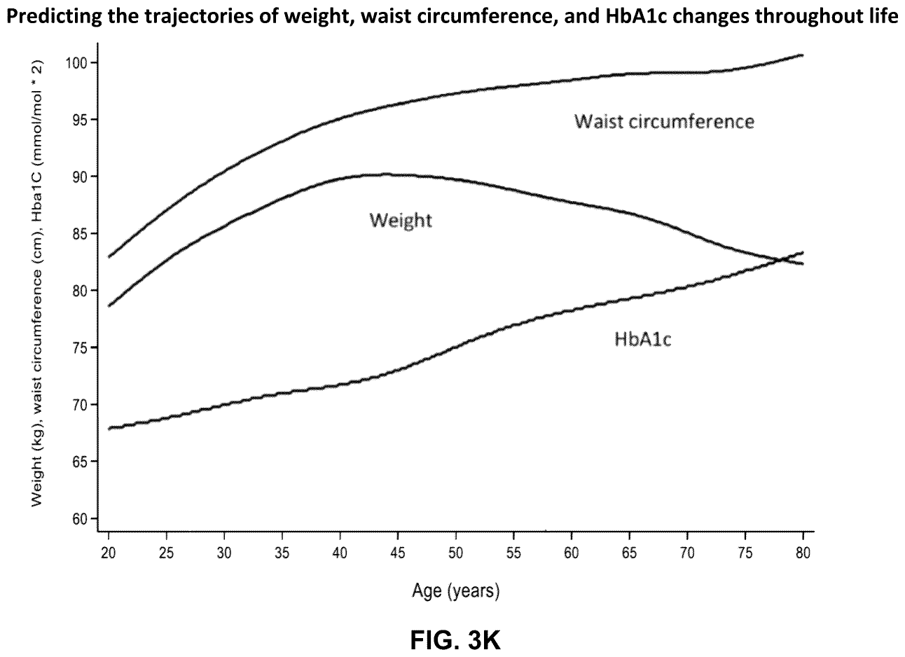

An illustrative example of the subject characteristic and/or measurement data is shown in FIG. 3A. It should be appreciated that this example is merely illustrative and that, in other embodiments, the subject characteristic and/or measurement data may include one or more values of one or more other characteristics and/or measurements than those shown in FIG. 3A. This may include any subject characteristics and/or measurements (whether clinical, physical, or biochemical) that are of clinical relevance (e.g., either as stated in objective clinical guidelines and/or in the judgment of the subject's clinician and/or clinical team).

It should be appreciated that it is not required that all of the aforementioned types of subject characteristic and/or measurement data be obtained for an individual in order for process 200 to be meaningfully applicable to the individual. Indeed, only a subset of the measurements may be available and be used to meaningfully perform the acts shown in FIG. 2B (e.g., to estimate LDL, SBP, and Lp(a) trajectories at acts 214-216) and process 200 more generally (e.g., to determine for each of multiple time intervals, one or more measures of risk that the subject develops cardiovascular disease to obtain multiple measures of risk corresponding to the multiple time intervals; determining, using the multiple measures of risk that the subject develops cardiovascular disease, benefit of administering to the subject one or more therapeutic interventions designed to reduce risk of cardiovascular disease by targeting one or more modifiable causes of the cardiovascular disease; and identifying, using the determined benefit of administering the one or more therapeutic interventions, at least one therapeutic intervention to recommend being administered to the subject).

For example, in some implementations, act 211 may involve obtaining only the subject's age, biological sex, and LDL, SBP, and DBP measurements. Additionally, an Lp(a) measurement may be obtained if available (if not, then act 216 may be omitted). Additionally, any of the characteristics

It should be noted that even if not all possible types of measurements are initially available, they may be added at a future point in time during longitudinal follow-up monitoring if they become available then. At such future time points, an individual being monitored may have multiple measured biomarker levels at specific time points on their individual health trajectory for “untreated” targets. The updated measurements for “treated” targets (LDL, SBP, or both) would be used to quantify the legacy benefit from earlier interventions to slow plaque progression and arterial wall injury accumulation, as described herein. This is part of the rationale for longitudinal monitoring and continuously dynamically updating inputs based on each person's evolving health trajectory of both treated and untreated exposures. With each update, the algorithms better learn each person's individual trajectory based on the increasing number of longitudinal measurements for each person.

This flexibility is by design—and reflects the ‘principle of parsimony’ that informs some of the design elements of the technology described herein. That is, we want to be able to use a minimal set of key features to predict evolving risk based on a person's evolving health trajectory to make the system available to as many people as possible around the world as inexpensively as possible. Adding additional features simply refines the individual absolute estimate of risk at any given time. Of course, these additional features may be quite important (particularly for extreme values)—and therefore add additional information, when available.

Importantly, the estimates of benefit are not impacted. These are based on randomized evidence for an increment of LDL or SBP lowering—with the magnitude of proportional benefit determined by the magnitude and duration of exposure (increasing as disease progression is slowed over time). Because benefit estimated from randomized evidence represents biological causes and effects, we can assess if the benefit of lowering LDL, SBP, or both is conditional on other exposures or biological processes. All of the randomized evidence (both randomized clinical trials and nature's randomized clinical trials) suggest that the benefit of lowering LDL, SBP, or both (and the increased risk caused by an increment of increased LDL, SBP, or both) is independent and therefore very similar regardless of any other exposures.

Returning to FIG. 2B, as described herein, act 212 may involve standardizing the subject characteristic and/or measurement data obtained at act 211. This may be done because raw measurements for the clinical, physical, and biochemical measurements used to characterize the current state of cardiometabolic health of a subject may vary widely in magnitude and may be measured on different scales. Indeed, the data obtained at act 211 may be first standardized in order to: (i) improve computational efficiency (e.g., of algorithms used downstream in process 200); (ii) stabilize gradient flow, and prevent exploding or vanishing gradients (e.g., in using various machine learning models used as part of process 200 and/or training such models for use as part of process 200); and (3) mitigate bias toward features with larger numeric ranges. As such, the standardization performed at act 212 is designed to improve computational efficiency and to preserve use of the same dense input feature vector for multiple different sequential deep and machine learning algorithms, which may be used as part of process 200, as described herein. Moreover, standardization of the raw measurements in the processes described herein (e.g., as implemented by a Deep Causal AI Agent) preserves the possibility of recovering an estimate of the magnitude of the effect size for each input feature.

The standardization may be performed in any suitable way. Indeed, numerous standardization methods are available. In some embodiments, all measured inputs may be converted into respective numeric values between 0 and 1 to improve computational efficiency and eliminate the bias from measuring biological parameters on different scales that can vary by 1000-fold. To this end, dichotomous variables may be encoded as 0 or 1; ordinal variables may be one-hot encoded to create a series of dummy variables with values of 0 or 1; and continuous variables (e.g., polygenic scores, such as the ACSVD-PGS) may be Min-Max standardized to continuous values between 0 and 1, as detailed below. These input values create an n-dimensional input vector.

Family history may be encoded as a binary (dichotomous) variable in some embodiments, but as an ordinal variable in other embodiments. Using family history of CHD as an example, the input indicating whether the subject has a family history of coronary heart disease (CHD) may be coded as a binary variable, and may be standardized as having the values “1” and “0” indicating the presence or absence of family history of CHD, respectively.

In other embodiments, the input indicating whether the subject has a family history of CHD may be coded as an ordinal variable. For example, the variable may take on one value (e.g., 0) to indicate that no first degree relatives have ever had a MI, stroke, or coronary revascularization procedure (which demonstrates a substantial atherosclerotic burden producing impaired blood flow requiring mechanical intervention to reduce the obstruction and restore enough blood flow to meet oxygen demands with exertion), another value (e.g., 1) to indicate that either the biological mother or father has had an MI, stroke or revascularization, another value (e.g., 2) to indicate that both the biological mother and father have had an MI, stroke, or revascularization, another value (e.g., 3) to indicate that both the biological mother and father, and one biological sibling have had an MI, stroke or revascularization, and yet another value (e.g., 4) to indicate that both the biological mother and father, and two or more biological siblings have had an MI, stroke or revascularization. This is important because family history has a dose-dependent effect on the risk of having a major atherosclerotic cardiovascular event. Capping the ordinal representation at four reflects extreme inherited predisposition of atherosclerotic cardiovascular disease (and because very little data is available for persons with more extreme family histories). Interestingly, the dose-response effect of family history and the dose response effect of polygenic predisposition (as measured by a crude first generation polygenic score) are independent and additive (multiplicative on the risk scale, and additive on the log-risk scale).

With respect to continuous variables, min-max scaling (sometimes termed “min-max normalization”) may be used. This technique may be particularly useful when input features are naturally bounded (e.g., as is the case with much biological data). In some embodiments, min-max scaling may be performed using the formula:

$${x}^{′} = \frac{x - {x}_{min}}{{x}_{max} - {x}_{min}}$$

where x is the original feature value, and xₘᵢₙ and xₘₐₓ are the minimum and maximum values of the feature (e.g., in a reference population or on a given scale). An illustrative example of the standardized values (of some subject characteristic and/or measurement data from FIG. 3A) is shown in FIG. 3B.

As described herein, act 213 may involve positionally encoding the standardized data obtained at act 212 (though it should be appreciated that data can be positionally encoded even without being standardized, in some embodiments).

In some embodiments, act 213 involves positionally encoding feature values using age of the subject as the position function. Positional encoding by age injects a biological context into each included feature and is designed to permit the value of each feature to be interpreted within the context of the age of the subject at which the feature value was measured. This formulation of positional encoding provides unique biological information about the likely trajectory of prior values for physiologic and biochemical features that may dynamically change value over time. The feature values (e.g., from act 212) may be used together with their positionally encoded versions (e.g., as output at act 213) for subsequent processing as described herein (e.g., with respect to acts 214, 215, and 216 for calculating LDL, SBP, and Lp(a) trajectories for the subject).

In more detail, the interpretation of the absolute value of a particular biological parameter may vary substantially depending on biological context, including the subject's age, at which the biological parameter was measured. For example, an SBP of 130 mmHg in a 25 year old man is 10 mmHg above the age-and-sex adjusted population median SBP level of 115 mmHg. By contrast, the same SBP level of 130 mmHg in a 65 year old man is 15 mmHg below the age and sex adjusted population median SBP level of 145 mmHg. Thus, the very same SBP level can have very different biological and physiological implications depending on the age and sex of the subject being evaluated.

Accordingly, in some embodiments, positional encoding may be added to the input features to provide a sense of the timing within the subject's evolving health, biological context, and/or age at which the feature was measured. To this end, the standardized values obtained at act 212 may be positionally encoded by age and the obtained positionally-encoded standardized values may be appended to the set of standardized values (which may be referred to herein as a feature vector or a dense input feature vector). The original standardized measurements are not thrown away; instead, they are retained to preserve any information that may be contained in a feature interpreted without regard to the timing during the person's cardiometabolic health trajectory at which the feature is measured.

Positional encoding by age may be performed using any of a variety of positional encoding methods. For example, sinusoidal encoding may be used. This encoding involves calculating the encoding using the following formula:

$$P \text{?} = sin ( \frac{pos}{1000 \text{?}} )$$

$$P \text{?} = cos ( \frac{pos}{1000 \text{?}} )$$

$$\text{?} \text{indicates text missing or illegible when filed}$$

whereby pos is the position in the sequence (age at measurement), d is the dimension of the positional encoding vector (e.g., matching the dimension of the input vector of standardized values returned from act 212), i is the index of the feature within the positional encoding vector. For even indices (2ᵢ), the above uses the sine function to encode the position. For odd indices (2ᵢ+1), the above uses the cosine function to encode the position. The term 10000^{(2/t)} defines a different frequency for each dimension; other constants may be used. Once the positional encoding is computed for each position in the input sequence, it is appended to the input feature vectors to obtain a positionally-encoded version of the standardized feature values to obtain the positionally encoded values, which in turn are appended to the standardized feature values (which are not discarded, as described above). That may be represented as Xᵢₙₚᵤₜ=X_{embedding}+PE. Thus, an input vector of standardized values having n values coming out of act 212 now has 2n values, with the positionally encoded values appended to it.

An illustrative example of computing a positional encoding of the standardized values (from FIG. 3B) is shown in FIG. 3C, where the positional encoding vector (0.231, 0.478, 0.687, etc.) is added to the standardized values (0.47, 0.5, 0, etc.) to obtain the positional encoding.

As described herein, act 214 may involve determining an LDL trajectory for the subject using the standardized and positionally-encoded feature values (values of clinical characteristics, physical measurements, and/or biochemical measurements obtained at act 211) obtained at acts 212 and 213. The LDL level trajectory for the subject may include an estimated LDL level for the subject for each of multiple prior ages of the subject (e.g., every age from birth to current age) and multiple future ages (up to the age 80 years, or other pre-defined age limit) of the subject.

In the context of biomarker (e.g., LDL, SBP, Lp(a), weight, waist circumference, Hba1c) trajectories, “prior ages” refers to ages of the subject prior to the most recent age at which the biomarker was measured for the subject, and “future ages” refers to ages of the subject subsequent to the most recent age at which the biomarker was measured for the subject. In other words, the words “prior” and “future” in this context refer to ages before and after a point in time at which a most recent measurement of the biomarker is available, which may or may not be the same as the current age of the subject (i.e. their age at the time of running a method as described herein). When the analysis of estimating a biomarker trajectory, part of process 200, is performed when the subject is aged the same as when the biomarker was last measured, then “the most recent age at which the biomarker was measured” is the subject's current age and “prior ages” and “future ages” are relative to the current age of the subject whose health is being analyzed.

In embodiments, the reference to estimates at “multiple prior ages” refers to estimates obtained for each of multiple ages of the subject between a predetermined age (e.g. birth) and the most recent age at which the biomarker was measured, or the earliest age at which a biomarker measurement is available for the subject. For example, multiple measurements of the biomarker at respective different ages may be available for a subject (also referred to as “historical measurements”), and estimates at multiple prior ages may be obtained between the predetermined age and the earliest age of the respective different ages. In embodiments, the reference to estimates a “multiple future ages” refers to estimates obtained for each of multiple ages of the subject between the most recent age at which the biomarker was measured and a predetermined cutoff age (e.g. 75, 80, 85 or 90 years old).

For example, in the context of the LDL level trajectory, “prior ages” refers to ages of the subject prior to the subject's age at which their LDL was most recently measured. As one specific example, determining an LDL trajectory for a subject that is 45 years old and whose LDL was measured at age 45, may involve estimating LDL levels from every age from birth until age 44 (these would be estimated levels for “prior ages”) and LDL levels from age 46 until age 80 (these would be estimated levels for “future ages”). As another specific example, determining an LDL trajectory for a subject that is 45 years old and whose LDL was measured at age 43, may involve estimating LDL levels from every age from birth until age 42 (these would be estimated levels for “prior ages”) and LDL levels from age 44 until age 80 (these would be estimated levels for “future ages”).

In embodiments where more than a single LDL measurement is available for a patient (e.g., longitudinal LDL inputs are available for multiple ages of the subject, whether or not consecutive), the LDL level trajectory may include estimated LDL levels for prior ages (relative to most recent age at which an LDL measurement is available) for which historical measurements are not available, and for ages following the most recent age for which an LDL measurement is available for the subject.

In some embodiments, estimating the LDL level trajectory for the subject comprises: (a) generating a feature vector representing the subject from the subject characteristic and/or measurement data (e.g., by encoding the subject characteristic and/or measurement data as described herein); and (b) processing the feature vector generated from the subject characteristic/measurement data using a third trained ML model (which may be referred to as “an LDL trajectory prediction machine learning model”) to obtain the LDL level trajectory for the subject, the third trained ML model having been trained to estimate an LDL level for the subject at each of the multiple prior ages and each of the multiple future ages (relative to the age at which the most recent LDL level measurement was made). The feature vector may include both standardized values of feature values obtained at act 212 and positionally encoded versions thereof obtained at act 213.

The third trained ML model may be of any suitable type. For example, the third trained ML model may be a neural network model, such as a neural network having a transformer-based architecture or a recurrent neural network (RNN) model. For example, the third trained ML model may be a recurrent neural network such as a long short-term memory (LSTM) neural network model (e.g., a bi-directional neural network model), a gated recurrent unit (GRU) neural network model (e.g., a bi-directional GRU neural network model), or an ODE-RNN model. An ODE-RNN model is a recurrent neural network having continuous-time hidden dynamics defined by ordinary differential equations (ODEs), as described for example in: Y. Rubanova, R. T. Chen, and D. K. Duvenaud, “Latent ordinary differential equations for irregularly-sampled time series,” in Advances in Neural Information Processing Systems (NeurIPS 2019) and in E. De Brouwer, J. Simm, A. Arany, and Y. Moreau, “GRU-ODE-Bayes: Continuous modeling of sporadically-observed time series” in Advances in Neural Information Processing Systems (NeurIPS 2019).

For example, in some embodiments, the third trained ML model may be a bi-directional LSTM (bi-LSTM) neural network model. In this case, processing the feature vector using the third trained ML model includes: (a) estimating an LDL level for the subject at each of the multiple future ages (e.g., up to the age 80 years, or other pre-defined age limit) using a forward pass of the bi-LSTM neural network model; and (b) estimating an LDL level for the subject at each of the multiple prior ages (e.g., at all prior ages down to birth) using a backward pass of the bi-LSTM neural network model. In some embodiments, the bi-LSTM model may have hundreds, thousands, or tens of thousands of parameters (e.g., at least 100, at least 1000, at least 2500, at least 5000, at least 10,000, at least 25,000, at least 50,000, at least 100,000, between 100 and 1000, between 1000 and 5000, between 2500 and 25,000, between 10,000 and 100,000, between 50,000 and 250,000 or any other range within these ranges).

Accordingly, in some embodiments, a bi-directional LSTM model may predict the trajectory of a subject's LDL (and, therefore, the subject's cumulative exposure to LDL). In the forward pass (left-to-right), the bi-LSTM predicts the LDL level at all future ages (up to the age 80 years, or other pre-defined age limit) based on the measured LDL level at the age of the subject at which the most recent LDL measurement was obtained (which may be the subject's current age if the prediction using the bi-LSTM is being performed in the same year as the year during which the most recent LDL measurement was obtained) and the combined effect of the other standardized and positionally-encoded input features. In the backward pass (right-to-left), the bi-LSTM predicts the LDL level at all previous ages from the age of the subject at which the most recent LDL measurement was obtained (which may be the subject's current age) going backward until (e.g., birth) using the same neural network.

The predicted LDL level at all ages (e.g., from birth or other pre-defined lower age limit to 80 or other pre-defined upper age limit) is then plotted to provide the predicted trajectory of LDL levels throughout life for the subject being evaluated. Examples of this are shown in FIGS. 3D and 3E.

In particular, FIG. 3D shows the predicted LDL cholesterol levels (in mmol/L) for all ages of the male subject whose data was shown in FIG. 3A. In this example, the person's LDL levels are predicted from their current age (40) up to age 80 years, and from their current age (40) back to birth. FIG. 3D shows the predicted LDL levels to reveal this person's predicted trajectory of changing LDL levels throughout life.

It is important to note that the trajectory of LDL throughout life differs substantially between persons with male and female biological sex—even among persons who have identical levels of all other features at the same ages. Therefore, in some embodiments, one ML (e.g., bi-LSTM, bi-GRU, ODE-RNN) model may be trained to predict LDL levels on data from persons with male biological sex and another ML (e.g., bi-LSTM, bi-GRU, ODE-RNN) model may be trained to predict LDL levels on data from persons with female biological sex. And, indeed, the analyses described herein may use biological sex-specific algorithms, in some embodiments, to achieve greater accuracy. FIG. 3E shows estimates of LDL levels for two persons with different biological sex.

In more detail with respect to embodiments where a bi-LSTM is used to estimate LDL trajectories, a long short-term memory (LSTM) network is a type of recurrent neural network (RNN) designed to effectively learn and capture long-term dependencies in sequential data (as exemplified here through tasks such as predicting the trajectory of how levels of LDL and SBP change throughout a subject's life).

LSTM networks are described by J. Schmidhuber and S. Hochreiter. (“Long short-term memory.” Neural Comput 9.8 (1997): 1735-1780). Bidirectional LSTM networks are described by A. Graves and J. Schmidhuber. (“Framewise phoneme classification with bidirectional LSTM and other neural network architectures.” Neural networks 18.5-6 (2005): 602-610). The neural network may be trained using any suitable neural network optimization software. The optimization software may be configured to perform neural network training by gradient descent, stochastic gradient descent, or in any other suitable way. In some embodiments, the Adam optimizer is used. The Adam optimizer is described by Kingma, D. and Ba, J. ((2015) Adam: A Method for Stochastic Optimization. Proceedings of the 3rd International Conference on Learning Representations (ICLR 2015)).

A bidirectional LSTM (bi-LSTM) model processes input sequences in both forward and backward directions, providing a richer context for considering both past and future information. This makes the model well-suited for tasks where understanding the full context of a sequence is important, including for the tasks described herein such as predicting the future and past levels of LDL (similarly, SBP and Lp(a)) from a single measured value (or multiple longitudinal values) of LDL (similarly, SBP and Lp(a)) to provide an estimate of total cumulative exposure to LDL (similarly, SBP and Lp(a)). Cumulative exposure to LDL, SBP, and Lp(a) are engineered features that have biological meaning that renders them informative biomarkers for determining risk that an individual develops cardiovascular disease and the benefit of treating same preventatively or otherwise.

In some embodiments, the bi-LSTM model for predicting a subject's LDL levels may be trained using participant data for at least 10,000 participants enrolled in one or more prospective studies, whereby for each of the at least 10,000 participants repeated longitudinal measures of LDL, HDL, SBP, weight, waist circumference, BMI, and/or HbA1c are available over at least 10, at least 20, at least 30, or at least 50 years of follow-up, with censoring applied at time lost to follow-up, death, a first cardiovascular event, or initiation of lipid lowering therapy.

In one illustrative embodiment, the bi-LSTM model for predicting a subject's LDL levels was trained on individual participant data from 16,235 participants enrolled in one of three (3) long-term prospective cohort studies (the Framingham Heart Study (FHS), the Framingham Offspring Study (FOS), and the Multi-Ethnic Study of Atherosclerosis (MESA)) for whom repeated longitudinal measures of LDL, HDL, SBP, weight, waist circumference, BMI, and HbA1c (or fasting plasma glucose) are available over 25-59 years of follow-up (participants censored at time lost to follow-up, death, or first atherosclerotic cardiovascular events, or initiation of lipid lowering therapy). Such training data may be used to train alternative model types examples of which are provided herein including GRU-type or ODE-RNN models, for example.

In some embodiments, the bi-LSTM may be trained using the mean-squared error (MSE) loss function. This loss may be calculated as follows.

First, the input sequence data are processed in both directions (forward and backward). In the forward direction, LSTM processes the input sequence from the left to right. In the backward direction, the bi-LSTM processes the input sequence from the right to left. At each time step t, the forward and backward hidden states [hₜ→ and hₜ←] are concatenated (or summed, depending on implementation) to form the full hidden state hₜ. The combined hidden state is then passed to the next layer or used for prediction during inference.

After processing the input sequence through the bi-LSTM layers, the output at each time step may be passed through a dense (fully-connected) layer or a series of layers to produce the final predictions f at each time step t. The bi-LSTM model outputs a sequence of predictions, one for each time step in the input sequence.

In turn, to calculate the loss during training, for each time step t in the sequence, the squared difference between the predicted and measured values is computed as:

$${( {y}_{t} - \text{?} )}^{2}$$

$$\text{?} \text{indicates text missing or illegible when filed}$$

where: yₜ is the true value at time step t and is ŷₜ the predicted value at time step t.

The MSE loss over the entire sequence is calculated by summing the squared errors across all time steps and taking the average:

$$MSE = \frac{1}{T} \text{?} {( \text{?} - \text{?} )}^{2}$$

$$\text{?} \text{indicates text missing or illegible when filed}$$

where T is the total number of time steps in the sequence.

Once the MSE loss is computed, backpropagation is used to calculate the gradients of the loss with respect to the model parameters (LSTM weights, biases, etc.). These gradients are then used to update the model parameters using an optimization algorithm such as stochastic gradient descent (SGD) or Adam.

In some embodiments, the LSTM models may be implemented using PyTorch's native libraries, which provide direct support with well-optimized functions for LSTM, including:
torch.nn—The module includes nn.LSTM for implementing the LSTM layer, as well as nn.LSTMCell for finer control over LSTM cell computations. Additional modules like nn.Linear may be used to map the LSTM outputs to desired output dimensions.
torch.optim—For defining optimization algorithms, such as Adam or SGD, to train the model.
torch.utils.data—This is useful for managing time-series datasets and implementing custom data loaders or transformers.

Returning to FIG. 2B, act 215 involves determining the SBP trajectory for the subject using a 4^{th} trained ML model using the standardized and positionally encoded feature values (values of clinical characteristics, physical measurements, and/or biochemical measurements obtained at act 211) obtained at acts 212 and 213. The SBP level trajectory for the subject may include an estimated SBP level for the subject for each of multiple prior ages of the subject (e.g., every age to 20 or other pre-defined lower age limit) and multiple future ages (up to the age 80 years, or other pre-defined upper age limit) of the subject. As noted above for LDL estimation, separate models may be used to estimate SBP levels for males and females.

In some embodiments, estimating the SBP level trajectory for the subject comprises: (a) generating a feature vector representing the subject from the subject characteristic and/or measurement data; and (b) processing the feature vector using a fourth trained ML model (which may be referred to as “an SBP trajectory prediction machine learning model”) to obtain the SBP level trajectory for the subject, the fourth trained ML model having been trained to estimate an SBP level for the subject at each of the multiple prior ages and each of the multiple future ages. The feature vector may include both standardized values of feature values obtained at act 212 and positionally encoded versions thereof obtained at act 213. The feature vector may be the same as the feature vector used by the third ML model to estimate the LDL level trajectory for the subject at act 214.

The fourth trained ML model may be of any suitable type including any of the types described above for the third trained ML model, including a recurrent neural network, such as a a bi-directional LSTM (bi-LSTM) neural network model, a bi-directional GRU neural network model, or an ODE-RNN model. In embodiments where the fourth trained ML model is a bi-LSTM, processing the feature vector using the fourth trained ML model comprises: estimating an SBP levels for the subject at each of the multiple future ages (relative to the age at which the most recent SBP measurement was taken) using a forward pass of the bi-LSTM model; and estimating an SBP level for the subject at each of the multiple prior ages using a backward pass of the bi-LSTM model. In some embodiments, the bi-LSTM model may have hundreds, thousands, or tens of thousands of parameters (e.g., at least 100, at least 1000, at least 2500, at least 5000, at least 10,000, at least 25,000, at least 50,000, at least 100,000, between 100 and 1000, between 1000 and 5000, between 2500 and 25,000, between 10,000 and 100,000, between 50,000 and 250,000 or any other range within these ranges).

The fourth ML model may be trained using similar training data as was used to train the third ML model for LDL level estimation, and may be trained using analogous training methods.

In more detail with respect to embodiments where a bi-LSTM is used to estimate SBP trajectories, the bi-LSTM may be designed to predict the SBP trajectory for an individual person which, in turn, may be used to estimate rate of rise and/or cumulative exposure to SBP for the individual. The forward pass (left-to-right) of this bi-LSTM predicts the SBP level at all future ages (up to the age 80 years, or other pre-defined upper age limit) based on the measured SBP level at the current age and the combined effect of the other input features. The backward pass (right-to-left) predicts the SBP level at all previous ages from the current age backwards to age 20 years (or other pre-defined lower age limit) using the same fully connected dense neural network. Of note, the SBP levels before age 20 are discounted because they evolve in response to the rapidly changing physiologic requirements during the rapid growth of childhood and adolescence, and because SBP levels are high enough to cause accumulating irreversible structural injury to the arterial wall during this time is exceedingly rare.

The trained bi-LSTM may be used to provide the predicted SBP level (e.g., from ages 20 to 80), which can then be plotted to provide the predicted trajectory of SBP levels throughout life for the subject being evaluated. Examples of this are shown in FIGS. 3G and 3H.

In particular, FIG. 3G shows the predicted SBP levels (in millimeters of mercury (mmHg)) for all ages of the male subject whose cardiometabolic health data was shown in FIG. 3A. The prediction was made using a 2n-dimensional dense input vector obtained from acts 212 and 213 (with n values being standardized values and n values being the positionally encoded versions thereof). As with LDL trajectories, it is important to note that the trajectory of SBP throughout life differs substantially between persons with male and female biological sex—even among persons who have identical levels of all other features at the same ages. Therefore, in some embodiments, one ML model (e.g., one bi-LSTM) may be trained to predict SBP levels on data from persons with male biological sex and another ML model (e.g., another bi-LSTM) may be trained on data from persons with female biological sex. And, indeed, the analyses described herein may use biological sex-specific algorithms, in some embodiments, to achieve greater accuracy. FIG. 3H shows estimates of SBP levels for two persons with different biological sex.

Returning to FIG. 2B, act 216 involves determining the Lp(a) trajectory for the subject. As will be described next, a machine learning approach is used to estimate this trajectory even though Lp(a) levels for each person are fairly stable throughout life, are largely inherited than acquire, and are not changed by diet, exercise, or other lifestyle choices. Despite such stability, it is not accurate to estimate cumulate Lp(a) levels by simply multiplying a person's Lp(a) level measured at any point in time by their age to obtain cumulative exposure to Lp(a) up to that age. Nor is it accurate to simply multiply a person's measured Lp(a) level by every age to obtain their Lp(a) at each age and sum (or integrate) them to calculate cumulative exposure to Lp(a).

The reason for why such simple approximations will not provide accurate estimates is that, for almost everyone, there is a slight inflection point between the ages of 50 to 60 at which Lp(a) levels begin to steadily rise over time (co-incident with the corresponding plateau and then fall of LDL levels over time). Importantly, this change in Lp(a) is detectable (or at least quantitatively and biologically meaningful) for people with extreme elevations of Lp(a). By contrast, for most persons, this inflection point and subsequent rise in Lp(a) is largely undetectable and biologically irrelevant because most people have very low Lp(a) levels. As a result, a gradual rise in Lp(a) of 20-30% beginning between ages 50 to 60 years for most people will translate into such small absolute changes in Lp(a) as to have an almost imperceptible clinical impact.

However, it is the people who inherit lifelong exposure to markedly elevated Lp(a) who experience the greatest biological impact on the risk of ASCVD events due to Lp(a) and therefore are the people who would benefit most from lowering Lp(a) when specific Lp(a)—lowering therapies become available in future; and these are the people who have been enrolled in all of the cardiovascular outcome randomized trials evaluating RNA-based (ASO and siRNA) therapies to reduce Lp(a).

Because Lp(a) almost certainly has a cumulative effect on the risk of ASCVD, like other atherogenic apoB-containing particles, it is important to estimate each person's cumulative exposure to Lp(a) as accurately as possible to both predict the increased risk caused by cumulative exposure to Lp(a) at all time points and at each level of accumulated plaque burden estimated by cumulative exposure to LDL (and to triangulate the results of randomized trials when completed to accurately predict the expected individual benefit from lowering Lp(a) over time based on the reduction in cumulative exposure to Lp(a)).

Accordingly, while it is possible to use crude estimates such as those described above to estimate the Lp(a) trajectory at act 216, more accurate estimates can be obtained by using a trained machine learning model, as described herein. Accordingly, in some embodiments, act 216 involves estimating an Lp(a) trajectory for the subject using a 5^{th} trained ML model using the standardized and positionally encoded feature values (values of clinical characteristics, physical measurements, and/or biochemical measurements obtained at act 211) obtained at acts 212 and 213. The feature values may include the subject's measured Lp(a) level(s), which are standardized (using Min-Max standardization) and positionally encoded.

The Lp(a) level trajectory for the subject may include an estimated Lp(a) level for the subject for each of multiple prior ages of the subject (e.g., multiple ages prior to the age at which the most recent Lp(a) level was measured down to birth or other pre-defined lower age limit). With respect to prior years, the Lp(a) trajectory may include an estimated Lp(a) level for the subject for those years where Lp(a) measurements are not available since it is possible that multiple Lp(a) measurements are available for the subject; the measured Lp(a) values rather than estimates may be used for the years in which the measurements were obtained. For example, if a subject is 40 years old and Lp(a) measurements are available for the ages of 40, 35, and 33, then the Lp(a) trajectory may include Lp(a) values for all ages 6-40, with the values at years 33, 35, and 40 being measured values and all other values being estimated using the 5^{th} trained ML model. As such, the Lp(a) trajectory may include both estimated and measured values.

The Lp(a) level trajectory for the subject may also include an estimated Lp(a) level for each of multiple future ages of the subject (e.g., multiple ages subsequent to the age at which the most recent Lp(a) level was measured up to the age of 80 years, or other pre-defined upper age limit).

As noted above for LDL and SBP estimation, separate models may be used to estimate Lp(a) levels for males and females. FIG. 3I shows an illustrative example of Lp(a) trajectories predicted for men and women (in various percentiles of the population) with each Lp(a) trajectory indicating Lp(a) levels as a function of age, in accordance with some embodiments of the technology described herein.

In some embodiments, estimating the Lp(a) level trajectory for the subject comprises: (a) generating a feature vector representing the subject from the subject characteristic and/or measurement data; and (b) processing the feature vector using a fifth trained ML model (which may be referred to as “an Lp(a) trajectory prediction machine learning model”) to obtain the Lp(a) level trajectory for the subject, the fifth trained ML model having been trained to estimate an Lp(a) level for the subject at each of the multiple prior ages and each of the multiple future ages. The feature vector may include both standardized values of feature values obtained at act 212 and positionally encoded versions thereof obtained at act 213. The feature vector may be the same as the feature vector used by the third ML model to estimate the LDL level trajectory for the subject at act 214.

The fifth trained ML model may be of any suitable type including any of the types described herein for the third and fourth trained ML models, including a recurrent neural network, such as a bi-directional LSTM (bi-LSTM) neural network model or a bi-directional GRU neural network model. In embodiments where the fifth trained ML model is a bi-directional (e.g., bi-LSTM or bi-GRU) neural network (BNN) model, processing the feature vector using the fifth trained ML model comprises: estimating an Lp(a) level for the subject at each of the multiple future ages (relative to the age at which the most recent Lp(a) measurement was taken) using a forward pass of the BNN model; and estimating an Lp(a) level for the subject at each of the multiple prior ages (relative to the age at which the most recent Lp(a) measurement was taken) using a backward pass of the BNN model. In some embodiments, the bi-LSTM model may have hundreds, thousands, or tens of thousands of parameters (e.g., at least 100, at least 1000, at least 2500, at least 5000, at least 10,000, at least 25,000, at least 50,000, at least 100,000, between 100 and 1000, between 1000 and 5000, between 2500 and 25,000, between 10,000 and 100,000, between 50,000 and 250,000 or any other range within these ranges).

The fifth trained ML model for estimating Lp(a) trajectories may be trained using training data generated from patient data for patients who have had their Lp(a) assessed once or multiple times (e.g., longitudinally). In some embodiments, the training data may be obtained from UK Biobank, including approximately 460,000 participants with at least one Lp(a) value measured at enrollment, and approximately 19,000 participants who had a second Lp(a) measured at a median of 4.6 years after enrollment. In addition, laboratory measurements research data are available on approximately 1,000,000 persons from a network of Lipid Specialty Clinical around the world organized by the European Atherosclerosis Society and the International Atherosclerosis Society. Among patients in these clinics, approximately 70% have had two or more Lp(a) values measured at different ages, excluding those measurements for which the repeated measures we obtained to assess the impact a therapeutic agent to lower Lp(a). This network of specialty clinics is growing and organized to collaborate on lipid research questions. The measurements of Lp(a) in these lipid specialty clinic patients, in some instances but not all, were directed by a protocol to assess if and how Lp(a) varies over time. Although some persons are referred into these clinics because of elevated Lp(a) and some even receive treatments that can modestly lower Lp(a) or plasmapheresis to substantially lower Lp(a) and everything else, the vast majority of participants in these clinics just have Lp(a) measured as part of an advanced lipid and lipoprotein panel assessment. Lp(a) is unique because it is largely independently inherited and therefore not confounded by the other lipid disorders or their treatments. The fifth trained ML model for estimating Lp(a) trajectories may be trained using analogous training methods to those used to train the third and fourth ML models.

As may be appreciated from the foregoing discussion, different types of ML models may be used to estimate LDL, SBP, and Lp(a) trajectories as part of process 200. Within the class of recurrent neural networks, a variety of approaches are possible including LSTM, GRU, and ODE-RNN. Among these, any of the approaches may be used. However, in the context of predicting SBP trajectories (as well as Lp(a) trajectories), it may be of interest to detect an inflection point above SBP (and Lp(a)) begins to rise independently over time due to a feedback cycle of increasing vascular injury. For this reason, an LSTM architecture may be preferrable to the GRU architecture because the individual gates at different timepoints in an LSTM may be interrogated for possible clues about this inflection point. By contrast, the bi-directional GRU (or GRU more generally) hidden gate is much less accessible at specific time stamps. Indeed, the LSTM and GRU internal gating mechanisms and memory architectures are different and because GRUs compress memory and gating behavior into fewer mechanisms, some of the temporal interpretability that LSTMs provide via distinct cell states (each state having input, forget, and output gates) is lost.

As between unidirectional vs. bi-directional RNN architectures, bi-directional architectures may be preferrable because using unidirectional architecture to predict LDL, SBP, or Lp(a) for only future years tends to underestimate the true biological effect of an exposure that produces irreversible structural injury that accumulates over time. That said, in some embodiments, unidirectional architectures may be used. In such embodiments, a simplifying assumption may then be made that an individual's LDL (SBP and/or Lp(a)) followed the average trajectory for persons of the same sex up to the subject's current age—and that the proportional difference in LDL (or SBP or Lp(a)) for this individuals compared to the median for LDL (or SBP or Lp(a)) among persons of the same biological sex and age in the reference difference was constant at all ages leading up to the current age. In such embodiments, the unidirectional RNN (e.g., LSTM, GRU, latent ODE GRU) would likely predict an unique future trajectory that is not the same ‘shape’ as the average trajectory of persons in the reference population. This would lead to assuming everyone has two different trajectories demarcated by the age at which they happened to input their LDL or SBP.

Neural networks having a transformer architecture may also be used instead of RNNs. For example, neural networks having a temporal fusion transformer (TFT) architecture may be used. TFT neural networks are described in Bryan Lim, Sercan Ö. Arik, Nicolas Loeff, Tomas Pfister, “Temporal Fusion Transformers for interpretable multi-horizon time series forecasting,” International Journal of Forecasting, Volume 37, Issue 4, 2021, pp. 1748-1764. However, such neural networks generally require very large data sets for training and may not perform as well as the RNN architectures where few longitudinal data sets are available that have repeated longitudinal measurements of biomarkers for individual participants at regular intervals over an extended number of years.

In some embodiments, the third ML model (an LDL trajectory prediction ML model), the fourth ML model (an SBP trajectory prediction ML model), and the fifth ML model (an Lp(a) trajectory prediction ML model) may all operate on a same input vector generated at act 213. However, in other embodiments, the input vectors provided to these models need not be the same. For example, some feature values (e.g., Lp(a)) may be omitted from the inputs provided to the third ML model or the fourth ML model, but may be provided to the fifth ML model, as aspects of the technology described herein are not limited to having the trajectory prediction models on the same exact input vector at acts 214-216.

After acts 214-216, where LDL, SBP, and Lp(a) level trajectories are computed for the subject (e.g., using two bi-directional LSTMs, GRUs, or other algorithms), the LDL trajectory may be used to compute the cumulative LDL exposure trajectory, the SBP trajectory may be used to compute the cumulative SBP exposure trajectory, and the Lp(a) trajectory may be used to compute the cumulative Lp(a) exposure trajectory, at act 217. In addition, at act 217, the instantaneous rate of rise of the SBP levels may be determined.

In some embodiments, the cumulative LDL exposure trajectory may be determined by summing the predicted LDL level at each age of the subject (or the area under the curve for the subject's LDL trajectory may be integrated) to iteratively compute the subject's cumulative exposure to LDL at every age (measured as Plaque Years of LDL in mmol/L, though other units may be used). An example cumulative LDL exposure trajectory, computed from the LDL levels shown in FIG. 3D, is shown in FIG. 3F.

Cumulative exposure to LDL is an important and dynamic biomarker that estimates the number of atherogenic LDL (and other apoB-containing lipoproteins) that have become trapped within the artery wall up to that point in time, and therefore represents the first biochemical parameter to estimate the size of a person's accumulated plaque burden and track their rate of plaque progression. This marker is a direct estimate of the number of LDL and other atherogenic apoB-containing lipoproteins that the arterial wall has been exposed to over time. And, therefore, it provides an indirect estimate of the number of LDL particles that have become trapped within the artery wall over time. As a result, cumulative exposure to LDL (measured in mmol/L of total Plaque Years of exposure to LDL) can be used to estimates the size of the accumulated atherosclerotic plaque burden at any point in time, the rate of plaque progression, and/or the expected size of the accumulated plaque burden at all future time points.

In some embodiments, the values for the predicted LDL levels and corresponding cumulative exposure to LDL at all ages may be used together with the other features already computed (e.g., at acts 212 and 213) for further processing (e.g., as part of act 220).

Turning now to the cumulative SBP exposure trajectory, in some embodiments, the cumulative SBP exposure trajectory may be determined by summing the predicted SBP level at each age of the subject (or the area under the curve for the subject's SBP trajectory may be integrated) to iteratively compute the subject's cumulative exposure to SBP at every age (measured mmHg-years, though other units can be used). An example cumulative SBP exposure trajectory, computed from the SBP levels shown in FIG. 3G, is shown in FIG. 3I.

Cumulative exposure to SBP is another bio-engineered feature that represents a dynamic biomarker that provides unique biological information. Like cumulative exposure to LDL, elevated SBP causes irreversible structural injury to the artery wall that accumulates over time. Specifically, elevated SBP causes non-laminar blood flow at vulnerable branch points within the arterial tree. The non-laminar flow, in turn, causes an increased flux of LDL and other atherogenic apoB-containing lipoproteins within the artery wall. In addition, the elevated SBP causes hypertrophy of vascular smooth muscle cells; which, in turn, causes the secretion of a greater concentration of the proteoglycans that trap LDL and other apoB-containing lipoproteins within the artery wall. This combination of increased flux of atherogenic lipoproteins into the artery wall and a greater concentration of proteoglycans that can trap the increased concentration of LDL within the artery wall results in progressively increasing focal SBP induced plaque accumulation over time at vulnerable branch points within the arteries. As a result, SBP has a cumulative effect on the development of atherosclerotic plaque at specific vulnerable points within the vascular tree. In addition, elevated SBP causes accumulating diffuse structural injury to the artery wall resulting in vascular stiffening and inflammation (arteriosclerosis), which reduces the capacity of the artery to tolerate the accumulated plaque burden by making it more likely that a thrombus overlying a disrupted atherosclerotic plaque will occlude the vessel at any level of accumulated plaque burden. Finally, elevated SBP also increases the risk of plaque disruption, thus increasing the risk of acute cardiovascular events. For these reasons, LDL and SBP appear to have independent, additive, causal, and cumulative effects on the risk of acute cardiovascular events. The bio-engineered cumulative exposure to SBP biomarker computed by the method described herein provides unique information by quantifying these biological effects.

In addition to determining the cumulative SBP exposure trajectory, the SBP levels predicted at act 215 may be used to predict the rate of rise in SBP for the individual person being evaluated (e.g., by estimating slope of the subject's SBP trajectory using any suitable method).

The predicted rate of rise in SBP is another unique bio-engineered feature. It can be combined with the measured SBP at the current age to predict whether a person is likely to develop hypertension, and the age at which person is likely to develop hypertension based on how rapidly SBP is predicted to rise with age.

This information is useful for informing selection of the optimal sequence of current and anticipated future actions needed to achieve the combined goal of preventing MI, stroke, hypertension, and/or diabetes.

In some embodiments, the values for the predicted SBP levels, rates of rise of SBP, and corresponding cumulative exposure to SBP at all ages, and positionally encoded (by age) versions thereof, may be used together with the other features already computed (e.g., at acts 212 and 213, LDL levels, and cumulative LDL levels) for further processing (e.g., as part of act 220).

In addition, as part of act 217, a cumulative Lp(a) exposure trajectory may be determined by summing the Lp(a) levels in the Lp(a) trajectory determined at act 216 (or the area under the curve for the subject's Lp(a) trajectory may be integrated) to iteratively compute the subject's cumulative Lp(a) exposure trajectory, which indicates for each of one or more ages of the subject (e.g., some or all of ages from 5 to 80), the subject's cumulative exposure to Lp(a) at that age.

In some embodiments, the values for the predicted Lp(a) levels and corresponding cumulative exposure to Lp(a), and positionally encoded (by age) versions thereof, may be used together with the other features already computed (e.g., at acts 212 and 213, LDL levels, cumulative LDL exposure levels, SBP levels, rates of rise of SBP, cumulative SBP exposure levels) for further processing (e.g., as part of act 220).

After act 217, process 200 proceeds to act 218 where one or more other trajectories may be determined for the subject.

For example, in some embodiments, at act 218, a weight trajectory for the subject may be determined, the weight trajectory for the subject comprising estimated weight of the subject for each of multiple prior ages of the subject and/or multiple future ages of the subject. This estimate may be obtained in any suitable way, for example, based on the assumption that the subject's current age-and-sex adjusted weight percentile remains constant throughout life.

As another example, in some embodiments, at act 218, a waist circumference trajectory for the subject may be determined, the waist circumference trajectory of the subject comprising the estimated waist circumference of the subject for each of multiple prior ages of the subject and/or multiple future ages of the subject. This estimate may be obtained in any suitable way, for example, based on the assumption that the subject's current age-and-sex adjusted waist circumference remains constant throughout life.

As yet another example, in some embodiments, at act 218, an HbA1c level trajectory for the subject may be determined, the HbA1c level trajectory of the subject comprising estimated HbA1c levels of the subject for each of multiple prior ages of the subject and/or multiple future ages of the subject. This estimate may be obtained in any suitable way, for example, based on the assumption that the subject's current age-and-sex adjusted HbA1c percentile remains constant throughout life.

An example of these trajectories for the subject (whose cardiometabolic data is shown in FIG. 3A) is shown in FIG. 3K.

After completion of acts 214-218, a number of novel biomarkers providing an assessment of the subject's current state of cardiometabolic health are provided. A trained machine learning model (e.g., a bi-LSTM, a bi-GRU neural network) was used to determine the size of the atherosclerotic plaque burden that has accumulated, and the rate at which the plaque burden is progressing. Another trained ML model (e.g., another bi-LSTM, bi-GRU neural network) was used to determine the rate at which SBP is rising over time, and how much structural injury caused by elevated SBP has accumulated. Yet another trained ML model (e.g., yet another bi-LSTM, bi-GRU neural network) was used to determine Lp(a) levels and cumulative Lp(a) levels. Additionally, estimates of the current weight, waist circumference, and HbA1c trajectories are obtained.

Next, at act 219, the various cardiometabolic data obtained and/or determined at acts 211-219 may be used for further processing. As described above, data may be stored, in memory (volatile or non-volatile), using any suitable data structure or data structures, and may be subsequently accessed when performing further processing (e.g., as part of act 220 of process 200). As another example, the data may be passed onto other software modules for processing (e.g., to software modules configured to implement the function of act 220 of process 200).

It should be appreciated that the process shown in FIG. 2B is illustrative and that there are variations.

For example, in the illustrated process, multiple biomarker trajectories are determined (e.g., LDL, SBP, Lp(a), cumulative LDL exposure, instantaneous rate of rise of SBP, cumulative SBP exposure, cumulative Lp(a) exposure, weight, waist circumference, and HbA1c). In other embodiments, only a subset (e.g., any subset) of these may be determined. For example, in some embodiments, the LDL and cumulative LDL exposure trajectories may be determined, but the SBP and cumulative SBP exposure trajectories may not be determined and therefore not used subsequently. As another example, in some embodiments, the LDL, cumulative LDL exposure, SBP, and cumulative SBP exposure trajectories may be determined, but the Lp(a) and cumulative Lp(a) trajectories may not be determined (or if the Lp(a) trajectories are determined, a simple approximation may be used instead of a machine learning model to do so—examples of such simple approximations are provided herein).

### Determining Measure(s) of Risk That Subject Develops Cardiovascular Disease

As shown in FIG. 2A, process 200 includes act 220 in which at least some (e.g., all) of the cardiometabolic health data obtained for the subject at act 210 may be used, together with a first trained ML model (e.g., a trained survival model), and for each of multiple time intervals (e.g., years), one or more measures of risk that the subject develops cardiovascular disease, thereby obtaining multiple measures of risk corresponding to the multiple time intervals.

In some embodiments, this may be performed in accordance with the illustrative process shown in FIG. 2C. As shown in FIG. 2C, act 220 may involve: (1) obtaining, at act 221, input feature data; (2) estimating, at act 222 and using the first trained ML model and the input feature data obtained at act 221, values indicative of log hazard ratios of risk of the subject having a cardiovascular event at respective levels of cumulative LDL exposure; (3) estimating, at act 223 and using the values indicative of the log hazard ratios, absolute instantaneous and cumulative hazard rates, and event rates, of the subject having a cardiovascular event at the respective levels of cumulative LDL exposure; and (4) estimating, at act 224, using the cumulative LDL exposure trajectory for the subject, absolute instantaneous and cumulative hazard rates, and event rates, of the subject having a cardiovascular event at the respective ones of the multiple time intervals (e.g., years). Aspects of some of the acts 221-224 are now further described in greater detail.

At act 221, the various cardiometabolic health data values for the subject (obtained at act 210, for example, as described with respect to FIG. 2B) may be accessed and pre-processed and/or organized, into the input feature data, for subsequent processing by the first ML model at act 222. Thus, in some embodiments, the process of FIG. 2C involves: (1) generating, at act 221, input feature data using at least some of the cardiometabolic health data for the subject; and (2) processing, at act 222, the input feature data using the first trained ML model to obtain, as output, the values indicative of the log hazard ratios for the risk of the subject having a cardiovascular event at the respective levels of cumulative LDL exposure. In some embodiments, values indicative of the log hazard ratios may be values of the log hazard ratios themselves or other values, from which the log hazard ratios may be (e.g., uniquely) determined.

First, aspects of generating the input feature data at act 221 are described. Following this is a description of how that data is processed using the first trained ML model as well as aspects of the first ML model.

With respect to the input feature data, in some embodiments, the input feature data may be generated using at least some (e.g., all) of the feature values part of the subject's cardiometabolic health data including the subject's characteristic and/or measurement data (e.g., including age, biological sex, family history, polygenic scores, etc.), LDL and cumulative LDL exposure trajectories, SBP and cumulative SBP exposure trajectories, Lp(a) and Lp(a) exposure trajectories, weight trajectory, waist circumference trajectory, HbA1c trajectory, and/or positionally encoded versions of one or more thereof.

For example, in some embodiments, the input feature data may comprise a set of input feature vectors (e.g., organized as a matrix or in any other suitable way), with each particular one of the input feature vectors corresponding to a particular cumulative LDL exposure level from among the respective levels of cumulative LDL exposure. In turn, the particular input feature vector in the set of input feature vectors that corresponds to the particular cumulative LDL exposure level may include: (a) subject characteristic and/or measurement data comprising: one or more values for one or more clinical characteristics of the subject, one or more values for one or more physical measurements of the subject, and/or one or more values for one or biochemical measurements of the subject; (b) an LDL level trajectory for the subject; (c) a cumulative LDL exposure trajectory for the subject (e.g., determined from the LDL level trajectory); (d) an SBP level trajectory for the subject indicating an estimated SBP level for the subject at each of the respective levels of cumulative LDL exposure; (e) a cumulative SBP exposure trajectory for the subject indicating an estimated cumulative SBP exposure for the subject at each of the respective levels of cumulative LDL exposure; (f) an Lp(a) trajectory for the subject indicating an estimated Lp(a) level for the subject at each of the respective levels of cumulative LDL exposure; (g) a cumulative Lp(a) exposure trajectory for the subject indicating an estimated cumulative Lp(a) exposure for the subject at each of the respective levels of cumulative LDL exposure; (h) a weight trajectory for the subject indicating an estimate of the subject's weight at each of the respective levels of cumulative LDL exposure; (i) a waist circumference trajectory for the subject indicating an estimate of the subject's waist circumference at each of the respective levels of cumulative LDL exposure; (j) an HbA1c level trajectory for the subject indicating an estimate of the subject's HbA1c level at each of the respective levels of cumulative LDL exposure; and/or (k) an age trajectory for the subject indicating an estimated age for the subject at each of the respective levels of cumulative LDL exposure. Additionally, the particular input feature vector may include: (1) a positionally encoded version of one or more of (a), (b), (d), (e), (f), (g), (h), (i), (j), and (k) obtained by positionally encoding the one or more of (a), (b), (d), (e), (f), (g), (h), (i), (j), and (k) by age of the subject at the particular cumulative LDL exposure level is predicted to occur. The subject's cumulative LDL exposure trajectory is not positionally encoded, in some embodiments.

Any combination of the foregoing features may be used to create the input feature data. Thus, a subset of the foregoing features may be used in some embodiments. For example, features (a), (b), (c), (d), and (e) may be used, along with their positional encoding, to constitute the input feature vectors. As another example, in some embodiments, all of the foregoing features, but without Lp(a) features (f) and (g) and their respective positional encodings may be used.

As a specific illustrative example, the first trained ML model may be a survival model that is trained to use cumulative exposure to LDL as the increment of follow-up (instead of age or time) at the time of inference in order to estimate the natural log hazard ratio at each level of cumulative exposure to LDL for the subject being evaluated. (Further aspects of such models are described herein).

In this example, the first trained ML model may take as input a matrix of feature vectors, with each vector calculated for a respective level of cumulative LDL exposure. A particular feature vector for a particular level of cumulative LDL exposure may be composed of two parts: (1) a set of time-invariant or fixed inputs that do not vary over time (e.g., sex, family history, polygenic scores, etc.); and (2) a set of time-varying or changing inputs whose values do change over time—either naturally like LDL, SBP, and Lp(a) levels, or due to lifestyle choices like weight or waist circumference or tobacco smoking, or as a biological consequence of lifestyle choices such as changes in HbA1c in response to excess energy balance by consuming more calories than one expends.

As a result, the values of the time-invariant features included in particular feature vectors for particular levels of cumulative LDL exposure are the same across the all the vectors. However, the positional encoding of these inputs (e.g., polygenic scores such as the ASCVD-PGS) does change at each level of cumulative exposure to LDL because the age at which the corresponding level of cumulative exposure occurs changes as plaque accumulates from trapping more LDL particles over time. In addition, the value of each time-varying feature and its positional encoding also changes at each level of cumulative exposure to LDL.

Thus, in this example, the input feature data may be generated (at act 221) by determining multiple feature vectors calculated for multiple respective levels of cumulative LDL exposure. To this end, first, a “mapping” function between age and cumulative exposure LDL levels may be obtained—this may be done as described with respect to FIG. 2B (e.g., using the output of the trained ML model (e.g., bi-LSTM, bi-GRU, ODE-RNN) that calculates the LDL trajectory and based on this, determining a cumulative LDL exposure trajectory which provides a cumulative LDL exposure level for each age). Then, at each level of cumulative LDL exposure, the corresponding input feature vector may include:
(1) standardized clinical characteristics, physical measurements, and biochemical measurements for all time-invariant features and their positional encodings by the age of the subject at which the corresponding level of cumulative exposure to LDL occurred (which varies for each person depending on how rapidly their cumulative exposure to LDL and corresponding plaque size are increasing and, therefore, is determined using the mapping between the subject's age and cumulative LDL exposure trajectory);
(2) standardized LDL level at the corresponding increment of cumulative LDL exposure based the subject's predicted trajectory of LDL levels and the positional encoding of the LDL level by the age of the subject at which the corresponding level of cumulative exposure to LDL occurred (in this example, the LDL levels are positionally encoded by the corresponding age at each level of cumulative exposure—but “cumulative exposure to LDL” levels are not, because this is the x-axis or survival ‘time’ variable in the ‘survival’ analysis);
(3) standardized SBP level at each increment of cumulative exposure to LDL based on the subject's predicted trajectory of SBP levels (and the corresponding ages at which those SBP levels occur to match or map the age at which the corresponding level of cumulative exposure to LDL occurs) as well as the positional encoding of the standardized SBP levels by the age of the subject at which the corresponding level of cumulative exposure to LDL occurred;
(4) cumulative exposure to SBP at each increment of cumulative exposure to LDL (using the corresponding ages at which those cumulative exposure to SBP levels occur to match or map the age at which the corresponding level of cumulative exposure to LDL occurs), and the positional encoding of cumulative exposure to SBP by the age of the subject at which the corresponding level of cumulative exposure to LDL occurred to capture the rate of rising SBP on the stability of the total accumulated plaque as estimated at each level of cumulative exposure to LDL (which turns out to be an important determinant of propensity for plaque disruption at vulnerable branch points and, as such, a powerful predictor of the instantaneous risk of having an acute cardiovascular event);
(5) standardized Lp(a) level at each increment of cumulative exposure to LDL based on the subject predicted trajectory of Lp(a) levels (and the corresponding ages at which those Lp(a) levels occur to match or map the age at which the corresponding level of cumulative exposure to LDL occurs) as well as the positional encoding of the standardized Lp(a) levels by the age of the subject at which the corresponding level of cumulative exposure to LDL occurred;
(6) cumulative exposure to Lp(a) at each increment of cumulative exposure to LDL (using the corresponding ages at which those cumulative exposure to Lp(a) levels occur to match or map the age at which the corresponding level of cumulative exposure to LDL occurs), and the positional encoding of cumulative exposure to Lp(a) by the age of the subject at which the corresponding level of cumulative exposure to LDL occurred;
(7) standardized weight at each increment of cumulative exposure to LDL based on the subject's predicted trajectory of weight (at the corresponding ages at which those weight levels are predicted to occur to match or map the age at which the corresponding level of cumulative exposure to LDL occurs) and the positional encoding of weight by the age of the subject at which the corresponding level of cumulative exposure to LDL occurred;
(8) standardized waist circumference at each increment of cumulative exposure to LDL based on the subject's predicted trajectory of waist circumference (at the corresponding ages at which those waist circumference levels are predicted to occur to match or map the age at which the corresponding level of cumulative exposure to LDL occurs) and the positional encoding of waist circumference by the age of the subject at which the corresponding level of cumulative exposure to LDL occurred;
(9) standardized HbA1c level at each increment of cumulative exposure to LDL based on the subject's predicted trajectory of HbA1c levels (at the corresponding ages at which those HbA1c levels are predicted to occur to match or map the age at which the corresponding level of cumulative exposure to LDL occurs) and the positional encoding of HbA1c level by the age of the subject at which the corresponding level of cumulative exposure to LDL occurred; and/or
(10) standardized age at each increment of cumulative exposure to LDL based on the age of the subject at which each increment of cumulative exposure to LDL occurs and the positional encoding of that age by the age of the subject at which the corresponding level of cumulative exposure to LDL occurred (as such, age is also being positionally encoded by age, which may help incorporate information indicating that the importance of the ‘age’ variable may not be the same at all ages).

It should be appreciated that while, in this example, each input feature vector may include each of the sets of values (1)-(10), any subset of these values may be used in other examples.

The set of input feature vectors for a subject, with each input feature vector determined for a respective level of the subject's cumulative LDL exposure, constitutes the input feature data for the subject that may then be passed on to and be processed by the first ML model to obtain, as output, the values indicative of the log hazard ratios for the risk of the subject having a cardiovascular event at the respective levels of cumulative LDL exposure. The set of input feature vectors may be organized into a matrix (with the vectors as rows or columns) or using any other suitable data structure(s), as aspects of the technology described herein are not limited in this respect.

As one example, a input feature vector may be determined for each level of cumulative exposure to LDL (the increment of survival), range from 50-350 Plaque Years of LDL in mmol/L. The 300 dense input vectors include the time invariant features (e.g., biological sex, family history), and the predicted levels of time varying exposures (e.g., SBP) at each level of cumulative exposure to LDL—and their positional encoding by age to preserve biological context. These features vectors may then be organized into a matrix (with the vectors as rows or columns) or using any other suitable data structure(s) and processed with a survival model (e.g., a causal survival deep neural network with piecemeal exponential models, as described herein) to determine log hazard ratios of the risk that the subject has a cardiovascular event at respective levels of cumulative LDL exposure.

Having discussed aspects of how input feature data is obtained at act 221, we now turn to a discussion of how that data may be processed by the first trained ML model to determine log hazard ratios of the risk that the subject has a cardiovascular event at respective levels of cumulative LDL exposure.

First, some aspects regarding the first ML model are described. In some embodiments, the first trained ML model may be a trained survival model with cumulative LDL exposure (instead of age) as the interval of follow-up. A survival model to predict risk relative to cumulative LDL levels, as described herein, is an important innovation in the cardiovascular space. The first trained ML model may be a survival deep neural network (DNN) model with cumulative LDL exposure as the interval of follow-up. Other types of survival models may be used as well, examples of which include a Cox proportional hazard model, a random forest of survival trees model, a gradient boosted machine (GBM) for survival trees model, a survival deep neural network (DNN) model; and/or an ensemble of any of these survival models. The key is that it is cumulative LDL exposure—rather than age—that serves as the interval of follow up in any implementation, whether that implementation is a neural network survival model or another type of survival model.

Moreover, the trained survival model may be of a change-point type, allowing for independent estimates of the hazard function in different intervals of follow-up. As one example, the first ML model may be a piecewise model (PM), for example, a piecewise exponential model (PEM). For example, the first ML model may be a survival DNN model (or other type of survival model examples of which are provided above) with PEM using cumulative LDL exposure as the interval of follow-up. In some embodiments, the trained survival DNN may be a fully-connected neural network having at least 3, at least 4, at least 5, or at least six fully connected layers; the parameters (e.g., weights) of the DNN may be different for each interval of follow-up (i.e., each interval of cumulative LDL exposure). In that sense, the first ML model (e.g., the trained survival DNN with PEM model) may be considered to be a set of DNN models having a common architecture (e.g., fully connected, ReLU non-linearities, at least 3, 4, 5, layers, etc.), but each trained to make a prediction over a particular interval of cumulative LDL exposure. Thus, the DNN models in the set have identical architecture but have different weights for different intervals of follow-up (though we refer to this as a single ML model here for clarity of exposition).

In some embodiments, the DNN with PEM model may have at least 10K, at least 25K, at least 50K, at least 100K, at least 500K, between 10K and 500K weight values for each interval of cumulative LDL exposure. In one illustrative example, the architecture of the DNN may comprise an input layer followed by 64, 128, 256, 128, 64, and 1-dimensional layers, all fully connected, with bias terms and ReLu non-linearities.

As such, in the PEM approach, the first ML model may be considered to include a separate survival model estimated at each increment of cumulative exposure to LDL (e.g., at each increasing plaque year measured in mmol/L). In turn, the output of the first ML model may be a log hazard ratio for the risk of the subject having a cardiovascular event at each level of cumulative exposure to LDL thus allowing for changes in exposures over time as plaque accumulates (i.e., time-varying inputs). Thus, in some examples, the first ML model may take in as input a set of input feature vectors (e.g., organized in a matrix) with one vector of input at each level of cumulative exposure to LDL (the piecemeal interval).

It should be noted that a survival model (e.g., DNN) with PEM, and with cumulative LDL exposure as the increment of follow up, is an important innovation and it is designed to predict how much an individual person's unique combination of exposures impacts the risk of having an acute cardiovascular event at all levels of accumulated plaque burden.

Cumulative exposure to LDL is used as the piecemeal interval of survival follow-up in this context because it provides an estimate of the size of the accumulated atherosclerotic plaque burden at any point in time, and because the size of the accumulated plaque burden, in turn, is the strongest determinant of the risk of having an acute atherosclerotic cardiovascular event.

However, the risk of having an acute cardiovascular event from a disrupted atherosclerotic plaque does not begin to increase in a measurable way until after the size of the accumulated plaque burden exceeds a specific threshold (measured in cumulative exposure to LDL). Furthermore, at all levels of plaque burden, the risk of having an acute cardiovascular depends not only on the size of the accumulated plaque burden but also on how much other exposures combine to impact: (i) the capacity of the artery to tolerate the accumulated plaque burden; (ii) the propensity for plaque disruption within the artery; and (iii) the inherited vulnerability to trapping atherosclerotic particles.

Therefore, the atherosclerotic plaque size threshold above which cardiovascular events begin to occur, and the risk of having an acute cardiovascular event at all levels of accumulated plaque burden varies substantially between individuals depending on their unique combination of other exposures.

This motivates using a survival model (e.g., a survival DNN) with PEM partitioned into increments of increasing cumulative exposure to LDL measured in Plaque Years of LDL (mmol/L)—which is used as an estimate of the size of the incrementally increasing plaque burden. As described herein, at each of these piecemeal increments of cumulative exposure to LDL, a fully-connected dense deep neural network (or another non-linear regression model) may be used to estimate the log hazard ratio of having a cardiovascular event at that size of accumulated plaque burden due to exposure to the combination of other features that impact the capacity of the artery to tolerate the accumulated plaque burden, conditional on surviving to that level of plaque burden without an event. The predicted log hazard ratios for the risks of having an atherosclerotic cardiovascular event at various levels of plaque burdens provides a direct estimate of the biological effect of how a subject's combination of exposures impacts the risk that the subject has a cardiovascular event at every level of accumulated plaque burden size. In this context, the cardiovascular event may be a fatal myocardial infarction (MI), a non-fatal MI, a fatal ischemic stroke, a non-fatal ischemic stroke, or a coronary revascularization.

Additional aspects of a survival deep neural network with piecewise exponential modeling are described next. As described herein, a Survival Deep Neural Network with a Piecewise Exponential Model (Survival DNN with PEM) is a neural network designed for survival analysis, which involves predicting the time until an event occurs (in this case, an atherosclerotic cardiovascular event including MI or stroke). Unlike standard regression tasks, survival analysis accounts for censored data, where the event of interest has not occurred for some individuals by the end of the observation period.

In a PEM, the follow-up is divided into intervals, and within each interval, the hazard rate (the rate at which the event occurs during that interval) is assumed to be constant (the exponential assumption). In some embodiments, the follow-up is divided into intervals of cumulative exposure to LDL to estimate the size of the accumulated plaque burden. As such, the survival DNN model is designed to predict the log hazard ratio (log HR) during each follow-up interval, i.e., at each level of size of the accumulated plaque burden measured in Plaque Years (mml/L) of cumulative exposure to LDL. The piecewise exponential model discretizes the time-to-event problem into intervals, allowing for flexible modeling of the hazard ratios over time, assuming only that the hazard rate is constant during each growing interval of plaque burden (interval of cumulative exposure to LDL), but may vary at different levels of plaque burden. This faithfully reflects the biology of atherosclerosis, where cardiovascular events begin to occur only after a specific accumulated plaque burden size accrues.

In some embodiments, the trained survival DNN with PEM model may be trained using participant data for at least 1 million (M) or 1.5 M participants enrolled in one or more prospective studies (e.g., IPD data from the UK Biobank (UKBB), the FinnGen Project, and the UK Clinical Practice Research Database (CPRD)), whereby for each of the at least 1 M or 1.5 M participants at least one LDL or SBP measurement (e.g., multiple LDL measurements and/or multiple SBP measurements) were available along with a recorded age or date at which a first episode of a fatal or non-fatal myocardial infarction (MI), fatal or non-fatal ischemic stroke, or coronary revascularization occurred, with censoring applied at time of last follow-up, death, or first cardiovascular event. The cardiovascular event may be a fatal myocardial infarction (MI), an episode of a non-fatal MI, a fatal ischemic stroke, an episode of a non-fatal ischemic stroke, or a coronary revascularization.

As one illustrative example, a survival DNN with PEM model was trained using individual participant data from 1,623,491 participants enrolled in one of three (3) long-term prospective biobank cohort studies for whom at least one LDL or SBP measurement was available, and for whom medical records were available recording age (date) at which the first episode of a fatal or non-fatal MI, fatal or non-fatal ischemic stroke, and coronary revascularization occurred. Participants were censored at time of last follow-up, death, or first atherosclerotic cardiovascular event. In addition, summary data from 2.6 million participants with LDL measurements and age (dates) of first atherosclerotic cardiovascular event were used for additional external validation experiments.

During training, the negative log-likelihood may be used as the loss function. In such an implementation, the Survival DNN with PEM predicts the log hazard ratio (log HRᵢ) for individual i based on their covariates (the input feature data for that individual); during each of K intervals (of cumulative exposure to LDL or equivalently at each measured size of the accumulated plaque burden).

In this case, the hazard rate λ_{k,i} for the ith participant in the K-th follow-up interval is given by:

$$\text{?} = \text{?} \cdot exp ( log ( H \text{?} ) ) = \text{?} \cdot H \text{?}$$

$$\text{?} \text{indicates text missing or illegible when filed}$$

where: λₖ is the baseline hazard rate for the kth time interval, log(HRᵢ) is the predicted log hazard ratio for individual I, and HRᵢ is the hazard ratio obtained by exponentiating the predicted log hazard ratio.

Calculations in the uncensored data case (event occurs) may be performed as follows. For an individual i who experiences the event at time tᵢ (in interval kᵢ), the log-likelihood contribution is based on: (1) the predicted hazard ratio HRᵢ multiplied by the baseline hazard λₖ; and (2) the cumulative hazard up to that time. Accordingly, the log-likelihood for uncensored data is given by:

$$log \text{?} = log ( \text{?} \cdot H \text{?} ) - \text{?} \cdot H \text{?} \cdot Δ {t}_{k}$$

$$\text{?} \text{indicates text missing or illegible when filed}$$

where: HRᵢ=exp(log(HRᵢ)) is the predicted hazard ratio for individual i, λ_{k,i} is the baseline hazard in interval Kᵢ, Δtₖ is the length of the kth interval, the first term in the summation represents the likelihood of the event occurring at time tᵢ, and the second term in the summation represents the cumulative hazard up to tᵢ, derived from the hazard in each interval.

Calculations in the censored data case (event does not occur) may be performed as follows. For censored individuals, the event has not occurred, so the likelihood of surviving beyond the censoring time is calculated. The log-likelihood contribution is based on the survival probability, which is related to the cumulative hazard according to:

$$log \text{?} = - \text{?} \cdot H \text{?} \cdot Δ \text{?}$$

$$\text{?} \text{indicates text missing or illegible when filed}$$

This represents the survival probability up to the censoring time, calculated using the cumulative hazard.

Putting the uncensored and censored components together provides the total log-likelihood for the entire dataset, which may be obtained as the sum of the log-likelihood contributions from all individuals, combining both uncensored and censored cases:

$$Log - Likelihood = \text{?} log \text{?}$$

$$\text{?} \text{indicates text missing or illegible when filed}$$

where N is the number of participants. The loss function is the negative log-likelihood given by:

$$Loss = \vert - \text{?} log \text{?}$$

$$\text{?} \text{indicates text missing or illegible when filed}$$

Once the negative log-likelihood loss is computed, backpropagation may be used to calculate the gradients of the loss with respect to the (e.g., DNN) model parameters (weights, biases, etc.). These gradients are then used to update the model parameters using an optimization algorithm such as stochastic gradient descent (SGD) or Adam.

In some embodiments, a survival deep neural network with PEM may be implemented using several libraries in PyTorch that support time-to-event data modelling, including:
1. torch.nn—Core PyTorch library for neural network layers like nn.Linear, nn.ReLU, and other activation layers for building the model architecture.
2. pycox—A package built on PyTorch specifically for survival analysis. It provides implementations of common survival loss functions (e.g., partial log-likelihood) and methods like PEM, enabling efficient training on time-to-event data.
3. lifelines—A survival analysis library (not based on PyTorch) that provides some additional methods for data handling, that may be useful for preprocessing survival data.

As one example, therefore, to learn the biology of how atherosclerosis develops and how atherosclerotic clinical events occur as a complication of the disruption of the underlying plaque with the resulting formation of a thrombus to seal the disrupted plaque, a causal survival DNN with PEM uses cumulative exposure to LDL as the increment of ‘survival’ or follow-up (instead of age or time) to estimate the size of the accumulated plaque burden. At each level (piecemeal) of cumulative exposure to LDL (plaque burden), a separate fully-connected survival DNN is generated to predict the natural log Hazard ratio (lnHR) of having an ASCVD event at that level of accumulated plaque burden caused by the combined effect of all other exposures at that point in time for the person under consideration (thus capturing the time-varying changes in these other exposures that impact the capacity of the artery to tolerate the accumulated plaque burden). The output of the causal survival DNN with PEM is a vector composed of the lnHR for the risk of having an ASCVD event at each level of cumulative exposure to LDL (or accumulated plaque burden) measured in Plaque Years of LDL in mmol/L caused by the combined effect of the changing levels of all other exposures for the person being evaluated.

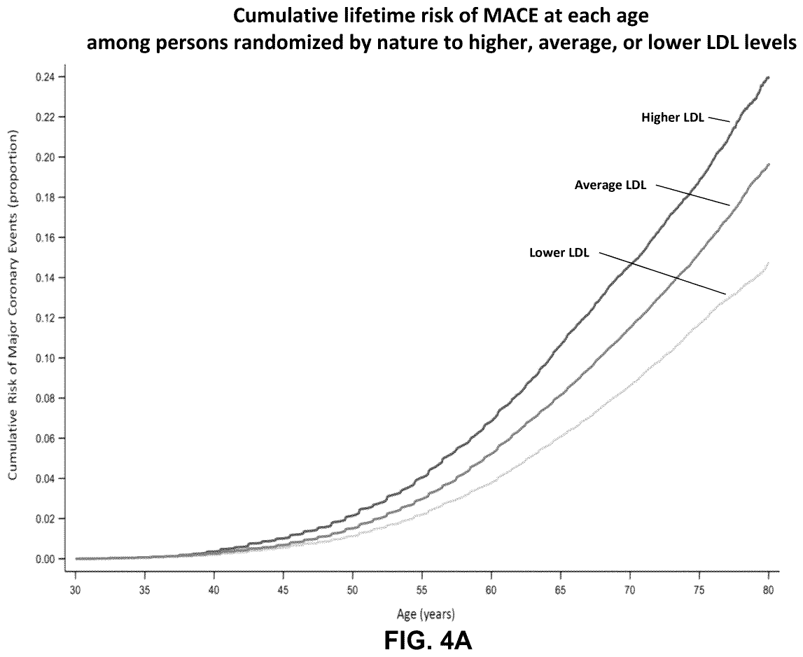

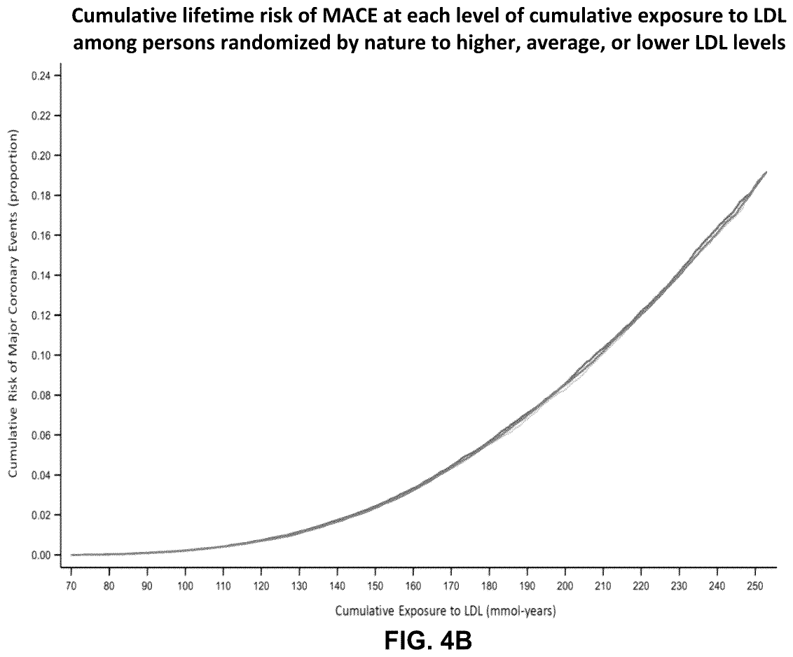

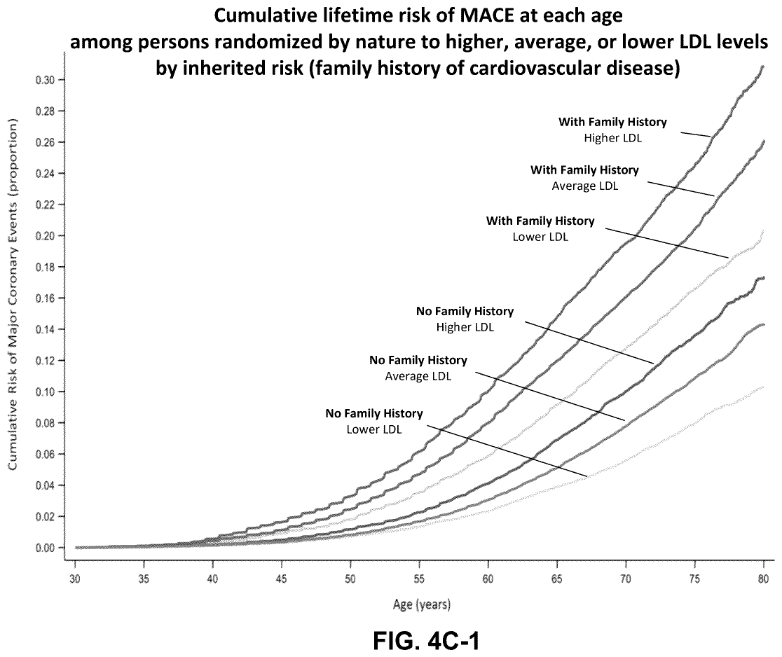

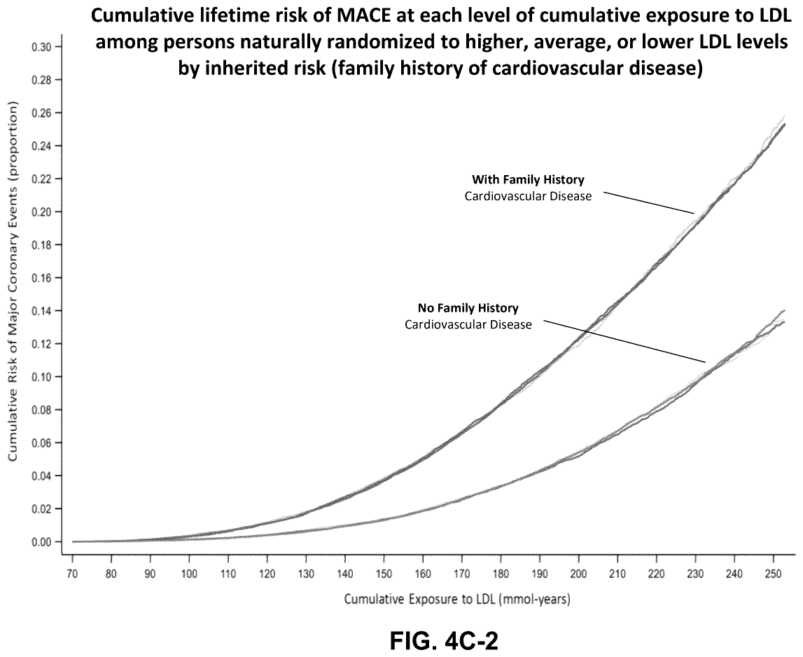

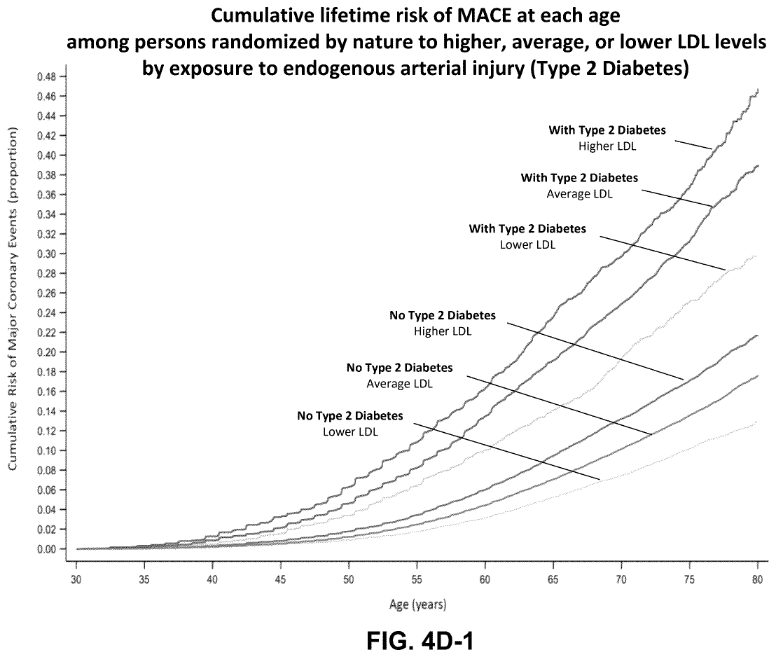

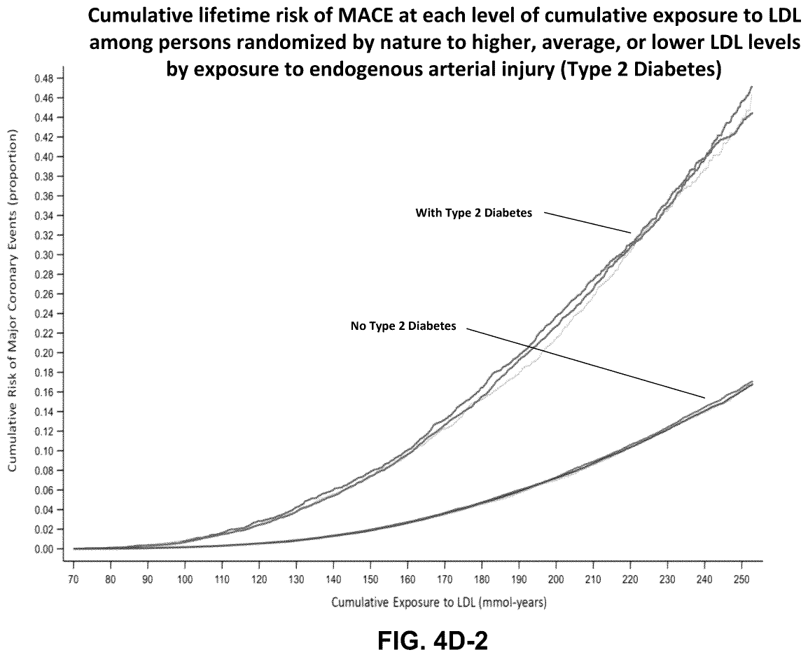

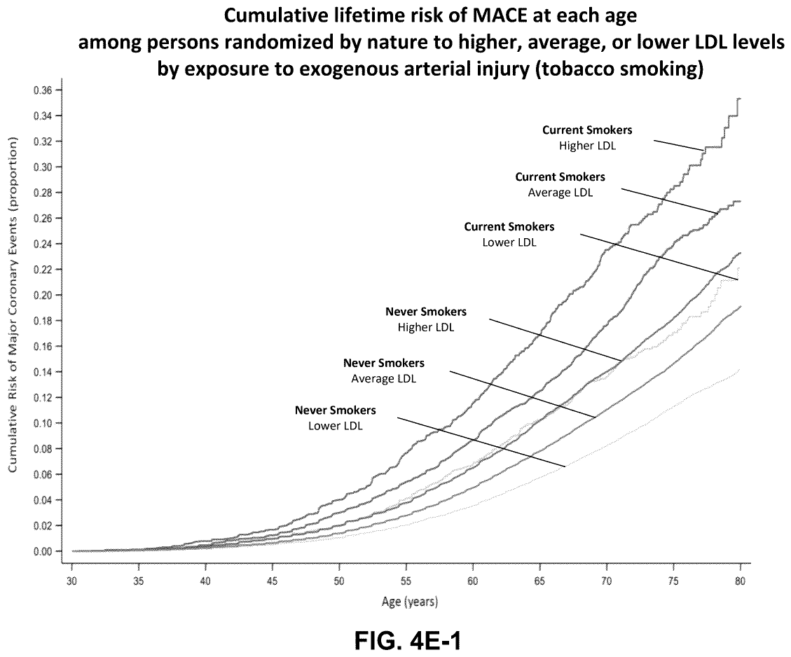

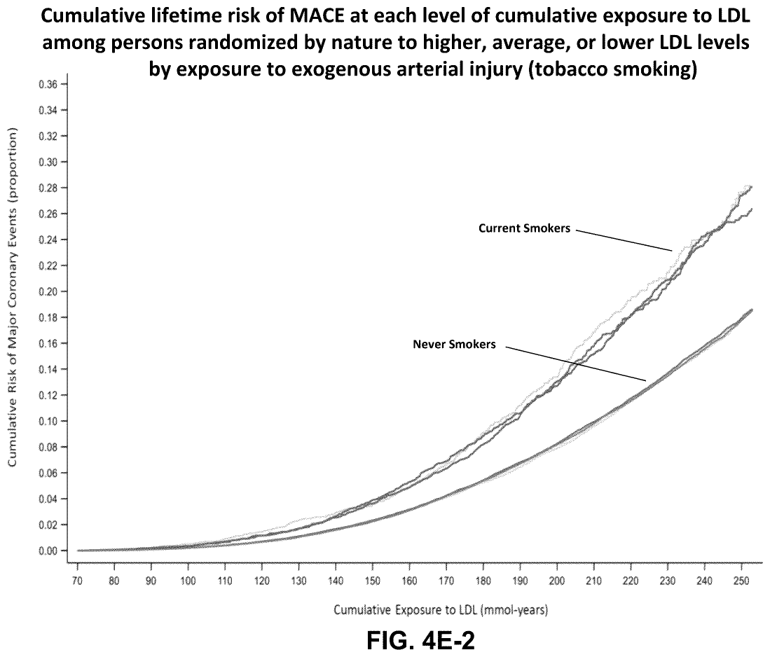

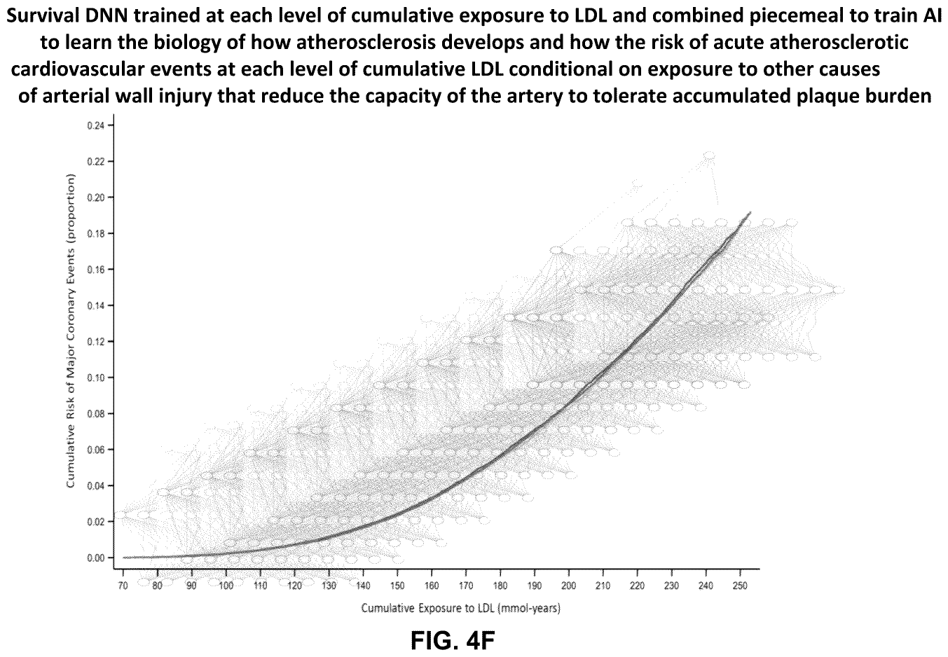

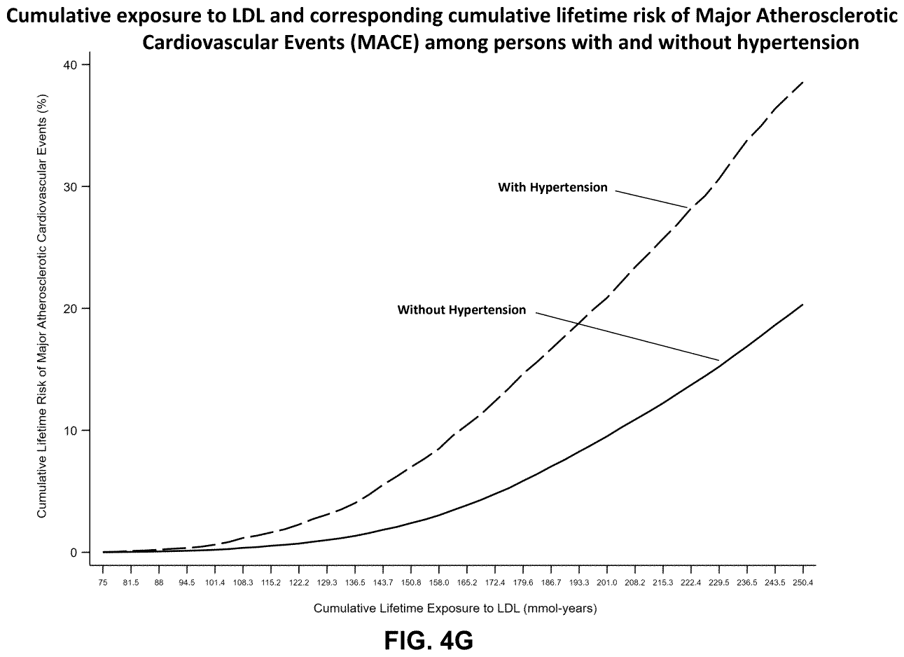

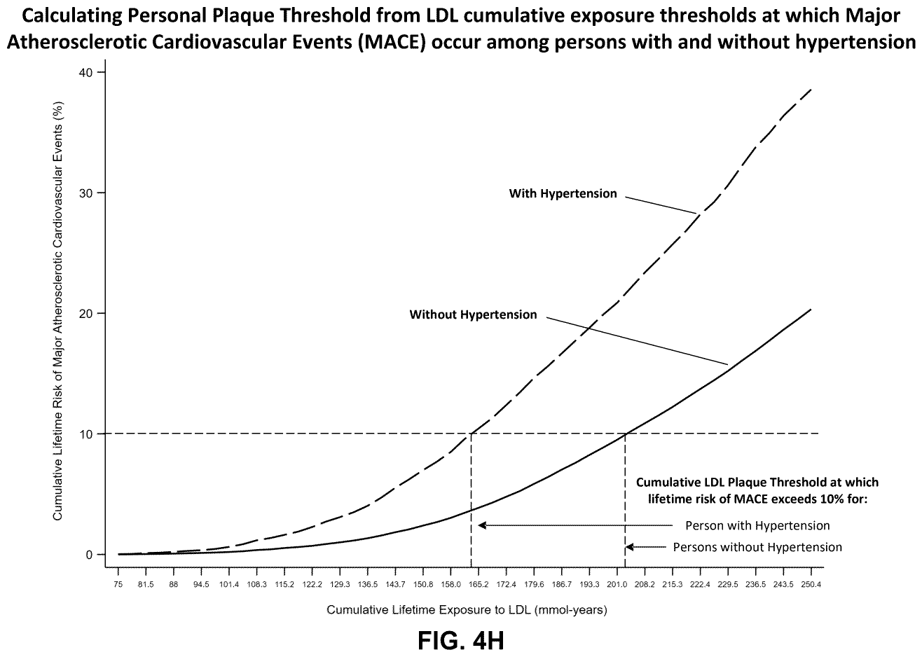

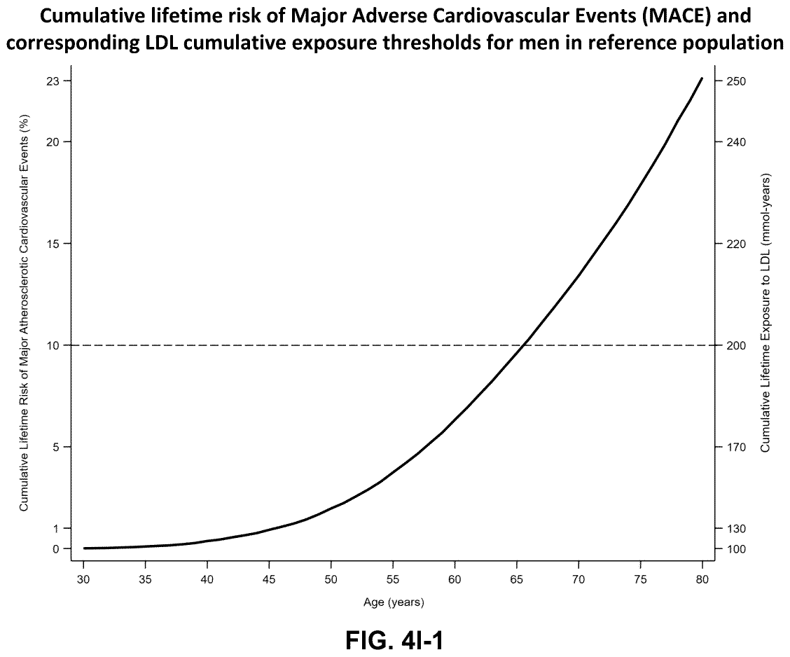

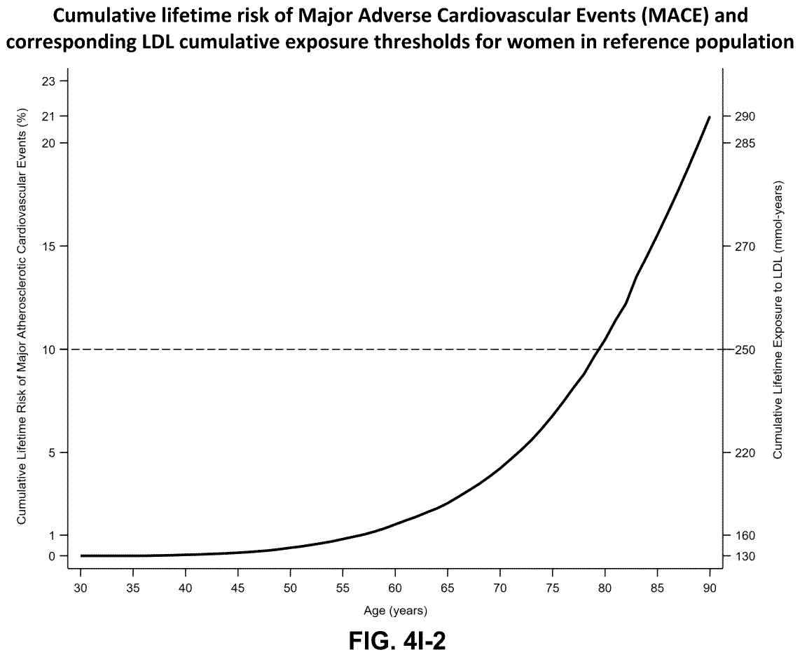

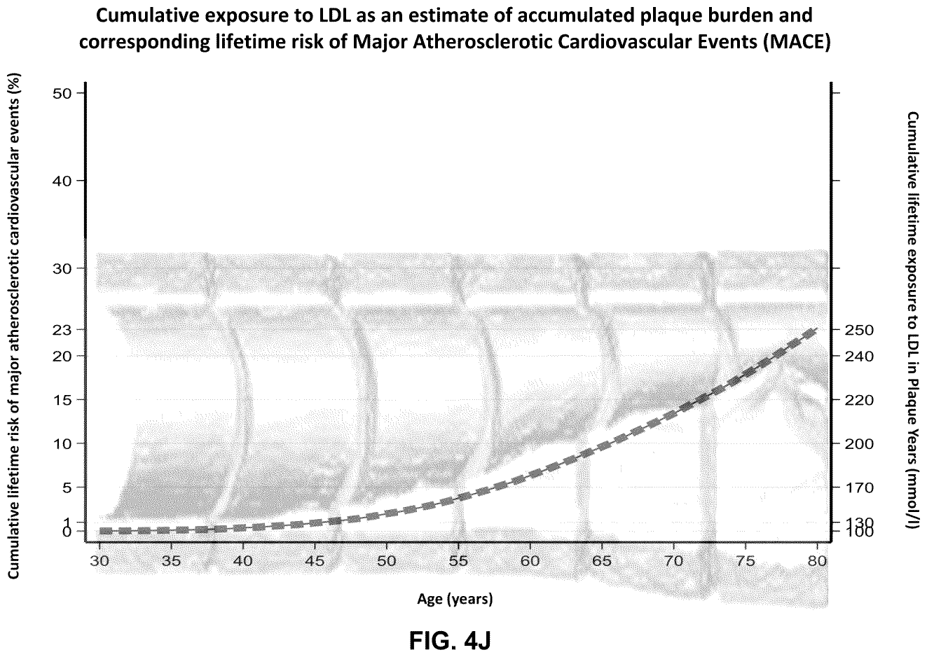

An illustration is provided in FIG. 4F which shows a survival DNN trained at each level of cumulative exposure to LDL and combined piecemeal to train AI to learn the biology of how atherosclerosis develops and how the risk of atherosclerotic cardiovascular events at each level of cumulative LDL conditional on exposure to other causes of arterial wall injury that reduce the capacity of the artery to tolerate accumulated plaque burden. Though it should be appreciated that, in other embodiments, a different model (instead of a DNN) may be used together with PEM to predict the log hazard ratio of having an ASCVD at each level of accumulated plaque burden.

FIGS. 4A, 4B, 4C-1, 4C-2, 4D-1, 4D-2, and 4E-1 and 4E-2 illustrate striking evidence justifying the use of increments of cumulative exposure to LDL as an estimate of increasing plaque burden size to train the algorithms to learn the biology of how atherosclerotic cardiovascular disease develops. In particular, evidence may be derived from naturally randomized causal evidence from Mendelian randomization. As shown in FIG. 4A, using genetic variants associated with LDL (and apoB) as instrumental variables, participants randomized by nature to higher LDL have higher plasma LDL, higher cumulative exposure to LDL and higher absolute cumulative risk of a major adverse cardiac event (MACE), such as an ASCVD event, at all ages as compared to persons randomized by nature to lower LDL.

However, when these three randomized groups are compared using cumulative exposure to LDL as the increment of survival follow-up—they have the same absolute cumulative lifetime risk of MACE (e.g., ASCVD events) at all levels of cumulative exposure to LDL regardless of the age at which the plaque size accrued, as shown in FIG. 4B. This demonstrates that the risk of atherosclerotic cardiovascular disease events is largely determined by the accumulated plaque size regardless of how fast or when that plaque size accrues—when all other exposures are the same.

However, the risk of having an ASCVD event at the same level of cumulative exposure to LDL varies based on other exposures that impact the capacity of the artery to tolerate the accumulated plaque burden—including the impact of family history—for example, inherited predisposition to trapping LDL (apoB) particles or propensity for plaque disruption, as shown in FIGS. 4C-1 and 4C-2. FIG. 4C-1 shows the cumulative risk of MACE at each age among persons randomized by nature to higher, average, or lower LDL levels by inherited risk (family history of cardiovascular disease). FIG. 4C-2 shows the cumulative lifetime risk of MACE at each level of cumulative exposure to LDL among persons naturally randomized to higher, average, or lower LDL levels by inherited risk (family history of cardiovascular disease).

The risk also varies due to other causes of endogenous injury to the artery wall—including T2D from chronically elevated HbA1c (glucose) that can cause negative remodeling leading to small-caliber, diffusely narrowed arteries, as shown in FIGS. 4D-1 and 4D-2. FIG. 4D-1 shows the cumulative lifetime risk of MACE at each age among persons randomized by nature to higher, average, or lower LDL levels by exposure to endogenous arterial injury (Type 2 Diabetes). FIG. 4D-2 shows the cumulative lifetime risk of MACE at each level of cumulative exposure to LDL among persons randomized by nature to higher, average, or lower LDL levels by exposure to endogenous arterial injury (Type 2 Diabetes).

The risk also varies due to other causes of exogenous injury to the artery wall—including tobacco smoking which increases the risk of having an ASCVD at all levels of cumulative exposure to LDL and corresponding size of accumulated plaque burden by increasing the probability of plaque disruption, as shown in FIGS. 4E-1 and 4E-2. FIG. 4E-1 shows cumulative lifetime risk of MACE at each age among persons randomized by nature to higher, average, or lower LDL levels by exposure to exogenous arterial injury (tobacco smoking). FIG. 4E-2 shows cumulative lifetime risk of MACE at each level of cumulative exposure to LDL among persons randomized by nature to higher, average, or lower LDL levels by exposure to exogenous arterial injury (tobacco smoking).

This evidence establishes that the survival models described herein, including the survival DNN with PEM, are “causal” models because this evidence and justification for using cumulative exposure to LDL as the piecemeal increment of survival to learn the biology of how atherosclerosis develops is based on the randomized—and therefore ‘causal’—evidence outlined above.

After act 222 is performed, the process of FIG. 2C proceeds to act 223, which involves estimating, using the values indicative of the log hazard ratios (obtained at act 222), absolute instantaneous rates, cumulative hazard rates, and event rates, of the subject having a cardiovascular event at the respective levels of cumulative LDL exposure.

In some embodiments, estimating, using the values indicative of the log hazard ratios, the cumulative lifetime risks of the subject having a cardiovascular event at the respective levels of cumulative LDL exposure comprises multiple steps, including:
(a) generating a data structure encoding a lifetable, the data structure comprising values indicating absolute instantaneous hazard rates of experiencing a cardiovascular event at the respective levels of cumulative LDL exposure, at average levels of all other exposures, in a reference population;
(b) determining the absolute instantaneous hazard rates of the subject having a cardiovascular event at the respective levels of cumulative LDL exposure by multiplying: (i) the values, from (a), indicating absolute instantaneous hazard rates of experiencing a cardiovascular event at the respective levels of cumulative LDL exposure, at average levels of all other exposures, in the reference population; and (ii) the values indicative of the log hazard ratios for risk of the subject having a cardiovascular event at the respective levels of cumulative LDL exposure;
(c) determining the cumulative hazard rates of the subject having a cardiovascular event at the respective levels of cumulative LDL using the absolute instantaneous hazard rates, determined at (b); and
(d) determining the cumulative event rates of the subject having a cardiovascular event at the respective levels of cumulative LDL using the absolute instantaneous hazard rates, determined at (b).

Aspects of determining instantaneous hazard rates at the respective levels of cumulative LDL exposure, at average levels of all other exposures, in a reference population are described in more detail in Example 8.

These computations, in turn, allow for identifying a so-called “personal plaque burden” threshold for the subject. The personal plaque burden threshold may indicate a level of cumulative plaque burden at which cardiovascular events are predicted to begin to occur for the subject, and can be identified by using the cumulative event rates of the subject having a cardiovascular event at the respective levels of cumulative LDL, which are determined at act 223.

Relatedly, the results of the computations at act 223, may be used to identify plaque levels at which cumulative lifetime risk is below a given threshold (e.g., 10%). For example, in some embodiments, act 223 may further include identifying, for a specified level of cumulative lifetime risk, a level of cumulative plaque burden at which risk of occurrence cardiovascular events for the subject is less than the specified level of cumulative lifetime risk.

Aspects of the computations performed at act 223 are described further below. These computations involve using the log hazard ratios determined at act 222 to estimate the absolute risk of having an acute cardiovascular event at all levels of accumulated plaque burden depending on a person's combination of other exposures. In some embodiments, this may be accomplished as follows.

First, a lifetable may be constructed of the absolute instantaneous hazard of experiencing an atherosclerotic cardiovascular event at all levels of cumulative exposure to LDL (plaque burden)—at the average levels of all other exposures—in a reference population. These data are derived from naturally randomized experiments using genetic instrumental variable LDL scores. These studies demonstrate that persons randomized by nature to higher lifelong exposure to LDL have a higher measured LDL level, a higher corresponding cumulative exposure to LDL, and a higher absolute cumulative risk of having an atherosclerotic cardiovascular event at all ages as compared to persons randomized by nature to lower LDL.

However, when using cumulative exposure to LDL as the increment of follow-up from the time of randomization, each of these groups has the same absolute cumulative risk of atherosclerotic cardiovascular events at the same cumulative exposure to LDL (accumulated plaque size) regardless of the age at which the cumulative exposure to LDL was achieved (as shown in FIGS. 4A-4B, for example). This naturally randomized biological evidence demonstrates that the absolute risk of atherosclerotic cardiovascular events is determined by the size of the accumulated plaque burden (measured in cumulative exposure to LDL), regardless of how rapidly the plaque burden is accumulating when all other exposures are equal. (Of note, the results of these naturally randomized trials also provides the motivation, evidence, and justification (as recognized by the inventor) for using cumulative exposure to LDL as the increment of follow-up in the causal survival DNN with PEM described above.).

Next, the absolute instantaneous hazard rate of having an atherosclerotic event for an individual person at each level of cumulative exposure to LDL (accumulated plaque burden) may be estimated by: multiplying the instantaneous hazard rate in the reference group, by the log hazard ratio derived from the first ML model (e.g., causal survival model with PEM, for example a DNN with PEM model) estimating how much a person's unique combination of exposures impacts the risk of having a cardiovascular event at each level of cumulative exposure to LDL (accumulated plaque burden).

Next, the absolute instantaneous hazard rates may be summed to compute the cumulative hazard rates and absolute cumulative event rates at each level of cumulative LDL exposure (plaque burden) for the person under evaluation, conditional on surviving without an event up to that level of plaque burden.

Next, the personal plaque threshold at which atherosclerotic cardiovascular events are predicted to occur for the person under evaluation may be determined based on the predicted absolute cumulative event rates for respective levels of cumulative LDL exposure. The level of cumulative exposure to LDL at which the size of the accumulated atherosclerotic plaque burden exceeds the threshold size at which atherosclerotic cardiovascular events begin to occur for a specific person may be considered their ‘personal plaque threshold.’ This may be visualized by plotting the predicted absolute cumulative event rates by cumulative exposure to LDL—the plot identifies the cumulative exposure to LDL corresponding to the size of the accumulated plaque burden at which atherosclerotic cardiovascular events are predicted to begin to occur for the person under study. That identified level of cumulative LDL exposure may be considered their ‘personal plaque threshold.’ It follows that this plot can also be used to identify the personal plaque threshold for any level of cumulative lifetime risk of atherosclerotic cardiovascular events by identifying the cumulative exposure to LDL and the corresponding size of the accumulated plaque burden at the any specified level of cumulative lifetime risk. It should be noted that the values for the personal plaque threshold for any cumulative rate of cardiovascular events (risk) can also be obtained directly from the lifetables described herein.

Overall, the cumulative exposure to LDL corresponding to a specific cumulative lifetime risk of cardiovascular events (all these quantities are now available as a result of computations performed in acts 222 and 223) may be used as a therapeutic target to personalize the prevention of cardiovascular events by providing guidance about how much a person's LDL level is to be reduced, in order to: slow their rate of plaque progression and/or to keep their cumulative exposure to LDL and the corresponding size of their accumulated plaque burden below the threshold required to achieve a desired level of cumulative lifetime risk. This, therefore, provides the basis for personalizing prevention by titrating each person's LDL, SBP, and exposure to other modifiable causes of disease to slow the rate of disease progression enough to keep each person below their selected personal plaque threshold (and thus keep their cumulative lifetime risk of having an atherosclerotic cardiovascular event below the selected personal plaque threshold target.)

An illustrative example of the results of calculations performed at act 223 is shown in FIG. 4G. As described herein, a subject's remaining cumulative lifetime risk of cardiovascular (e.g., atherosclerotic cardiovascular) events at all levels of cumulative exposure to LDL (accumulated plaque size) caused by the combined effect of their levels of all other exposures at the time that level of cumulative exposure to LDL accrues may be estimated by: a) first constructing a lifetable of the instantaneous hazard of having a cardiovascular event at each increment of cumulative exposure to LDL derived from the naturally randomized evidence (for persons with average levels of all other exposures in the reference population); and then, b) multiplying the instantaneous hazard rate at each increment of cumulative exposure to LDL by the exponentiated form of the corresponding log hazard ratio (i.e., the hazard ratio) for a cardiovascular event caused by the subject's combination of all other exposures at that level of cumulative exposure to LDL derived from the output of the first ML model (e.g., causal survival DNN with PEM). The cumulative hazard rates and cumulative event rates may then be calculated using standard lifetable methods and the cumulative lifetime risk can be plotted as shown in FIG. 4G (shown for a subject with and without hypertension). FIG. 4G shows the cumulative exposure to LDL and corresponding lifetime risk of MACE among persons with and without hypertension.

FIG. 4H illustrates calculating personal plaque thresholds from LDL cumulative exposure thresholds at which lifetime risk of MACE occur among persons with and without hypertension. As shown in FIG. 4H, a specific threshold for a person with and without hypertension is calculated by identifying the cumulative exposure to LDL above which lifetime risk of MACE exceeds the person's lifetime risk goal (e.g., 10% as shown in FIG. 4H).

Returning back to FIG. 2C, after act 223 is performed, the process of FIG. 2C proceeds to act 224, which involves estimating, using the cumulative LDL exposure trajectory for the subject, absolute instantaneous rates (determined at 223), cumulative hazard rates (determined at 223), and event rates (determined at 223), of the subject having a cardiovascular event at the respective ones of the multiple time intervals (e.g., years).

In some embodiments, this is performed by determining ages at which the respective levels of cumulative LDL exposure occur for the subject by using the cumulative LDL exposure trajectory for the subject (obtained, e.g., by using the first ML model for predicting the person's trajectory of LDL levels over time). This enables understanding the cumulative lifetime risk of cardiovascular events as a function of age for the subject. For example, as shown in FIGS. 4I-1 and 4I-2, the lifetable of cumulative lifetime risk of cardiovascular events may be plotted against the subject's age. FIG. 4I-1 shows the cumulative lifetime risk of MACE and corresponding LDL cumulative exposure thresholds for men in a reference population. FIG. 4I-2 shows the cumulative lifetime risk of MACE and corresponding LDL cumulative exposure thresholds for women in a reference population.

Aspects of mapping “risk by cumulative LDL exposure” to “risk by age” are now further described. In particular, mapping the age at which each corresponding level of cumulative exposure to LDL occurs for a subject may be performed by using the output from the LDL bi-LSTM (or any other type of model that predicts the subject's trajectory of LDL levels over time). The resulting lifetable of risk by age provides the predicted absolute instantaneous hazard rates, cumulative hazard rates, and cumulative event rates at each age for the subject based on the size of the accumulated plaque burden (cumulative exposure to LDL), their rate of plaque progression, and their combination of other exposures.

The creation of a lifetable of absolute hazards by age enables: communicating risk in a clinically useful way, computing updated predictions of remaining lifetime risk over time as a person survives previous risk intervals without experiencing an event, and computing the benefit of reducing exposure to the modifiable causes of disease by both magnitude, duration, and/or timing of intervention. In addition, the lifetable of absolute hazards by age allow for the cataloging and quantification of the legacy benefit derived from earlier interventions to reduce exposure to the modifiable causes of disease. This prevents forgetting of the benefit of earlier interventions and enables accurate prediction of remaining lifetime risk (of cardiovascular events) and benefit (of particular interventions or sequences of interventions), as well as guiding the appropriate selection of optimal sequence of interventions required to achieve the desired therapeutic goals, and accounting for the accruing benefit of earlier interventions to reduce exposure to the modifiable causes of disease.

In summary, at the completion of the process shown in FIG. 2C, the results of the survival analysis (e.g., using a survival DNN with PEM) using cumulative exposure to LDL as the interval of follow-up (as described with respect to act 222), combined with the lifetable analyses informed by the results of the naturally randomized trials of cumulative exposure to LDL (as described with respect to act 223) provide the predicted risk of developing an atherosclerotic cardiovascular event based on the size of a person's accumulated plaque burden, and their combination of other exposures that impact the capacity of the artery to tolerate the accumulated plaque burden, at all future time points up to any age of the subject (with the mapping from cumulative LDL levels to age performed as described with respect to act 224).

FIG. 4J provides an illustration of a subject's cumulative lifetime risk of having a cardiovascular event at any point in time as a function of the size of the accumulated plaque and the combined effect of other causes of arterial wall injury that determine the capacity of the artery to tolerate the accumulated plaque burden.

In addition, in some embodiments, the predictions the SBP level trajectory and the rate of rise of SBP (e.g., computed as described with reference to FIG. 2B) may be used to provide predictions for the risk of developing hypertension and the age at which hypertension is likely to occur.

### Determining Benefit of Administering Therapeutic Intervention(s) to Reduce Risk That Subject Develops Cardiovascular Disease

As shown in FIG. 2A, process 200 includes act 230 that involves determining, using the multiple measures of risk (obtained at act 220) that the subject develops cardiovascular disease and at least one second trained ML model (e.g., at least one trained deep neural network), the benefit of administering to the subject one or more therapeutic interventions designed to reduce risk of cardiovascular disease by targeting one or more modifiable causes of the cardiovascular disease (e.g., LDL levels, SBP levels). For example, the at least one second trained ML model may include one trained ML model (e.g., a first c-DNN-ODE) to estimate the benefit of administering to the subject one or more interventions for lowering LDL and another trained ML model (e.g., a second c-DNN-ODE) to estimate the benefit of administering to the subject one or more interventions for lowering SBP. In some embodiments, the at least one second trained ML model may include yet another trained ML model (e.g., a third c-DNN-ODE) to estimate the benefit of administering to the subject an intervention for lowering both LDL and SBP.

In some embodiments, this may be performed in accordance with the illustrative process shown in FIG. 2D. As shown in FIG. 2D, act 230 may involve: (1) generating, at act 231, one or more therapeutic intervention sequences; (2) filtering, at act 232, the generated sequences using a domain expert clinical translation heuristic; and (3) determining, at act 233, the benefit of the filtered intervention sequence(s) using the at least one second trained ML model.

Before further describing each of the acts 231-233, which involve evaluating benefit of multiple sequences of interventions, we begin by describing how the benefit of targeting one or more modifiable causes of disease may be quantified and estimated. We will then return to a discussion of the acts 231-233.

### Determining Benefit of Lowering LDL or SBP for Subject

In some embodiments, the benefit of targeting one or more modifiable causes of disease for a subject may be quantified and estimated. In some embodiments, the benefit of lowering LDL or SBP for a subject may be quantified and estimated, for example, by determining the expected proportional and/or absolute reduction in risk of cardiovascular events for the subject if the LDL or SBP were lowered (e.g., responsive to a particular therapeutic intervention sequence of one or more therapeutic interventions designed to target LDL and/or SBP levels).

In some embodiments, determining the expected proportional and/or absolute reduction in risk involves two distinct steps. The first step involves obtaining estimates of the magnitude of the hazard ratio per unit lower LDL, or per unit lower SBP, during each year of life (or other interval of follow-up) among participants randomized by nature to lifelong exposure to lower LDL or lower SBP, respectively. This may be performed using an innovative type of ML model described herein, which in some embodiments may be a causal DNN for ordinary differential equations (c-DNN-ODE) model. The second step involves combining the estimates provided by the c-DNN-ODE model(s) (which relate to benefits of lowering LDL and/or SBP in a population) with the predicted instantaneous hazard rates that the subject experiences a cardiovascular event in each interval of follow up from the subject's current age to age 80 or other upper threshold age (these hazard rates relate to the subject specifically and have been determined as already described with respect to FIG. 2C).

With respect to the first step, in some embodiments, at least one machine learning model (e.g., the at least one second trained ML model referenced in FIGS. 2A and 2D) is trained on causal data from two sources: (1) randomized trials of LDL and SBP lowering therapies, respectively and (2) Mendelian randomization studies with individual participant follow-up data evaluating genetic variants associated with lower LDL (apoB) or SBP, respectively. Each such ML model may be a deep neural network model and, for example, may be a causal DNN ODE (c-DNN-ODE) model as described herein. Thus, in some embodiments, the at least one second ML model may include a c-DNN-ODE model (or other type of model) for estimating the benefit of lowering LDL and another c-DNN-ODE model (or other type of model) for estimating the benefit of lowering SBP. Yet another c-DNN-ODE model (or other type of model) may be used for estimating the benefit lowering LDL and SBP.

In some embodiments, a c-DNN-ODE model includes a deep neural network to estimate a function describing a smooth curve that can be used to compute the instantaneous hazard ratio for a standard increment of lowering a biomarker associated with a modifiable cause of disease that causes accumulating irreversible structural injury over time, for example the instantaneous hazard ratio of lowering LDL (1 mg/dL) or lowering SBP (1 mmHg), during each interval of treatment over time (without the need to specify the closed form of the function). The DNN is further combined an ordinary differential equation solver to estimate how much the benefit of lowering LDL or SBP changes during infinitesimally small increments of increasing duration of treatment.

In this way, the c-DNN-ODE model transforms the idea of passing data through a fixed sequence of layers in a neural network into a continuous process of transformations between layers where data evolves smoothly over time (analogous to a system governed by a differential equation), and therefore enables quantifying how much the magnitude of the instantaneous hazard ratio for an increment of lower LDL or SBP changes over time—and this dynamically changing hazard ratio provides a quantitative estimate of the expected proportional clinical benefit of sustained LDL or SBP lowering over time.

The intuition behind the c-DNN-ODE model (or other model that can be trained on similar data) is that the results of randomized trials can be reanalyzed by constructing a lifetable to recover the instantaneous hazard rates for the desired outcome event (or composite event) in either treatment arm at every interval of follow-up. The recovery of the instantaneous hazard rates permits the computation of the corresponding instantaneous hazard ratio within each increment of follow-up. In turn, combining the observed instantaneous hazard ratios during each interval of follow-up with the corresponding absolute difference in LDL or SBP between the randomized treatment arms observed during the same increment of follow-up permits computation of the instantaneous hazard ratio per unit lower LDL, or per unit lower SBP, during each increment of follow-up during the trial.

Combining the estimated hazard ratio per unit lower LDL, or per unit lower SBP, during each increment of follow-up from multiple randomized trials in an inverse variance-weighted meta-analysis provides a robust estimate of the magnitude of the instantaneous hazard ratio per unit lower LDL, or per unit lower SBP, during each treatment interval. Moreover, plotting the magnitude of the summary hazard ratio during each interval of follow-up provides a visual and quantitative assessment of how the hazard ratio changes over time—thus providing a direct estimate of how much the benefit of lowering LDL or SBP increases over time.

Extending this same intuition to the analysis of nature's randomized trials using individual participant data from Mendelian randomization studies evaluating genetic variants associated with lower LDL (apoB) or SBP, respectively, permits the construction of lifetables containing the instantaneous hazards and instantaneous hazard ratios during each year of life (or other interval of follow-up) among participants randomized by nature to lifelong exposure to lower LDL or lower SBP, respectively, beginning at birth until age 80 years of age. In particular, combining the observed instantaneous hazard ratios during each interval of follow-up with the corresponding absolute difference in LDL or SBP observed between the groups randomized by nature to higher or lower LDL or SBP, respectively, during the same increment of follow-up, permits computation of the instantaneous hazard ratio per unit lower LDL, or per unit lower SBP, during each year of life. Moreover, combining the results of numerous different Mendelian randomization studies evaluating hundreds of different genetic variants associated with lower LDL or lower SBP, respectively, in an inverse variance-weighted meta-analysis provides a more robust estimate of the magnitude of the hazard ratio per unit lower LDL, or per unit lower SBP, during each year of life (or other interval of follow-up) among participants randomized by nature to lifelong exposure to lower LDL or lower SBP, respectively.

In some embodiments, the c-DNN-ODE takes as input the estimated instantaneous hazard ratios ordered by duration of follow-up from the analyses of both the randomized trials and Mendelian randomization studies; and processes these data to differentiate a continuous series of infinitesimally small increments of increasing follow-up time to provide a continuous estimate of the magnitude of the instantaneous hazard ratio for a one unit increment of lower LDL, or lower SBP, for any duration of intervention.

Finally, the time-averaged hazard ratio for a one unit lower LDL, or SBP, for all durations of sustained intervention of follow-up may be computed by iteratively calculating the weighted average of the c-DNN-ODE estimated instantaneous hazards in sequence from the start of therapy.

Accordingly, in some embodiments, the c-DNN-ODE model may be used to determine a vector of the ‘time-averaged’ log hazard ratio for a standardized decrement of LDL (1 mmol/L or other suitable unit) or SBP (10 mmHg or other suitable unit) (or both) during the corresponding duration of intervention extending from 1 month to 80 years.

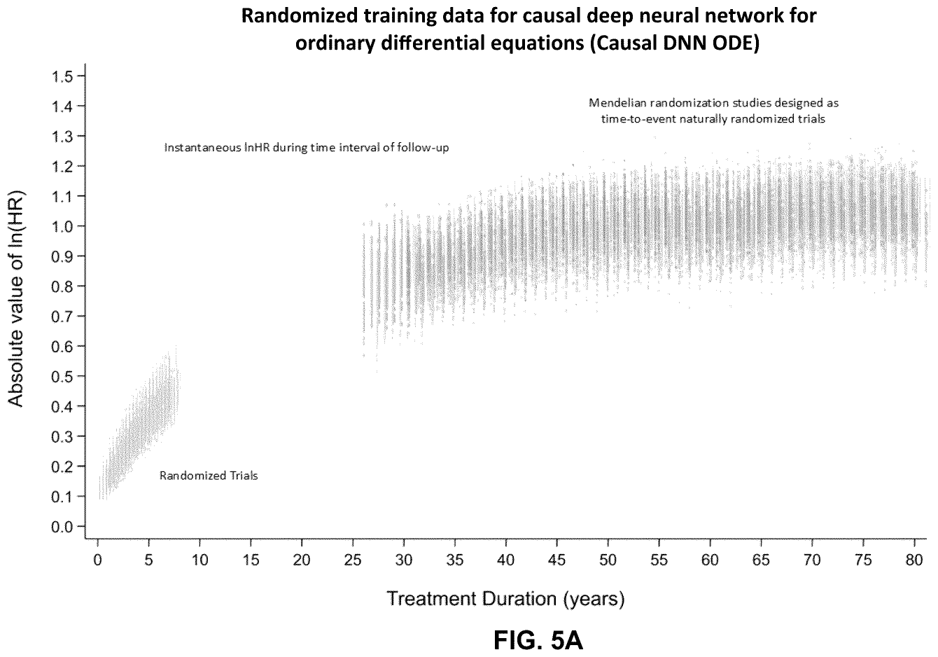

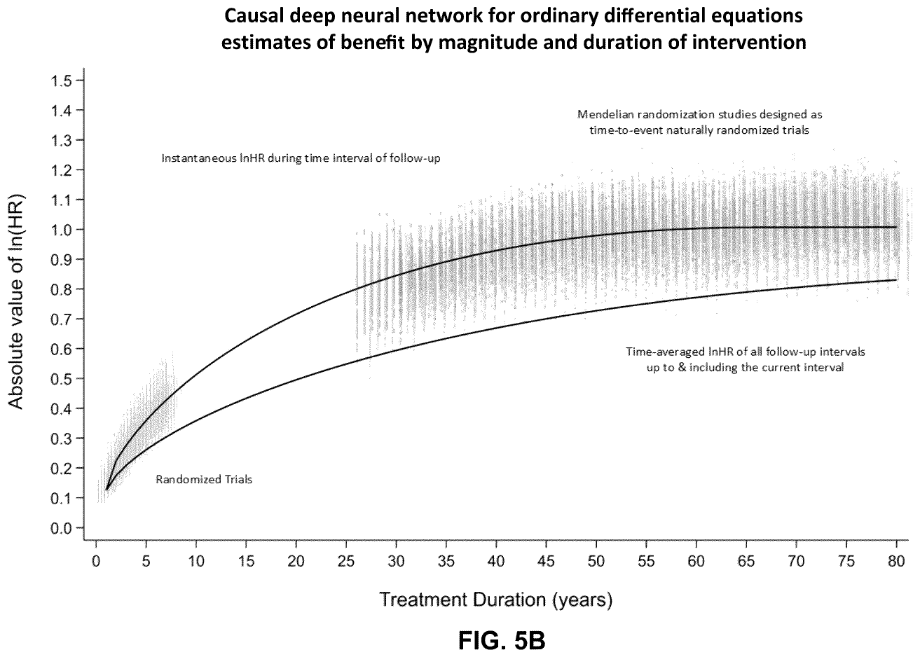

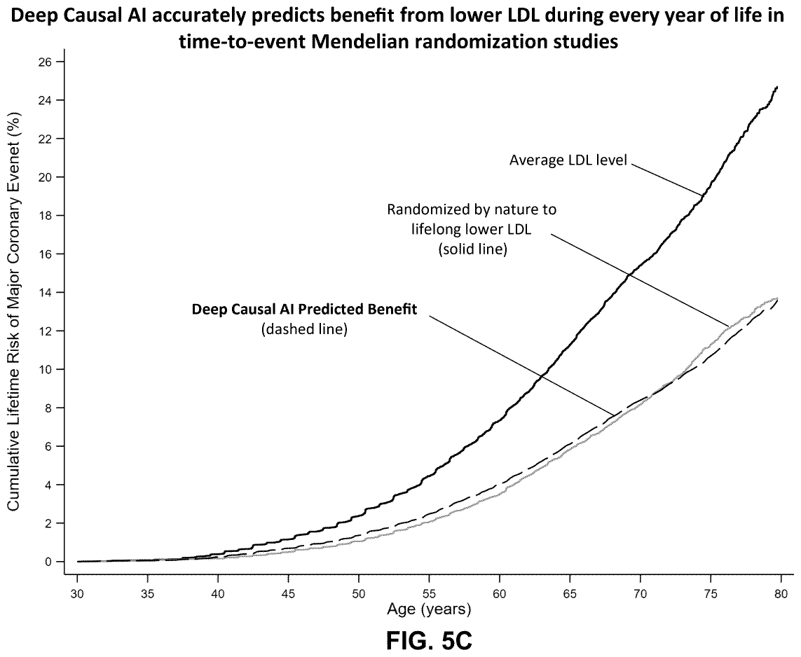

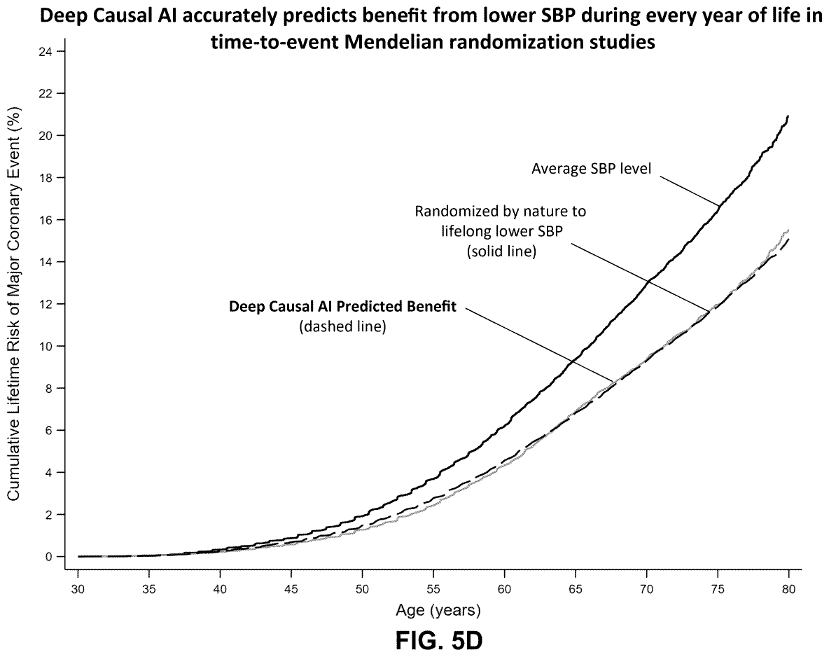

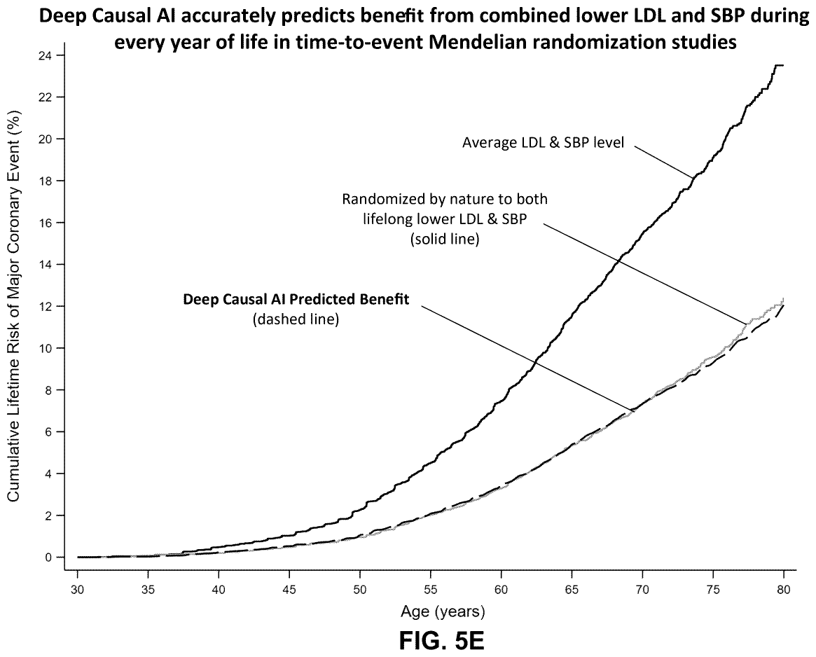

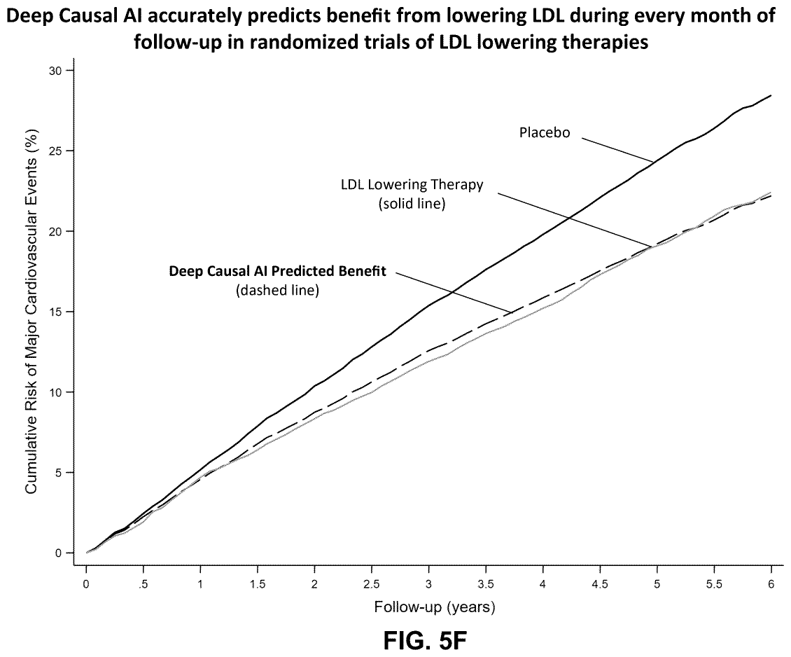

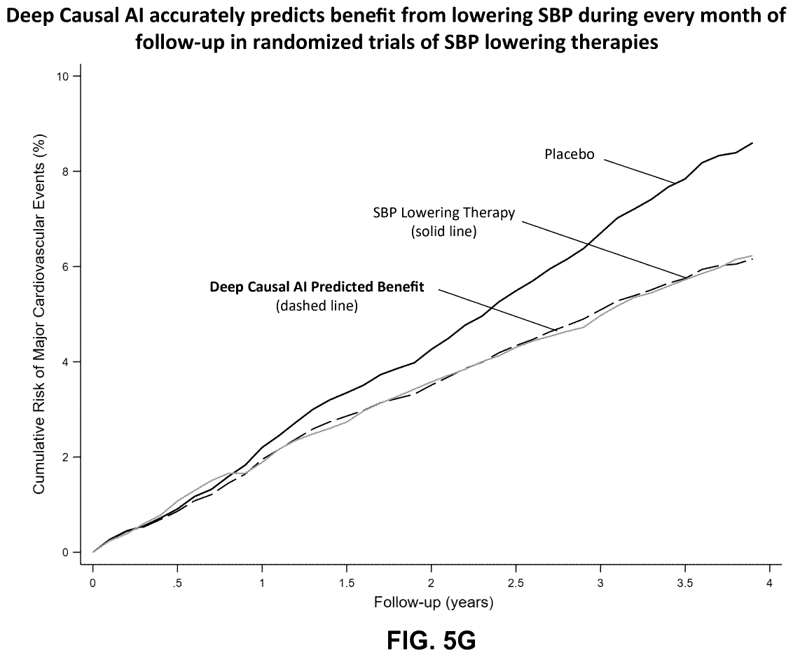

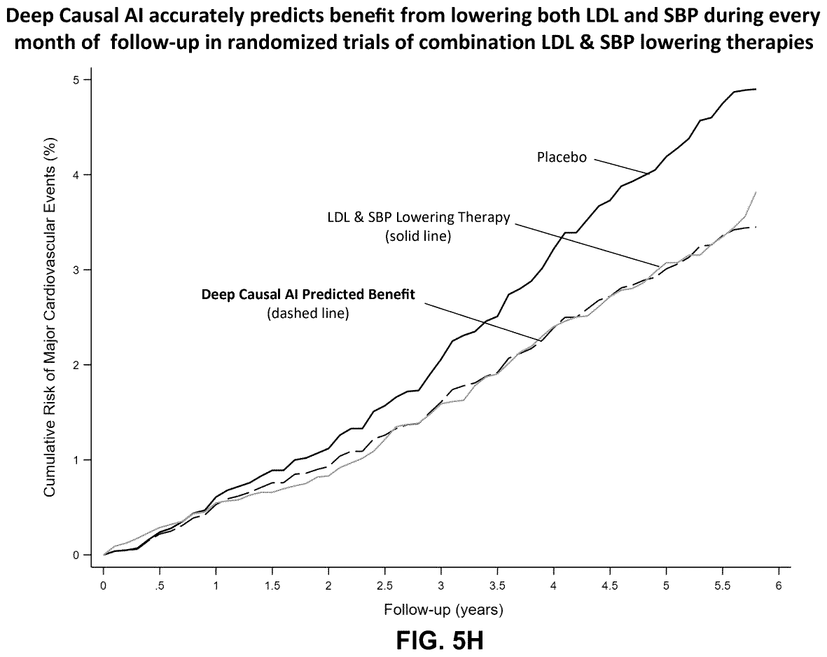

Aspects of the foregoing are illustrated in FIGS. 5A-5H. To begin, as shown in FIG. 5A, to estimate the benefit of reducing exposure to a modifiable cause of disease that causes irreversible structural injury that accumulates over time (such as LDL or SBP), a causal deep neural network for ordinary differential equations (c-DNN-ODE) is trained on randomized training data comprising the instantaneous hazard ratio of a standardized increment of lower LDL (or SBP) during every month of follow-up in randomized clinical trials RCTs (extending from 1-6 years), and every year of follow-up from Mendelian randomization studies extending from age 25-80 years. FIG. 5A illustrates these data by plotting the absolute value of the log hazard ratios against treatment duration (in years in this example). Note that despite the numerous randomized clinical trials and Mendelian randomization studies used to generate the training data, there is a gap in the data between 7 and 25 years, which influences the type of model that may be trained using such data without systematically underestimating the magnitude of the causal estimate of effect. Notably, the c-DNN-ODE method described herein is designed to avoid such systematic underestimates, whereas conventional machine learning models that are trained using regularization (e.g., 11, 12, Lasso, etc.) undesirably produce these underestimates.

FIG. 5B then shows the instantaneous log hazard ratio in each time-interval of follow-up (up through age 80) as estimated by the c-DNN-ODE model and the time-averaged log hazard ratio of all follow-up intervals up to and including the current interval. These non-linear curves show that the c-DNN-ODE “learned” the benefit of lowering LDL by magnitude and duration. Again, different c-DNN-ODE models would be used to predict benefits of lowering LDL and SBP.

FIGS. 5C-5H show empirical evidence validating this approach on randomized data by empirically demonstrating that it accurately estimates the benefit of life long exposure to lower LDL at every age of life in Mendelian randomization studies designed as naturally randomized trials.

FIGS. 5C-5E show empirical evidence validating the approach on data from Mendelian randomization studies.

FIG. 5C shows that the c-DNN-ODE model accurately estimates the benefit of life long exposure to lower LDL at every age of life in Mendelian randomization studies designed as naturally randomized trials. The average LDL level is shown by the solid black curve, the c-DNN-ODE estimate is shown by the dashed line and accurately tracks the lower cumulative lifetime risk line shown by the gray solid line.

FIG. 5D shows that the c-DNN-ODE model accurately estimates the benefit of life long exposure to lower SBP at every age of life in Mendelian randomization studies designed as naturally randomized trials. The average SBP level is shown by the solid black curve, the c-DNN-ODE estimate is shown by the dashed line and accurately tracks the lower cumulative lifetime risk line shown by the gray solid line.

FIG. 5E shows that the c-DNN-ODE model accurately estimates the benefit of life long exposure to combined lower LDL and SBP at every age of life in Mendelian randomization studies designed as naturally randomized trials. The average LDL and SBP level is shown by the solid black curve, the c-DNN-ODE estimate is shown by the dashed line and accurately tracks the lower cumulative lifetime risk line shown by the gray solid line.

FIGS. 5F-5H show empirical evidence validating the approach on data from randomized trials. FIG. 5F shows that the c-DNN-ODE model accurately estimates the benefit of pharmacologic short-term LDL lowering started later in life during every month of follow-up in randomized trials of LDL lowering therapies. The solid black line indicates a placebo, the dashed line is the c-DNN-ODE estimate and accurately tracks the LDL lowering therapy line shown by the gray solid line.

FIG. 5G shows that the c-DNN-ODE model accurately estimates the benefit of pharmacologic short-term SBP lowering started later in life during every month of follow-up in randomized trials of SBP lowering therapies. The solid black line indicates a placebo, the dashed line is the c-DNN-ODE estimate and accurately tracks the SBP lowering therapy line shown by the gray solid line.

FIG. 5H shows that the c-DNN-ODE model accurately estimates the benefit of pharmacologic short-term LDL and SBP lowering started later in life during every month of follow-up in randomized trials of combined LDL and SBP lowering therapies. The solid black line indicates a placebo, the dashed line is the c-DNN-ODE estimate and accurately tracks the LDL and SBP lowering therapy line shown by the gray solid line.

Before we proceed further to explain how the estimates provided by the c-DNN-ODE model (quantifying benefits of lowering LDL and/or SBP in a population) are combined with the predicted instantaneous hazard rates that the subject experiences a cardiovascular event in each interval of follow up, additional aspects of the c-DNN-ODE model are described.

### Additional Aspects Relating to the c-DNN-ODE Model

The c-DNN-ODE model is designed to find a function that describes the change in one variable (Y) over time caused by another variable (X) by modelling continuous changes in the data as it passes between layers of the neural network to capture the non-linear dynamics of how the causal effect of X on Y changes over time. In the context of the technology described herein, the biological cause and effect may be modelled using the observed log hazard ratio during every month (or other interval) of follow-up in randomized trials evaluating LDL or SBP lowering therapies, respectively, standardized for a one-unit absolute observed difference in LDL or SBP during the corresponding time interval; and the observed log HR during every year of life among participants randomized by nature to higher or lower LDL or SBP, respectively, standardized for a one-unit absolute observed difference in LDL or SBP during the corresponding time interval. Each unit lower LDL or SBP represents proportionally less incremental change from the previous time unit, and thus the log hazard ratio eventually approaches an asymptote quantified benefit measured as an instantaneous log hazard ratio.

The c-DNN-ODE model may be designed to quantify how much the proportional reduction in cardiovascular events caused by lowering LDL or SBP changes over time. By using a DNN for ODE, we model both the relationship between x and y; and the dynamics of the rate of change (the derivative, dy/dx). This differential information allows the network to infer a likely “true curve” representing the relationship governed by the biological impact of slowing the trajectory of atherosclerosis by reducing the number of atherogenic lipoproteins that become trapped within the artery wall over time, incorporating the inherent variability of data points.

The resultant DNN-ODE model is causal because it is trained exclusively on randomized data from: (1) randomized trials of LDL and SBP lowering therapies, and (2) Mendelian randomization studies evaluating genetic variants associated LDL and SBP designed as naturally randomized trials. The c-DNN-ODE model not only matches the predicted values with experimental results, but also learns the trajectory of y over x, constrained by a smooth, plausible curve that reflects a governing law (biological effect). By simultaneously learning the derivative (or instantaneous slope) of the change in y at each value of x along with predictions of the output, the network constrains itself to a more consistent and smooth shape that aligns with the expected non-linear biological effect of reducing LDL or SBP on the underlying biological processes of atherosclerotic plaque progression and the propensity of the accumulated plaque to physically disrupt.

Using the ODE approach, the network is regularized by focusing not only on fitting y values, but also on matching the rate of change of y over x. This focus on differential information helps smooth out experimental variability, leading the model to converge toward the most likely true, smooth curve that represents the biological relationship. This robustness to variability enhances the model's capacity to generalize, providing a clearer picture of the actual underlying curve. In addition, when learning a function by fitting randomized causal experimental data, using an ODE solver eliminates the need for other regularization techniques (including L1 and L2 regularization methods). Though it should be appreciated that, in other embodiments, L1 or L2 or other type of regularization methods may be used instead of or in addition to an ODE solver.

That said, one benefit of the ODE approach relative to some other types of regularization methods is that some regularization methods shrinks estimates to reduce sensitivity to outliers, thereby biasing the predicted true causal effects toward the null and, therefore, systematically underestimating both the proportional and absolute magnitude of the benefit of reducing exposure to a modifiable cause of disease, which in turn may lead to systematically underestimating how much relative or proportional benefit increases over time. Thus, combining an ODE with a DNN reduces or outright eliminates the potential for learning estimates of the true causal effects that are systematically biased toward the null.

This is an especially important point because, although the training data available is derived from hundreds of randomized clinical trials and thousands of naturally randomized trials, there is little data in the gap between years 7 and 25 (e.g., as can be seen in FIGS. 5A and 5B). Thus, it is important to avoid systematically underestimating the magnitude of the causal estimate of effect by using regularization techniques common to most machine learning and deep learning methods.

As described above, in some embodiments, the c-DNN-ODE may be a DNN-ODE model trained on randomized data from: (a) randomized trials of LDL and SBP lowering therapies, and (b) Mendelian randomization studies evaluating genetic variants associated with lower LDL and SBP designed as naturally randomized trials.

In some embodiments, the c-DNN-ODE may be trained using randomized data obtained from: (a) participant data for at least 1 million (M) or 1.5 M participants enrolled in one or more prospective studies, whereby for each of the at least 1 M or 1.5 M participants at least one LDL or SBP measurement were available along with a recorded age or date at which a first episode of a fatal or non-fatal myocardial infarction (MI), fatal or non-fatal ischemic stroke, or coronary revascularization occurred, with censoring applied at time of last follow-up, death, or first cardiovascular event; and (b) participant data from at least 250K or 500K participants enrolled in at least 25, 50, or 75 randomized trials evaluating LDL or BP lowering therapies that provided time-to-event curves, and measurements of absolute difference in LDL or SBP between randomized groups in the randomized trials.

In one illustrative example, the c-DNN-ODE may be trained using individual participant data (IPD) from 1,623,491 participants enrolled in one of three (3) long-term prospective biobank cohort studies for whom at least one LDL or SBP measurements was available, and for whom medical records were available recording age (date) at which the first episode of a fatal or non-fatal MI, fatal or non-fatal ischemic stroke, or coronary revascularization occurred. Participants were censored at time of last follow-up, death, or first cardiovascular event. Data from 527,512 participants enrolled in 76 randomized trials evaluating LDL or BP lowering therapies that provided time-to-event curves, and measurements of the absolute difference in LDL or SBP between the randomized groups. In addition, summary data from 2.6 million participants with the age (dates) at which a first atherosclerotic cardiovascular event was recorded, was used for additional external validation experiments.

In more detail, the training data may include training data derived from randomized trials and Mendelian randomization studies. In particular, for randomized trials, the training data may include individual participant data (IPD) and/or summary level data from all published randomized trials of LDL or SBP lowering therapies that met the following criteria: randomized, double blinded cardiovascular outcomes trial comparing an LDL or SBP lowering therapy, respectively, to either placebo or usual care; at least 1000 participants randomized to each treatment arm; at least 1 year of follow-up; provided data including cumulative event curves on cumulative rates of major adverse cardiovascular events; provided data on absolute magnitude of the difference in LDL or SBP between the treatment arms at more than one point in the trial. For Mendelian randomization studies, the training data may include IPD from UK Biobank and FinnGen Project—using genetic variants associated with LDL (and both directionally and proportionally concordant changes in apoB) or SBP to construct instrumental variables.

Further details regarding generating training data for training of the c-DNN-ODE, in some example implementations, are as follows. For an given randomized trial, we can use the reported cumulative event curve to recover the instantaneous hazard of having an outcome event during any interval of follow-up since randomization conditional on surviving to that interval without having had an event in both the treatment and intervention groups. Based on the number of participants surviving to any given time point without an event, we can recreate the lifetable and calculate both the instantaneous hazards and corresponding standard errors for all follow-up intervals. Comparing the instantaneous hazards during each time interval, we can obtain the instantaneous log hazard (lnHR) estimating the benefit of intervention during that time interval (and corresponding standard errors) for the observed difference in absolute reduction of the biomarker targeted by the intervention during the same interval of time. By dividing the instantaneous lnHR (and its SE) by the absolute magnitude of the achieved biomarker reduction achieved during each interval, we obtain the predicted benefit of a 1 unit (sustained) change in that biomarker during each interval.

This process can be repeated for hundreds or LDL and SBP lowering trials. In the combined dataset, we can obtain an overall average summary estimate of the instantaneous lnHR (and corresponding standard error) for a one unit change in the targeted biomarker at each interval of follow-up by combining the lnHR (SE) for a one unit change in biomarker in all of the studies (that contributed data up to and including this interval) by calculating the inverse variance weight average (equivalent to a meta-analysis of each time interval).

We can extend this intuition to obtain the same information for longer durations of sustained interventions from nature's randomized trials. This is accomplished by conducting a Mendelian randomization study designed as a longitudinal time-to-event naturally randomized trial using participant level data (or event curve data if necessary). This study design provides a lifetable of instantaneous hazards of the risk of having an outcome event during any interval of follow-up from the time of randomization (birth, or more precisely—conception).

For each interval of follow-up we can obtain the causal effect of the intervention (genetic instrument) on the risk of having an outcome event during that interval from the ratio of instantaneous hazards to produce a lnHR for that interval. From the number of participants at risk during each interval (from the lifetable), we can obtain the SE of the lnHR during each interval. By obtaining the corresponding magnitude absolute reduction in a biomarker during each interval caused by the genetic variant (or genetic instrumental variable composed of more than one independently inherited variant), we can obtain the lnHR for a one unit change in the biomarker due to the intervention during that interval by dividing the lnHR (and its SE) by the absolute change in the biomarker observed during that interval.

This process can be repeated for thousands of genetic variants associated with LDL (directionally consistent and proportionally consistent with changes in apoB) or SBP as instruments of both randomization and effect; and for hundreds of instrumental variable genetic LDL or SBP scores limited to independently inherited variants meeting specific inclusion criteria. In the combined dataset, we can obtain an overall average summary estimate of the instantaneous lnHR (and SE) for a one unit change in the targeted biomarker at each interval of follow-up by combining the lnHR (SE) for a one unit change in biomarker in all of the studies (that contributed data up to and including this interval) by calculating the inverse variance weight average (equivalent to a meta-analysis of each time interval).

The resulting final combined dataset is composed exclusively of randomized evidence estimating the magnitude of the causal effect of sustained reductions in LDL or SBP or both at all time intervals from 1 month to 80 years of follow-up from the time of randomization. Because the data is derived exclusively from randomized and therefore unbiased evidence, the included effect estimates represent unconfounded causal estimates of effect that permit the training of ‘casual’ DNN for ODE. In other words, training a DNN for ODE using these training data is what renders the resulting model ‘causal.’ In some embodiments, the DNN for ODE model trained may have the architecture as described in “Neural Ordinary Differential Equations”, Chen, R. T. Q., Rubanova, Y., Bettencourt, J. and Duvenaud, D. K, in Advances in Neural Information Processing Systems, volume 31, 2018.

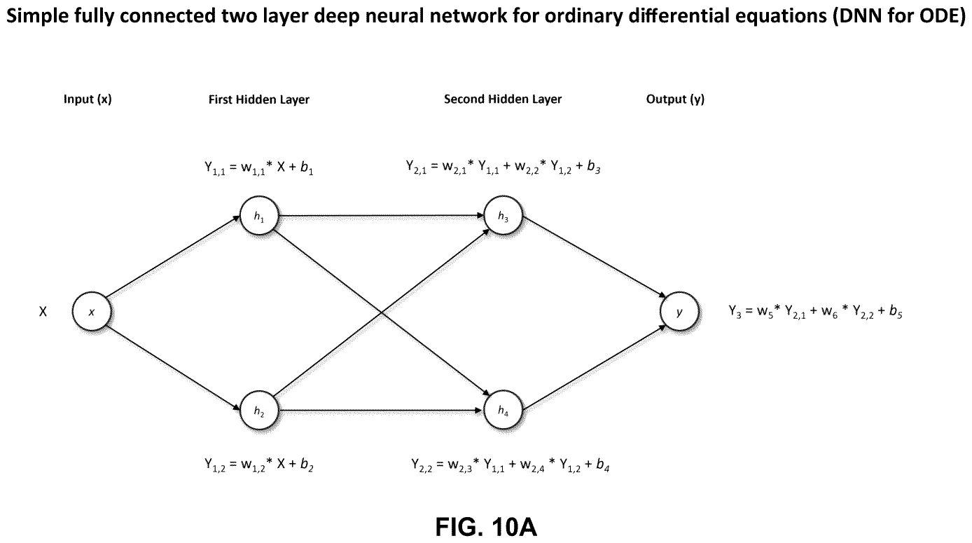

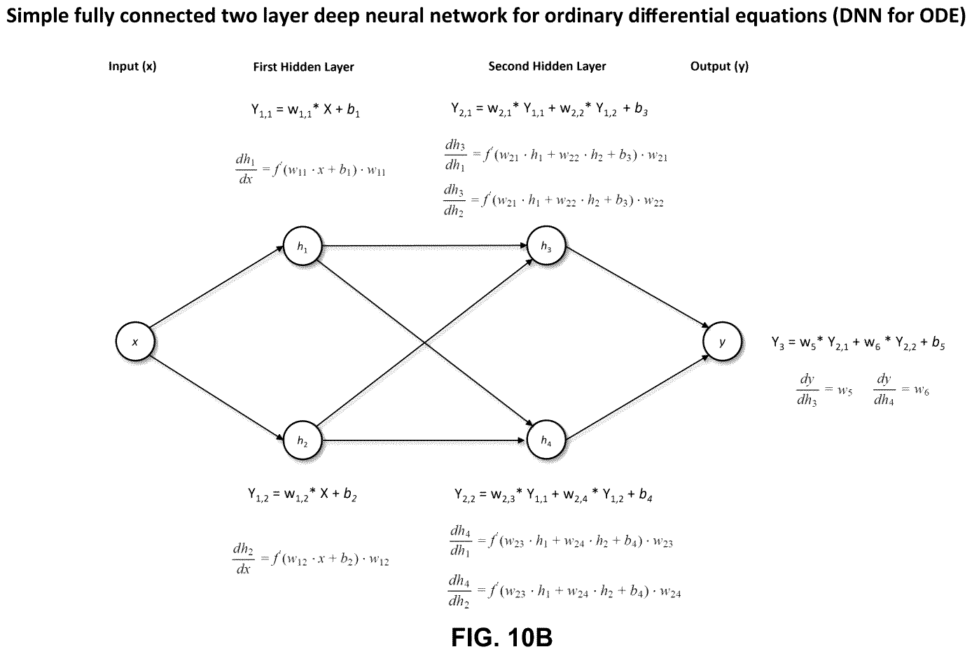

Further details regarding aspects of calculations with a c-DNN-ODE model and training c-DNN-ODE models are as follows. FIG. 10A shows a simple fully-connected two-layer deep neural network for ordinary differential equations (DNN for ODE). In a DNN for ODE, both a predicted value of y, and the derivative or instantaneous slope of the change in y at a given value of x are calculated at each node as shown in FIG. 10B.

The overall derivative of

$$\frac{dy}{dx}$$

for the change in Y for a given X can be calculated from the partial derivatives at each node using the chain rule:

$$\frac{dy}{dx} = \frac{dy}{{dh}_{3}} \cdot \frac{{dh}_{3}}{{dh}_{1}} \cdot \frac{{dh}_{1}}{dx} + \frac{dy}{{dh}_{3}} \cdot \frac{{dh}_{3}}{{dh}_{2}} \cdot \frac{{dh}_{2}}{dx} + \frac{dy}{{dh}_{4}} \cdot \frac{{dh}_{4}}{{dh}_{\text{?}}} \cdot \frac{{dh}_{1}}{dx} + \frac{dy}{{dh}_{4}} \cdot \frac{{dh}_{4}}{{dh}_{2}} \cdot \frac{{dh}_{2}}{dx}$$

$$\text{?} \text{indicates text missing or illegible when filed}$$

Note that the partial derivatives at each node are weighted by the same learned weights as those used to predict the value of Y at each node; and f( ) represents the activation function of the node output (e.g., ReLU, sigmoid, etc.), so that all derivatives are computed on the activated node output.

One possible loss function for training a DNN-ODE model may be the sum of the squared differences between the predicted and actual values. Because the model simultaneously calculates both the predicted output y, and the derivative or the rate of change in y with respect to x, the loss function seeks to minimize the mean squared error from the absolute difference between the predicted and observed data for both of these parameters.

For example, the loss function may be calculated as follows:
the neural network takes X as input and outputs Y and the rate of change of Y to fit a function:

$$\frac{dy}{dt} = f ( t , y )$$

during each epoch, a numerical integration method (like the Euler method or Runge-Kutta) is used to reconstruct the value of Y at different points along X

$$y ( {t}_{1} ) = O D E - Solver ( f , {x}_{0} , {\text{?}}_{0} , {t}_{1} )$$

$$\text{?} \text{indicates text missing or illegible when filed}$$

the predicted and observed values of Y, and the predicted and observed rate of change in Y at each value of X, are then compared to calculate the mean squared error as:

$$Loss = \sum ( {( {y}_{pred} - {y}_{true} )}^{2} + α \cdot {( \frac{{dy}_{pred}}{dx} - \frac{{dy}_{true}}{dx} )}^{2} )$$

where: y_{pred} and yₜᵣᵤₑ and are the predicted and actual y values and

$$\frac{{dy}_{pred}}{dx}$$

are the predicted

$$\frac{{dy}_{true}}{dx}$$

and actual slopes, and α is a weighting factor to balance the contribution of the two terms.
The network weights and biases are updated using backpropagation to simultaneously minimize the combined squared errors for both the predicted values of Y and the instantaneous rate of change in Y.

In some embodiments, the c-DNN-ODE may be implemented using a combination of standard PyTorch modules along with specialized libraries for solving differential equation, including: (1) torch.nn (e.g., for building the base layers of the neural network, e.g., nn.Linear, nn.Sequential, etc.); (2) torchdiffeq—An external library specifically for solving differential equations with PyTorch. This library provides solvers like torchdiffeq.odeint that can handle neural ODEs. The ODE function can be defined as a neural network and passed to odeint to integrate overtime; and (3) Autograd (automatic differentiation)—PyTorch's autograd functionality is essential for computing gradients in a neural ODE. It enables the computation of the derivatives of loss with respect to model parameters.

In some embodiments, the c-DNN-ODE model may be a fully connected deep neural network predicting Y and the instantaneous rate of change in Y. The deep neural network may have at least 3, 4, or 5 fully-connected layers. In one illustrative example, the architecture of the DNN may comprise an input layer (e.g., one dimension specifying the duration of time) followed by 8, 16, 8, and 1-dimensional layers, all fully connected, with bias terms and ReLu non-linearities.

Notwithstanding the foregoing discussion of c-DNN-ODE models, it should be appreciated that, in some embodiments, one or more other types of models may be trained on these types of training data (including data from randomized trials and Mendelian randomization studies) instead of a c-DNN-ODE model. For example, the at least one second ML model may comprise a non-linear regression model, an adaptive basis function regression model, a neural network regression model, a deep neural network regression model, a logistic regression model, a polynomial regression model, a decision tree regression model, a random forest regression model, and/or a gradient boosted decision tree regression model, with any one of these models being regularized in any suitable way. We provide a few comments regarding such alternatives next.

One example of an alternative method is a polynomial regression using the log hazard ratio lnHR as the dependent variable and increment of follow-up time as the independent variable. However, although this approach may be used, there are drawbacks to using this approach and which may limit its usefulness. First, selection of the order of the polynomial has to be limited to those that produce a biologically plausible curve without too many inflection points. Second, the most biologically plausible curve generally is not the one with the greatest r-squared value (i.e., it is not the one that minimizes the mean-squared error between the observed and predicted points on the curve). Selecting the correct polynomial therefore is a matter of judgement informed by domain area expertise, and generally requires an iterative process of trial and error. Naively minimizing the loss function would produce biologically implausible curves by overfitting the data (particularly when using the raw data for the lnHR at all time points in both randomized trials and Mendelian randomization studies designed as longitudinal time-to-event naturally randomized trials; rather the inverse variance weighted meta-analysis or summary estimate of effect, or lnHR, at each increment of follow-up).

Alternatively, one can combine polynomial regression with other methods. For example, a gradient boosted machine can be used to predict the lnHR at each time point, but cannot make predictions for intervention follow-up times ranging from 6 or 7 years from RCTs) to 30 years (where the naturally randomized trials begin estimate of lnHR at yearly or other interval time up to age 80). As a result, gradient boosted machines and other tree-based methods cannot make estimates or create a continuous curve predicting proportion benefit (lnHR) by magnitude and duration of sustained intervention. One can attempt to solve this problem by running two gradient boosted machines: one on the trial data for intervention durations from 0-7 years, and one on the naturally randomized trials between years 30 to 80. We could then plot the two curves, and then try to connect the curves using polynomial regression or other similar method. While possible, it is difficult to have confidence in the shape of the curve estimating benefit during the missing years where data is not available (e.g., years 7-30); and the resulting curve is highly dependent on the order of the selected polynomial. And even if the order of the polynomial were selected by a statistical software package to minimize the loss or maximize fit, it may produce a biologically and clinically implausible curve. This hybrid method exacerbates the limitations of polynomial regression because the gradient boosted machines often overfit the data when modelling two separate situations (short term trials, and long-term naturally randomized trials)—compared to using a single gradient boosted machine on the entire data set. Finally, the gradient boosted machine also uses a L1 or L2 regularization to avoid this overfitting problem. However, these regularization methods are mere shrinkage functions that minimize the impact of outliers on overfitting—and systematically bias the results toward the null, thus systematically underestimating the causal effects of the magnitude of the benefit of the intervention. This is exactly what we wish to avoid. The entire point of creating a ‘causal’ model trained exclusively on randomized and therefore ‘causal’ data is to estimate true magnitude of the ‘causal’ effects so that we can accurately design and predict the results of RCTs, and make recommendations about the interventions needed to slow disease to prevent clinical events and accurately predict the benefit of those intervention—is to have accurate, unbiased estimates of biological cause and effect. Other ML and DL methods, including elastic net regression, DNN, random forests, and the like, suffer from the same drawbacks described above for either polynomial regression, gradient boosted machines, or both. In summary, though such alternatives may be employed, they have some drawbacks.

By contrast, the ‘causal’ DNN for ODE described herein (‘causal’ because it is trained exclusively on randomized and therefore ‘causal’ data) can take as input either all of the raw data, or the inverse variance-weighted summary estimates of the lnHR at each increment of follow-up as the dependent variable and time as the independent variable (with or without other inputs—all of which should be equally distributed because we are exclusively using randomized data and therefore they are unlikely to provide material information unless they are strong effect modifiers) and produce a smoothly differentiated curve that also spans the gap of absent data (e.g., as shown in FIGS. 5A and 5B); and does so without underestimating the causal magnitude of the benefit of the intervention by avoiding a regularization shrinkage function, and instead using differentiation as the method to minimize errors (by simultaneously minimizing the error between the predicted and observed lnHR, and the predicted and observed change in lnHR from the previous increment or the differentials).

Given the foregoing description of how estimates quantifying benefits of lowering LDL and/or SBP may be obtained by a c-DNN-ODE model or other model, we next describe how such estimates may be combined with the predicted instantaneous hazard rates that the subject experiences a cardiovascular event in each interval of follow up.

In particular, in some embodiments, the expected benefit of lowering LDL or SBP over any time interval for the individual person under consideration may be computed as follows.

First, we access values of the predicted instantaneous hazard rates of experiencing a cardiovascular event during each interval of follow-up from the current age to age 80 years in the lifetable of risk by age constructed for that person as described herein including with reference to FIG. 2C (e.g., by using outputs from the bi-LSTM(s) and survival DNN with PEM using cumulative LDL as the increment of analysis).

These values are then multiplied by the c-DNN-ODE estimated time-averaged instantaneous log hazard ratio for a one unit lower LDL or SBP corresponding to the duration of treatment at that interval of follow-up (or age), and also adjusted for the expected absolute magnitude of the reduction in LDL or SBP in response to the recommended intervention using the Wald ratio of effect estimates method (see e.g., Example 7). Different c-DNN-ODEs would be used for LDL and SBP benefit estimates. That is, one c-DNN-ODE would be used to estimate time-averaged instantaneous log hazard ratios for a one unit lower LDL and another c-DNN-ODE would be used to estimate time-averaged instantaneous log hazard ratios for a one unit lower LDL SBP.

Next, the estimated cumulative hazard of experiencing a cardiovascular event, and the corresponding cumulative event rate, at each age of follow-up, conditional on surviving to an interval without experiencing an event, may then be computed using standard lifetable analysis of the treatment adjusted predictions.

Finally, the expected clinical benefit of lowering LDL or SBP for the individual person under consideration is computed as follows. The predicted proportional reduction in the risk of experiencing a cardiovascular event at any duration of follow-up may be computed as the ratio of the predicted cumulative hazard at that duration of follow-up (age) adjusted for the magnitude and duration of LDL or SBP lowering, to the expected cumulative hazard without intervention to lower LDL or SBP. And the predicted absolute reduction in the risk of events may be computed as the absolute difference between the predicted absolute cumulative event rate at a specific duration of follow-up (age) adjusted for the magnitude and duration of LDL or SBP lowering, to the expected absolute cumulative event rate at the same duration of follow-up without intervention to lower LDL or SBP.

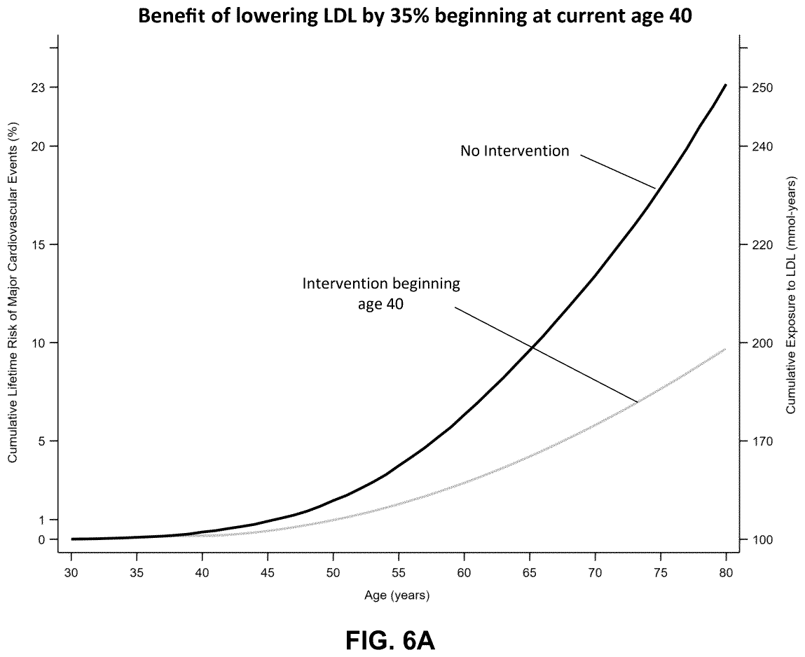

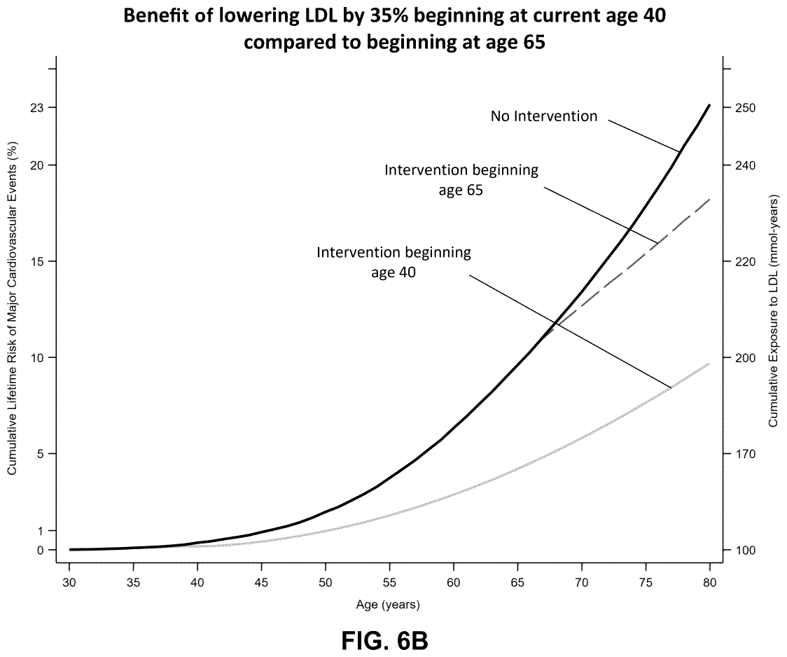

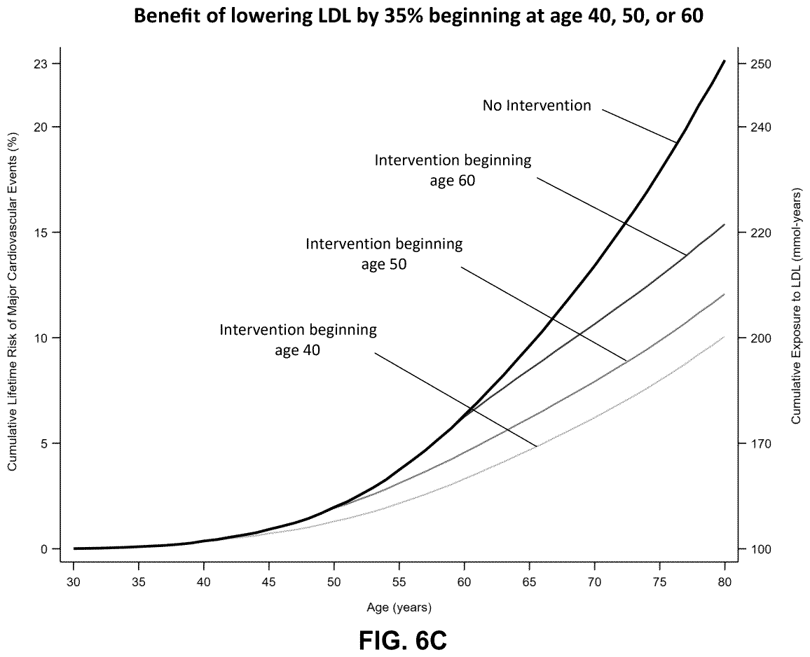

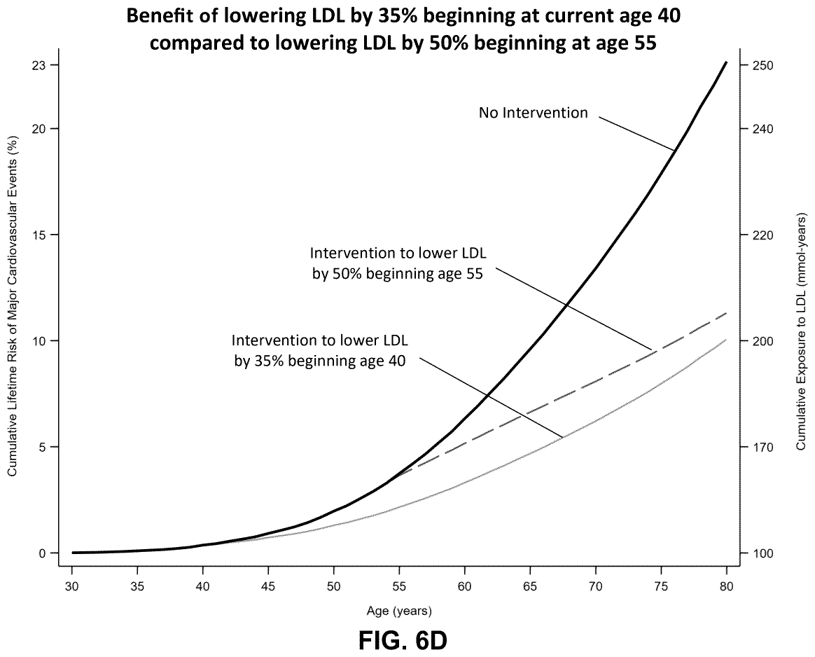

FIGS. 6A-6D illustrate these computations. For example, FIG. 6A shows the benefit of lowering LDL by 35% beginning at the subject's current age of 40 years. To estimate the benefit of a specific LDL intervention beginning at the current age (e.g. once-yearly intervention to lower LDL expected to achieve a 35% ‘time-averaged’ reduction in LDL) and extending until age 80 (or any other duration), the transposed lifetable of instantaneous hazards for a cardiovascular event during each year of life caused the accumulated plaque burden and combined effect of other causes of arterial wall injury at every point in time for the person being evaluated, is multiplied by the exponentiated form of the log hazard ratio for a 1 (one) mmol/L, adjusted for the expected absolute magnitude of LDL reduction for a 35% proportional reduction in LDL based on the person's baseline untreated LDL level, during each increment of treatment—where the time-averaged log hazard ratio derived from the c-DNN-ODE model increases as the duration of treatment increases. The intervention-adjusted ‘on-treatment’ instantaneous hazard rates are then combined using standard lifetable methods to calculate the predicted cumulative hazard and cumulative event rates at each age for the person being evaluated for both the treated and untreated states. The resulting cumulative event rates with and without treatment are shown in FIG. 6A.

For comparison, the expected reduction in lifetime risk from lowering LDL using the same once-yearly treatment beginning when 10-year risk exceeds 10% (according to current guidelines) is shown in FIG. 6B.

As described herein including in the next section, such analysis may be repeated for multiple different intervention sequences—constrained by the imposed domain specific heuristic that once started a therapy is continued, or increased in tensity, or another therapy is added to enhance clinical translation. For example, FIG. 6C compares benefit of therapeutic intervention sequences for lowering LDL that vary by the age of the subject at which the therapeutic intervention sequence commences. As another example, FIG. 6D compares the benefit of therapeutic intervention sequences that vary by age of the subject at which they begin as well as by the magnitude of intervention.

### Estimating How Much Additional Cumulative Reduction in LDL, SBP, or Both is Needed to Overcome Increased Risk Caused by Amount of Inherited Lp(a)

As described herein, the causal effect of cumulative exposure to Lp(a) at all levels of cumulative exposure to LDL (accumulated plaque burden) may be incorporated into the risk assessment framework described in this disclosure. This enables obtaining a quantitatively precise estimate of how much the biological casual effect of the amount of Lp(a) inherited by each person increases the risk of cardiovascular at every point in time.

As described above with respect to FIG. 2C, this causal effect can be obtained by including the positionally encoded (by age) predicted cumulative Lp(a) level (determined using an Lp(a) trajectory estimated as described herein) at every age in the survival model executed at every level of cumulative exposure to LDL; and then adding them together in a piecemeal manner (as with the other predicted exposure levels at level of cumulative exposure to LDL as a representation of the accumulated plaque burden at every point in time) using the a survival model (e.g., a DNN) with PEM or any other survival model described herein.

This enables determining, for a particular subject, how much their inherited Lp(a) levels increase their risk of ASCVD events at all ages, and providing the particular subject with guidance on how additional LDL or SBP reductions over time (reductions in cumulative exposure to LDL, SBP, or both) is needed to specifically overcome their increased inherited risk caused by the amount of circulating Lp(a) that they have inherited.

This may be implemented as follows. First, the predicted cumulative event curves may be constructed with and without Lp(a). That may be done using the methods described with respect to FIG. 2C with and without Lp(a) as part of the input feature data at act 221. Next, the magnitude of additional cumulative reduction in LDL needed to overcome the increased risk of MACE caused by cumulative exposure to Lp(a) at each point in time is calculated (conditional on the age at which LDL lowering is started). In addition, the magnitude of additional cumulative reduction in SBP needed to overcome the increased risk of MACE caused by cumulative exposure to Lp(a) at each point in time is calculated (conditional on the age at which SBP lowering is started). Finally, the magnitude of additional cumulative reduction to the combination of both LDL and SBP needed to overcome the increased risk of MACE caused by cumulative exposure to Lp(a) at each point in time is calculated (conditional on the age at which LDL and SBP lowering is started).

These calculations may be performed using any of a variety of simple quantitative algorithms by summing the required reductions in cumulative exposure needed to overcome the effect of Lp(a) at each point in time, calculating the incremental reduction in cumulative exposure at each point time relative to the previous time point, and then summing the required incremental reductions in cumulative exposure to LDL.

This is equivalent to the process of allowing a person to set a lifetime risk goal (or a level cardiovascular health they which to achieve—construed as the maximum cumulative lifetime risk of MACE a person is willing to tolerate), and then solving for the magnitude of cumulative reductions in LDL, SBP, or both needed to achieve this goal.

Here the goal is equivalent to the difference in a person's predicted cumulative lifetime risk of MACE when including Lp(a) in the calculations to estimate remaining lifetime risk, and when excluding Lp(a) as an input when calculating the estimated remaining cumulative lifetime risk for that person as follows: the cumulative event curve plots (and lifetable estimates) are compared at each age for: a) cumulative lifetime risk of heart attack or stroke without considering Lp(a) levels; b) cumulative lifetime risk of heart attack or stroke now including cumulative Lp(a) levels; and c) cumulative lifetime risk of heart attack or stroke both considering cumulative Lp(a) levels at each increment of time and the required reductions in cumulative exposure to LDL, SBP, or both required to overcome the increased risk caused by Lp(a).

As may be appreciated from the foregoing, in one example implementation, this functionality may be achieved as follows.

First, the measured value of Lp(a) is used as an input. The measured Lp(a) level is standardized in the same way as all other inputs (e.g., min-max); noting that Lp(a) levels are very asymmetrically distributed in the population and can vary by 1000-fold between individuals. Then, the standardized Lp(a) levels are then positionally encoded by age and added to the vector of inputs. That vector of inputs may be processed, in parallel, by three machine learning models (examples of which are provided herein) cumulative exposures to LDL, SBP, and Lp(a), respectively, as described herein (e.g., with reference to acts 214-216 of FIG. 2B).

Next, the vector, now including the standardized estimate of Lp(a) levels at all ages, the corresponding positionally encoded predicted Lp(a) level at all ages, and the standardized and positionally encoded predicted cumulative exposure to Lp(a) at all ages, is then passed into the remaining stack of algorithms—where they contribute their time-dependent biological cause and effect to the estimates cumulative lifetime risk of MACE at all ages; and the corresponding predicted benefit of reducing LDL, SBP, or both beginning at all ages by magnitude, duration and timing of lowering LDL, SBP, or both.

Finally, after all current outputs, the option is presented to the user of the platform to be presented with estimates of how much their inherited level of Lp(a) increases their remaining lifetime risk of MACE at all ages; and how much additional reductions in LDL, SBP, or both are required to specifically overcome the increased risk caused by their inherited burden of Lp(a). This information translates Lp(a) levels into clinically useful information that can be immediately used to guide the timing and intensity of lowering LDL and SBP to further personalize the prevention of ASCVD events.

### Additional Implementation Detail

In some embodiments, the translation of Lp(a) to ASCVD may be performed as follows.

First for Lp(a), we may estimate remaining lifetime risk under two scenarios. First, when including a person's inherited risk due to Lp(a)—‘exposure to Lp(a)’ and ‘inherited Lp(a)’ is referred to herein interchangeably because—unlike other causes of ASCVD like lipoproteins such as LDL, or other exposures like SBP—Lp(a) levels not changed by diet, exercise, or other lifestyle choices. Instead, the magnitude of exposure to Lp(a) is largely determined by each person's genetics explaining why Lp(a) levels remain relative constant for most of life, unlike LDL or SBP which have characteristic shapes of their trajectories over time. As a result, our exposure to Lp(a) is determined almost entirely by how much Lp(a) we inherit.

This gives us two estimates of remaining lifetime risk of MACE: (1) including the causal effect of circulating Lp(a) over time (i.e. cumulative exposure to Lp(a)); and (2) not including the causal effect of circulating Lp(a).

The difference between these two estimates of cumulative remaining lifetime risk of MACE provides an estimate of the magnitude of the effect caused by how much Lp(a) a person inherits (i.e. their cumulative exposure to Lp(a)) at all time points: (i) the instantaneous hazard of MACE at any age or time interval; and (ii) the cumulative hazard of MACE until age 80 (or other arbitrarily selected age limit).

This estimated magnitude of increased risk caused by Lp(a) can be expressed as a proportional risk (hazard ratio), or an absolute increase in risk (difference in absolute instantaneous or cumulative hazard during any time interval or up to any duration of follow-up). Note that the causal effect of Lp(a) is generally restricted to how much it increases the risk of MACE because, unlike LDL or SBP, it is not normally distributed where some people have higher or lower levels than ‘average’. Instead, Lp(a) has an extreme rightward-skewed distribution that can vary by 1000-fold among individuals. The population median (not mean because of the extremely skewed distribution) is very low, around 15-20 nmol/L. Even for persons in the lowest 10% who have Lp(a) of 3-5 nmol/L (about ⅓ to ¼ of the population median), the absolute difference is small—only 12-15 nmol/L. Because very large absolute differences in Lp(a) of 100-300 nmol/L are required to produce a meaningful and therefore measurable causal effect on the risk of MACE, these very small absolute differences are too small to have any material impact on risk. Therefore, only those persons with Lp(a) levels elevated above the median have an increased risk of MACE caused by Lp(a); and the magnitude of that causal effect is proportional to the magnitude of the absolute increase in circulating Lp(a), thus explaining why only a small proportion of the population of a substantially increased lifetime risk of MACE caused by their inherited cumulative burden of circulating Lp(a).

The absolute difference in remaining cumulative lifetime risk of MACE when considering Lp(a) and not considering Lp(a) thereby provides an estimate of the increased risk of MACE that must be overcome to specifically eliminate the increased risk specifically caused by a person's exposure to Lp(a) over time.

With this formulation, the problem is very similar to the problem of predicting how much we need to lower LDL, SBP, or both to reduce a person's cumulative remaining lifetime risk of MACE to achieve a specific target or goal (e.g. keeping a person's cumulative lifetime risk of MACE less than 5% at all ages up to age 80). This formulation allows us to translate a person's Lp(a) level into clinically actionable information by estimating how much we need to lower LDL, SBP, or both—conditional on when we start to lower LDL or SBP—to specifically overcome the increased risk caused by how much Lp(a) they inherited (their exposure to Lp(a) over time throughout life) In some embodiments, this may be accomplished as follows. First we construct a lifetable estimating the instantaneous hazard for MACE at all ages for a person using all exposures including Lp(a). Next, we construct a second column in the lifetable that estimates the instantaneous hazards at all ages for the same person including all exposures except Lp(a).

Then, we sum the instantaneous hazards to obtain two estimates of cumulative remaining lifetime for this persons: (i) one including the causal effect of Lp(a); (ii) one that does not include the casual effect of Lp(a). The difference in cumulative remaining lifetime risk between these two estimates simply becomes a sub-goal which is defined as: the remaining cumulative lifetime risk goal needed to overcome the causal effect of the amount of Lp(a) that a person has inherited.

In turn, we now simply iterate through the solution space but here to specifically find the amount we must reduce LDL, SBP, or both to achieve this specific proportional and absolute reduction in risk. The magnitude of which reduction will depend on when we start lowering LDL, SBP, or both because the proportional risk reduction (and therefore corresponding absolute reduction in instantaneous and cumulative hazards of MACE) depend on the magnitude and duration of when we start lowering LDL, SBP, or both; and because the corresponding absolute residual risk will depend on when we started because the residual rate of MACE depends on how much atherosclerosis and arterial wall injury has already accumulated before we start to lower LDL and SBP (and any legacy benefits form earlier interventions to lower LDL and SBP). Effectively we want to calculate how much we need to lower LDL, SBP, or both so that a person's two estimates of remaining cumulative lifetime risk of MACE are the same: (i) cumulative lifetime risk of MACE for this person when considering the causal effect of their inherited burden of Lp(a) conditional on the selected magnitude and timing of lowering LDL, SBP, or both and continuing that intervention until age 80 years (or other arbitrary age limit); and (ii) cumulative lifetime risk of MACE for this person when considering all the same other exposures EXCEPT Lp(a).

This information translates a person's Lp(a) into immediately actionable clinical information even before we have effective therapies to specifically and potently lower Lp(a) by telling a person and their physician how much they need to lower LDL, SBP, or both beginning now (or any future age) to specifically overcome the increased inherited risk of MACE caused by how much Lp(a) they inherited.

### Estimating How Much Additional Cumulative Reduction in LDL, SBP, or Both is Needed to Overcome Increased Risk Caused by Person's Inherited Genetic Risk of ASCVD

As described herein, a person's inherited risk of ASCVD may be represented, albeit crudely, by the ASCVD polygenic score (ASCVD-PGS). The ASCVD polygenic score is a fixed exposure that is immutable and so it is not meaningful to estimate the benefit of reducing a polygenic score. On the other hand, it is possible to translate a polygenic score into clinically useful information that can provide additional information to refine and further guide clinical care individualized to each person.

To this end, a PGS may be translated into clinically useful information by estimating how much additional cumulative reductions in LDL, SBP, or both are needed to overcome the excess risk caused by a person's inherited genetic risk of ASCVD as represented by the ASCVD-PGS. The magnitude of cumulative reductions in LDL, SBP, or both needed to overcome polygenic predisposition will depend on the magnitude, timing, and duration of therapy lowering LDL, SBP, or both—conditional on when the therapy was started.

By incorporating the (non-causal) effect of inherited polygenic risk at all levels of cumulative exposure to LDL (accumulated plaque burden), we can obtain a quantitatively precise estimate of how much polygenic predisposition to ASCVD inherited by each person increases the risk of cardiovascular at every point in time. This non-causal effect may be obtained by simply including the ASCVD-PGS at every age in the survival model executed at every level of cumulative exposure to LDL; and then adding them together in a piecemeal manner (as with the other predicted exposure levels at level of cumulative exposure to LDL as a representation of the accumulated plaque burden at every point in time).

Here, we positionally encode the ASCVD-PGS by age because we are agnostic to whether its biological impact varies with age. The revised predicted cumulative event curves with and without considering ASCVD-PGS are then constructed using the techniques described with reference to FIG. 2C. Next, the magnitude of additional cumulative reduction in LDL needed to overcome the increased risk of MACE associated with (but not necessarily caused by the ASCVD-PGS) at each point in time is calculated (conditional on the age at which LDL lowering, SBP lowering, or both is started).

This calculation may be performed using any variety of simple quantitative algorithms by summing the required reductions in cumulative exposure needed to overcome the effect of inherited predisposition as represented in one axis by the ASCVD-PGS at each point in time, calculating the incremental reduction in cumulative exposure at each point time relative to the previous time point, and then summing the required incremental reductions in cumulative exposure to LDL, SBP, or both.

The method for calculating the additional reduction in cumulative exposure to LDL, SBP, or both needed to overcome a person's polygenic predisposition is calculated using the same methods as described above for overcoming the increased inherited risk due to Lp(a), as described in the foregoing section.

Finally, the cumulative event curve plots (and lifetable estimates) are compared at each age for: a) cumulative lifetime risk of heart attack or stroke without considering ASCVD-PGS;
b) cumulative lifetime risk of heart attack or stroke now considering ASCVD-PGS; and
c) cumulative lifetime risk of heart attack or stroke both considering ASCVD-PGS at each increment of time and the required reductions in cumulative exposure to LDL, SBP, or both required to overcome the increased risk due to a person's inherited genetic predisposition as represented by their unique combination of genotypes at the variants included in the ASCVD-PGS.

As may be appreciated from the foregoing, in one example implementation, this functionality may be achieved as follows. First, the calculated or provided ASCVD-PGS, and/or other PGS(s), is used as an input. Next, the ASCVD-PGS is standardized in the same way as all other inputs (e.g., min-max); noting that ASCVD-PGS values are already standardized to have a mean of zero (0) and a SD of 1; but are now converted to the same scale as all other inputs. The ASCVD-PGS value is then positionally encoded by age and added to the vector of inputs.

After the cumulative exposures to LDL, SBP, and Lp(a) are estimated; and the trajectory of other exposures are estimated; a matrix of input vectors (one for each age), including the standardized ASCVD-PGS value and the ASCVD-PGS value positionally encoded at each age, are passed into the remaining stack of algorithms—where the ASCVD-PGS contributes to the estimates cumulative lifetime risk of MACE at all ages (agnostic to causality); and the corresponding predicted benefit of reducing LDL, SBP, or both beginning at all ages by magnitude, duration and timing of lowering LDL, SBP, or both.

Finally, the option may be presented to the user of the platform to be presented with estimates of how much their inherited level of polygenic score (ASCVD-PGS) increases their remaining lifetime risk of MACE at all ages; and how much additional reductions in LDL, SBP, or both are required to specifically overcome the increased risk caused by their inherited predisposition to ASCVD as represented in one dimension by their polygenic score (ASCVD-PGS). This information translates a person's inherited polygenic predisposition to ASCVD into clinically useful information that can be used to guide the timing and intensity of lowering LDL and SBP to further personalize the prevention of ASCVD events.

### Additional Implementation Detail

The process for translating polygenic scores into clinically useful information is exactly the same as the process described above with respect to Lp(a) (in the section also called “Additional Implementation Detail”), but with a small caveat. A current generation polygenic score (PGS) is a very crude construction that measures the association (odds ratio; relative risk; or HR) between a very large number of genetic variants and an outcome. The magnitude of these associations are measured by the beta coefficient for each variant. The beta coefficients are simply summed together to obtain a PGS for each person. All or most genetic variants, which are very highly correlated with each other because they are so close on the various genes, are included in the PGS. Therefore, current-generation PGS are not valid instrumental variables, which requires including only independently inherited variants associated with a biomarker or outcome to serve as a proxy ‘instrument’ to assess the level of the outcome or biomarker.

By construction and design, simply summing the beta coefficients for each variant produces a metric with a normal distribution (which for convenience can be centered around 0 with a symmetrical SD). Because a PGS is by design normally distributed, an equal number of people will have higher or lower than average PGS.

By convention, and for mathematical validity, we assume the mean PGS is associated with no increased or decreased risk compared to the average risk in the population conditional on the same level of all other exposures. Therefore, unlike Lp(a), persons with lower than average PGS will have a lower remaining lifetime risk of MACE than ‘average’ and persons with higher than average PGS will have a higher than average risk. The practical effect of this observation is that persons with higher than average PGS will have a higher than average instantaneous risk during all time intervals resulting in a higher corresponding cumulative remaining lifetime hazard of MACE as compared to persons with average PGS. Therefore, we want to solve for much we have to lower LDL, SBP, or both to specifically overcome the increased risk caused (or more precisely apparently caused) by a person's PGS, conditional on when we start to lower LDL, SBP, or both. This is the same problem for how to translate Lp(a) into clinically useful information.

On the other hand, persons with lower than average PGS will have a lower than average instantaneous risk during all time intervals resulting in a lower corresponding cumulative remaining lifetime hazard of MACE as compared to persons with average PGS. Therefore, the problem specification changes. In this case, we no longer want to solve for how much more we need to lower LDL, SBP, or both to overcome the increased risk due to a person's inherited polygenic burden (as measured crudely by their PGS), but instead, we can calculate how much less we need to lower LDL, SBP, or both to achieve a specific goal because they appear to be less genetically vulnerable based on inherited polygenic predisposition to experience a MACE.

Computationally, we perform the same calculations as above, but return a negative number which represents how much less aggressive we need to be to lower LDL, SBP, or both because this person's instantaneous and cumulative remaining lifetime hazard of MACE is HIGHER when we ignore their PGS.

That is, their instantaneous hazard of MACE during every time interval, and their corresponding cumulative remaining lifetime hazard of MACE is higher when we ignore their PGS. Including the effect of their PGS allows us to lower their predicted cumulative remaining lifetime risk of MACE. Thus, we may be less aggressive at lowering LDL, SBP, or both than we would calculate if we didn't include their PGS in the analysis.

Thus providing an additional layer of precision, in some embodiments, when individualizing guidance on how to personalize the prevention of MACE by lowering LDL to slow the progression of atherosclerosis, and lowering SBP to slow the progression of atherosclerosis at vulnerable branch points, reduce the propensity of the cumulated plaque to disrupt at any point in time, and reduce the accumulation of arterial wall injury to maximize the capacity of the artery to tolerate the accumulated plaque burden.

### Determining Benefit of Sequences of Therapeutic Interventions

Returning now to the illustrative process shown in FIG. 2D, acts 231-233 may be performed to estimate the benefit of one or more sequences of therapeutic interventions.

A sequence of therapeutic interventions may be of any suitable type. For example, the sequence of therapeutic interventions may be designed to lower LDL, to lower SBP, or to lower both LDL and SBP.

Therapeutic intervention sequences may differ from one another based on type or types of therapeutics utilized, amounts of the therapeutic or the therapeutics administered, and/or timing of administering the therapeutic or therapeutics.

A particular therapeutic intervention sequence may be defined by information specifying, for each particular therapeutic intervention in the sequence, a type of therapeutic or therapeutics part of the particular therapeutic intervention, an amount of the therapeutic or the therapeutics to administer as part of the particular therapeutic intervention, and timing information indicating when to administer the particular therapeutic intervention. For example, a particular therapeutic intervention may include a sequence one or more, optionally annual or bi-annual, administrations of an intervention selected from a DNA-based therapy (e.g., gene therapy, antisense therapy, etc.), RNA-based therapy (e.g., mRNA, siRNA, miRNA, etc.), protein-based therapy (e.g., peptide therapy, antibody therapy, hormone therapy, enzyme therapy, etc.), and pharmacological therapy (e.g., small molecule drug therapy). Example therapies are described herein including in the section titled: “Therapeutic Intervention(s) and Additional Uses.”

To estimate the benefit of multiple sequences of therapeutic interventions, the therapeutic sequences to be evaluated are first generated, which is done at act 231. This may be done in any suitable way, for example, by enumerating a set of therapeutic intervention sequences by varying an age of the subject for when to begin an intervention therapeutic sequence, the duration of the intervention therapeutic sequence, the types of therapeutic or therapeutics used as part of the therapeutic intervention sequence, and/or the timings of therapeutic interventions in the therapeutic intervention sequence.

After the multiple sequences of therapeutic interventions are generated at act 231, one or more such sequences may be filtered out from subsequent evaluation at act 232. This may help to limit the number of potential combinations and sequences of LDL and SBP lowering therapies to be evaluated. For example, in some embodiments, only those therapeutic intervention sequences that continue the current intervention, intensify the current intervention, and/or add another intervention are considered. As another example, discontinuous intervention sequences that start and stop therapeutic interventions in random order may be filtered out from subsequent consideration. Additionally or alternatively, any other suitable clinical considerations may be used to identify which therapeutic intervention sequences are clinically plausible (and therefore should be considered for the benefit they provide) and which ones are not (and therefore should be eliminated from subsequent consideration). The set of one or more such clinical considerations, encoded into one or more rules that can be used (e.g., in a computer implemented setting) to filter out one or more therapeutic intervention sequences, may be referred to as a “domain expert clinical translation heuristic.”

It should be appreciated that, instead of generating multiple sequences of therapeutic interventions and filtering one or more sequences using the rule(s), the rule(s) may be used to generate the multiple sequences of therapeutic interventions that comply with the rule(s) ab initio, so that the filtering step may be omitted.

Next, the process of FIG. 2D proceeds to act 233, where the benefit of administering each the filtered therapeutic intervention sequence(s) may be evaluated.

In some embodiments, determining the respective benefits (e.g., as reflected by reductions in risk, a value function, etc.) of administering each of the multiple therapeutic intervention sequences to the subject comprises determining, for each particular therapeutic intervention sequence under consideration, expected proportional reduction and/or absolute reduction in risk of cardiovascular events for the subject in response to the particular therapeutic intervention sequence. This produces multiple expected proportional and/or absolute reductions in risk for the considered therapeutic intervention sequences, which may be stored for subsequent use and/or used in selecting a specific therapeutic sequence to recommend administering (or actually administering) to the subject.

Therefore, in some embodiments, as part of act 233, determining the benefit of a particular therapeutic intervention sequence (under consideration from among one of the filtered therapeutic intervention sequences) may involve determining the expected proportional reduction and/or absolute reduction in risk of cardiovascular events for the subject in response to the particular therapeutic intervention sequence. This may be done by using at least one second ML model (e.g., at least one c-DNN-ODE model) as described herein.

For example, in some embodiments, determining the expected proportional reduction and/or the absolute reduction in risk of cardiovascular events for the subject in response to the particular therapeutic intervention sequence comprises:
(a) for each particular interval of follow-up for the subject from the age of the subject at which the particular therapeutic intervention sequence is to commence to an upper threshold age (e.g., 80),
   (i) determining, using the multiple measures of risk (e.g., obtained at act 220), a predicted instantaneous hazard rate of the subject having a cardiovascular event at the particular interval of follow up;
   (ii) determining, using the at least one second ML model (e.g., at least one c-DNN-ODE model), a time-averaged instantaneous log hazard ratio for a one unit lower LDL and/or SBP corresponding to duration of treatment (e.g., in accordance with the particular therapeutic intervention sequence) at the particular interval of follow-up for the subject;
   (iii) determining an intervention-adjusted instantaneous hazard for the particular interval of follow-up by multiplying the instantaneous hazard rate of the subject determined at (a)(i) with the time-averaged instantaneous log hazard ratio determined at (a)(ii), thereby obtaining multiple intervention-adjusted instantaneous hazards for intervals of follow-up evaluated at (a);
(b) determining, using the multiple intervention-adjusted instantaneous hazards, intervention-adjusted cumulative hazard rates and cumulative event rates of cardiovascular events for intervals of follow-up for the subject from the age of the subject at which the particular therapeutic intervention sequence is to commence to the upper threshold age;
(c) determining predicted proportional reductions in the risk of experiencing a cardiovascular event as ratios of the intervention-adjusted cumulated hazard rates and the cumulative hazard rates that are not adjusted for the particular therapeutic intervention sequence; and
(d) determining predicted absolute reductions in the risk of experiencing a cardiovascular event as absolute differences between the intervention-adjusted cumulative event rates and the cumulative event rates that are not adjusted for the particular therapeutic intervention sequence.

As described herein, the at least one second trained ML model may be at least one causal DNN for Ordinary Differential Equations (c-DNN-ODE) model (e.g., at least one DNN-ODE model that is causal because it is trained on randomized data), a non-linear regression model, an adaptive basis function regression model, a neural network regression model, a deep neural network regression model, a logistic regression model, a polynomial regression model, a decision tree regression model, a random forest regression model, and/or a gradient boosted decision tree regression model.

In some embodiments, where the c-DNN-ODE model is used, in response to input indicating a duration of sustained intervention, optionally specified in intervals of months or years, the c-DNN-ODE model is trained to provide: output indicating magnitude of an instantaneous log hazard ratio of benefit of lowering LDL or SBP by one unit for each interval of the duration of sustained intervention, and rate of change, with respect to the input indicating the duration of sustained intervention, of the magnitude of the instantaneous log hazard ratio of benefit of lowering LDL or SBP by one unit for each interval of the duration of sustained intervention.

### Identifying Therapeutic Intervention(s) to be Administered to Subject

As shown in FIG. 2A, process 200 includes act 240 that involves using the determined benefit of administering the one or more therapeutic interventions to identify at least one therapeutic intervention (e.g., a particular sequence of interventions) to recommend to be administered to the subject.

In some embodiments, this may be performed in accordance with the illustrative process shown in FIG. 2E. As shown in FIG. 2E, act 240 may involve: (1) identifying, at act 241, one or more therapeutic intervention sequences to evaluate using a domain expert policy guideline for selecting an intervention sequence for a treatment goal; (2) determining a sequence score, at act 242, for each therapeutic intervention sequence (identified at act 241); and (3) selecting, at act 243, an intervention sequence for the subject based on the determined sequence scores (at act 242).

As described above, with respect to act 230, the benefits of numerous therapeutic intervention sequences may be evaluated. The process illustrated in FIG. 2E is for selecting, from among all such sequences, at least one therapeutic intervention sequence to recommend administering to the subject.

As a first step in this process, at act 241, the set of therapeutic sequences under consideration may be further filtered based on the subject's treatment goals. The treatment goals may be personalized to the subject thereby facilitating discovery of the optimal sequence of therapeutic interventions that personalizes the prevention of cardiovascular disease to the subject under consideration. To this end, a domain expert policy guideline may be used as a mechanism to inject “clinical expertise” into the therapeutic intervention sequence selection process such that the recommended sequence of actions achieves an explicitly defined overall goal that is consistent with personal preferences, local clinical practice guidelines, and/or other clinical or policy objectives. The domain expert policy guideline may have parameters which may be set to default values or other values to customize the guideline to the subject thereby guiding selection of a sequence of therapeutic interventions that is personalized to the subject.

As one example, the default parameters of the domain expert policy guideline may be set as follows.

The domain expert policy guideline may define a goal of preventing development of cardiometabolic (e.g., cardiovascular) disease. The goal may be defined as one or more of:
1. Maintaining a cumulative lifetime risk of experiencing a cardiovascular event (e.g., an atherosclerotic cardiovascular event) of less than 5% at all ages up to age 80 years (both the desired cumulative event rate threshold and the age or duration of follow-up may be changed to permit consideration of both short-term and long-term benefits; and different intensities of short-term or long-term therapeutic goals);
2. Preventing the development of hypertension (with a customizable SBP threshold for the diagnosis of hypertension);
3. Preventing the development of T2D (with a customizable HbA1c threshold to initiate interventions to either lower, or prevent further rises, in HbA1c); and/or
4. Lowering LDL, SBP, weight, and HbA1c by just the amount each person needs—when they need it—to personalize the prevention of cardiovascular events (including MI and stroke), hypertension, and T2D; using the fewest total intervention units possible (e.g., to maximize clinical and economic return on investment).

Additionally, the domain expert policy guideline may indicate a preference for an initial type of intervention in order to achieve the desired goal. That goal, for example, may be to lower LDL by the amount needed to slow the rate of plaque progression enough to keep the predicted size of the accumulated plaque burden at age 80 years below the selected personal plaque threshold (e.g., the cumulative exposure to LDL at which the cumulative lifetime risk of atherosclerotic cardiovascular events is predicted to reach 5% for the person under consideration). As described herein, this personal plaque threshold (measured in cumulative exposure to LDL (Plaque Years of LDL in mmol/L) needed to keep the cumulative lifetime risk of cardiovascular events below 5% is conditional on the subject's current LDL level, the existing size of their accumulated plaque burden (defined by their current cumulative exposure to LDL), their predicted rate of plaque progression, and their exposure to other features that reduce the capacity of the artery wall to tolerate the accumulated plaque burden.

Additionally, the domain expert policy guideline may indicate one or more subsequent intervention(s) to be added in furtherance of achieving the desired goal.

As one example, the domain expert policy guideline may indicate conditions in which adding an SBP lowering intervention should be considered. For example, adding an SBP lowering intervention may be considered: (a) when needed to ensure that the remaining lifetime risk of cardiovascular (e.g., atherosclerotic cardiovascular) events remains below the selected threshold of 5% at all ages up to age 80 years, by protecting the artery wall from additional accumulating irreversible structural injury caused by elevated SBP; (b) when the subject's SBP exceeds 130 mmHg to prevent further rises in SBP and thus prevent the development of hypertension; or (c) whichever of (a) and (b) comes first, thus ensuring the prevention of both cardiovascular events and hypertension.

As another example, the domain expert policy guideline may indicate conditions in which an intervention to prevent weight gain or increased adiposity (e.g., a Nutrient Stimulated Hormone—NuSH, or other intervention), by preventing further excess energy balance to thus prevent further rises in HbA1c, should be considered. For example, adding such an intervention may be considered: (a) when needed to ensure that the remaining lifetime risk of cardiovascular events remains below the selected threshold of 5% at all ages up to age 80 years, by protecting the artery wall from additional accumulating irreversible structural injury caused by elevated circulating glucose levels; (b) when HbA1c levels exceeds 5.7% to 6.0% (depending on age) to prevent further rises in HbA1c and thus prevent the development of T2D; or (c) whichever comes first, thus ensuring the prevention of both atherosclerotic cardiovascular events and T2D.

It should also be appreciated that although the default settings of the domain expert policy guideline focus on lowering LDL, SBP, and/or HbA1c—the guideline may be extended to include additional interventions that reduce Lp(a) and other interventions (including therapies directed against triglyceride-rich apoB-containing lipoproteins, as well as the effects of diet, and exercise).

Accordingly, in some embodiments, at act 241, one or more of the set of therapeutic sequences under consideration may be further filtered in accordance with the applicable domain expert policy guideline. For example, any of therapeutic sequences under consideration that are inconsistent with the domain expert policy guideline may be removed. For instance, if a particular therapeutic intervention sequence begins with an SBP lowering therapeutic intervention, whereas the guideline states that an LDL lowering therapeutic intervention is to be administered first, the particular therapeutic sequence may be removed from the set of therapeutic intervention sequences being considered.

Next, at act 242, the multiple therapeutic intervention sequences (not including any sequences filtered out at act 241) may be scored to obtain a respective set of therapeutic intervention scores. In turn, at act 243, a therapeutic intervention sequence may be selected to be recommended for the subject based on the scores calculated at act 242. For example, the therapeutic intervention sequence having the highest score may be selected. As another example, a number of higher-scoring therapeutic intervention sequences (e.g., any sequences with a score above a threshold score) may be selected and presented, for example, to a clinician who may make a further selection from among them.

Returning to act 242, in some embodiments, determining a sequence score for a specific therapeutic intervention sequence may be done in any of a number of ways. For example, a sequence score for a specific therapeutic intervention sequence may be determined using a value function, as described herein. However, it is worth first noting that, indeed, the overall problem of selecting an optimal intervention sequence may be considered in the context of a reinforcement learning (RL) framework as described herein including in Example 5, with the value function at act 242 serving as the value function within the RL framework. We briefly describe the RL framework now before describing the value function further; additional aspects of the RL framework are described in Example 5. Alternative example ways of determining sequence scores for specific therapeutic intervention sequences are also described in Example 5.

As described in Example 5, the RL framework may be realized using a RL model-dependent (MD) proximal policy optimization (PPO) algorithm constrained by the domain expert defined policy guideline. The policy is model dependent in that the value function depends on the various ML algorithms and models described herein. The algorithm is a “proximal policy” optimization algorithm in that it seeks to discover the first step in the optimal sequence of actions needed to achieve the desired goal of personalizing the prevention of cardiometabolic (e.g., cardiovascular) disease for the subject. The objective of the RL algorithm then is to discover that first step, and then: use the observed reductions in the targeted cause of disease achieved in response to the recommended action, in combination with the biological changes in other exposures not targeted by the recommended action, to learn how the person's cardiometabolic health is evolving over time. That information, in turn, can be used to adjust the estimates of risk (e.g., of a cardiovascular event) and benefit (e.g., of various therapeutic treatments) to either: reinforce the recommended next action in the previously selected sequence of interventions (i.e., keep going with the previously selected sequence of therapeutic interventions) or adjust the next recommended action to ensure the desired goal is being achieved (i.e., adjusting the previously selected sequence of therapeutic interventions).

In some embodiments, the number of sequences therapeutic interventions may be computationally manageable such that a score for each therapeutic intervention sequence may be computed (by brute force) using the value function. However, in other embodiments, when the number of possible sequences is too vast for exhaustive search, an approximate search technique may be used. For example, a Markov chain tree search (MCTS) algorithm may be utilized. For example, to create a more computationally feasible solution space, an MCTS algorithm may be used to randomly generate a large number of possible sequences of interventions to lower LDL, SBP, or both for consideration.

Returning now to the value function, evaluating a value function for a specific therapeutic intervention sequence generates a specific sequence score for that sequence.

In some embodiments, the value function has a reward component, which reflects a measure of reward for the specific therapeutic intervention sequence. For example, the measure of reward may be specified in terms of the intervention-adjusted cumulative hazard rates (adjusted for the specific intervention). For instance, the measure of reward may be specified as a (optionally, weighted by discount factors) sum of the intervention-adjusted cumulative hazard rates for intervals of follow-up for the subject from the age of the subject at which the specific therapeutic intervention sequence is to commence to an upper threshold age (e.g., 80). The intervention-adjusted cumulative hazard rates may be computed at act 230 (e.g., using the c-DNN ODE model(s)), as described herein.

Accordingly, in some embodiments, the predicted cumulative hazards of experiencing an atherosclerotic cardiovascular event at all ages between the current age and 80 years are summed (or integrated to produce the area under the cumulative event curve) and the objective is to minimize this value and therefore minimize the cumulative lifetime risk of having a cardiovascular event.

In addition, in some embodiments, where the measure of reward is determined as a sum of rewards over multiple time intervals, the rewards in the sum of rewards corresponding to later time intervals during the specific therapeutic intervention sequence may be discounted relative to earlier time intervals during the specific therapeutic intervention sequence.

Such discounting may be designed to prioritize preventing the development of cardiometabolic disease early in life because early onset of disease has the greatest clinical and economic costs. In addition, the discounting may be designed to prioritize (strategy A) early interventions that produce modest, sustained reductions in LDL over time to slow the progression of atherosclerosis and thus minimize the residual risk of cardiovascular events caused by the accumulated plaque burden present at any point in time over (strategy B) more aggressive LDL lowering, and combinations of LDL and SBP interventions, started later in life. This is because strategy B allows atherosclerotic plaque and arterial wall injury to accumulate early in life, leading to a larger plaque burden, and more irreversible structural injury to the artery wall accumulating early in life before therapy is initiated, which leads to a higher residual risk and a corresponding higher absolute rate of events that occur earlier in life, even though the total cumulative event rate by age 80 years may be very similar (because more aggressive later treatment prevents fewer early events but a greater number of later events, as compared to modest early sustained reductions in LDL).

In addition, in some embodiments, the value function may have a penalty component to penalize the number of treatments and/or treatment magnitude. In this way, therapeutic intervention sequences having a greater number of treatments and/or a stronger intensity of treatments are penalized and have lower scores than other therapeutic intervention sequences, for achieving clinical goal(s) of treating the subject.

For example, in some embodiments, a penalty may be added for each intervention that is used during every year that the intervention is used. The penalty may be designed to achieve the goal of lowering LDL, SBP, weight, and HbA1c by only the amount that each person needs, when they need it, to achieve the desired goal using the fewest number of treatments. The penalty may also be designed to prioritize the use of novel combination therapies (which are penalized as a single unit) and novel therapies that ensure compliance, and thus produce the maximum absolute reduction in the targeted modifiable cause of disease and greatest corresponding reductions in clinical events.

In addition, the penalty may also be designed to prevent selection of the sequence of interventions defined by lowering LDL, SBP, weight, and HbA1c by the maximum amount beginning at the current age and extending until age 80 years. Although this particular sequence of interventions will produce the greatest proportional and absolute reductions in the risk of atherosclerotic cardiovascular events while preventing hypertension and T2D, it would not achieve the goal of lowering LDL, SBP, weight, and HbA1c by only the amount needed—and only when needed—to personalize prevention for the person under consideration. It would also represent the sequence of actions that consumes the maximum amount of resources, thus reducing the economic return on investment.

An illustrative example of a value function is provided in the Example 5, which is reproduced for reference below:

$${V ( s )}_{i} = {\sum }_{age = i}^{80} {( ( ( - 100 ) * cumHaz ) * ( discount ) )}_{i} - {\sum }_{age = i}^{80} ( {( no , Rx )}_{i} )$$

Example 5 also includes illustrative examples of calculating this value function for particular examples of clinical strategies, including an example of selecting one therapeutic intervention sequence over another on the basis of the scores determined for the two sequences using this value function.

As may be appreciated from the foregoing discussion of the process shown in FIG. 2E (relating to act 240 of process 200 shown in FIG. 2A), after a therapeutic intervention sequence is selected, information about that intervention sequence, and more generally any of the results of the biomarker, risk, and benefit calculations performed during acts 210-240 may be presented to one or more users (e.g., the subject, the subject's doctor or clinical team, an insurer, etc.).

In some embodiments, the output provided may include: information about the intervention sequence selected, an indication of how the subject's risk of a cardiovascular event is changed as a result of the specific therapeutic intervention sequence, and/or an accompanying narrative explanation. The narrative explanation may be generated by prompting a language model, for example a large language model, with predetermined prompts and values of various types of predictions (e.g., biomarker trajectories, measures of risk, measures of benefit of intervention, personalized plaque threshold, etc.) determined using the methods described herein. The narrative explanation may include reasoning and biological rationale of why the subject is at risk for cardiovascular disease, how the subject can minimize their risk, how much the subject will benefit from the specific therapeutic intervention sequence, a recommended immediate action, and/or a description of one or more expected subsequent actions to be take and when to take them based on the subject's current state of cardiovascular health.

In some embodiments, the output may be provided via a graphical user interface (GUI), for example, as shown in FIGS. 7A, 7B, and 7C. For example, as shown in FIG. 7A, the GUI may provide a predicted remaining cumulative lifetime risk of having an ASCVD at all ages, the rate of rising SBP and the corresponding age at which HTN is likely to develop, and the personal weight threshold T2D at which HbA1c is like to exceed 6.5%. In addition, as shown in FIG. 7B, the GUI may provide information describing the intervention(s) needed to achieve the desired goal, including one or multiple specific actions to take depending on when the interventions commence (e.g., now vs. later).

The techniques described herein are flexible and may be adapted to a variety of purposes—including guiding precision health, designing clinical trials, monitoring the success of interventions, conducting economic analyses, and designing bespoke financial & insurance instruments. For example, continuing the example of FIGS. 7A and 7B, a bespoke goal for cumulative lifetime risk of ASCVD events may be set, conditional on also preventing HTN and diabetes, and FIG. 7C illustrates alternative recommendations for interventions that may be provided responsive to the set goal.

In this example, the recommended action for the subject is the specific action that the subject should take immediately (and may include observation or other non-pharmacologic therapies), conditional on the recommended future combination and sequence of additional interventions anticipated to start only when needed. This is the optimal proximal policy (or immediate action) that maximizes the expected future cumulative benefits. For example, the initial action may be to reduce LDL by 33% (1.2 mmol/L for this person) to slow plaque progression enough to keep this person below their personal plaque threshold at which cumulative lifetime risk of ASCVD events exceed 5%, with the plan to continue this therapy indefinitely and further protect the artery wall to maximize the capacity of the artery to tolerate the permitted rate of plaque progression by adding an SBP lowering therapy to lower SBP by 5-10 mmHg beginning in 15 years at age 55 when the person's SBP is predicted to rise to 135 mmHg, and adding a NuSH or other dietary intervention to prevent further weight gain by preventing excess energy balance and thus prevent further rises in HbA1c when the person's HbA1c exceeds 6%. Preventing further rises in SBP beyond 135 mmHg (or other selected level) prevents the development of hypertension, while preventing further rises in HbA1c above 6% prevents the development of T2D. Both of these interventions also protect the artery wall thus obviating the need for more intense LDL lowering by maximizing the capacity of the artery wall to tolerate the accumulating plaque burden.

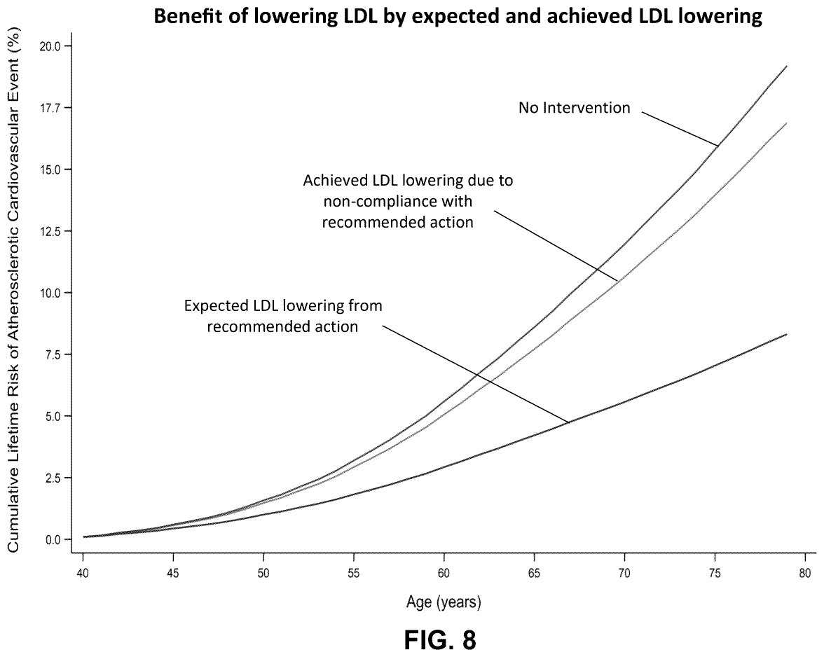

The recommended action (and subsequent combination and sequence of recommended actions over time) are based on the expected absolute reductions over time of the targeted modifiable causes of disease (and the predicted evolution in all other exposures as a person's cardiometabolic health evolves over time to maximize the expected cumulative benefit (cumulative value function). However, the realized clinical benefit depends on the achieved absolute reduction in the modifiable causes of disease in response to the recommended action(s). Thus, the clinical and economic benefit of a recommended action is determined by the achieved absolute reduction in LDL (or SBP), which in turn depends on compliance with the recommended intervention(s), as shown in FIG. 8.

### Longitudinal Monitoring of Subject

As described herein, the process 200 may be used to monitor a subject over time and adjust the interventions for the subject. Such monitoring and adjustments may be needed to take into account the achieved absolute reduction in the modifiable cause of disease targeted by the recommended intervention and the absolute changes in other exposures not targeted by the recommended interventions(s) due to aging and the evolving biology of how common diseases develop during the same interval of longitudinal follow-up.

To this end, after act 240 is completed, process 200 may proceed to decision block 250, where it is determined that the analysis of acts 210-240 is to be repeated, for example, after some amount of time passes (e.g., a year, multiple months, a period of time between visits of the subject to their clinician, etc.). When it is determined that the analysis of acts 210-240 is not to be repeated, process 200 ends, as previously described.

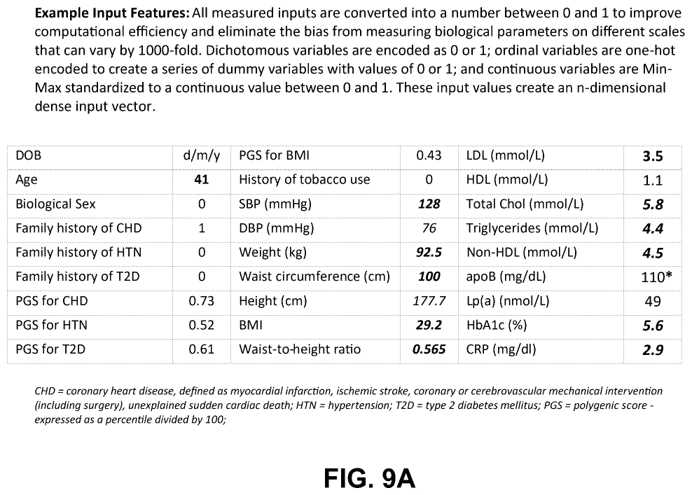

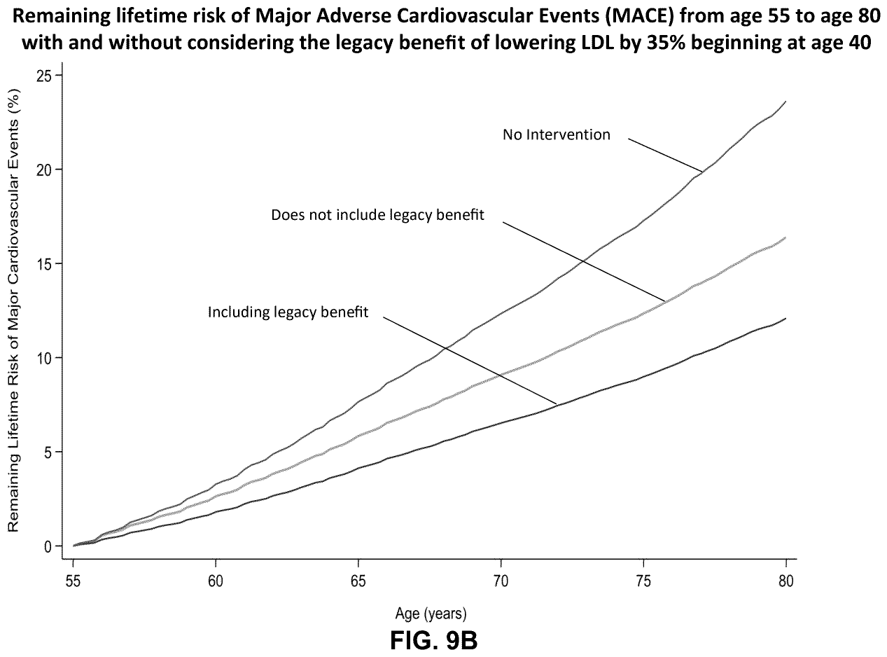

On the other hand, when it is determined that the analysis of acts 210-240 is to be repeated, process 200 returns to act 210, where updated cardiometabolic health data is obtained for the subject. The updated cardiometabolic data may reflect various changes to the subject, some of which are due to achieved absolute reductions in modifiable disease causes (e.g., LDL levels, SBP levels, HbA1c levels, etc.) and other changes due to aging and/or development of other diseases. FIG. 9A shows an example of updated cardiometabolic measurements obtained for the subject.

The changes in the measured clinical, biometric, and biochemical features over the previous time interval may then be used to perform updated predictive and prescriptive analyses. The updated cardiometabolic measurements allow for the learning of the individual trajectories of each person more precisely based on the observed changes over time thus creating more precise individualized estimates of each person's projected trajectories of non-targeted levels of LDL, apoB Lp(a), SBP, weight, waist circumference, and HbA1c. These more precise estimates of how each person's cardiometabolic health is evolving over time, in turn, inform more precise predictions of risk and the expected benefit of specific interventions that are used to longitudinally monitor cardiometabolic health over time and make updated recommendations to ensure that cardiometabolic health is being preserved.

As such, the next iteration of the process involves, at act 220, determining, using at least some of the updated cardiometabolic health data, the first trained machine learning (ML) model and for each of multiple second time intervals, one or more updated measures of risk that the subject develops cardiovascular disease to obtain multiple updated measures of risk corresponding to the multiple second time intervals. Next process 200 involves, at act 230, determining, using the multiple updated measures of risk that the subject develops cardiovascular disease and the at least one second trained ML model (e.g., a c-DNN-ODE for predicting benefit LDL reduction, a c-DNN-ODE for predicting benefit of SBP reduction, a c-DNN-ODE for predicting benefit of reduction of both LDL and SBP, etc.), benefit of administering to the subject one or more second therapeutic interventions designed to reduce risk of cardiovascular disease by targeting one or more modifiable causes of the cardiovascular disease. Then, process 200 involves, at act 240, identifying, using the determined benefit of administering the one or more therapeutic interventions, at least one second therapeutic intervention to recommend to be administered to the subject. The second therapeutic intervention may involve continuing administering the initial therapeutic intervention sequence identified the first time through the process 200 (e.g., during the first time a subject's cardiometabolic data is analyzed) and/or adding a new therapeutic to the initial therapeutic intervention sequence. Other modifications to the initial therapeutic intervention sequence are also envisaged.

Thus, updated values of a person's clinical characteristics, physical biometrics, and biochemical measurements may be used to provide: (1) an updated assessment of their current state of cardiometabolic health; (2) an updated prediction of the risk of cardiometabolic disease over all subsequent time intervals based on the updated state of cardiometabolic health and the reductions in the modifiable causes of disease achieved in response to any prior interventions; (3) an updated prediction of the expected benefit of interventions designed to reduce exposure to the modifiable causes of disease based on the updated state of cardiometabolic health and the reductions in the modifiable causes of disease achieved in response to prior interventions—including consideration of the legacy benefit from the achieved absolute reductions in the modifiable causes of disease in response to earlier interventions (legacy benefit of prior interventions should be considered in subsequent iterations of analysis as shown in FIG. 9B and described in a detailed example in Example 6); and (5) updated recommendations about the optimal timing, type, intensity, combination, and sequence of actions needed to ensure the prevention of coronary heart disease (CHD), such as myocardial infarction (MI), angina, and coronary artery stenosis; cerebrovascular disease, such as a transient ischemic attack, ischemic stroke, and carotid artery stenosis; and, hypertension.

After another iteration of process 200 (e.g., after the second time acts 210-240 are performed), updated output may be provided by the software performing process 200. The updated output may include, for example: (1) an updated recommendation for the optimal next immediate action (e.g., continue the current therapeutic intervention sequence, intensify the current intervention sequence, add another intervention to the sequence); and/or (2) an updated narrative explaining the reasoning and biological rationale for why a person is at risk, how their cardiometabolic health is evolving, how to reduce their risk of cardiometabolic disease, how much they have benefited from previous actions, how much they would benefit from subsequent actions to prevent disease, and/or updated recommendations for the optimal sequence of actions needed to prevent cardiovascular disease.

The process 200 may repeat iteratively at regular follow-up intervals to monitor cardiometabolic health, and adjust guidance about the optimal recommended actions and sequences of actions to promote the prevention of MI, stroke, and hypertension, and potentially T2D based on the person's evolving cardiometabolic health and achieved reductions in the modifiable causes of disease in response to the recommended actions.

### Therapeutic Intervention(s) and Additional Uses

Aspects of the technology relates to interventions used to lower modifiable causes (risk factors) of disease, such as modifiable causes of cardiometabolic disease, including cardiovascular disease (e.g., atherosclerotic cardiovascular disease (ASCVD)). In some embodiments, one or more interventions are used to prevent or treat ASCVD, which is caused by plaque buildup in arterial walls. ASCVD includes the following conditions: coronary heart disease (CHD), such as myocardial infarction (MI), angina, and coronary artery stenosis; cerebrovascular disease, such as a transient ischemic attack, ischemic stroke, and carotid artery stenosis.

Modifiable causes (or risk factors) of disease herein include but are not limited to cholesterol, blood pressure, blood glucose, and weight, among others. Biomarkers associated with modifiable causes of diseases, including cardiometabolic diseases, such as ASCVD and diabetes, can be targeted to modify those causes. For example, such biomarkers include physical measurements, such as systolic blood pressure (SBP), diastolic blood pressure (DBP), weight, waist circumference, height, and the derived measurements of body mass index (BMI), and waist-to-height ratio, as well as biochemical measurements, such as plasma low density lipoprotein (LDL) (apoB) level, lipoprotein(a) (Lp(a)) level, high density lipoprotein (HDL) level, triglycerides (TG) level, and HbA1c level.

The present disclosure is not limited by the type of intervention used to alter a modifiable cause of a disease. Therapeutic (including preventative) interventions encompass a diverse array of modalities designed to improve health outcomes and lower risk of developing cardiovascular disease. These interventions can be broadly categorized based on the nature of treatment-whether medical (e.g., pharmacological, surgical, etc.), or lifestyle-based—and can be selected according to the specific condition, therapeutic goals, and patient health profile.

For example, medications are often prescribed to slow the progression of ASCVD. Statins effectively lower LDL cholesterol levels and reduce cardiovascular events. Antihypertensive drugs, such as ACE inhibitors, ARBs, beta-blockers, and calcium channel blockers, help control high blood pressure, a significant modifiable cause of ASCVD. Antiplatelet agents like aspirin may be recommended to prevent blood clots and reduce the risk of heart attack and stroke. For individuals whose cholesterol levels remain high despite statin therapy, PCSK9 inhibitors offer an additional option to lower LDL cholesterol effectively.

In cases where lifestyle changes and medications are insufficient, medical procedures may be used. Percutaneous coronary intervention (PCI), also known as angioplasty, is a minimally invasive procedure where a balloon is inserted to open narrowed arteries, often followed by stent placement to maintain the artery's openness. For more severe cases, coronary artery bypass grafting (CABG) creates a new pathway around blocked arteries using vessel grafts, improving blood flow to the heart muscle.

The present disclosure also provides a myriad of therapeutic modalities that may be used to modify a cause of disease (e.g., modify the level of a biomarker associated with a modifiable cause of disease), including DNA-based therapies, RNA-based therapies, protein-based therapies, cell-based therapies, and small molecule drugs.

In some embodiments, an intervention is a DNA-based therapy. DNA-based therapies include gene therapy, where functional genes are delivered to replace or repair defective ones, and genome editing techniques like CRISPR-Cas9, TALENs, and zinc-finger nucleases, which allow precise modifications of the genome to correct genetic abnormalities. Emerging epigenetic therapies aim to modulate gene expression without altering the DNA sequence itself, providing a reversible approach to managing diseases.

In some embodiments, an intervention is an RNA-based therapy. RNA-based therapies leverage messenger RNA (mRNA), small interfering RNA (siRNA), and antisense oligonucleotides (ASOs) to regulate protein production. mRNA vaccines and therapeutics deliver genetic instructions to produce therapeutic proteins or trigger immune responses. siRNA and ASOs specifically target and degrade harmful RNA sequences or block their translation.

In some embodiments, an intervention is a protein-based therapy. Protein-based therapies include monoclonal antibodies, therapeutic proteins, and enzyme replacement therapies. Monoclonal antibodies are engineered to target specific antigens. Therapeutic proteins, such as insulin, replace or supplement deficient or malfunctioning proteins in patients with conditions like diabetes. Enzyme replacement therapies provide functional enzymes to patients with enzyme deficiencies.

In some embodiments, an intervention is a cell-based therapy. Cell-based therapies involve the use of living cells to restore, replace, or enhance biological functions. These include stem cell therapies and immune cell-based therapies. Other examples include mesenchymal stem cells and induced pluripotent stem cells (iPSCs). In some embodiments, an intervention is a small molecule drug. Small molecule drugs include low molecular weight compounds that can modulate biological pathways by interacting with specific molecular targets, such as enzymes, receptors, or ion channels. These drugs are widely used for their ability to penetrate cells and affect intracellular processes, making them effective in treating a variety of diseases, including cardiometabolic diseases.

### Therapeutic Interventions for Lowering LDL, AGT, and Lp(a) Levels

In some embodiments, the present disclosure relates to compositions and methods for modulating modifiable causes of cardiovascular disease, including low-density lipoprotein (LDL), angiotensinogen (AGT), and/or lipoprotein(a) (Lp(a)). Elevated levels of LDL, AGT, and/or Lp(a) have been associated with increased risk of adverse cardiovascular outcomes, including atherosclerotic cardiovascular disease, coronary artery disease, stroke, and heart failure. As described herein, therapeutic intervention targeting one or more of these modifiable causes of disease can improve cardiometabolic health and slow the progression of cardiovascular disease.

In some embodiments, a therapeutic intervention comprises one or more small interfering RNA (siRNA) molecules designed to reduce the expression of a target gene in hepatocytes (or other target tissues) thereby lowering circulating levels of LDL, AGT, and/or Lp(a), and thereby improving cardiometabolic health and limiting progression of cardiovascular disease.

### LDL-Lowering Interventions

In some embodiments, a therapeutic intervention reduces circulating LDL levels. LDL-lowering can be achieved by any suitable modality, including, without limitation, small-molecule drugs, biologics (e.g., antibodies, peptides, fusion proteins), lipid-lowering agents (e.g., statins, ezetimibe), RNA-targeted therapeutics (e.g., siRNA, antisense oligonucleotides), gene-editing or gene-regulatory systems (e.g., CRISPR/Cas systems, base editors, epigenome editors), viral vectors (e.g., AAV, lentiviral vectors), genome-integrating or non-integrating nucleic acid delivery vehicles, vaccines, cell-based therapies, dietary interventions, or combinations thereof. Non-limiting examples of small-molecule drugs that lower cholesterol include statins (e.g., atorvastatin, simvastatin, rosuvastatin, pravastatin, lovastatin, fluvastatin, pitavastatin), ezetimibe, bempedoic acid, and fibrates (e.g., fenofibrate, gemfibrozil). Non-limiting examples of statins include atorvastatin, simvastatin, lovastatin, pravastatin, rosuvastatin, fluvastatin, and pitavastatin. Non-limiting examples of antibodies include alirocumab and evolocumab.

In some embodiments, a therapeutic intervention inhibits PCSK9 expression or activity, upregulates LDL receptor (LDLR) expression, enhances LDL clearance, reduces LDL production, or otherwise modulates LDL metabolism.

In some embodiments, the target gene is PCSK9 (proprotein convertase subtilisin/kexin type 9), the hepatic expression of which reduces LDL receptor recycling and thereby elevates LDL-cholesterol (LDL-C).

Silencing PCSK9 expression via siRNA increases LDL receptor density on hepatocytes and enhances LDL clearance, thereby lowering LDL levels.

One commercially available agent is Inclisiran (brand name Leqvio®), a GalNAc-conjugated double-stranded siRNA targeting PCSK9 mRNA, administered subcutaneously (initial dose followed by 3 months and then every 6 months) and shown to reduce LDL-C by ~50% or more in clinical trials. Other siRNA agents are described in International Publication No. 2025/096355, the siRNA agents of which are incorporated herein by reference. Other siRNA agents targeting PCSK9 can be used.

In some embodiments, an LDL-targeting siRNA is administered in combination with statins, ezetimibe, PCSK9 monoclonal antibodies, or other lipid-lowering agents.

In some embodiments, a composition comprising a GalNAc-conjugated siRNA targeting AGT mRNA, is administered subcutaneously quarterly, semi-annually, or annually, in patients with hypertension, such as those not achieving target blood pressure on standard therapy.

### AGT-Lowering Interventions

In some embodiments, a therapeutic intervention reduces circulating or tissue levels of AGT (angiotensinogen). AGT is a precursor in the renin-angiotensin system (RAS), and elevated AGT levels contribute to vasoconstriction, hypertension, and end-organ damage. Accordingly, reducing AGT levels or activity represents a therapeutic approach to improving cardiovascular health, lowering blood pressure, and limiting progression of cardiovascular risk.

AGT-lowering interventions can employ any suitable modality, including, without limitation, small-molecule inhibitors, antisense oligonucleotides, siRNA molecules, CRISPR-based gene editors, RNA base editors, transcriptional modulators, viral or non-viral delivery systems, monoclonal antibodies, peptide inhibitors, or combinations thereof.

In some embodiments, the target gene is AGT, the precursor of angiotensin I/angiotensin II in the renin-angiotensin system. Elevated AGT levels are associated with hypertension and cardiovascular risk. Accordingly, an siRNA that reduces AGT mRNA expression in the liver reduces AGT protein synthesis, attenuates downstream angiotensin II production, lowers blood pressure, and thereby limits progression of cardiovascular disease.

One example in development is Zilebesiran (also known as ALN-AGT01), a GalNAc-conjugated siRNA agent targeting AGT. It is investigational for hypertension and aims to provide long-duration antihypertensive effect with infrequent dosing. Other siRNA agents targeting AGT can be used.

In some embodiments, AGT-lowering siRNA is administered in patients with hypertension, cardiometabolic disease, or cardiovascular disease, and may be combined with other RAS modulators (ACE inhibitors, ARBs), lipid-lowering agents, or lifestyle interventions.

In some embodiments, a composition comprising a GalNAc-conjugated siRNA targeting PCSK9 mRNA, is administered subcutaneously quarterly, semi-annually, or annually, optionally in combination with statin therapy, to reduce LDL-C in patients, for example, with heterozygous familial hypercholesterolemia or established atherosclerotic cardiovascular disease (ASCVD).

### Lp(a)-Lowering Interventions

In some embodiments, the present disclosure provides therapeutic intervention for reducing plasma Lp(a) levels. Elevated Lp(a) levels are an independent heritable risk factor for atherosclerotic cardiovascular disease, aortic stenosis, and related disorders. Without being bound by theory, lowering Lp(a) is believed to reduce vascular inflammation, plaque accumulation, and cardiovascular event incidence.

Suitable modalities for Lp(a) reduction include, without limitation, antisense oligonucleotides, siRNA molecules, CRISPR-based nucleic acid editing or regulation systems targeting apolipoprotein(a) (LPA), monoclonal antibodies or peptide therapeutics, viral or non-viral gene therapy vectors, or combinations thereof.

In some embodiments, the target gene is LPA (encoding apolipoprotein(a)), which contributes to circulating levels of Lipoprotein(a) [Lp(a)]. Elevated Lp(a) is an independent heritable cardiovascular risk factor associated with atherosclerosis, aortic stenosis, and other cardiovascular outcomes. Reducing LPA mRNA expression via siRNA reduces the production of apo(a), lowers Lp(a) levels, and potentially the progression of cardiovascular disease.

One investigational agent is Zerlasiran (candidate siRNA targeting LPA) which in a phase 2 trial (ALPACAR-360) achieved reductions of ~80%+ in time-averaged Lp(a) over −36 weeks. Other siRNA agents targeting LPA can be used.

In some embodiments, Lp(a)-targeting siRNA is used in individuals with elevated Lp(a) (e.g., >50 mg/dL) and optionally can be combined with LDL-lowering, AGT-lowering or other cardiometabolic interventions.

In some embodiments, a composition comprising a GalNAc-conjugated siRNA targeting LPA mRNA, is administered subcutaneously quarterly, semi-annually, or annually in patients with elevated Lp(a) and optionally high residual cardiovascular risk despite LDL-C lowering.

### Combination Therapies

In some embodiments, therapeutic interventions targeting LDL, AGT, and/or Lp(a) are used in combination to achieve additive or synergistic effects. Combination approaches can include concurrent, sequential, or alternating administration of multiple modalities or agents. In some embodiments, combination therapy reduces the severity, or progression of atherosclerosis, myocardial infarction, ischemic stroke, heart failure, or other cardiometabolic or cardiovascular conditions to a greater extent than either intervention alone.

In some embodiments, the siRNA agent is co-formulated or co-administered with other nucleic acid therapeutics (e.g., antisense oligonucleotides), small molecules, biologics, or lifestyle/dietary interventions.

In some embodiments, the LDL-lowering siRNA, AGT-lowering siRNA, and/or Lp(a)-lowering siRNA is administered in a combination therapy regimen to treat or prevent cardiometabolic disease, including but not limited to atherosclerosis, heart failure, hypertension, dyslipidemia and elevated Lp(a).

There are numerous uses of the technology provided herein including, but not limited to, the following.

Aspects of the technology provide a mechanism and infrastructure for establishing the field of precision health, with the goal of predicting and preventing disease to extend the healthy lifespan, by longitudinally monitoring cardiometabolic health, predicting the risk of cardiometabolic disease over any time horizon, predicting the benefit of specific therapies to reduce risk, discovering the optimal timing, intensity, and sequence of interventions useful to each individual to personalize prevention, and providing iteratively updated guidance based on each individual's compliance with the recommend actions, achieved reductions in the modifiable causes of disease, and evolving cardiometabolic health.

Thus, in some embodiments, a method comprises longitudinally monitoring cardiometabolic health. In some embodiments, a method comprises predicting the risk of cardiometabolic disease. In some embodiments, a method comprises predicting the benefit of specific therapies to reduce risk. In some embodiments, a method comprises implementing the optimal timing, intensity, and sequence of interventions useful to each individual to personalize prevention. In some embodiments, a method comprises providing iteratively updated guidance based on each individual's compliance with the recommended actions, achieved reductions in the modifiable causes of disease, and/or evolving cardiometabolic health.

Aspects of the technology provide a mechanism and infrastructure for designing randomized trials and generating clinical evidence, by identifying a population of potential study participants, predicting the absolute risk of developing a cardiometabolic event over any time horizon for each potential study participant, predicting the expected benefit in response to a specific therapy over any time horizon (both short-term and long-term) for each potential study participant, identifying the optimal study population to enroll in the trial based on the predicted absolute risk over the trial duration and the predicted proportional and absolute reductions in response to the therapy under study, iteratively monitoring the progress of the trial to ensure compliance with treatment allocation, precisely predicting the event rate in either treatment arm at every month of follow-up, demonstrating agreement between predicted and observed event rates in either treatment arm at every point of follow-up to build intuition and trust for predicting the expected benefit of longer durations of follow-up (thus providing a mechanism to capture both short-term and long-term benefit in a short-term trial), and longitudinally monitoring study participants following completion of the trial to generate long-term evidence required by regulators for approving treatments to prevent future events by slowing the progression of disease, focusing on demonstrating that the predicted and observed event rates agree at every future time point based on the achieved magnitude of reduction in the therapeutically targeted biomarker and the duration of therapy.

In some embodiments, a method comprises identifying a population of potential study participants. In some embodiments, a method comprises predicting the absolute risk of developing a cardiometabolic (e.g., event over any time horizon) for each potential study participant. In some embodiments, a method comprises predicting the expected benefit in response to a specific therapy over any time horizon (e.g., short-term and/or long-term) for each potential study participant. In some embodiments, a method comprises identifying the optimal study population to enroll in the trial. In some embodiments, a method comprises iteratively monitoring the progress of the trial. In some embodiments, a method comprises predicting the event rate in one or more treatment arms. In some embodiments, a method comprises demonstrating agreement between predicted and observed event rates in one or more treatment arms. In some embodiments, a method comprises longitudinally monitoring study participants following completion of the trial.

Aspects of the technology provide a mechanism and infrastructure for monitoring the effectiveness and establishing longitudinal pay-for-performance reimbursement schedules for therapies designed to prevent future events by slowing the trajectory of disease, by identifying populations of persons who would benefit from a therapy to prevent disease, longitudinally monitoring the absolute achieved reduction of the modifiable cause of disease targeted by the therapy, predicting the expected reduction in clinical events over any time horizon (including both short-term and long-term) in response to the achieved absolute reduction in the targeted modifiable cause of disease and the duration of therapy, translating the expected reduction in clinical events into a quantitative estimate of the economic benefit, setting the reimbursement schedule to provide longitudinal payments as pay-for-performance based on the achieved absolute cumulative reductions in the modifiable cause of disease targeted by the therapy over time and the corresponding expected clinical benefit, longitudinally monitoring individuals receiving the treatment to compare the predicted and observed event rates to adjust reimbursement rates and improve accuracy of the predicted clinical benefit, thus providing a mechanism to iteratively improve implementation of the therapy to maximize the achieved absolute cumulative reductions in the targeted biomarker and thus maximize reductions in clinical events, reimbursements for the therapy, and return on investment for the payor.

In some embodiments, a method comprises identifying populations of persons who would benefit from a therapy to prevent disease. In some embodiments, a method comprises longitudinally monitoring the absolute achieved reduction of the modifiable cause of disease targeted by the therapy. In some embodiments, a method comprises predicting the expected reduction in clinical events over any time horizon.

In some embodiments, a method comprises translating the expected reduction in clinical events into a quantitative estimate of the economic benefit. In some embodiments, a method comprises setting the reimbursement schedule to provide longitudinal payments. In some embodiments, a method comprises longitudinally monitoring individuals receiving the treatment to compare the predicted and observed event rates.

Aspects of the technology provide a mechanism and infrastructure for informing, monitoring, and iteratively improving economic analyses and health policy, by predicting the risk of cardiometabolic disease and the absolute rate of clinical events over any time horizon within a target population, predicting the absolute reduction in clinical events and expected costs of specific therapies designed to reduce risk over any time horizon, incorporating the increasing clinical benefit over time for therapies that are designed to prevent clinical events by slowing the progression of chronic diseases thus capturing the true magnitude of their clinical and economic value within economic analyses for the first time, predicting the optimal timing, intensity, and sequence of interventions useful to each individual in a target population to personalize the prevention cardiometabolic disease and cardiometabolic events thereby providing a mechanism to model the optimal combinations of preventative therapies required by each individual designed minimize cost, maximize clinical benefit, and return on investment, providing a mechanism to iteratively improve economic analyses by incorporating longitudinally measured data on observed absolute reduction in events to supplement the predicted event rates and thus improve model performance, and providing a mechanism to iteratively improve implementation of health policies by longitudinally monitoring the target population to ensure milestones (including achieved absolute reductions in the targeted modifiable causes of disease) are being met to maximize reductions in clinical events and return on investment.

In some embodiments, a method comprises predicting the risk of cardiometabolic disease and the absolute rate of clinical events over any time horizon within a target population. In some embodiments, a method comprises predicting the absolute reduction in clinical events and expected costs of specific therapies designed to reduce risk over any time horizon. In some embodiments, a method comprises predicting the optimal timing, intensity, and sequence of interventions useful to each individual in a target.

Aspects of the technology provide a mechanism and infrastructure for designing dynamically priced insurance instruments that can be used to modify behavior and improve cardiometabolic health, by predicting the risk of experiencing a clinical cardiometabolic disease event over any time horizon for each individual in the insured population including both short-term risk (e.g. monthly or yearly) and longer-term risk, pricing the insurance instrument for each individual based on their predicted absolute short-term and longer-term risk of experiencing a clinical event, predicting the proportional and absolute reduction in clinical events expected in response to all levels of reduction in the modifiable causes of disease over the desired time horizon, selecting a desired level of risk of clinical events for each individual in the insured population, offering specific therapeutic options to each individual to achieve the required absolute reductions in the modifiable causes of disease used to achieve the desired level of risk of experiencing a clinical event over the desired time horizon, and offering a graduated and steeply discounted insurance premium to an individual based on their achieved reduction in the targeted modifiable cause(s) of disease and corresponding predicted proportional reduction in the risk of clinical events, thus using the dynamically priced insurance instrument to modify behavior, reduce clinical events, minimize insurance provider exposure, and maximize profitability.

For example, an insurance instrument can be priced to reflect the dynamically changing instantaneous, short-term, or -long-term risks of having a cardiovascular event, developing hypertension, or developing type 2 diabetes (T2D) over any time interval—thus permitting dynamic pricing of an insurance instrument as lever to motivate behavior to improve cardiometabolic health and thus reduce financial exposure of the insurance instrument user in a tangible way. By constructing a cardiovascular health score that dynamically adjusts over time both with and without a specific intervention or set of interventions as a person's health journey evolves, the insurance instrument issuer can further reduce financial exposure by further dynamically discounting the insurance product conditional on the subject taking and adhering to a recommended course of action, such as one of the recommended courses of actions identified in accordance with embodiments of the technology described herein.

Indeed, methods of determining cardiometabolic health metrics described herein (such as e.g. LDL level trajectories, SBP level trajectories, absolute instantaneous hazard rates of a subject having a cardiovascular event at one or more time intervals, intervention-adjusted instantaneous hazard rates, and metrics derived therefrom such as cumulative trajectories, cumulative hazard rates, proportional reductions in risk metrics, and rates of rise of cardiometabolic health metrics) may be used in the context of methods for pricing an insurance instrument. While the problem of pricing an insurance instrument may be administrative in nature, the present disclosure provides technical solutions to this problem by providing new methods of determining cardiometabolic health metrics for use in such methods. Such methods represent innovative technical solutions to the problem of pricing an insurance instrument, which can be used instead of prior art solutions, and which may be associated with various benefits such as more accurate predictions of cardiometabolic health, in turn resulting in improved pricing determinations. The proposed solutions are solutions that are innovative solutions to the problem of improving pricing of insurance instruments which a technically qualified person may be tasked to provide a technical implementation for.

In some embodiments, a method comprises predicting the risk of experiencing a clinical cardiometabolic disease event over any time horizon for each individual in an insured population. In some embodiments, a method comprises pricing the insurance instrument for each individual. In some embodiments, a method comprises predicting the proportional and absolute reduction in clinical events expected in response to one or more (or all) levels of reduction in modifiable causes of disease over the desired time horizon. In some embodiments, a method comprises selecting a desired level of risk of clinical events for each individual in a (e.g., an insured) population. In some embodiments, a method comprises offering specific therapeutic options to each individual.

### Additional Embodiments

The present disclosure further includes the embodiments described in the following numbered paragraphs:
1. A method for identifying an intervention for a subject in furtherance of preventing development of cardiovascular disease in the subject, the method comprising:
using at least one computer hardware processor to perform:
obtaining cardiometabolic health data for the subject;
determining, using at least some of the cardiometabolic health data, a first trained machine learning (ML) model and for each of multiple time intervals, one or more measures of risk that the subject develops cardiovascular disease to obtain multiple measures of risk corresponding to the multiple time intervals;
determining, using the multiple measures of risk that the subject develops cardiovascular disease and at least one second trained ML model, benefit of administering to the subject one or more therapeutic interventions designed to reduce risk of cardiovascular disease by targeting one or more modifiable causes of the cardiovascular disease; and
identifying, using the determined benefit of administering the one or more therapeutic interventions, at least one therapeutic intervention to recommend being administered to the subject.
2. The method of paragraph 1, wherein obtaining the cardiometabolic health data for the subject comprises obtaining subject characteristic and/or measurement data comprising:
one or more values for one or more clinical characteristics of the subject,
one or more values for one or more physical measurements of the subject, and/or
one or more values for one or biochemical measurements of the subject.
3. The method of paragraph 2, wherein the one or more values for one or more clinical characteristics comprise values for one or more demographic characteristics of the subject, one or more genetic characteristics of the subject, one or more family history characteristics of the subjects, one or more comorbidities, and/or risk factors.
4. The method of paragraph 2 or 3, wherein the subject characteristic and/or measurement data comprises one or more values for one or more clinical characteristics of the subject selected from the group consisting of: age, biological sex, family history of coronary heart disease (CHD), family history of hypertension (HTN), family history of type 2 diabetes (T2D), polygenic score for ASCVD, polygenic score for CHD, polygenic score for HTN, polygenic score for T2D, polygenic score for body mass index (BMI), inherited predisposition or predispositions, and history of tobacco use.
5. The method of paragraph 2, wherein the one or more values for one or more physical measurements of the subject comprise one or more values for physical measurements of quantities that are risk factors for cardiometabolic disease and/or physiological measurements selected from blood pressure measurements and measurements indicative of adiposity.
6. The method of any one of paragraphs 2-5, wherein the subject characteristic and/or measurement data comprises one or more values for one or more physical measurements of the subject selected from the group consisting of systolic blood pressure (SBP), diastolic blood pressure (DBP), weight, waist circumference, height, body mass index (BMI), and waist-to-height ratio.
7. The method of any one of paragraphs 2-6, further comprising measuring the one or more values for the one or more physical measurements of the subject or receiving the one or more values via a user interface and/or electronic health records of the subject.
8. The method of any one of paragraphs 2-8, wherein the one or more values for one or more biochemical measurements of the subject comprise one or more values for biochemical measurements of quantities that are risk factors for cardiovascular disease and/or biochemical measurements selected from measurements of one or more biochemical markers, optionally a protein, lipid or lipoprotein, in a blood, serum, and/or plasma sample from the subject.
9. The method of any one of paragraphs 2-8, wherein the subject characteristic and/or measurement data comprises one or more values for one or more biochemical measurements of the subject selected from the group consisting of low-density lipoprotein (LDL) level, high-density lipoprotein (HDL) level, total cholesterol level, triglyceride (TG) level, non-HDL cholesterol level, apolipoprotein (apoB) level, lipoprotein (a) (Lp(a)) level, hemoglobin A1c (HbA1c) level, and c-reactive protein (CRP) level.
10. The method of paragraph 8 or 9, further comprising:
obtaining a biological sample of the subject; and
determining, by processing the biological sample, the one or more values for the one or more biochemical measurements of the subject.
11. The method of paragraph 8 or 9, further comprising:
determining, by processing a biological sample previously obtained from the subject, the one or more values for the one or more biochemical measurements of the subject.
12. The method of any one of paragraphs 1-11, wherein the cardiometabolic health data includes a value indicating the subject's age, a value indicating the subject's biological sex, a value indicating the subject's LDL level, a value indicating the subject's SBP, a value indicating the subject's DBP, and, optionally, a value indicating the subject's Lp(a) level.
13. The method of any one of paragraphs 2-12, wherein obtaining the cardiometabolic health data further comprises:
estimating an LDL level trajectory for the subject using the subject characteristic and/or measurement data,
wherein the LDL level trajectory for the subject comprises an estimated LDL level for the subject for each of multiple prior ages of the subject and multiple future ages of the subject,
wherein the multiple prior ages and the multiple future ages are, respectively, before and
after the subject's age at which the most recent LDL measurement of the subject was made.
14. The method of paragraph 13, further comprising:
estimating, using the LDL level trajectory, a cumulative LDL exposure trajectory for the subject with respect to a set of ages,
wherein the cumulative LDL exposure trajectory comprises an estimated cumulative LDL exposure level for the subject at each age in the set of ages.
15. The method of paragraph 13 or 14, wherein estimating the LDL level trajectory for the subject comprises:
encoding the subject characteristic and/or measurement data into a feature vector; and
estimating the LDL level trajectory for the subject by processing the feature vector using a third trained ML model that has been trained to estimate an LDL level for the subject at each of the multiple prior ages and each of the multiple future ages,
wherein the multiple prior ages and the multiple future ages are, respectively, before and
after the subject's age at which the most recent LDL measurement of the subject was made.
16. The method of paragraph 15, wherein the third trained ML model has been trained to estimate LDL levels for the subject using training data comprising, for each of a plurality of participants, repeated longitudinal measures of LDL levels and values for the one or more clinical characteristics and/or the one or more physical measurements and/or the one or more biochemical measurements over at least 10, at least 20, at least 30, or at least 50 years of follow-up.
17. The method of paragraph 15 or 16, wherein encoding the subject characteristic and/or measurement data into the feature vector comprises:
standardizing at least some of values in the subject characteristic and/or measurement data to obtain an initial feature vector; and
positionally encoding at least some feature values in the initial feature vector by the subject's age or ages at which the at least some of feature values were measured to obtain the feature vector representing the subject.
18. The method of paragraph 17, wherein performing the standardizing comprises:
performing min-max standardizing the at least some of the values, for continuous variables, in the subject characteristic and/or measurement data, and/or
numerically encoding any dichotomous or ordinal values in the subject characteristic and/or measurement data.
19. The method of paragraph 17 or 18, wherein the positionally encoding comprises:
generating a positional encoding of the initial feature vector using sinusoidal encoding of at least some elements of the initial feature vector, wherein the sinusoidal encoding using age in years as position; and
generating the feature vector by appending the positional encoding of the initial feature vector to the initial feature vector.
20. The method of any one of paragraphs 15-19, wherein the third trained ML model is a neural network model.
21. The method of paragraph 20, wherein the third trained ML model has a transformer architecture, optionally a temporal fusion transformer (TFT) architecture.
22. The method of paragraph 20, wherein the third trained ML model is a recurrent neural network (RNN) model.
23. The method of paragraph 22, wherein the third trained ML model is a long short-term memory (LSTM) neural network model or a gated recurrent unit (GRU) neural network model.
24. The method of paragraph 22, wherein the third trained ML model is a bi-directional LSTM (bi-LSTM) model, a bi-directional GRU model, or an ODE-RNN model.
25. The method of paragraph 24, wherein:
the third trained ML model is the bi-LSTM model, and
processing the feature vector using the third trained ML model comprises:
estimating an LDL level for the subject at each of the multiple future ages using a forward pass of the bi-LSTM model; and
estimating an LDL level for the subject at each of the multiple prior ages using a backward pass of the bi-LSTM model.
26. The method of paragraph 25, wherein the third trained ML model was trained using participant data for a plurality (optionally, at least 10,000) of participants enrolled in one or more prospective studies, whereby for each of the plurality of participants repeated longitudinal measures of LDL, HDL, SBP, weight, waist circumference, BMI, and/or HbA1c are available over at least 10, at least 20, at least 30, or at least 50 years of follow-up.
27. The method of 26, wherein the participant data was censored by censoring applied at time lost to follow-up, death, a first cardiovascular event, or initiation of lipid lowering therapy.
28. The method of any one of paragraphs 2-27, wherein obtaining the cardiometabolic health data further comprises:
estimating an SBP level trajectory for the subject using the subject characteristic and/or measurement data,
wherein the SBP level trajectory for the subject comprises an estimated SBP level for the subject for each of multiple prior ages of the subject and multiple future ages of the subject,
wherein the multiple prior ages and the multiple future ages are, respectively, before and after the subject's age at which the most recent SBP measurement of the subject was made.
29. The method of paragraph 28, further comprising:
estimating, using the SBP level trajectory, a cumulative SBP exposure trajectory for the subject with respect to a set of ages, wherein the cumulative SBP exposure trajectory comprises an estimated cumulative SBP exposure level for the subject at each age in the set of ages; and
optionally, estimating rates of rise in SBP for the subject at each age in the set of ages.
30. The method of paragraph 28 or 29, wherein estimating the SBP level trajectory for the subject comprises:
encoding the subject characteristic and/or measurement data into a second feature vector; and
estimating the SBP level trajectory for the subject by processing the second feature vector using a fourth trained ML model that has been trained to estimate an SBP level for the subject at each of the multiple prior ages and each of the multiple future ages.
31. The method of paragraph 30, wherein the fourth trained ML model has been trained to estimate SBP levels for the subject using training data comprising, for each of a plurality of participants, repeated longitudinal measures of SBP levels and values for the one or more clinical characteristics and/or the one or more physical measurements and/or the one or more biochemical measurements over at least 10, at least 20, at least 30, or at least 50 years of follow-up.
32. The method of paragraph 30 or 31, wherein encoding the subject characteristic and/or measurement data into the second feature vector comprises:
standardizing at least some of values in the subject characteristic and/or measurement data to obtain an initial second feature vector; and
positionally encoding at least some feature values in the initial second feature vector by the subject's age or ages at which the at least some of the feature values were measured to obtain the second feature vector representing the subject.
33. The method of paragraph 32, wherein performing the standardizing comprises:
performing min-max standardizing the at least some of the values, for continuous variables, in the subject characteristic and/or measurement data; and/or
numerically encoding any dichotomous or ordinal values in the subject characteristic and/or measurement data.
34. The method of paragraph 32, wherein the positionally encoding comprises:
generating a positional encoding of the initial second feature vector using sinusoidal encoding of at least some elements of the initial second feature vector, wherein the sinusoidal encoding using age in years as position; and
generating the feature vector by appending the positional encoding of the initial second feature vector to the initial second feature vector.
35. The method of any one of paragraphs 30-34, wherein the fourth trained ML model is a neural network model.
36. The method of paragraph 35, wherein the fourth trained ML model has a transformer architecture, optionally a temporal fusion transformer (TFT) architecture.
37. The method of paragraph 35, wherein the fourth trained ML model is a recurrent neural network (RNN) model.
38. The method of paragraph 37, wherein the fourth trained ML model is a long short-term memory (LSTM) neural network model or a gated recurrent unit (GRU) neural network model.
39. The method of paragraph 37, wherein the fourth trained ML model is a bi-directional LSTM (bi-LSTM) model, a bi-directional GRU model, or an ODE-RNN model.
40. The method of paragraph 39, wherein:
the fourth trained ML model is the bi-LSTM model, and
processing the second feature vector using the fourth trained ML model comprises:
estimating an SBP level for the subject at each of the multiple future ages using a forward pass of the bi-LSTM model; and
estimating an SBP level for the subject at each of the multiple prior ages using a backward pass of the bi-LSTM model.
41. The method of paragraph 40, wherein the fourth trained ML model was trained using participant data for a plurality (optionally, at least 10,000) of participants enrolled in one or more prospective studies whereby for each of the plurality of participants repeated longitudinal measures of LDL, HDL, SBP, weight, waist circumference, BMI, and/or HbA1c are available over at least 10, at least 20, at least 30, or at least 50 years of follow-up.
42. The method of paragraph 41, wherein the participant data was censored by censoring applied at time lost to follow-up, death, a first cardiovascular event, or initiation of lipid lowering therapy.
43. The method of any one of paragraphs 2-42, wherein obtaining the cardiometabolic health data further comprises:
estimating an Lp(a) level trajectory for the subject using the subject characteristic and/or measurement data,
wherein the Lp(a) level trajectory for the subject comprises an estimated Lp(a) level for the subject for each of multiple prior ages of the subject and multiple future ages of the subject,
wherein the multiple prior ages and the multiple future ages are, respectively, before and
after the subject's age at which the most recent Lp(a) measurement of the subject was made.
44. The method of paragraph 43, further comprising:
   estimating, using the Lp(a) level trajectory, a cumulative Lp(a) exposure trajectory for the subject with respect to a set of ages,
wherein the cumulative Lp(a) exposure trajectory comprises an estimated cumulative Lp(a) exposure level for the subject at each age in the set of ages.
45. The method of paragraph 43 or 44, wherein estimating the Lp(a) level trajectory for the subject comprises:
encoding the subject characteristic and/or measurement data into a third feature vector; and
estimating the Lp(a) level trajectory for the subject by processing the third feature vector using a fifth trained ML model that has been trained to estimate an Lp(a) level for the subject at each of the multiple prior ages and each of the multiple future ages.
46. The method of paragraph 45, wherein the fifth trained ML model has been trained to estimate Lp(a) levels for the subject using training data comprising, for each of a plurality of participants, repeated longitudinal measures of Lp(a) levels and values for the one or more clinical characteristics and/or the one or more physical measurements and/or the one or more biochemical measurements over multiple years of follow-up.
47. The method of paragraph 45 or 46, wherein encoding the subject characteristic and/or measurement data into the third feature vector comprises:
standardizing at least some of values in the subject characteristic and/or measurement data to obtain an initial third feature vector; and
positionally encoding at least some feature values in the initial third feature vector by the subject's age or ages at which the at least some of feature values were measured to obtain the third feature vector representing the subject.
48. The method of paragraph 47, wherein performing the standardizing comprises:
performing min-max standardizing the at least some of the values, for continuous variables, in the subject characteristic and/or measurement data, and/or
numerically encoding any dichotomous or ordinal values in the subject characteristic and/or measurement data.
49. The method of paragraph 47 or 48, wherein the positionally encoding comprises:
generating a positional encoding of the initial third feature vector using sinusoidal encoding of at least some elements of the initial feature vector, wherein the sinusoidal encoding using age in years as position; and
generating the third feature vector by appending the positional encoding of the initial third feature vector to the initial third feature vector.
50. The method of any one of paragraphs 45-49, wherein the fifth trained ML model is a neural network model.
51. The method of paragraph 50, wherein the fifth trained ML model is a recurrent neural network (RNN) model.
52. The method of paragraph 51, wherein:
the fifth trained ML model is a bi-LSTM model, and
processing the feature vector using the third trained ML model comprises:
estimating an Lp(a) level for the subject at each of the multiple future ages using a forward pass of the bi-LSTM model; and
estimating an Lp(a) level for the subject at each of the multiple prior ages using a backward pass of the bi-LSTM model.
53. The method of any one of paragraphs 2-52, wherein obtaining the cardiometabolic health data further comprises:
estimating a weight trajectory for the subject using the subject characteristic and/or measurement data,
wherein the weight trajectory for the subject comprises estimated weight of the subject for each of multiple ages of the subject.
54. The method of paragraph 53, wherein estimating the weight trajectory is performed assuming that the subject's current age-and-sex adjusted weight percentile remains constant throughout life.
55. The method of any one of paragraphs 2-54, wherein obtaining the cardiometabolic health data further comprises:
estimating a waist circumference trajectory for the subject using the subject characteristic and/or measurement data,
wherein the waist circumference trajectory for the subject comprises estimated waist circumference of the subject for each of multiple ages of the subject.
56. The method of paragraph 55, wherein estimating the waist circumference trajectory is performed assuming that the subject's current age-and-sex adjusted waist circumference percentile remains constant throughout life.
57. The method of any one of paragraphs 2-56, wherein obtaining the cardiometabolic health data further comprises:
   estimating an HbA1c level trajectory for the subject using the subject characteristic and/or measurement data,
wherein the HbA1c level trajectory for the subject comprises estimated HbA1c levels of the subject for each of multiple ages of the subject.
58. The method of paragraph 57, wherein estimating the HbA1c level trajectory is performed assuming that the subject's current age-and-sex adjusted HbA1c percentile remains constant throughout life.
59. The method of any one of paragraphs 2-58, wherein obtaining the cardiometabolic health data further comprises using the subject characteristic and/or measurement data to:
estimate an LDL level trajectory and a cumulative LDL exposure trajectory for the subject;
estimate an SBP level trajectory and a cumulative LDL exposure trajectory for the subject;
estimate and Lp(a) level trajectory and a cumulative Lp(a) exposure trajectory for the subject;
estimate a weight trajectory for the subject;
estimate a waist circumference trajectory for the subject; and
estimate an HbA1c level trajectory for the subject.
60. The method of paragraph 1 or any other preceding paragraph, wherein determining the one or more measures of risk that the subject develops cardiovascular disease, for each of the multiple time intervals, to obtain the multiple measures of risk corresponding to the multiple time intervals, comprises:
(a) estimating, using the first trained ML model and the at least some of the cardiometabolic health data including a cumulative LDL exposure trajectory for the subject, values indicative of log hazard ratios for risk of the subject having a cardiovascular event at respective levels of cumulative LDL exposure; and
(b) estimating, using the values indicative of the log hazard ratios,
(i) absolute instantaneous hazard rates of the subject having a cardiovascular event at the respective levels of cumulative LDL exposure, and
(ii) cumulative lifetime risks of the subject having a cardiovascular event at the respective levels of cumulative LDL exposure;
61. The method of paragraph 60, further comprising:
(c) estimating, using the cumulative LDL exposure trajectory for the subject, the multiple measures of risk to include:
(i) absolute instantaneous hazard rates of the subject having a cardiovascular event at respective ones of the multiple time intervals, and
(ii) cumulative lifetime hazard and event rates of the subject having a cardiovascular event at the respective ones of the multiple time intervals.
62. The method of paragraph 61, wherein the multiple time intervals correspond to multiple ages of the subject such that the multiple measures of risk comprise:
absolute instantaneous hazard rates of the subject having a cardiovascular event at each of the multiple ages of the subject; and
cumulative lifetime risks of the subject having a cardiovascular event at each of the multiple ages of the subject.
63. The method of any one of paragraphs 60-62, wherein estimating, using the first trained ML model and the at least some of the cardiometabolic health data, the values indicative of the log hazard ratios for risk of the subject having a cardiovascular event at the respective levels of cumulative LDL exposure comprises:
encoding the at least some of the cardiometabolic health data to obtain input feature data; and
estimating, by processing the input feature data using the first trained ML model, the values indicative of the log hazard ratios for the risk of the subject having a cardiovascular event at the respective levels of cumulative LDL exposure.
64. The method of paragraph 63, wherein the input feature data comprises:
a set of input feature vectors, each particular one of the input feature vectors corresponding to a particular cumulative LDL exposure level from among the respective levels of cumulative LDL exposure,
wherein the particular input feature vector in the set of input feature vectors that corresponds to the particular cumulative LDL exposure level, comprises:
(a) subject characteristic and/or measurement data comprising:
one or more values for one or more clinical characteristics of the subject,
one or more values for one or more physical measurements of the subject, and/or
one or more values for one or biochemical measurements of the subject;
(b) an LDL level trajectory for the subject;
(c) a cumulative LDL exposure trajectory for the subject;
(d) an SBP level trajectory for the subject indicating an estimate of the subject's SBP level at each of the respective levels of cumulative LDL exposure;
(e) a cumulative SBP exposure trajectory for the subject indicating an estimate of the subject's cumulative SBP exposure at each of the respective levels of cumulative LDL exposure;
(f) an Lp(a) level trajectory for the subject indicating an estimate of the subject's Lp(a) at each level of the respective levels of cumulative LDL exposure;
(g) a cumulative Lp(a) exposure trajectory for the subject indicating an estimate of the subject's cumulative Lp(a) exposure at each level of the respective levels of cumulative LDL exposure;
(h) a weight trajectory for the subject indicating an estimate of the subject's weight at each of the respective levels of cumulative LDL exposure;
(i) a waist circumference trajectory for the subject indicating an estimate of the subject's waist circumference at each of the respective levels of cumulative LDL exposure;
(j) an HbA1c level trajectory for the subject indicating an estimate of the subject's HbA1c level at each of the respective levels of cumulative LDL exposure;
(k) an age trajectory for the subject indicating an estimated age for the subject at each of the respective levels of cumulative LDL exposure; and/or
(l) a positionally encoded version of one or more of (a), (b), (d), (e), (f), (g), (h), (i), (j), and (k) obtained by positionally encoding the one or more of (a), (b), (d), (e), (f), (g), (h), (i), (j), and (k) by age of the subject at which the particular cumulative LDL exposure level is predicted to occur,
optionally wherein values in the set of input feature vectors are organized in at least one data structure representing a matrix.
65. The method of paragraph 64, wherein generating the input feature data comprises:
generating a first input vector using the subject characteristic and/or measurement data and positional encodings associated with the subject characteristic and/or measurement data;
processing the first input vector using a first recurrent neural network (RNN) to obtain the LDL level trajectory for the subject; and
processing the first input vector using a second RNN to obtain the SBP level trajectory for the subject,
wherein the first RNN is different from the second RNN, and wherein the first and second RNNs are different from the first trained ML model.
66. The method of any one of paragraphs 60-65, wherein the first machine learning model has been trained using training data comprising, for each of a plurality of participants enrolled in one or more prospective studies, multiple LDL and/or multiple SBP measurements along with a recorded age or date at which a first cardiovascular event occurred, optionally wherein the first cardiovascular event is a first episode of a fatal or non-fatal myocardial infarction (MI), fatal or non-fatal ischemic stroke, or coronary revascularization.
67. The method of paragraph 60 or any other preceding paragraph, wherein the first trained ML model is a trained survival model using cumulative LDL exposure as an interval of follow-up.
68. The method of paragraph 67, wherein the trained survival model using cumulative LDL exposure as an interval of follow-up comprises one of:
a Cox proportional hazard model;
a random forest of survival trees model;
a gradient boosted machine (GBM) for survival trees model;
a survival deep neural network (DNN) model; and/or
an ensemble of any of these survival models.
69. The method of paragraph 67, wherein the first trained ML model is a trained survival deep neural network (DNN) model using cumulative LDL exposure as the interval of follow-up.
70. The method of paragraph 67, wherein the first trained ML model is a trained survival model with piecewise exponential models (PEM) using cumulative LDL exposure as the interval of follow-up.
71. The method of paragraph 70, wherein the first trained ML model is a trained survival DNN model with piecewise exponential models (PEM) using cumulative LDL exposure as the interval of follow-up.
72. The method of paragraph 71, wherein the trained survival DNN model with PEM comprises a fully connected neural network having at least 3, at least 4, at least 5, or at least six fully connected layers, optionally wherein the trained survival DNN with PEM uses rectified linear units (ReLUs) for non-linearities.
73. The method of paragraph 71, wherein the hazard rate in each interval of follow-up is assumed to be constant and the trained survival DNN with PEM comprises a different set of trained weights for each interval of follow-up, wherein each interval of follow-up is an interval of cumulative LDL exposure.
74. The method of paragraph 73, wherein the interval of cumulative LDL exposure is one mmol/year.
75. The method of paragraph 71, wherein the trained survival DNN with PEM was trained using participant data for at least 1 million (M) or 1.5 M participants enrolled in one or more prospective studies, whereby for each of the at least 1 M or 1.5 M participants at least one LDL or SBP measurement were available along with a recorded age or date at which a first episode of a fatal or non-fatal myocardial infarction (MI), fatal or non-fatal ischemic stroke, or coronary revascularization occurred, with censoring applied at time of last follow-up, death, or first cardiovascular event.
76. The method of any one of paragraphs 69-74, wherein the trained survival DNN has at least 1K, 5K, 10K, at least 25K, at least 50K, at least 100K, at least 500K, between 10K and 500K weight values for each interval of cumulative LDL exposure.
77. The method of paragraph 63, wherein processing the input feature data using the first trained ML model comprises:
processing the input feature data with a trained survival deep neural network (DNN) with piecewise exponential modeling (PEM) model to calculate log hazard ratios for the risk of the subject having a cardiovascular event at the respective levels of cumulative LDL exposure.
78. The method of any one of paragraphs 60-77, wherein the cardiovascular event is a fatal myocardial infarction (MI), an episode of a non-fatal MI, a fatal ischemic stroke, an episode of a non-fatal ischemic stroke, or a coronary revascularization.
79. The method of paragraph 60, wherein estimating, using the values indicative of the log hazard ratios, the cumulative lifetime risks of the subject having a cardiovascular event at the respective levels of cumulative LDL exposure comprises:
(a) generating a data structure encoding a lifetable, the data structure comprising values indicating absolute instantaneous hazard rates of experiencing a cardiovascular event at the respective levels of cumulative LDL exposure in a reference population;
(b) determining the absolute instantaneous hazard rates of the subject having a cardiovascular event at the respective levels of cumulative LDL exposure using:
(i) the values, from (a), indicating absolute instantaneous hazard rates of experiencing a cardiovascular event at the respective levels of cumulative LDL exposure in the reference population; and
(ii) the values indicative of the log hazard ratios for risk of the subject having a cardiovascular event at the respective levels of cumulative LDL exposure; and
(c) determining the cumulative hazard rates of the subject having a cardiovascular event at the respective levels of cumulative LDL using the absolute instantaneous hazard rates, determined at (b); and (d) determining the cumulative event rates of the subject having a cardiovascular event at the respective levels of cumulative LDL using the absolute instantaneous hazard rates, determined at (b).
80. The method of paragraph 79, further comprising:
identifying a personal plaque burden threshold for the subject by using the cumulative event rates of the subject having a cardiovascular event at the respective levels of cumulative LDL,
wherein the personal plaque burden threshold indicates a level of cumulative plaque burden at which cardiovascular events are predicted to begin to occur for the subject.
81. The method of paragraph 79, further comprising:
identifying, for a specified level of cumulative lifetime risk, a level of cumulative plaque burden at which risk of occurrence cardiovascular events for the subject is less than the specified level of cumulative lifetime risk.
82. The method of paragraph 81, further comprising:
identifying one or more therapeutic interventions to recommend to be administered to the subject using the level of cumulative plaque burden at which the risk of occurrence of cardiovascular events for the subject is less than the specified level of cumulative lifetime risk and, optionally, recommending administration, administering, and/or commencing administering the identified one or more therapeutic interventions.
83. The method of paragraph 61, wherein estimating, using the cumulative lifetime risks of the subject having a cardiovascular event at the respective levels of cumulative LDL exposure, the cumulative lifetime risks of the subject having a cardiovascular event at the respective ones of the multiple time intervals, comprises:
determining ages at which the respective levels of cumulative LDL exposure occur for the subject by using the cumulative LDL exposure trajectory for the subject.
84. The method of paragraph 1 or any other preceding paragraph, wherein the cardiometabolic health data for the subject includes the subject's latest measured SBP level and estimates of rate of change of SBP levels for the subject, the method further comprising predicting an age at which the subject is likely to develop hypertension.
85. The method of paragraph 1 or any other preceding paragraph,
wherein the one or more therapeutic interventions comprises a particular therapeutic intervention sequence, the particular therapeutic sequence indicating magnitude, duration, and timing of one or more interventions selected from among interventions designed to lower LDL, to lower SBP, or to lower both LDL and SBP, and wherein determining the benefit of administering to the subject the one or more therapeutic interventions comprises:
determining, using the multiple measures of risk and the at least one second trained ML model, benefit of administering the particular intervention sequence to the subject.
86. The method of paragraph 85, wherein the particular therapeutic intervention sequence comprises a sequence of one or more, optionally annual or bi-annual, administrations of a DNA-based therapeutic intervention, an RNA-based therapeutic intervention, a protein-based therapeutic intervention, or a pharmacological therapeutic intervention.
87. The method of paragraph 85 or 86, wherein determining the benefit of administering the particular therapeutic intervention sequence to the subject comprises:
determining expected proportional reduction and/or absolute reduction in risk of cardiovascular events for the subject in response to the particular therapeutic intervention sequence.
88. The method of paragraph 87, further comprising:
determining, based on the expected proportional and/or absolute reduction in risk, optionally when the reduction is greater than a threshold reduction, that the particular therapeutic intervention sequence is to be recommended for administration to the subject and, optionally, recommending administration, administering, and/or commencing administering the particular therapeutic intervention to the subject, optionally, wherein determining that the particular therapeutic intervention sequence is to be recommended for administration to the subject comprises determining expected proportional and/or absolute reduction in risk for each of multiple therapeutic intervention sequences different from one another and selecting the particular therapeutic intervention sequence based on the determined expected proportional and/or absolute reductions in risk.
89. The method of paragraph 85 or any other preceding paragraph, wherein the at least one second trained ML model was trained on training data comprising randomized data from:
(a) randomized trials of LDL and/or SBP lowering therapies, and
(b) Mendelian randomization studies evaluating genetic variants associated with lower LDL and/or SBP designed as naturally randomized trials.
90. The method of paragraph 89, wherein the training data comprises, for each of a plurality of participants in said trials, at least one LDL or SBP measurement along with a recorded age or date at which a first cardiovascular event occurred.
91. The method of paragraph 89 or 90, wherein the at least one second trained ML model was trained on randomized data obtained from:
(a) participant data for at least 1 million (M) or 1.5 M participants enrolled in one or more prospective studies, whereby for each of the at least 1 M or 1.5 M participants at least one LDL or SBP measurement were available along with a recorded age or date at which a first episode of a fatal or non-fatal myocardial infarction (MI), fatal or non-fatal ischemic stroke, or coronary revascularization occurred, with censoring applied at time of last follow-up, death, or first cardiovascular event; and
(b) participant data from at least 250K or 500K participants enrolled in at least 25, 50, or 75 randomized trials evaluating LDL or BP lowering therapies that provided time-to-event curves, and measurements of absolute difference in LDL or SBP between randomized groups in the randomized trials.
92. The method of any one of paragraphs 87-91,
wherein the multiple measures of risk comprise absolute instantaneous hazard rates, cumulative hazard rates, and cumulative event rates of the subject having a cardiovascular event at respective ones of the multiple time intervals, wherein the cumulative hazard rates and the cumulative event rates are not adjusted for the particular therapeutic intervention sequence,
wherein determining the expected proportional reduction and/or the absolute reduction in the risk of cardiovascular events for the subject in response to the particular therapeutic intervention sequence comprises:
(a) for each particular interval of follow-up for the subject from the subject's current age to an upper threshold age,
(i) determining, using the multiple measures of risk, a predicted instantaneous hazard rate of the subject having a cardiovascular event at the particular interval of follow up;
(ii) determining, using the at least one second ML model, a time-averaged instantaneous log hazard ratio for a one unit lower LDL or SBP corresponding to duration of treatment at the particular interval of follow-up for the subject;
(iii) determining an intervention-adjusted instantaneous hazard for the particular interval of follow-up by multiplying the instantaneous hazard rate of the subject determined at (a)(i) with the time-averaged instantaneous log hazard ratio determined at (a)(ii), thereby obtaining multiple intervention-adjusted instantaneous hazards for intervals of follow-up evaluated at (a);
(b) determining, using the multiple intervention-adjusted instantaneous hazards, intervention-adjusted cumulative hazard rates and cumulative event rates of cardiovascular events for intervals of follow-up for the subject from the subject's current age to the upper threshold age; and
(c) determining predicted proportional reductions in the risk of experiencing a cardiovascular event as ratios of the intervention-adjusted cumulated hazard rates and the cumulative hazard rates that are not adjusted for the particular therapeutic intervention sequence.
93. The method of paragraph 92, further comprising:
(d) determining predicted absolute reductions in the risk of experience of experiencing a cardiovascular event as absolute differences between the intervention-adjusted cumulative event rates and the cumulative event rates that are not adjusted for the particular therapeutic intervention sequence.
94. The method of paragraph 92, wherein determining the intervention-adjusted instantaneous hazard for the particular interval of follow-up further comprises additionally adjusting the intervention-adjusted instantaneous hazard for an expected absolute magnitude of the reduction in LDL or SBP in response to the particular therapeutic intervention sequence using a Wald ratio of effect estimates method.
95. The method of paragraph 85 or any other preceding paragraph, wherein the at least one second trained ML model comprises a non-linear regression model, an adaptive basis function regression model, a neural network regression model, a deep neural network regression model, a DNN with ordinary differential equations regression model, a logistic regression model, a polynomial regression model, a decision tree regression model, a random forest regression model, and/or a gradient boosted decision tree regression model.
96. The method of paragraph 95, wherein the at least one second ML model comprises a deep neural network (DNN) model.
97. The method of paragraph 96, wherein the DNN model is a causal DNN for Ordinary Differential Equations (c-DNN-ODE) model, optionally, wherein the c-DNN-ODE model has at least 3, 4, or 5 fully-connected layers.
98. The method of paragraph 97, wherein, in response to input indicating a duration of sustained intervention, optionally specified in intervals of months or years, the c-DNN-ODE model is trained to provide, output indicating magnitude of an instantaneous log hazard ratio of benefit of lowering LDL or SBP by one unit for each interval of the duration of sustained intervention.
99. The method of paragraph 98, wherein the output further indicates rate of change, with respect to the input indicating the duration of sustained intervention, of the magnitude of the instantaneous log hazard ratio of benefit of lowering LDL or SBP by one unit for each interval of the duration of sustained intervention.
100. The method of paragraph 1 or any other preceding paragraph,
wherein the one or more therapeutic interventions comprise multiple therapeutic intervention sequences, and
wherein determining the benefit of administering to the subject the one or more therapeutic interventions comprises:
determining, using the multiple measures of risk and the at least one second trained ML model, respective benefits of administering each of the multiple therapeutic intervention sequences to the subject.
101. The method of paragraph 100, wherein each of the multiple therapeutic intervention sequences is a therapeutic intervention sequence of one or more therapeutic interventions designed to lower LDL, to lower SBP, or to lower both LDL and SBP.
102. The method of paragraph 100 or 101, wherein the multiple therapeutic interventions sequences vary from one another based on type or types of therapeutics utilized, amounts of the therapeutic or the therapeutics administered, and/or timing of administering the therapeutic or therapeutics.
103. The method of any one of paragraphs 100-102, wherein the multiple therapeutic intervention sequences include a first therapeutic intervention sequence of one or more therapeutic intervention, the first therapeutic intervention sequence defined by information specifying, for each particular therapeutic intervention in the first therapeutic intervention sequence,
a type of therapeutic or therapeutics part of the particular therapeutic intervention, an amount of the therapeutic or the therapeutics to administer as part of the
particular therapeutic intervention, and
timing information indicating when to administer the particular therapeutic intervention.
104. The method of any one of paragraphs 100-103, wherein the multiple therapeutic intervention sequences comprise one or more sequences of one or more, optionally annual or bi-annual, administrations of a DNA-based therapeutic intervention, an RNA-based therapeutic intervention, a protein-based therapeutic intervention, or a pharmacological therapeutic intervention.
105. The method of any one of paragraphs 100-104, further comprising:
generating the multiple therapeutic intervention sequences; and after generating at least one or all, of the multiple therapeutic intervention sequences, begin determining respective benefits of administering each of the multiple therapeutic intervention sequences to the subject.
106. The method of paragraph 105, wherein generating the multiple therapeutic intervention sequences comprises:
enumerating a set of therapeutic intervention sequences by varying an age of the subject for when to begin an intervention therapeutic sequence, the duration of the intervention therapeutic sequence, the types of therapeutic or therapeutics used as part of the therapeutic intervention sequence, and/or the timings of therapeutic interventions in the therapeutic intervention sequence 107. The method of paragraph 106, further comprising:
filtering one or more therapeutic intervention sequences from the set of therapeutic intervention sequences using a domain expert clinical translation heuristic to obtain the multiple therapeutic intervention sequences, the domain expert clinical translation heuristic encoding a set of one or more rules indicating permitted order, intensification, and/or discontinuation of treatments in a therapeutic intervention sequence.
108. The method of paragraph 107, wherein the one or more rules include a rule indicating that discontinuous therapeutic intervention sequences that start and stop interventions more than a threshold number of times are not permitted, and the filtering comprises removes any such therapeutic intervention sequences from the set of therapeutic intervention sequences.
109. The method of paragraph 106, wherein the multiple therapeutic intervention sequences include only those therapeutic intervention sequences, in which over time, the same therapeutic intervention is continued or intensified, or a new therapeutic intervention is added.
110. The method of any one of paragraphs 100-109, wherein determining the respective benefits of administering each of the multiple therapeutic intervention sequences to the subject comprises:
for each particular therapeutic intervention sequence of the multiple therapeutic intervention sequences:
determining expected proportional reduction and/or absolute reduction in risk of cardiovascular events for the subject in response to the particular therapeutic intervention sequence,
thereby obtaining multiple expected proportional and/or absolute reductions in risk corresponding to the multiple therapeutic intervention sequences and, optionally, storing the obtained multiple expected proportional and/or absolute reductions in risk corresponding to the multiple therapeutic intervention sequences.
111. The method of paragraph 110, wherein the identifying, using the determined benefit of administering the one or more therapeutic interventions, at least one therapeutic intervention to recommend to be administered to the subject comprises:
selecting, from among the multiple therapeutic intervention sequences, a specific therapeutic intervention sequence to recommend to be administered to the subject, the selecting being performed using at least some of the multiple expected proportional and/or absolute reductions in risk corresponding to the multiple therapeutic intervention sequences,
the method, optionally, further comprising: recommending the specific therapeutic intervention sequence and/or administering or commencing administering the specific therapeutic intervention sequence.
112. The method of paragraph 110 or 111,
wherein the multiple measures of risk comprise absolute instantaneous hazard rates, cumulative hazard rates, and cumulative event rates of the subject having a cardiovascular event at respective ones of the multiple time intervals, wherein the cumulative hazard rates and the cumulative event rates are not adjusted for the particular therapeutic intervention sequence, and
wherein determining the expected proportional reduction and/or the absolute reduction in risk of cardiovascular events for the subject in response to the particular therapeutic intervention sequence comprises:
(a) for each particular interval of follow-up for the subject from the age of the subject at which the particular therapeutic intervention sequence is to commence to an upper threshold age,
(i) determining, using the multiple measures of risk, a predicted instantaneous hazard rate of the subject having a cardiovascular event at the particular interval of follow up;
(ii) determining, using the at least one second ML model, a time-averaged instantaneous log hazard ratio for a one unit lower LDL or SBP corresponding to duration of treatment at the particular interval of follow-up for the subject;
(iii) determining an intervention-adjusted instantaneous hazard for the particular interval of follow-up by multiplying the instantaneous hazard rate of the subject determined at (a)(i) with the time-averaged instantaneous log hazard ratio determined at (a)(ii), thereby obtaining multiple intervention-adjusted instantaneous hazards for intervals of follow-up evaluated at (a);
(b) determining, using the multiple intervention-adjusted instantaneous hazards, intervention-adjusted cumulative hazard rates and cumulative event rates of cardiovascular events for intervals of follow-up for the subject from the age of the subject at which the particular therapeutic intervention sequence is to commence to the upper threshold age; and
(c) determining predicted proportional reductions in the risk of experiencing a cardiovascular event as ratios of the intervention-adjusted cumulated hazard rates and the cumulative hazard rates that are not adjusted for the particular therapeutic intervention sequence.
113. The method of paragraph 112, further comprising:
(d) determining predicted absolute reductions in the risk of experience of experiencing a cardiovascular event as absolute differences between the intervention-adjusted cumulative event rates and the cumulative event rates that are not adjusted for the particular therapeutic intervention sequence.
114. The method of paragraph 112 or 113, wherein the at least one trained second ML model comprises a causal DNN for Ordinary Differential Equations (c-DNN-ODE) model, a non-linear regression model, an adaptive basis function regression model, a neural network regression model, a deep neural network regression model, a logistic regression model, a polynomial regression model, a decision tree regression model, a random forest regression model, and/or a gradient boosted decision tree regression model.
115. The method of any one of paragraphs 100-114, wherein the at least one second trained ML model comprises a causal DNN for Ordinary Differential Equations (c-DNN-ODE) model.
116. The method of paragraph 115, wherein, in response to input indicating a duration of sustained intervention, optionally specified in intervals of months or years, the c-DNN-ODE model is trained to provide:
output indicating magnitude of an instantaneous log hazard ratio of benefit of lowering LDL or SBP by one unit for each interval of the duration of sustained intervention, and rate of change, with respect to the input indicating the duration of sustained intervention, of the magnitude of the instantaneous log hazard ratio of benefit of lowering LDL or SBP by one unit for each interval of the duration of sustained intervention.
117. The method of paragraph 115 or 116, wherein the c-DNN-ODE model was trained on randomized data from:
(a) randomized trials of LDL and/or SBP lowering therapies, and
(b) Mendelian randomization studies evaluating genetic variants associated with lower LDL and/or SBP designed as naturally randomized trials.
118. The method of paragraph 117, wherein the training data comprises, for each of a plurality of participants in said trials, at least one LDL or SBP measurement along with a recorded age or date at which a first cardiovascular event occurred.
119. The method of paragraph 117 or 118, wherein the c-DNN-ODE model was trained on randomized data obtained from:
(a) participant data for at least 1 million (M) or 1.5 M participants enrolled in one or more prospective studies, whereby for each of the at least 1 M or 1.5 M participants at least one LDL or SBP measurement were available along with a recorded age or date at which a first episode of a fatal or non-fatal myocardial infarction (MI), fatal or non-fatal ischemic stroke, or coronary revascularization occurred, with censoring applied at time of last follow-up, death, or first cardiovascular event; and
(b) participant data from at least 250K or 500K participants enrolled in at least 25, 50, or 75 randomized trials evaluating LDL or BP lowering therapies that provided time-to-event curves, and measurements of absolute difference in LDL or SBP between randomized groups in the randomized trials.
120. The method of paragraph 110, wherein identifying, using the determined benefit of administering the one or more therapeutic interventions, at least one therapeutic intervention to recommend to be administered to the subject comprises:
selecting, from among the multiple therapeutic intervention sequences, a specific therapeutic intervention sequence to recommend to be administered to the subject.
121. The method of paragraph 120, wherein the selecting is performed using a domain expert policy guideline specifying one or more rules for selecting therapeutic intervention sequences for an explicitly defined overall treatment goal for the subject.
122. The method of paragraph 121, further comprising: removing one or more of the multiple therapeutic intervention sequences that are inconsistent with the domain expert policy guideline.
123. The method of paragraph 120, wherein the selecting comprises:
determining a respective sequence score for each of at least some of the multiple therapeutic intervention sequences to obtain a plurality of sequence scores; and selecting the specific therapeutic intervention sequence based on the determined plurality of sequence scores.
124. The method of paragraph 123, wherein determining the respective sequence score for each of the at least some of the multiple therapeutic intervention sequences to obtain the plurality of sequence scores comprises using a value function to assign sequence scores to therapeutic intervention sequences including using the value function to assign a specific sequence score to the specific therapeutic intervention sequence.
125. The method of paragraph 124, wherein using the value function to assign the specific sequence score to the specific therapeutic intervention sequence comprises calculating a measure of reward for specific therapeutic intervention sequence.
126. The method of paragraph 125, wherein using the value function to assign the specific sequence score to the specific therapeutic intervention sequence comprises calculating a penalty for each of one or more interventions used in the specific therapeutic intervention sequence in furtherance of penalizing therapeutic intervention sequences having a greater number of treatments and/or a stronger intensity of treatments, than other therapeutic interventions sequences, for achieving clinical goal(s) of treating the subject.
127. The method of paragraph 126, wherein the measure of reward is determined as a sum of rewards over multiple time intervals, optionally years, and wherein using the value function to assign the specific sequence score to the specific therapeutic intervention sequence comprises discounting the rewards, in the sum of rewards, corresponding to later time intervals during the specific therapeutic intervention sequence relative to earlier time intervals during the specific therapeutic intervention sequence.
128. The method of paragraph 125,
wherein the determined benefit of administering the one or more therapeutic interventions comprises:
intervention-adjusted cumulative hazard rates for intervals of follow-up for the subject from the age of the subject at which the specific therapeutic intervention sequence is to commence to the upper threshold age; and wherein the measure of reward is determined using the intervention-adjusted cumulative hazard rates.
129. The method of paragraph 125, wherein the determined benefit of administering the one or more therapeutic interventions comprises:
predicted proportional reductions in the risk of experiencing a cardiovascular event, due to the specific therapeutic intervention sequence, for intervals of follow-up for the subject from the age of the subject at which the specific therapeutic intervention sequence is to commence to the upper threshold age,
wherein the measure of reward is determined using the predicted proportional reductions in the risk.
130. The method of paragraph 124, wherein determining the respective sequence score for each of the at least some of the multiple therapeutic intervention sequences comprises selecting the at least some of the multiple therapeutic intervention sequences as a subset of the multiple therapeutic intervention sequences for which to determine a respective sequence score using a Markov chain tree search (MCTS) algorithm.
131. The method of paragraph 124, wherein determining the respective sequence score for each of the at least some of the multiple therapeutic intervention sequences comprises determining a respective sequence score for each of the multiple therapeutic intervention sequences.
132. The method of paragraph 120, further comprising:
providing an output indicating the selected specific therapeutic intervention sequence.
133. The method of paragraph 132, wherein the output provides an indication of how the subject's risk of a cardiovascular event is changed as a result of the specific therapeutic intervention sequence.
134. The method of paragraph 132, wherein the output includes text explaining: reasoning and biological rationale of why the subject is at risk for cardiovascular disease, how the subject can minimize their risk, how much the subject will benefit from the specific therapeutic intervention sequence, a recommended immediate action, a description of one or more expected subsequent actions to be take and when to take them based on the subject's current state of cardiovascular health.
135. The method of paragraph 134, further comprising generating the text using a large language model (LLM) by prompting the LLM with predetermined prompts and values of one or more quantities determined in performance the method of paragraph 134.
136. The method of paragraph 132, wherein the output further indicates the subject's LDL trajectory, SBP trajectory, Lp(a) trajectory, cumulative LDL exposure trajectory, cumulative SBP exposure trajectory, cumulative Lp(a) trajectory, and/or determined multiple measures of risk that the subject develops cardiovascular risk.
136. The method of paragraph 120, further comprising:
commencing administration or administering to the subject the specific therapeutic intervention sequence.
137. Repeating the method of paragraphs 1 to 136 using updated cardiometabolic health data.
138. The method of paragraph 1 or any other paragraph 2 to 136, further comprising:
after a specified time period,
obtaining updated cardiometabolic health data for the subject;
determining, using at least some of the updated cardiometabolic health data, the first trained machine learning (ML) model and for each of multiple second time intervals, one or more updated measures of risk that the subject develops cardiovascular disease to obtain multiple updated measures of risk corresponding to the multiple second time intervals;
determining, using the multiple updated measures of risk that the subject develops cardiovascular disease and the at least one second trained ML model, benefit of administering to the subject one or more second therapeutic interventions designed to reduce risk of cardiovascular disease by targeting one or more modifiable causes of the cardiovascular disease; and
identifying, using the determined benefit of administering the one or more therapeutic interventions, at least one second therapeutic intervention to recommend to be administered to the subject.
139. The method of paragraph 138, wherein the updated cardiometabolic health data includes updated subject characteristic and/or measurement data comprising:
one or more updated values for one or more clinical characteristics of the subject,
one or more updated values for one or more physical measurements of the subject, and/or
one or more updated values for one or biochemical measurements of the subject.
140. The method of paragraph 139,
wherein prior to obtaining the updated cardiometabolic health data for the subject, a particular therapeutic intervention of the at least one identified therapeutic intervention was administered to the subject, and
wherein the one or more updated values for the one or more physical and/or biochemical measurements of the subject include one or more updated values reflecting achieved absolute reduction in a modifiable cause of disease targeted by the particular therapeutic intervention.
141. The method of paragraph 139, wherein the one or more updated values for one or more physical measurements of the subject include one or more updated values for one or more physical measurements of the subject selected from the group consisting of systolic blood pressure (SBP), diastolic blood pressure (DBP), weight, waist circumference, height, body mass index (BMI), and waist-to-height ratio.
142. The method of paragraph 139, wherein the one or more updated values for one or more biochemical measurements of the subject include one or more updated values for one or more biochemical measurements of the subject selected from the group consisting of low-density lipoprotein (LDL) level, high-density lipoprotein (HDL) level, total cholesterol level, triglyceride (TG) level, non-HDL cholesterol level, apolipoprotein (apoB) level, lipoprotein (a) (Lp(a)) level, hemoglobin A1c (HbA1c) level, and c-reactive protein (CRP) level.
143. The method of paragraph 139, further comprising:
estimating an updated LDL trajectory for the subject using the updated subject characteristic and/or measurement data and a third ML model that has been trained to estimate an LDL level for the subject at each of multiple ages.
144. The method of paragraph 143, further comprising:
estimating an updated SBP trajectory for the subject using the updated subject characteristic and/or measurement data and a fourth ML model that has been trained to estimate an SBP level for the subject at each of multiple ages.
145. The method of paragraph 144, wherein the third ML model and fourth ML models are trained RNNs, optionally bi-directional LSTMs.
146. The method of paragraph 138, wherein determining the one or more updated measures of risk that the subject develops cardiovascular disease, for each of the multiple second time intervals, to obtain the updated multiple measures of risk corresponding to the multiple second time intervals, comprises:
(a) estimating, using the first trained ML model and the at least some of the updated cardiometabolic health data including an updated cumulative LDL exposure trajectory for the subject, updated values indicative of log hazard ratios for risk of the subject having a cardiovascular event at respective levels of cumulative LDL exposure;
(b) estimating, using the updated values indicative of the log hazard ratios,
(i) updated absolute instantaneous hazard rates of the subject having a cardiovascular event at the respective levels of cumulative LDL exposure, and
(ii) updated cumulative lifetime risks of the subject having a cardiovascular event at the respective levels of cumulative LDL exposure; and
(c) estimating, using the updated cumulative LDL exposure trajectory for the subject, the updated multiple measures of risk to include:
(i) updated absolute instantaneous hazard rates of the subject having a cardiovascular event at respective ones of the multiple second time intervals, and
(ii) updated cumulative lifetime hazard and event rates of the subject having a cardiovascular event at the respective ones of the multiple second time intervals.
147. The method of paragraph 140, wherein determining, using the multiple updated measures of risk that the subject develops cardiovascular disease and the at least one second trained ML model, the benefit of administering to the subject one or more second therapeutic interventions designed to reduce risk of cardiovascular disease by targeting one or more modifiable causes of the cardiovascular disease is performed based on information indicative of the achieved absolute reduction in the modifiable cause of disease targeted by the particular therapeutic intervention.
148. The method of paragraph 1 or any other preceding paragraph, further comprising:
administering and/or commencing administering to the subject the at least one identified therapeutic intervention,
wherein the at least one identified therapeutic intervention is a DNA-based therapeutic intervention, an RNA-based therapeutic intervention, a protein-based therapeutic intervention, or a pharmacological therapeutic intervention.
149. The method of paragraph 1 or any other preceding paragraph, wherein the at least one identified therapeutic intervention is LDL-lowering siRNA, AGT-lowering siRNA, or a combination therapy of LDL-lowering siRNA and AGT-lowering siRNA.
150. A method for identifying an intervention for a subject in furtherance of preventing development of cardiovascular disease in the subject, the method comprising:
using at least one computer hardware processor to perform:
obtaining cardiometabolic health data for the subject;
determining, using at least some of the cardiometabolic health data, a survival model with cumulative LDL exposure as interval of follow-up, and for each of multiple time intervals, one or more measures of risk that the subject develops cardiovascular disease to obtain multiple measures of risk corresponding to the multiple time intervals;
determining, using the multiple measures of risk that the subject develops cardiovascular disease, benefit of administering to the subject one or more therapeutic interventions designed to reduce risk of cardiovascular disease by targeting one or more modifiable causes of the cardiovascular disease; and identifying, using the determined benefit of administering the one or more therapeutic interventions, at least one therapeutic intervention to recommend to be administered to the subject.
151. A method for identifying an intervention for a subject in furtherance of preventing development of cardiovascular disease in the subject, the method comprising:
using at least one computer hardware processor to perform:
obtaining cardiometabolic health data for the subject, the cardiometabolic health data comprising clinical, physical, and biochemical measurements of the subject, the obtaining further comprising estimating a cumulative LDL exposure trajectory for the subject using a trained ML model and the physical and biochemical measurements;
determining, using at least some of the cardiometabolic health data including the cumulative LDL exposure trajectory and for each of multiple time intervals, one or more measures of risk that the subject develops cardiovascular disease to obtain multiple measures of risk corresponding to the multiple time intervals;
determining, using the multiple measures of risk that the subject develops cardiovascular disease, benefit of administering to the subject one or more therapeutic interventions designed to reduce risk of cardiovascular disease by targeting one or more modifiable causes of the cardiovascular disease; and
identifying, using the determined benefit of administering the one or more therapeutic interventions, at least one therapeutic intervention to recommend to be administered to the subject.
152. A method of estimating a biomarker for a subject in furtherance of preventing development of cardiovascular disease in the subject, the method comprising:
obtaining cardiometabolic health data for the subject, the cardiometabolic health data
comprising subject characteristic and/or measurement data comprising:
one or more values for one or more clinical characteristics of the subject,
one or more values for one or more physical measurements of the subject, and/or
one or more values for one or biochemical measurements of the subject;
estimating, using a trained machine learning (ML) model and the subject characteristic and/or measurement data, an LDL level trajectory for the subject, wherein the LDL level trajectory for the subject comprises an estimated LDL level for the subject for each of multiple prior ages of the subject and multiple future ages of the subject; and
determining a cumulative LDL exposure trajectory for the subject as the biomarker for use in identifying a therapeutic intervention for the subject in furtherance of preventing development of cardiovascular disease in the subject.
153. A method of estimating a biomarker for a subject in furtherance of preventing development of cardiovascular disease in the subject, the method comprising:
obtaining cardiometabolic health data for the subject, the cardiometabolic health data
comprising subject characteristic and/or measurement data comprising:
one or more values for one or more clinical characteristics of the subject,
one or more values for one or more physical measurements of the subject, and/or
one or more values for one or biochemical measurements of the subject;
estimating, using a trained machine learning (ML) model and the subject characteristic and/or measurement data, an SBP level trajectory for the subject, wherein the SBP level trajectory for the subject comprises an estimated SBP level for the subject for each of multiple prior ages of the subject and multiple future ages of the subject; and
determining a cumulative SBP exposure trajectory for the subject as the biomarker for use in identifying a therapeutic intervention for the subject in furtherance of preventing development of cardiovascular disease in the subject.
154. A method of estimating a biomarker for a subject in furtherance of preventing development of cardiovascular disease in the subject, the method comprising:
obtaining cardiometabolic health data for the subject, the cardiometabolic health data
comprising subject characteristic and/or measurement data comprising:
one or more values for one or more clinical characteristics of the subject,
one or more values for one or more physical measurements of the subject, and/or
one or more values for one or biochemical measurements of the subject;
estimating, using a trained machine learning (ML) model and the subject characteristic and/or measurement data, an Lp(a) level trajectory for the subject, wherein the Lp(a) level trajectory for the subject comprises an estimated Lp(a) level for the subject for each of multiple prior ages of the subject and multiple future ages of the subject; and
determining a cumulative Lp(a) exposure trajectory for the subject as the biomarker for use in identifying a therapeutic intervention for the subject in furtherance of preventing development of cardiovascular disease in the subject.
155. A method of determining one or more measures of risk that a subject develops cardiovascular disease, the method comprising, for each of multiple time intervals:
using at least one computer hardware processor to perform:
(a) estimating, using a trained cardiovascular risk prediction machine learning model and cardiometabolic health data for the subject including a cumulative LDL exposure trajectory for the subject, values indicative of log hazard ratios for risk of the subject having a cardiovascular event at respective levels of cumulative LDL exposure, wherein the cardiovascular risk prediction machine learning model has been trained using training data comprising, for each of a plurality of participants enrolled in one or more prospective studies, multiple LDL measurements along with a recorded age or date at which a first cardiovascular event occurred, optionally wherein the first cardiovascular event is a first episode of a fatal or non-fatal myocardial infarction (MI), fatal or non-fatal ischemic stroke, or coronary revascularization; and
(b) estimating, using the values indicative of the log hazard ratios,
(i) absolute instantaneous hazard rates of the subject having a cardiovascular event at the respective levels of cumulative LDL exposure, and
(ii) cumulative lifetime risks of the subject having a cardiovascular event at the respective levels of cumulative LDL exposure;
156. A method of determining an expected proportional reduction and/or absolute reduction in risk of cardiovascular events for a subject in response to a particular therapeutic intervention sequence, the particular therapeutic sequence indicating magnitude, duration, and timing of one or more interventions associated with a reduction of LDL level and/or SBP level over an interval of follow up for the subject, the method comprising:
using at least one computer hardware processor to perform:
determining multiple measures of risk of cardiovascular events for the subject comprising absolute instantaneous hazard rates, cumulative hazard rates, and cumulative event rates of the subject having a cardiovascular event at respective ones of multiple time intervals, wherein the cumulative hazard rates and the cumulative event rates are not adjusted for the particular therapeutic intervention sequence, and
determining the expected proportional reduction and/or the absolute reduction in the risk of cardiovascular events for the subject in response to the particular therapeutic intervention sequence using a method that comprises:
(a) for each particular interval of follow-up for the subject from the subject's current age to an upper threshold age,
(i) determining, using the multiple measures of risk, a predicted instantaneous hazard rate of the subject having a cardiovascular event at the particular interval of follow up;
(ii) determining, using a benefit prediction machine learning model, a time-averaged instantaneous log hazard ratio for a one unit lower LDL or SBP corresponding to duration of treatment at the particular interval of follow-up for the subject;
(iii) determining an intervention-adjusted instantaneous hazard for the particular interval of follow-up by multiplying the instantaneous hazard rate of the subject determined at (a)(i) with the time-averaged instantaneous log hazard ratio determined at (a)(ii),
thereby obtaining multiple intervention-adjusted instantaneous hazards for intervals of follow-up evaluated at (a);
(b) determining, using the multiple intervention-adjusted instantaneous hazards, intervention-adjusted cumulative hazard rates and cumulative event rates of cardiovascular events for intervals of follow-up for the subject from the subject's current age to the upper threshold age; and
(c) determining predicted proportional reductions in the risk of experiencing a cardiovascular event as ratios of the intervention-adjusted cumulated hazard rates and the cumulative hazard rates that are not adjusted for the particular therapeutic intervention sequence.
157. A method for pricing an insurance instrument for a subject based, the method comprising: using at least one computer hardware processor to perform:
obtaining cardiometabolic health data for the subject;
determining, using at least some of the cardiometabolic health data and for each of multiple time intervals, one or more measures of risk that the subject develops a disease to obtain multiple measures of risk corresponding to the multiple time intervals; and pricing the insurance instrument for the subject based on the multiple measures of risk corresponding to the multiple time intervals.
158. The method of paragraph 157, further comprising:
determining, using the multiple measures of risk that the subject develops the disease,
benefit of administering to the subject one or more therapeutic interventions designed to reduce risk of the disease by targeting one or more modifiable causes of the disease,
wherein pricing the insurance instrument for the subject is further based on the determined benefit of administering to the subject the one or more therapeutic interventions.
159. The method of paragraph 158, further comprising:
identifying, using the determined benefit of administering the one or more therapeutic interventions, at least one therapeutic intervention to recommend to be administered to the subject; and
monitoring the subject's compliance with administration of the at least one therapeutic intervention,
wherein pricing the insurance instrument for the subject is further based on the subject's compliance with the administration of the at least one therapeutic intervention.
160. A system, comprising:
at least one computer hardware processor; and
at least one non-transitory computer readable storage medium storing software comprising:
(a) a cardiometabolic health data module comprising processor-executable instructions that, when executed by the at least one computer hardware processor, cause the at least one computer hardware processor to perform obtaining cardiometabolic health data for a subject;
(b) a risk assessment module comprising processor-executable instructions that, when executed by the at least one computer hardware processor, cause the at least one computer hardware processor to perform determining, using at least some of the cardiometabolic health data and for each of multiple time intervals, one or more measures of risk that the subject develops disease to obtain multiple measures of risk corresponding to the multiple time intervals;
(c) a therapeutic intervention benefit assessment module comprising processor executable instructions that, when executed by the at least one computer hardware processor, cause the at least one computer hardware processor to perform determining, using the multiple measures of risk that the subject develops the disease, benefit of administering to the subject one or more therapeutic interventions designed to reduce risk of the disease by targeting one or more modifiable causes of the disease; and
(d) a therapeutic intervention selection module comprising processor executable instructions that, when executed by the at least one computer hardware processor, cause the at least one computer hardware processor to perform identifying, using the determined benefit of administering the one or more therapeutic interventions, at least one therapeutic intervention to recommend to be administered to the subject.
161. The system of paragraph 160, further comprising a machine learning model datastore configured to store one or more trained ML models to be used by one or more of the cardiometabolic health data module, the risk assessment module, the therapeutic intervention benefit assessment module, and/or the therapeutic intervention selection module.
162. The system of paragraph 161, wherein the ML model datastore stores:
(a) parameters for a first ML model to be used by the risk assessment module, optionally wherein the first ML model is a survival DNN-ODE model;
(b) parameters for at least one second ML model to be used by the therapeutic intervention benefit assessment module, optionally wherein the at least one second ML model includes one or more c-DNN-ODE models; and/or
(c) parameters for one or more ML models to be used by the cardiometabolic health data module, optionally the one or more ML models comprising one or more bi-directional LSTM models to estimate one or more trajectories of biomarker levels.
163. The system of any one of paragraphs 160-162,
wherein the software is cloud-based software deployed as a service, and/or
wherein the software further comprises a user interface module configured to generate one or more user interfaces through which users can interact with the software.
164. The system of paragraph 163, wherein the software implements a Deep Causal AI Agent.
165. The system of paragraph 163, wherein the software is configured to guide timing and/or intensity of therapeutic interventions by personalizing prevention of cardiometabolic disease, monitor compliance of a subject with administration of any recommended therapeutic interventions, and/or price financial instruments, optionally insurance, for the subject.
166. A system, comprising:
at least one computer hardware processor; and at least one non-transitory computer readable storage medium storing processor-executable instructions that, when executed by the at least one processor, cause the at least one computer hardware processor to perform the method of any one of the foregoing method paragraphs.
167. At least one non-transitory computer readable storage medium storing processor-executable instructions that, when executed by at least one processor, cause the at least one processor to perform the method of any one of the foregoing method paragraphs.
168. A method for identifying an intervention for a subject in furtherance of preventing development of cardiovascular disease in the subject, the method comprising:
using at least one computer hardware processor to perform:
obtaining cardiometabolic health data for the subject;
determining, using at least some of the cardiometabolic health data, a first trained machine learning (ML) model and for each of multiple time intervals, one or more measures of risk that the subject develops cardiovascular disease to obtain multiple measures of risk corresponding to the multiple time intervals;
determining, using the multiple measures of risk that the subject develops cardiovascular disease and at least one second ML model, benefit of administering to the subject one or more therapeutic interventions designed to reduce risk of cardiovascular disease by targeting one or more modifiable causes of the cardiovascular disease; and
identifying, using the determined benefit of administering the one or more therapeutic interventions, at least one therapeutic intervention to recommend being administered to the subject.
169. The method of paragraph 168, wherein the cardiometabolic health data comprises:
one or more values for one or more clinical characteristics of the subject;
one or more values for one or more physical measurements of the subject; and/or
one or more values for one or more biochemical measurements of the subject;
170. The method of paragraph 169, wherein the one or more values for one or more clinical characteristics comprise:
(i) values for one or more demographic characteristics of the subject, one or more genetic characteristics of the subject, one or more family history characteristics of the subjects, one or more comorbidities, and/or risk factors;
(ii) the one or more values for one or more physical measurements of the subject comprise one or more values for physical measurements of quantities that are risk factors for cardiometabolic disease and/or physiologic al measurements selected from blood pressure measurements and measurements indicative of adiposity; and/or
(iii) the one or more values for one or more biochemical measurements of the subject comprise one or more values for biochemical measurements of quantities that are risk factors for cardiovascular disease and/or biochemical measurements selected from measurements of one or more biochemical markers.
171. The method of paragraph 168, wherein the cardiometabolic health data includes a value indicating the subject's age, a value indicating the subject's biological sex, a value indicating the subject's LDL level, a value indicating the subject's SBP, and a value indicating the subject's DBP.
172. The method of paragraph 169, wherein obtaining the cardiometabolic health data further comprises:
encoding the cardiometabolic health data for the subject into a first feature vector;
estimating an LDL level trajectory for the subject by processing the first feature vector using an LDL trajectory prediction machine learning (ML) model that has been trained to estimate an LDL level for a subject at each of multiple prior ages and each of multiple future ages using training data comprising for each of a plurality of participants, repeated longitudinal measures of LDL levels and values for the one or more clinical characteristics and/or the one or more physical measurements and/or the one or more biochemical measurements over at least 10, at least 20, at least 30, or at least 50 years of follow-up,
wherein the LDL level trajectory for the subject comprises an estimated LDL level for the subject for each of multiple prior ages of the subject and multiple future ages of the subject,
wherein the multiple prior ages and the multiple future ages are, respectively, before and after the subject's age at which a most recent LDL measurement of the subject was made.
173. The method of paragraph 172, further comprising:
estimating, using the LDL level trajectory, a cumulative LDL exposure trajectory for the subject with respect to a set of ages, wherein the cumulative LDL exposure trajectory comprises an estimated cumulative LDL exposure level for the subject at each age in the set of ages.
174. The method of paragraph 172, wherein the LDL trajectory prediction ML model is a bi-directional LSTM (bi-LSTM) model, and
processing the first feature vector using the LDL trajectory prediction ML model comprises: estimating an LDL level for the subject at each of the multiple future ages using a forward pass of the bi-LSTM model; and
estimating an LDL level for the subject at each of the multiple prior ages using a backward pass of the bi-LSTM model.
175. The method of paragraph 172, wherein obtaining the cardiometabolic health data further comprises:
encoding the cardiometabolic health data for the subject into a second feature vector;
estimating an SBP level trajectory for the subject by processing the second feature vector using an SBP trajectory prediction ML model that has been trained to estimate an SBP level for a subject at each of multiple prior ages and each of multiple future ages using training data comprising, for each of a plurality of participants, repeated longitudinal measures of SBP levels and values for the one or more clinical characteristics and/or the one or more physical measurements and/or the one or more biochemical measurements over at least 10, at least 20, at least 30, or at least 50 years of follow-up,
wherein the SBP level trajectory for the subject comprises an estimated SBP level for the subject for each of multiple prior ages of the subject and multiple future ages of the subject, and
wherein the SBP trajectory prediction ML model is a bi-LSTM model.
176. The method of paragraph 168, wherein determining the one or more measures of risk that the subject develops cardiovascular disease, for each of the multiple time intervals, to obtain the multiple measures of risk corresponding to the multiple time intervals, comprises:
(a) estimating, using the first trained ML model and the at least some of the cardiometabolic health data including a cumulative LDL exposure trajectory for the subject, values indicative of log hazard ratios for risk of the subject having a cardiovascular event at respective levels of cumulative LDL exposure; and
(b) estimating, using the values indicative of the log hazard ratios,
(i) absolute instantaneous hazard rates of the subject having a cardiovascular event at the respective levels of cumulative LDL exposure, and
(ii) cumulative lifetime risks of the subject having a cardiovascular event at the respective levels of cumulative LDL exposure;
(c) estimating, using the cumulative LDL exposure trajectory for the subject, the multiple measures of risk to include:
(i) absolute instantaneous hazard rates of the subject having a cardiovascular event at respective ones of the multiple time intervals, and
(ii) cumulative lifetime hazard and event rates of the subject having a cardiovascular event at the respective ones of the multiple time intervals.
177. The method of paragraph 176, wherein estimating, using the first trained ML model and the at least some of the cardiometabolic health data, the values indicative of the log hazard ratios for risk of the subject having a cardiovascular event at the respective levels of cumulative LDL exposure comprises:
encoding the at least some of the cardiometabolic health data to obtain input feature data; and
estimating, by processing the input feature data using the first trained ML model, the values indicative of the log hazard ratios for the risk of the subject having a cardiovascular event at the respective levels of cumulative LDL exposure.
178. The method of paragraph 177, wherein the input feature data comprises:
a set of input feature vectors, each particular one of the input feature vectors corresponding to a particular cumulative LDL exposure level from among the respective levels of cumulative LDL exposure,
wherein the particular input feature vector in the set of input feature vectors that corresponds to the particular cumulative LDL exposure level, comprises:
(a) subject characteristic and/or measurement data comprising:
one or more values for one or more clinical characteristics of the subject,
one or more values for one or more physical measurements of the subject, and/or
one or more values for one or biochemical measurements of the subject;
(b) an LDL level trajectory for the subject;
(c) a cumulative LDL exposure trajectory for the subject;
(d) an SBP level trajectory for the subject indicating an estimate of the subject's SBP level at each of the respective levels of cumulative LDL exposure;
(e) a cumulative SBP exposure trajectory for the subject indicating an estimate of the subject's cumulative SBP exposure at each of the respective levels of cumulative LDL exposure;
(f) an Lp(a) level trajectory for the subject indicating an estimate of the subject's Lp(a) level at each level of the respective levels of cumulative LDL exposure;
(g) a cumulative Lp(a) exposure trajectory for the subject indicating an estimate of the subject's a cumulative Lp(a) exposure at each level of the respective levels of cumulative LDL exposure;
(h) a weight trajectory for the subject indicating an estimate of the subject's weight at each of the respective levels of cumulative LDL exposure;
(i) a waist circumference trajectory for the subject indicating an estimate of the subject's waist circumference at each of the respective levels of cumulative LDL exposure;
(j) an HbA1c level trajectory for the subject indicating an estimate of the subject's HbA1c level at each of the respective levels of cumulative LDL exposure;
(k) an age trajectory for the subject indicating an estimated age for the subject at each of the respective levels of cumulative LDL exposure; and/or
(l) a positionally encoded version of one or more of (a), (b), (d), (e), (f), (g), (h), (i), (j), and (k) obtained by positionally encoding the one or more of (a), (b), (d), (e), (f), (g), (h), (i), (j), and (k) by age of the subject at which the particular cumulative LDL exposure level is predicted to occur.
179. The method of paragraph 168, wherein the first machine learning model has been trained using training data comprising, for each of a plurality of participants enrolled in one or more prospective studies, multiple LDL and/or multiple SBP measurements along with a recorded age or date at which a first cardiovascular event occurred, wherein the first cardiovascular event is a first episode of a fatal or non-fatal myocardial infarction (MI), fatal or non-fatal ischemic stroke, or coronary revascularization.
180. The method of paragraph 168, wherein the first trained ML model is a trained survival model using cumulative LDL exposure as an interval of follow-up.
181. The method of paragraph 180, wherein the first trained ML model is a trained survival DNN model with piecewise exponential models (PEM) using cumulative LDL exposure as the interval of follow-up, wherein the trained survival DNN model comprises a different set of trained weights for each interval of follow-up, wherein each interval of follow-up is an interval of cumulative LDL exposure.
182. The method of paragraph 168,
wherein the one or more therapeutic interventions comprises a particular therapeutic intervention sequence, the particular therapeutic sequence indicating magnitude, duration, and timing of one or more interventions selected from among interventions designed to lower LDL, to lower SBP, or to lower both LDL and SBP, and
wherein determining the benefit of administering to the subject the one or more therapeutic interventions comprises:
determining, using the multiple measures of risk and the at least one second trained ML model, benefit of administering the particular intervention sequence to the subject,
wherein determining the benefit of administering the particular therapeutic intervention sequence to the subject comprises:
determining expected proportional reduction and/or absolute reduction in risk of cardiovascular events for the subject in response to the particular therapeutic intervention sequence, and
wherein the particular therapeutic intervention sequence comprises a sequence of one or more, annual or bi-annual, administrations of a DNA-based therapeutic intervention, an RNA-based therapeutic intervention, a protein-based therapeutic intervention, or a pharmacological therapeutic intervention.
183. The method of paragraph 182,
wherein the multiple measures of risk comprise absolute instantaneous hazard rates, cumulative hazard rates, and cumulative event rates of the subject having a cardiovascular event at respective ones of the multiple time intervals, wherein the cumulative hazard rates and the cumulative event rates are not adjusted for the particular therapeutic intervention sequence,
wherein determining the expected proportional reduction and/or the absolute reduction in the risk of cardiovascular events for the subject in response to the particular therapeutic intervention sequence comprises:
(a) for each particular interval of follow-up for the subject from the subject's current age to an upper threshold age,
(i) determining, using the multiple measures of risk, a predicted instantaneous hazard rate of the subject having a cardiovascular event at the particular interval of follow up;
(ii) determining, using the at least one second ML model, a time-averaged instantaneous log hazard ratio for a one unit lower LDL or SBP corresponding to duration of treatment at the particular interval of follow-up for the subject;
(iii) determining an intervention-adjusted instantaneous hazard for the particular interval of follow-up by multiplying the instantaneous hazard rate of the subject determined at (a)(i) with the time-averaged instantaneous log hazard ratio determined at (a)(ii),
thereby obtaining multiple intervention-adjusted instantaneous hazards for intervals of follow-up evaluated at (a);
(b) determining, using the multiple intervention-adjusted instantaneous hazards, intervention-adjusted cumulative hazard rates and cumulative event rates of cardiovascular events for intervals of follow-up for the subject from the subject's current age to the upper threshold age; and
(c) determining predicted proportional reductions in the risk of experiencing a cardiovascular event as ratios of the intervention-adjusted cumulated hazard rates and the cumulative hazard rates that are not adjusted for the particular therapeutic intervention sequence.
184. The method of paragraph 183,
wherein the at least one second trained ML model comprises a causal DNN for Ordinary Differential Equations (c-DNN-ODE) model, wherein the c-DNN-ODE model was trained on randomized data from:
(a) randomized trials of LDL and/or SBP lowering therapies, and
(b) Mendelian randomization studies evaluating genetic variants associated with lower LDL and/or SBP designed as naturally randomized trials.
185. The method of paragraph 168, further comprising:
administering and/or commencing administering to the subject the at least one identified therapeutic intervention,
wherein the at least one identified therapeutic intervention is a DNA-based therapeutic intervention, an RNA-based therapeutic intervention, a protein-based therapeutic intervention, or a pharmacological therapeutic intervention.
186. The method of paragraph 185, wherein the at least one identified therapeutic intervention is LDL-lowering siRNA, AGT-lowering siRNA, or a combination therapy of LDL-lowering siRNA and AGT-lowering siRNA.
187. A system, comprising:
at least one computer hardware processor; and
at least one non-transitory computer-readable storage medium storing processor executable instructions that, when executed by the at least one computer hardware processor, cause the at least one computer hardware processor to perform a method for identifying an intervention for a subject in furtherance of preventing development of cardiovascular disease in the subject, the method comprising:
obtaining cardiometabolic health data for the subject;
determining, using at least some of the cardiometabolic health data, a first trained machine learning (ML) model and for each of multiple time intervals, one or more measures of risk that the subject develops cardiovascular disease to obtain multiple measures of risk corresponding to the multiple time intervals;
determining, using the multiple measures of risk that the subject develops cardiovascular disease and at least one second ML model, benefit of administering to the subject one or more therapeutic interventions designed to reduce risk of cardiovascular disease by targeting one or more modifiable causes of the cardiovascular disease; and
identifying, using the determined benefit of administering the one or more therapeutic interventions, at least one therapeutic intervention to recommend being administered to the subject.
188. At least one non-transitory computer-readable storage medium storing processor executable instructions that, when executed by at least one computer hardware processor, cause the at least one computer hardware processor to perform a method for identifying an intervention for a subject in furtherance of preventing development of cardiovascular disease in the subject, the method comprising:
obtaining cardiometabolic health data for the subject;
determining, using at least some of the cardiometabolic health data, a first trained machine learning (ML) model and for each of multiple time intervals, one or more measures of risk that the subject develops cardiovascular disease to obtain multiple measures of risk corresponding to the multiple time intervals;
determining, using the multiple measures of risk that the subject develops cardiovascular
disease and at least one second ML model, benefit of administering to the subject one or more therapeutic interventions designed to reduce risk of cardiovascular disease by targeting one or more modifiable causes of the cardiovascular disease; and
identifying, using the determined benefit of administering the one or more therapeutic interventions, at least one therapeutic intervention to recommend being administered to the subject.
189. A method for estimating a cumulative LDL exposure trajectory for a subject, the method comprising:
using at least one computer hardware processor to perform:
obtaining cardiometabolic health data for a subject comprising: one or more values for one or more clinical characteristics of the subject, and/or one or more values for one or more physical measurements of the subject, and/or one or more values for one or more biochemical measurements of the subject;
encoding the cardiometabolic health data for the subject into a feature vector;
estimating an LDL level trajectory for the subject by processing the feature vector using an LDL trajectory prediction machine learning (ML) model that has been trained to estimate an LDL level for a subject at each of multiple prior ages and each of multiple future ages using training data comprising for each of a plurality of participants, repeated longitudinal measures of LDL levels and values for the one or more clinical characteristics and/or the one or more physical measurements and/or the one or more biochemical measurements over at least 10, at least 20, at least 30, or at least 50 years of follow-up, wherein the LDL level trajectory for the subject comprises estimated LDL levels for the subject including an estimated LDL level for each of multiple prior ages of the subject and multiple future ages of the subject;
estimating, using the LDL level trajectory, a cumulative LDL exposure trajectory for the subject with respect to a set of ages, wherein the cumulative LDL exposure trajectory comprises an estimated cumulative LDL exposure level for the subject at each age in the set of ages; and
outputting the estimate cumulative LDL exposure trajectory for the subject.
190. The method of paragraph 189, wherein the one or more values for one or more clinical characteristics comprise:
(i) values for one or more demographic characteristics of the subject, one or more genetic characteristics of the subject, one or more family history characteristics of the subjects, one or more comorbidities, and/or risk factors;
(ii) the one or more values for one or more physical measurements of the subject comprise one or more values for physical measurements of quantities that are risk factors for cardiometabolic disease and/or physiologic al measurements selected from blood pressure measurements and measurements indicative of adiposity; and/or
(iii) the one or more values for one or more biochemical measurements of the subject comprise one or more values for biochemical measurements of quantities that are risk factors for cardiovascular disease and/or biochemical measurements selected from measurements of one or more biochemical markers.
191. The method of paragraph 189, wherein the cardiometabolic health data includes a value indicating the subject's age, a value indicating the subject's biological sex, a value indicating the subject's LDL level, a value indicating the subject's SBP, and a value indicating the subject's DBP.
192. The method of paragraph 189, wherein encoding the cardiometabolic health data for the subject into a feature vector comprises:
standardizing at least some of values in the cardiometabolic health data to obtain an initial feature vector; and
positionally encoding at least some feature values in the initial feature vector by the subject's age or ages at which the at least some of feature values were measured to obtain the feature vector.
193. The method of paragraph 192, wherein performing the standardizing comprises:
performing min-max standardizing the at least some of the values, for continuous variables, in the subject characteristic and/or measurement data, and/or
numerically encoding any dichotomous or ordinal values in the subject characteristic and/or measurement data.
194. The method of paragraph 192, wherein the positionally encoding comprises:
generating a positional encoding of the initial feature vector using sinusoidal encoding of at least some elements of the initial feature vector, wherein the sinusoidal encoding using age in years as position; and
generating the feature vector by appending the positional encoding of the initial feature vector to the initial feature vector.
195. The method of paragraph 189, wherein the LDL trajectory prediction ML model is a recurrent neural network model or a neural network model having a transformer architecture.
196. The method of paragraph 195, wherein the LDL trajectory prediction ML model is a recurrent neural network model, wherein the recurrent neural network model is a bi-directional LSTM (bi-LSTM) model, a bi-directional GRU model, or an ODE-RNN model.
197. The method of paragraph 196, wherein:
the LDL trajectory prediction ML model is the bi-LSTM model, and
processing the feature vector using the LDL trajectory prediction ML model comprises:
estimating an LDL level for the subject at each of multiple future ages using a forward pass of the bi-LSTM model; and
estimating an LDL level for the subject at each of multiple prior ages using a backward pass of the bi-LSTM model.
198. The method of paragraph 197, wherein the bi-LSTM model has at least 5,000 parameters.
199. The method of paragraph 189, further comprising:
encoding the cardiometabolic health data for the subject into a second feature vector;
estimating an SBP level trajectory for the subject by processing the second feature vector using an SBP trajectory prediction ML model that has been trained to estimate an SBP level for a subject at each of multiple prior ages and each of multiple future ages using training data comprising, for each of a plurality of participants, repeated longitudinal measures of SBP levels and values for the one or more clinical characteristics and/or the one or more physical measurements and/or the one or more biochemical measurements over at least 10, at least 20, at least 30, or at least 50 years of follow-up,
wherein the SBP level trajectory for the subject comprises an estimated SBP level for the subject for each of multiple prior ages of the subject and multiple future ages of the subject.
200. The method of paragraph 199, further comprising:
encoding the cardiometabolic health data for the subject into a third feature vector;
estimating an Lp(a) level trajectory for the subject by processing the third feature vector using an Lp(a) trajectory prediction ML model that has been trained to estimate Lp(a) levels for the subject at each of the multiple prior ages and each of the multiple future ages using training data comprising, for each of a plurality of participants, repeated longitudinal measures of Lp(a) levels and values for the one or more clinical characteristics and/or the one or more physical measurements and/or the one or more biochemical measurements over multiple years of follow-up,
wherein the Lp(a) level trajectory for the subject comprises an estimated L(p) level for the subject for each of multiple prior ages of the subject and multiple future ages of the subject.
201. The method of paragraph 189, further comprising:
determining, using at least some of the cardiometabolic health data including the cumulative LDL exposure trajectory, a first trained machine learning (ML) model and for
each of multiple time intervals, one or more measures of risk that the subject develops cardiovascular disease to obtain multiple measures of risk corresponding to the multiple time intervals;
determining, using the multiple measures of risk that the subject develops cardiovascular
disease and at least one second ML model, benefit of administering to the subject one or more therapeutic interventions designed to reduce risk of cardiovascular disease by targeting one or more modifiable causes of the cardiovascular disease; and
identifying, using the determined benefit of administering the one or more therapeutic
interventions, at least one therapeutic intervention to recommend being administered to the subject.
202. The method of paragraph 201, wherein determining the one or more measures of risk that the subject develops cardiovascular disease, for each of the multiple time intervals, to obtain the multiple measures of risk corresponding to the multiple time intervals, comprises:
(a) estimating, using the first trained ML model and the at least some of the cardiometabolic health data including the cumulative LDL exposure trajectory for the subject, values indicative of log hazard ratios for risk of the subject having a cardiovascular event at respective levels of cumulative LDL exposure; and
(b) estimating, using the values indicative of the log hazard ratios,
(i) absolute instantaneous hazard rates of the subject having a cardiovascular event at the respective levels of cumulative LDL exposure, and
(ii) cumulative lifetime risks of the subject having a cardiovascular event at the respective levels of cumulative LDL exposure;
(c) estimating, using the cumulative LDL exposure trajectory for the subject, the multiple measures of risk to include:
(i) absolute instantaneous hazard rates of the subject having a cardiovascular event at respective ones of the multiple time intervals, and
(ii) cumulative lifetime hazard and event rates of the subject having a cardiovascular event at the respective ones of the multiple time intervals.
203. The method of paragraph 202, wherein the first trained ML model is a trained survival model using cumulative LDL exposure as an interval of follow-up.
204. The method of paragraph 203, wherein the first trained ML model is a trained survival DNN model with piecewise exponential models (PEM) using cumulative LDL exposure as the interval of follow-up, wherein the trained survival DNN model comprises a different set of trained weights for each interval of follow-up, wherein each interval of follow-up is an interval of cumulative LDL exposure.
205. The method of paragraph 189, further comprising:
administering and/or commencing administering to the subject the at least one identified therapeutic intervention,
wherein the at least one identified therapeutic intervention is a DNA-based therapeutic intervention, an RNA-based therapeutic intervention, a protein-based therapeutic intervention, or a pharmacological therapeutic intervention.
206. The method of paragraph 205, wherein the at least one identified therapeutic intervention is LDL-lowering siRNA, AGT-lowering siRNA, or a combination therapy of LDL-lowering siRNA and AGT-lowering siRNA.
207. A system, comprising:
at least one computer hardware processor; and
at least one non-transitory computer-readable storage medium storing processor executable instructions that, when executed by the at least one computer hardware processor, cause the at least one computer hardware processor to perform a method for estimating a cumulative LDL exposure trajectory for a subject, the method comprising:
obtaining cardiometabolic health data for a subject comprising: one or more values for one or more clinical characteristics of the subject, and/or one or more values for one or more physical measurements of the subject, and/or one or more values for one or more biochemical measurements of the subject;
encoding the cardiometabolic health data for the subject into a feature vector;
estimating an LDL level trajectory for the subject by processing the feature vector using an LDL trajectory prediction machine learning (ML) model that has been trained to estimate am LDL level for a subject at each of multiple prior ages and each of multiple future ages using training data comprising for each of a plurality of participants, repeated longitudinal measures of LDL levels and values for the one or more clinical characteristics and/or the one or more physical measurements and/or the one or more biochemical measurements over at least 10, at least 20, at least 30, or at least 50 years of follow-up, wherein the LDL level trajectory for the subject comprises estimated LDL levels for the subject including an estimated LDL level for each of multiple prior ages of the subject and multiple future ages of the subject;
estimating, using the LDL level trajectory, a cumulative LDL exposure trajectory for the subject with respect to a set of ages, wherein the cumulative LDL exposure trajectory comprises an estimated cumulative LDL exposure level for the subject at each age in the set of ages; and outputting the estimate cumulative LDL exposure trajectory for the subject.
208. At least one non-transitory computer-readable storage medium storing processor executable instructions that, when executed by at least one computer hardware processor, cause the at least one computer hardware processor to perform a method for estimating a cumulative LDL exposure trajectory for a subject, the method comprising:
obtaining cardiometabolic health data for a subject comprising: one or more values for
one or more clinical characteristics of the subject, and/or one or more values for one or more physical measurements of the subject, and/or one or more values for one or more biochemical measurements of the subject;
encoding the cardiometabolic health data for the subject into a feature vector;
estimating an LDL level trajectory for the subject by processing the feature vector using
an LDL trajectory prediction machine learning (ML) model that has been trained to estimate am LDL level for a subject at each of multiple prior ages and each of multiple future ages using training data comprising for each of a plurality of participants, repeated longitudinal measures of LDL levels and values for the one or more clinical characteristics and/or the one or more physical measurements and/or the one or more biochemical measurements over at least 10, at least 20, at least 30, or at least 50 years of follow-up,
wherein the LDL level trajectory for the subject comprises estimated LDL levels for the subject including an estimated LDL level for each of multiple prior ages of the subject and multiple future ages of the subject;
estimating, using the LDL level trajectory, a cumulative LDL exposure trajectory for the subject with respect to a set of ages, wherein the cumulative LDL exposure trajectory comprises an estimated cumulative LDL exposure level for the subject at each age in the set of ages; and outputting the estimate cumulative LDL exposure trajectory for the subject.
209. A computer-implemented method, comprising:
obtaining cardiometabolic health data for a subject comprising: one or more values for one or more clinical characteristics of the subject, and/or one or more values for one or more physical measurements of the subject, and/or one or more values for one or more biochemical measurements of the subject;
encoding the cardiometabolic health data for the subject into a first feature vector;
estimating an LDL level trajectory for the subject by processing the first feature vector using an LDL trajectory prediction machine learning (ML) model that has been trained to estimate an LDL level for a subject at each of multiple prior ages and each of multiple future ages using training data comprising for each of a plurality of participants, repeated longitudinal measures of LDL levels and values for the one or more clinical characteristics and/or the one or more physical measurements and/or the one or more biochemical measurements over at least 10, at least 20, at least 30, or at least 50 years of follow-up,
wherein the LDL level trajectory for the subject comprises an estimated LDL level for the subject for each of multiple prior ages of the subject and multiple future ages of the subject.
210. The method of paragraph 209, further comprising:
encoding the cardiometabolic health data for the subject into a second feature vector;
estimating an SBP level trajectory for the subject by processing the second feature vector using an SBP trajectory prediction ML model that has been trained to estimate an SBP level for a subject at each of multiple prior ages and each of multiple future ages using training data comprising, for each of a plurality of participants, repeated longitudinal measures of SBP levels and values for the one or more clinical characteristics and/or the one or more physical measurements and/or the one or more biochemical measurements over at least 10, at least 20, at least 30, or at least 50 years of follow-up,
wherein the SBP level trajectory for the subject comprises an estimated SBP level for the subject for each of multiple prior ages of the subject and multiple future ages of the subject.
211. The method of paragraph 209 or 210, further comprising:
encoding the cardiometabolic health data for the subject into a third feature vector;
estimating an Lp(a) level trajectory for the subject by processing the third feature vector using an Lp(a) trajectory prediction ML model that has been trained to estimate an Lp(a) level for the subject at each of the multiple prior ages and each of the multiple future ages using training data comprising, for each of a plurality of participants, repeated longitudinal measures of Lp(a) levels and values for the one or more clinical characteristics and/or the one or more physical measurements and/or the one or more biochemical measurements over multiple years of follow-up.
212. The method of any one of paragraphs 209-211, wherein:
(i) the one or more values for one or more clinical characteristics comprise values for one or more demographic characteristics of the subject, one or more genetic characteristics of the subject, one or more family history characteristics of the subjects, one or more comorbidities, and/or risk factors; and/or
(ii) the one or more values for one or more physical measurements of the subject comprise one or more values for physical measurements of quantities that are risk factors for cardiometabolic disease and/or physiologic al measurements selected from blood pressure measurements and measurements indicative of adiposity; and/or
(iii) the one or more values for one or more biochemical measurements of the subject comprise one or more values for biochemical measurements of quantities that are risk factors for cardiovascular disease and/or biochemical measurements selected from measurements of one or more biochemical markers, optionally a protein, lipid or lipoprotein, in a blood, serum, and/or plasma sample from the subject.
213. The method of any one of paragraphs 209-212, wherein:
(i) the one or more clinical characteristics of the subject are selected from the group consisting of: age, biological sex, family history of coronary heart disease (CHD), family history of hypertension (HTN), family history of type 2 diabetes (T2D), polygenic score for ASCVD, polygenic score for CHD, polygenic score for HTN, polygenic score for T2D, polygenic score for body mass index (BMI), inherited predisposition or predispositions, and history of tobacco use; and/or
(ii) the one or more physical measurements of the subject are selected from the group consisting of systolic blood pressure (SBP), diastolic blood pressure (DBP), weight, waist circumference, height, body mass index (BMI), and waist-to-height ratio; and/or
(iii) the one or more biochemical measurements of the subject are selected from the group consisting of low-density lipoprotein (LDL) level, high-density lipoprotein (HDL) level, total cholesterol level, triglyceride (TG) level, non-HDL cholesterol level, apolipoprotein (apoB) level, lipoprotein (a) (Lp(a)) level, hemoglobin A1c (HbA1c) level, and c-reactive protein (CRP) level.
214. The method of any one of paragraphs 209-213, wherein the cardiometabolic health data includes a value indicating the subject's age, a value indicating the subject's biological sex, a value indicating the subject's LDL level, a value indicating the subject's SBP, a value indicating the subject's DBP, and, optionally, a value indicating the subject's Lp(a) level.
215. The method of any one of paragraphs 209-211, wherein generating the first, second, and/or third feature vector representing the subject comprises:
obtaining an initial feature vector using at least some of the cardiometabolic health data for the subject, optionally by standardizing at least some of values in the subject characteristic and/or measurement data to obtain an initial feature vector; and
positionally encoding the initial feature vector by age of the subject associated with the values in the cardiometabolic health data for the subject to obtain the a feature vector representing the subject.
216. The method of paragraph 215, wherein obtaining the initial feature vector using at least some of the cardiometabolic health data for the subject comprises standardizing at least some of values in the cardiometabolic health data to obtain an initial feature vector, wherein performing the standardizing comprises:
performing min-max standardizing the at least some of the values, for continuous variables, in the subject characteristic and/or measurement data, and/or
numerically encoding any dichotomous or ordinal values in the subject characteristic and/or measurement data.
217. The method of any of paragraphs 215-216, wherein the positionally encoding comprises:
generating a positional encoding of the initial feature vector using sinusoidal encoding of at least some elements of the initial feature vector, wherein the sinusoidal encoding using age in years as position; and
generating the feature vector by appending the positional encoding of the initial feature vector to the initial feature vector.
218. The method of any one of paragraphs 209-217, wherein the LDL trajectory prediction ML model has been trained using training data comprising for each of the plurality of participants, repeated longitudinal measures of LDL levels and feature vectors derived from values for the one or more clinical characteristics and/or the one or more physical measurements and/or the one or more biochemical measurements using at least positional encoding by age of the participant associated with the respective values, optionally, wherein the positional encoding is sinusoidal encoding.
219. The method of any one of paragraphs 209-218, wherein the LDL trajectory prediction machine learning model was trained using participant data for at least 10,000 participants enrolled in one or more prospective studies, comprising for each of the at least 10,000 participants repeated longitudinal measures of LDL, HDL, SBP, weight, waist circumference, BMI, and/or HbA1c over at least 10, at least 20, at least 30, or at least 50 years of follow-up.
220. The method of any one of paragraphs 209-219, wherein the LDL trajectory prediction ML model is a neural network model, optionally a recurrent neural network model or a neural network model having a transformer architecture, optionally a temporal fusion transformer (TFT) architecture.
221. The method of any one of paragraphs 209-220, wherein the LDL trajectory prediction ML model is a bi-directional LSTM (bi-LSTM) model, a bi-directional GRU model, or an ODE-RNN model.
222. The method of paragraph 221, wherein:
the LDL trajectory prediction ML model is the bi-LSTM model, and
processing the first feature vector using the LDL trajectory prediction ML model comprises:
estimating an LDL level for the subject at each of the multiple future ages using a forward pass of the bi-LSTM model; and
estimating an LDL level for the subject at each of the multiple prior ages using a backward pass of the bi-LSTM model.
223. The method of any one of paragraphs 209-222, further comprising:
estimating, using the LDL level trajectory, a cumulative LDL exposure trajectory for the subject with respect to a set of ages, wherein the cumulative LDL exposure trajectory comprises an estimated cumulative LDL exposure level for the subject at each age in the set of ages.
224. The method of any one of paragraphs 210-223, wherein the SBP trajectory prediction ML model has been trained using training data comprising for each of the plurality of participants, repeated longitudinal measures of SBP levels and feature vectors derived from values for the one or more clinical characteristics and/or the one or more physical measurements and/or the one or more biochemical measurements using at least positional encoding by age of the participant associated with the respective values, optionally, wherein the positional encoding is sinusoidal encoding.
225. The method of any one of paragraphs 210-224, wherein the SBP trajectory prediction machine learning model was trained using participant data for at least 10,000 participants enrolled in one or more prospective studies, comprising for each of the at least 10,000 participants repeated longitudinal measures of LDL, HDL, SBP, weight, waist circumference, BMI, and/or HbA1c over at least 10, at least 20, at least 30, or at least 50 years of follow-up.
226. The method of any one of paragraphs 210-225, wherein the SBP trajectory prediction ML model is a neural network model, optionally a recurrent neural network model or a neural network model having a transformer architecture, optionally a temporal fusion transformer (TFT) architecture.
227. The method of any one of paragraphs 210-226, wherein the SBP trajectory prediction ML model is a bi-directional LSTM (bi-LSTM) model, a bi-directional GRU model, or an ODE-RNN model.
228. The method of paragraph 227, wherein:
the SBP trajectory prediction ML model is the bi-LSTM model, and
processing the second feature vector using the SBP trajectory prediction ML model comprises:
estimating an SBP level for the subject at each of the multiple future ages using a forward pass of the bi-LSTM model; and
estimating an SBP level for the subject at each of the multiple prior ages using a backward pass of the bi-LSTM model.
229. The method of any one of paragraphs 210-228, further comprising:
estimating, using the SBP level trajectory, a cumulative SBP exposure trajectory for the subject with respect to a set of ages, wherein the cumulative SBP exposure trajectory comprises an estimated cumulative SBP exposure level for the subject at each age in the set of ages; and
optionally, estimating rates of rise in SBP for the subject at each age in the set of ages
230. The method of any one of paragraphs 211-229, wherein the Lp(a) trajectory prediction ML model is a neural network model, optionally a recurrent neural network model or a neural network model having a transformer architecture, optionally a temporal fusion transformer (TFT) architecture.
231. The method of any one of paragraphs 211-230, wherein the Lp(a) trajectory prediction ML model is a bi-directional LSTM (bi-LSTM) model, a bi-directional GRU model, or an ODE-RNN model.
232. The method of paragraph 231, wherein:
the Lp(a) trajectory prediction ML model is the bi-LSTM model, and
processing the third feature vector using the Lp(a) trajectory prediction ML model comprises:
estimating an Lp(a) level for the subject at each of the multiple future ages using a forward pass of the bi-LSTM model; and
estimating an Lp(a) level for the subject at each of the multiple prior ages using a backward pass of the bi-LSTM model.
233. The method of any one of paragraphs 211-232, further comprising:
estimating, using the Lp(a) level trajectory, a cumulative Lp(a) exposure trajectory for the subject with respect to a set of ages, wherein the cumulative Lp(a) exposure trajectory comprises an estimated cumulative SBP exposure level for the subject at each age in the set of ages.
234. The method of any one of the paragraphs 209-233, further comprising:
estimating a weight trajectory for the subject using the cardiometabolic health data, wherein the weight trajectory for the subject comprises estimated weight of the subject for each of multiple ages of the subject, optionally wherein estimating the weight trajectory is performed assuming that the subject's current age-and-sex adjusted weight percentile remains constant throughout life; and/or
estimating a waist circumference trajectory for the subject the cardiometabolic health data, wherein the waist circumference trajectory for the subject comprises estimated waist circumference of the subject for each of multiple ages of the subject, optionally wherein estimating the waist circumference trajectory is performed assuming that the subject's current age-and-sex adjusted waist circumference percentile remains constant throughout life; and/or
estimating an HbA1c level trajectory for the subject using the cardiometabolic health data, wherein the HbA1c level trajectory for the subject comprises estimated HbA1c levels of the subject for each of multiple ages of the subject, optionally wherein estimating the HbA1c level trajectory is performed assuming that the subject's current age-and-sex adjusted HbA1c percentile remains constant throughout life.
235. A computer-implemented method of determining one or more measures of risk that a subject develops cardiovascular disease, the method comprising, for each of multiple time intervals:
(a) estimating, using a trained cardiovascular risk prediction machine learning model and cardiometabolic health data for the subject including a cumulative LDL exposure trajectory for the subject, values indicative of log hazard ratios for risk of the subject having a cardiovascular event at respective levels of cumulative LDL exposure, wherein the cardiovascular risk prediction machine learning model has been trained using training data comprising, for each of a plurality of participants enrolled in one or more prospective studies, multiple LDL measurements along with a recorded age or date at which a first cardiovascular event occurred, optionally wherein the first cardiovascular event is a first episode of a fatal or non-fatal myocardial infarction (MI), fatal or non-fatal ischemic stroke, or coronary revascularization; and
(b) estimating, using the values indicative of the log hazard ratios,
(i) absolute instantaneous hazard rates of the subject having a cardiovascular event at the respective levels of cumulative LDL exposure, and
(ii) cumulative lifetime risks of the subject having a cardiovascular event at the respective levels of cumulative LDL exposure; 236. The method of paragraph 235, further comprising:
(c) estimating, using the cumulative LDL exposure trajectory for the subject, the multiple measures of risk to include:
(i) absolute instantaneous hazard rates of the subject having a cardiovascular event at respective ones of the multiple time intervals, and
(ii) cumulative lifetime hazard and event rates of the subject having a cardiovascular event at the respective ones of the multiple time intervals.
237. The method of any one of paragraphs 235-236,
wherein the cumulative LDL exposure trajectory has been obtained using the method of any of paragraphs 209-234, and/or wherein the method comprises obtaining the cumulative LDL exposure trajectory using the method of any of paragraphs 209-234; and/or
wherein step (a) uses cardiometabolic health data further comprising a cumulative SBP exposure trajectory that has been obtained using the method of any of paragraphs 210-234, and/or wherein step (a) uses cardiometabolic health data further comprising a cumulative SBP exposure trajectory and the method comprises obtaining the cumulative SBP exposure trajectory using the method of any of paragraphs 210-234; and/or wherein step (a) uses cardiometabolic health data further comprising a cumulative Lp(a) exposure trajectory that has been obtained using the method of any of paragraphs 211-234, and/or wherein step (a) uses cardiometabolic health data further comprising a cumulative Lp(a) exposure trajectory and the method comprises obtaining the cumulative SBP exposure trajectory using the method of any of paragraphs 211-234.
238. The method of any one of paragraphs 235-237, wherein the multiple time intervals correspond to multiple ages of the subject such that the multiple measures of risk comprise:
absolute instantaneous hazard rates of the subject having a cardiovascular event at each of the multiple ages of the subject; and
cumulative lifetime risks of the subject having a cardiovascular event at each of the multiple ages of the subject.
239. The method of any one of paragraphs 235-238, wherein estimating, using the trained cardiovascular risk prediction ML model and the at least some of the cardiometabolic health data, the values indicative of the log hazard ratios for risk of the subject having a cardiovascular event at the respective levels of cumulative LDL exposure comprises:
encoding the at least some of the cardiometabolic health data to obtain input feature data; and
estimating, by processing the input feature data using the cardiovascular risk prediction ML model, the values indicative of the log hazard ratios for the risk of the subject having a cardiovascular event at the respective levels of cumulative LDL exposure,
optionally, wherein processing the input feature data comprises using a trained survival deep neural network (DNN) with piecewise exponential modeling (PEM) model to calculate log hazard ratios for the risk of the subject having a cardiovascular event at the respective levels of cumulative LDL exposure.
240. The method of paragraph 239, wherein the input feature data comprises:
a set of input feature vectors, each particular one of the input feature vectors corresponding to a particular cumulative LDL exposure level from among the respective levels of cumulative LDL exposure,
wherein the particular input feature vector in the set of input feature vectors that corresponds to the particular cumulative LDL exposure level, comprises:
(a) subject characteristic and/or measurement data comprising: one or more values for one or more clinical characteristics of the subject, one or more values for one or more physical measurements of the subject, and/or one or more values for one or biochemical measurements of the subject;
(b) an LDL level trajectory for the subject;
(c) a cumulative LDL exposure trajectory for the subject;
(d) an SBP level trajectory for the subject indicating an estimate of the subject's SBP level at each of the respective levels of cumulative LDL exposure;
(e) a cumulative SBP exposure trajectory for the subject indicating an estimate of the subject's cumulative SBP exposure at each of the respective levels of cumulative LDL exposure;
(f) an Lp(a) level trajectory for the subject indicating an estimate of the subject's Lp(a) level at each level of the respective levels of cumulative LDL exposure;
(g) a cumulative Lp(a) exposure trajectory for the subject indicating an estimate of the subject's cumulative Lp(a) exposure at each level of the respective levels of cumulative LDL exposure;
(h) a weight trajectory for the subject indicating an estimate of the subject's weight at each of the respective levels of cumulative LDL exposure;
(i) a waist circumference trajectory for the subject indicating an estimate of the subject's waist circumference at each of the respective levels of cumulative LDL exposure;
(j) an HbA1c level trajectory for the subject indicating an estimate of the subject's HbA1c level at each of the respective levels of cumulative LDL exposure;
(k) an age trajectory for the subject indicating an estimated age for the subject at each of the respective levels of cumulative LDL exposure; and/or
(l) a positionally encoded version of one or more of (a), (b), (d), (e), (f), (g), (h), (i), (j), and (k) obtained by positionally encoding the one or more of (a), (b), (d), (e), (f), (g), (h), (i), (j), and (k) by age of the subject at which the particular cumulative LDL exposure level is predicted to occur, optionally, wherein values in the set of input feature vectors are organized in at least one data structure representing a matrix.
241. The method of paragraph 240, wherein generating the input feature data comprises:
generating a first input vector using the subject characteristic and/or measurement data and positional encodings associated with the subject characteristic and/or measurement data;
processing the first input vector using a first recurrent neural network (RNN) to obtain the LDL level trajectory for the subject, where the first RNN has been trained to estimate an LDL level for a subject at each of multiple prior ages and each of multiple future ages using training data comprising for each of a plurality of participants, repeated longitudinal measures of LDL levels and values for the one or more clinical characteristics and/or the one or more physical measurements and/or the one or more biochemical measurements over at least 10, at least 20, at least 30, or at least 50 years of follow-up; and
processing the first input vector using a second RNN to obtain the SBP level trajectory for the subject, where the second RNN has been trained to estimate an SBP level for a subject at each of multiple prior ages and each of multiple future ages using training data comprising, for each of a plurality of participants, repeated longitudinal measures of SBP levels and values for the one or more clinical characteristics and/or the one or more physical measurements and/or the one or more biochemical measurements over at least 10, at least 20, at least 30, or at least 50 years of follow-up.
242. The method of any one of paragraphs 235-241, wherein the trained cardiovascular risk prediction machine learning model is a trained survival model using cumulative LDL exposure as an interval of follow-up,
optionally wherein the trained survival model using cumulative LDL exposure as an interval of follow-up comprises one of: (i) a Cox proportional hazard model; (ii) a random forest of survival trees model; (iii) a gradient boosted machine (GBM) for survival trees model; (iv)
a survival deep neural network (DNN) model; and/or (v) an ensemble of any of these survival models.
243. The method of paragraph 242, wherein the trained cardiovascular risk prediction machine learning model is a trained survival deep neural network (DNN) model using cumulative LDL exposure as the interval of follow-up.
244. The method of paragraph 242 or 243, wherein the trained cardiovascular risk prediction machine learning model is a trained survival model with piecewise exponential models (PEM) using cumulative LDL exposure as the interval of follow-up.
245. The method of paragraph 244, wherein the trained cardiovascular risk prediction machine learning model is a trained survival DNN model with piecewise exponential models (PEM) using cumulative LDL exposure as the interval of follow-up
optionally, wherein the hazard rate in each interval of follow-up is assumed to be constant and the trained survival DNN with PEM comprises a different set of trained weights for each interval of follow-up, wherein each interval of follow-up is an interval of cumulative LDL exposure.
246. The method of any one of paragraphs 235-245, wherein the trained cardiovascular risk prediction machine learning model was trained using participant data for at least 1 million (M) or 1.5 M participants enrolled in one or more prospective studies, whereby for each of the at least 1 M or 1.5 M participants at least one LDL or SBP measurement were available along with a recorded age or date at which a first episode of a fatal or non-fatal myocardial infarction (MI), fatal or non-fatal ischemic stroke, or coronary revascularization occurred.
247. The method of any one of paragraphs 235-245, wherein estimating, using the values indicative of the log hazard ratios, the cumulative lifetime risks of the subject having a cardiovascular event at the respective levels of cumulative LDL exposure comprises:
(a) generating a data structure encoding values indicating absolute instantaneous hazard rates of experiencing a cardiovascular event at the respective levels of cumulative LDL exposure in a reference population;
(b) determining the absolute instantaneous hazard rates of the subject having a cardiovascular event at the respective levels of cumulative LDL exposure using:
(i) the values, from (a), indicating absolute instantaneous hazard rates of experiencing a cardiovascular event at the respective levels of cumulative LDL exposure in the reference population; and
(ii) the values indicative of the log hazard ratios for risk of the subject having a cardiovascular event at the respective levels of cumulative LDL exposure; and
(c) determining the cumulative hazard rates of the subject having a cardiovascular event at the respective levels of cumulative LDL using the absolute instantaneous hazard rates, determined at (b); and
(d) determining the cumulative event rates of the subject having a cardiovascular event at the respective levels of cumulative LDL using the absolute instantaneous hazard rates, determined at (b).
248. The method of paragraph 247, further comprising:
identifying a personal plaque burden threshold for the subject by using the cumulative event rates of the subject having a cardiovascular event at the respective levels of cumulative LDL, wherein the personal plaque burden threshold indicates a level of cumulative plaque burden at which cardiovascular events are predicted to begin to occur for the subject; and/or
249. The method of paragraph 248, further comprising: identifying, for a specified level of cumulative lifetime risk, a level of cumulative plaque burden at which risk of occurrence cardiovascular events for the subject is less than the specified level of cumulative lifetime risk.
250. A computer-implemented method of determining an expected proportional reduction and/or absolute reduction in risk of cardiovascular events for a subject in response to a particular therapeutic intervention sequence, the particular therapeutic sequence indicating magnitude, duration, and timing of one or more interventions associated with a reduction of LDL level and/or SBP level over an interval of follow up for the subject, the method comprising:
determining multiple measures of risk of cardiovascular events for the subject comprising absolute instantaneous hazard rates, cumulative hazard rates, and cumulative event rates of the subject having a cardiovascular event at respective ones of multiple time intervals, wherein the cumulative hazard rates and the cumulative event rates are not adjusted for the particular therapeutic intervention sequence, and
determining the expected proportional reduction and/or the absolute reduction in the risk of cardiovascular events for the subject in response to the particular therapeutic intervention sequence using a method that comprises:
(a) for each particular interval of follow-up for the subject from the subject's current age to an upper threshold age,
(i) determining, using the multiple measures of risk, a predicted instantaneous hazard rate of the subject having a cardiovascular event at the particular interval of follow up;
(ii) determining, using a benefit prediction machine learning (ML) model, a time-averaged instantaneous log hazard ratio for a one unit lower LDL or SBP corresponding to duration of treatment at the particular interval of follow-up for the subject, wherein the benefit prediction machine learning model has been trained using training data from randomized trials of LDL lowering therapies and/or randomized trials of SBP therapies and Mendelian randomization studies evaluating genetic variants associated with lower LDL and/or lower SBP, said training data comprising for each of a plurality of participants in said trials, at least one LDL or SBP measurement along with a recorded age or date at which a first cardiovascular event occurred;
(iii) determining an intervention-adjusted instantaneous hazard for the particular interval of follow-up by multiplying the instantaneous hazard rate of the subject determined at (a)(i) with the time-averaged instantaneous log hazard ratio determined at (a)(ii),
thereby obtaining multiple intervention-adjusted instantaneous hazards for intervals of follow-up evaluated at (a);
(b) determining, using the multiple intervention-adjusted instantaneous hazards,
intervention-adjusted cumulative hazard rates and cumulative event rates of cardiovascular events for intervals of follow-up for the subject from the subject's current age to the upper threshold age; and
(c) determining predicted proportional reductions in the risk of experiencing a cardiovascular event as ratios of the intervention-adjusted cumulated hazard rates and the cumulative hazard rates that are not adjusted for the particular therapeutic intervention sequence.
251. The method of paragraph 250, further comprising:
(d) determining predicted absolute reductions in the risk of experience of experiencing a cardiovascular event as absolute differences between the intervention-adjusted cumulative event rates and the cumulative event rates that are not adjusted for the particular therapeutic intervention sequence.
252. The method of paragraph 250 or 251, wherein the multiple measures of risk not adjusted for the particular therapeutic intervention have been obtained using the method of any of paragraphs 235-249; and/or wherein the method comprises determining the multiple measures of risk of cardiovascular events for the subject not adjusted for the particular therapeutic intervention using the method of any of paragraphs 235-249.
253. The method of paragraph 250, wherein determining the intervention-adjusted instantaneous hazard for the particular interval of follow-up further comprises additionally adjusting the intervention-adjusted instantaneous hazard for an expected absolute magnitude of the reduction in LDL or SBP in response to the particular therapeutic intervention sequence using a Wald ratio of effect estimates method.
254. The method of any one of paragraphs 250-253, wherein the benefit prediction machine learning model a non-linear regression model, an adaptive basis function regression model, a neural network regression model, a deep neural network regression model, a DNN with ordinary differential equations regression model, a logistic regression model, a polynomial regression model, a decision tree regression model, a random forest regression model, and/or a gradient boosted decision tree regression model.
255. The method of any one of paragraphs 250-254, wherein the benefit prediction ML model comprises a deep neural network (DNN) model.
256. The method of paragraph 255, wherein the DNN model is a causal DNN for Ordinary Differential Equations (c-DNN-ODE) model, optionally, wherein the c-DNN-ODE model has at least 3, 4, or 5 fully-connected layers.
257. The method of paragraph 256, wherein, in response to input indicating a duration of sustained intervention, optionally specified in intervals of months or years, the c-DNN-ODE model is trained to provide, output indicating magnitude of an instantaneous log hazard ratio of benefit of lowering LDL or SBP by one unit for each interval of the duration of sustained intervention.
258. The method of paragraph 257, wherein the output further indicates rate of change, with respect to the input indicating the duration of sustained intervention, of the magnitude of the instantaneous log hazard ratio of benefit of lowering LDL or SBP by one unit for each interval of the duration of sustained intervention.
259. The method of any one of paragraphs 250-258, wherein the benefit prediction ML model was trained on randomized data obtained from:
(a) participant data for at least 1 million (M) or 1.5 M participants enrolled in one or more prospective studies, whereby for each of the at least 1 M or 1.5 M participants at least one LDL or SBP measurement were available along with a recorded age or date at which a first episode of a fatal or non-fatal myocardial infarction (MI), fatal or non-fatal ischemic stroke, or coronary revascularization occurred, with censoring applied at time of last follow-up, death, or first cardiovascular event; and
(b) participant data from at least 250K or 500K participants enrolled in at least 25, 50, or 75 randomized trials evaluating LDL or BP lowering therapies that provided time-to-event curves, and measurements of absolute difference in LDL or SBP between randomized groups in the randomized trials.
260. Repeating the method of paragraphs 209 to 259 using updated cardiometabolic health data.
261. A computer-implemented method for pricing an insurance instrument for a subject, the method comprising:
obtaining cardiometabolic health data for the subject including a cumulative LDL exposure trajectory for the subject;
determining multiple measures of risk that the subject develops cardiovascular disease using a method comprising:
(a) estimating, using a trained cardiovascular risk prediction machine learning model and the cardiometabolic health data for the subject, values indicative of log hazard ratios for risk of the subject having a cardiovascular event at respective levels of cumulative LDL exposure, wherein the cardiovascular risk prediction machine learning model has been trained using training data comprising, for each of a plurality of participants enrolled in one or more prospective studies, at least one LDL along with a recorded age or date at which a first cardiovascular event occurred, optionally wherein the first cardiovascular event is a first episode of a fatal or non-fatal myocardial infarction (MI), fatal or non-fatal ischemic stroke, or coronary revascularization occurred;
(b) estimating, using the values indicative of the log hazard ratios,
(i) absolute instantaneous hazard rates of the subject having a cardiovascular event at the respective levels of cumulative LDL exposure, and
(ii) cumulative lifetime risks of the subject having a cardiovascular event at the respective levels of cumulative LDL exposure; and
(c) estimating, using the cumulative LDL exposure trajectory for the subject, multiple measures of risk to include:
(i) absolute instantaneous hazard rates of the subject having a cardiovascular event at respective ones of multiple time intervals, and
(ii) cumulative lifetime hazard and event rates of the subject having a cardiovascular event at the respective ones of the multiple time intervals; and pricing the insurance instrument for the subject based on the multiple measures of risk corresponding to the multiple time intervals.
262. The method of paragraph 261, wherein determining multiple measures of risk that the subject develops cardiovascular disease is performed using the method of any of paragraphs 235-249.
263. A computer-implemented method for pricing an insurance instrument for a subject, the method comprising:
obtaining cardiometabolic health data for a subject comprising: one or more values for one or more clinical characteristics of the subject, and/or one or more values for one or more physical measurements of the subject, and/or one or more values for one or more biochemical measurements of the subject;
generating a first feature vector representing the subject from the cardiometabolic health data for the subject, and/or a second feature vector representing the subject from the cardiometabolic health data for the subject; and
performing one or both of:
estimating an LDL level trajectory for the subject by processing the first feature vector using a LDL trajectory prediction machine learning model that has been trained to estimate an LDL level for a subject at each of multiple prior ages and each of multiple future ages using training data comprising for each of a plurality of participants, repeated longitudinal measures of LDL levels and values for the one or more clinical characteristics and/or the one or more physical measurements and/or the one or more biochemical measurements over at least 10, at least 20, at least 30, or at least 50 years of follow-up, wherein the LDL level trajectory for the subject comprises an estimated LDL level for the subject for each of multiple prior ages of the subject and multiple future ages of the subject; and
estimating an SBP level trajectory for the subject by processing the second feature vector using an SBP trajectory prediction machine learning model that has been trained to estimate an SBP level for a subject at each of multiple prior ages and each of multiple future ages using training data comprising, for each of a plurality of participants, repeated longitudinal measures of SBP levels and values for the one or more clinical characteristics and/or the one or more physical measurements and/or the one or more biochemical measurements over at least 10, at least 20, at least 30, or at least 50 years of follow-up, wherein the SBP level trajectory for the subject comprises an estimated SBP level for the subject for each of multiple prior ages of the subject and multiple future ages of the subject; and
pricing the insurance instrument for the subject based on the estimated LDL level trajectory and/or the estimated SBP level trajectory.
264. The method of paragraph 263, wherein the method comprises performing the method of any of paragraphs 210-235.
265. A computer-implemented method for pricing an insurance instrument for a subject, the method comprising:
determining an expected risk of cardiovascular events for a subject in response to a therapeutic intervention associated with a reduction of LDL level and/or SBP level over an interval of follow up for the subject using a method comprising:
(a) determining multiple measures of risk of cardiovascular events for the subject comprising absolute instantaneous hazard rates, cumulative hazard rates, and cumulative event rates of the subject having a cardiovascular event at respective ones of multiple time intervals, wherein the cumulative hazard rates and the cumulative event rates are not adjusted for the therapeutic intervention;
(b) for each particular interval of follow-up for the subject from the subject's current age to an upper threshold age,
(i) determining, using the multiple measures of risk, a predicted instantaneous hazard rate of the subject having a cardiovascular event at the particular interval of follow up;
(ii) determining, using a benefit prediction machine learning model, a time-averaged instantaneous log hazard ratio for a one unit lower LDL or SBP corresponding to duration of treatment at the particular interval of follow-up for the subject, wherein the benefit prediction machine learning model has been trained using training data from randomized trials of LDL lowering therapies and/or randomized trials of SBP therapies, and/or Mendelian randomization studies evaluating genetic variants associated with lower LDL and/or lower SBP, said training data comprising for each of a plurality of participants in said trials, at least one LDL or SBP measurement along with a recorded age or date at which a first cardiovascular event occurred;
(iii) determining an intervention-adjusted instantaneous hazard for the particular interval of follow-up by multiplying the instantaneous hazard rate of the subject determined at (a)(i) with the time-averaged instantaneous log hazard ratio determined at (b)(ii), thereby obtaining multiple intervention-adjusted instantaneous hazards for intervals of follow-up evaluated at (b);
(c) determining, using the multiple intervention-adjusted instantaneous hazards, intervention-adjusted cumulative hazard rates and cumulative event rates of cardiovascular events for intervals of follow-up for the subject from the subject's current age to the upper threshold age; and
(d) pricing the insurance instrument for the subject using the intervention-adjusted cumulative hazard rates and cumulative event rates of cardiovascular events obtained at (c).
266. The method of paragraph 265, wherein the method comprises performing the method of any of paragraphs 250-259.
267. A computer-implemented method for pricing an insurance instrument for a subject, the method comprising:
determining the value of one or more cardiometabolic health metrics associated with the subject using the method of any preceding paragraph; and
pricing the insurance instrument for the subject based on the results of said determining.
268. A system, comprising:
at least one computer hardware processor; and
at least one non-transitory computer readable storage medium storing processor-executable instructions that, when executed by the at least one processor, cause the at least one processor to perform the method of any one of the foregoing method paragraphs.
269. At least one non-transitory computer readable storage medium storing processor-executable instructions that, when executed by at least one processor, cause the at least one processor to perform the method of any one of the foregoing method paragraphs.
270. A computer-implemented method, comprising:
obtaining cardiometabolic health data for a subject comprising: one or more values for one or more clinical characteristics of the subject, and/or one or more values for one or more physical measurements of the subject, and/or one or more values for one or more biochemical measurements of the subject;
encoding the cardiometabolic health data for the subject into a first feature vector;
estimating an SBP level trajectory for the subject by processing the first feature vector using an SBP trajectory prediction ML model that has been trained to estimate an SBP level for a subject at each of multiple prior ages and each of multiple future ages using training data comprising, for each of a plurality of participants, repeated longitudinal measures of SBP levels and values for the one or more clinical characteristics and/or the one or more physical measurements and/or the one or more biochemical measurements over at least 10, at least 20, at least 30, or at least 50 years of follow-up,
wherein the SBP level trajectory for the subject comprises an estimated SBP level for the subject for each of multiple prior ages of the subject and multiple future ages of the subject.
271. The method of paragraph 270, further comprising:
encoding the cardiometabolic health data for the subject into a second feature vector;
estimating an LDL level trajectory for the subject by processing the second feature vector using an LDL trajectory prediction machine learning (ML) model that has been trained to estimate an LDL level for a subject at each of multiple prior ages and each of multiple future ages using training data comprising for each of a plurality of participants, repeated longitudinal measures of LDL levels and values for the one or more clinical characteristics and/or the one or more physical measurements and/or the one or more biochemical measurements over at least 10, at least 20, at least 30, or at least 50 years of follow-up,
wherein the LDL level trajectory for the subject comprises an estimated LDL level for the subject for each of multiple prior ages of the subject and multiple future ages of the subject.
272. The method of paragraph 270 or 271, wherein:
(i) the one or more values for one or more clinical characteristics comprise values for one or more demographic characteristics of the subject, one or more genetic characteristics of the subject, one or more family history characteristics of the subjects, one or more comorbidities, and/or risk factors; and/or
(ii) the one or more values for one or more physical measurements of the subject comprise one or more values for physical measurements of quantities that are risk factors for cardiometabolic disease and/or physiologic al measurements selected from blood pressure measurements and measurements indicative of adiposity; and/or
(iii) the one or more values for one or more biochemical measurements of the subject comprise one or more values for biochemical measurements of quantities that are risk factors for cardiovascular disease and/or biochemical measurements selected from measurements of one or more biochemical markers, optionally a protein, lipid or lipoprotein, in a blood, serum, and/or plasma sample from the subject.
273. The method of any one of paragraphs 270-272, wherein:
(i) the one or more clinical characteristics of the subject are selected from the group consisting of: age, biological sex, family history of coronary heart disease (CHD), family history of hypertension (HTN), family history of type 2 diabetes (T2D), polygenic score for ASCVD, polygenic score for CHD, polygenic score for HTN, polygenic score for T2D, polygenic score for body mass index (BMI), inherited predisposition or predispositions, and history of tobacco use; and/or
(ii) the one or more physical measurements of the subject are selected from the group consisting of systolic blood pressure (SBP), diastolic blood pressure (DBP), weight, waist circumference, height, body mass index (BMI), and waist-to-height ratio; and/or
(iii) the one or more biochemical measurements of the subject are selected from the group consisting of low-density lipoprotein (LDL) level, high-density lipoprotein (HDL) level, total cholesterol level, triglyceride (TG) level, non-HDL cholesterol level, apolipoprotein (apoB) level, lipoprotein (a) (Lp(a)) level, hemoglobin A1c (HbA1c) level, and c-reactive protein (CRP) level.
274. The method of paragraph 270, wherein encoding the cardiometabolic health data to obtain the first feature vector, comprises:
obtaining an initial feature vector using at least some of the cardiometabolic health data for the subject by standardizing at least some of values in the subject characteristic and/or measurement data to obtain an initial feature vector; and
positionally encoding the initial feature vector by age of the subject associated with the values in the cardiometabolic health data for the subject to obtain the a feature vector representing the subject, wherein the positionally encoding comprises:
generating a positional encoding of the initial feature vector using sinusoidal encoding of at least some elements of the initial feature vector, wherein the sinusoidal encoding uses age in years as position; and
generating the feature vector by appending the positional encoding of the initial feature vector to the initial feature vector.
275. The method of paragraph 271, wherein the LDL trajectory prediction ML model has been trained using training data comprising for each of the plurality of participants, repeated longitudinal measures of LDL levels and feature vectors derived from values for the one or more clinical characteristics and/or the one or more physical measurements and/or the one or more biochemical measurements using at least positional encoding by age of the participant associated with the respective values, optionally, wherein the positional encoding is sinusoidal encoding.
276. The method of any one of paragraphs 271-275, wherein the LDL trajectory prediction ML model is a bi-directional LSTM (bi-LSTM) model, a bi-directional GRU model, or an ODE-RNN model.
277. The method of paragraph 276, wherein:
the LDL trajectory prediction ML model is the bi-LSTM model, and
processing the first feature vector using the LDL trajectory prediction ML model comprises:
estimating an LDL level for the subject at each of the multiple future ages using a forward pass of the bi-LSTM model; and
estimating an LDL level for the subject at each of the multiple prior ages using a backward pass of the bi-LSTM model.
278. The method of any one of paragraphs 271-277, further comprising:
estimating, using the LDL level trajectory, a cumulative LDL exposure trajectory for the subject with respect to a set of ages, wherein the cumulative LDL exposure trajectory comprises an estimated cumulative LDL exposure level for the subject at each age in the set of ages.
279. The method of any one of paragraphs 270-278, further comprising:
estimating a weight trajectory for the subject using the cardiometabolic health data, wherein the weight trajectory for the subject comprises estimated weight of the subject for each of multiple ages of the subject, optionally wherein estimating the weight trajectory is performed assuming that the subject's current age-and-sex adjusted weight percentile remains constant throughout life; and/or
estimating a waist circumference trajectory for the subject the cardiometabolic health data, wherein the waist circumference trajectory for the subject comprises estimated waist circumference of the subject for each of multiple ages of the subject, optionally wherein estimating the waist circumference trajectory is performed assuming that the subject's current age-and-sex adjusted waist circumference percentile remains constant throughout life; and/or
estimating an HbA1c level trajectory for the subject using the cardiometabolic health data, wherein the HbA1c level trajectory for the subject comprises estimated HbA1c levels of the subject for each of multiple ages of the subject, optionally wherein estimating the HbA1c level trajectory is performed assuming that the subject's current age-and-sex adjusted HbA1c percentile remains constant throughout life.
280. A computer-implemented method of determining one or more measures of risk that a subject develops cardiovascular disease, the method comprising, for each of multiple time intervals:
(a) estimating, using a trained cardiovascular risk prediction machine learning model and cardiometabolic health data for the subject including a cumulative LDL exposure trajectory for the subject, values indicative of log hazard ratios for risk of the subject having a cardiovascular event at respective levels of cumulative LDL exposure, wherein the cardiovascular risk prediction machine learning model has been trained using training data comprising, for each of a plurality of participants enrolled in one or more prospective studies, multiple LDL measurements along with a recorded age or date at which a first cardiovascular event occurred, optionally wherein the first cardiovascular event is a first episode of a fatal or non-fatal myocardial infarction (MI), fatal or non-fatal ischemic stroke, or coronary revascularization; and
(b) estimating, using the values indicative of the log hazard ratios,
(i) absolute instantaneous hazard rates of the subject having a cardiovascular event at the respective levels of cumulative LDL exposure, and
(ii) cumulative lifetime risks of the subject having a cardiovascular event at the respective levels of cumulative LDL exposure; and
(c) estimating, using the cumulative LDL exposure trajectory for the subject, the multiple measures of risk to include:
(i) absolute instantaneous hazard rates of the subject having a cardiovascular event at respective ones of the multiple time intervals, and
(ii) cumulative lifetime hazard and event rates of the subject having a cardiovascular event at the respective ones of the multiple time intervals.
281. The method of paragraph 280,
wherein the cumulative LDL exposure trajectory has been obtained using the method of any of paragraphs 271-279, and/or wherein the method comprises obtaining the cumulative LDL exposure trajectory using the method of any of paragraphs 271-279; and/or
wherein step (a) uses cardiometabolic health data further comprising a cumulative SBP exposure trajectory that has been obtained using the method of any of paragraphs 271-279, and/or wherein step (a) uses cardiometabolic health data further comprising a cumulative SBP exposure trajectory and the method comprises obtaining the cumulative SBP exposure trajectory using the method of any of paragraphs 271-279; and/or
wherein step (a) uses cardiometabolic health data further comprising a cumulative Lp(a) exposure trajectory that has been obtained using the method of any of paragraphs 271-279, and/or wherein step (a) uses cardiometabolic health data further comprising a cumulative Lp(a) exposure trajectory and the method comprises obtaining the cumulative SBP exposure trajectory using the method of any of paragraphs 271-279.
282. The method of paragraph 280, wherein estimating, using the trained cardiovascular risk prediction machine learning model and the at least some of the cardiometabolic health data, the values indicative of the log hazard ratios for risk of the subject having a cardiovascular event at the respective levels of cumulative LDL exposure comprises:
encoding the at least some of the cardiometabolic health data to obtain input feature data; and
estimating, by processing the input feature data using the cardiovascular risk prediction ML model, the values indicative of the log hazard ratios for the risk of the subject having a cardiovascular event at the respective levels of cumulative LDL exposure,
optionally, wherein processing the input feature data comprises using a trained survival deep neural network (DNN) with piecewise exponential modeling (PEM) model to calculate log hazard ratios for the risk of the subject having a cardiovascular event at the respective levels of cumulative LDL exposure.
283. A computer-implemented method of determining an expected proportional reduction and/or absolute reduction in risk of cardiovascular events for a subject in response to a particular therapeutic intervention sequence, the particular therapeutic sequence indicating magnitude, duration, and timing of one or more interventions associated with a reduction of LDL level and/or SBP level over an interval of follow up for the subject, the method comprising:
determining multiple measures of risk of cardiovascular events for the subject comprising absolute instantaneous hazard rates, cumulative hazard rates, and cumulative event rates of the subject having a cardiovascular event at respective ones of multiple time intervals, wherein the cumulative hazard rates and the cumulative event rates are not adjusted for the particular therapeutic intervention sequence, and determining the expected proportional reduction and/or the absolute reduction in the risk of cardiovascular events for the subject in response to the particular therapeutic intervention sequence using a method that comprises:
(a) for each particular interval of follow-up for the subject from the subject's current age to an upper threshold age,
(i) determining, using the multiple measures of risk, a predicted instantaneous hazard rate of the subject having a cardiovascular event at the particular interval of follow up;
(ii) determining, using a benefit prediction machine learning model, a time-averaged instantaneous log hazard ratio for a one unit lower LDL or SBP corresponding to duration of treatment at the particular interval of follow-up for the subject, wherein the benefit prediction machine learning model has been trained using training data from randomized trials of LDL lowering therapies and/or randomized trials of SBP therapies and Mendelian randomization studies evaluating genetic variants associated with lower LDL and/or lower SBP, said training data comprising for each of a plurality of participants in said trials, at least one LDL or SBP measurement along with a recorded age or date at which a first cardiovascular event occurred;
(iii) determining an intervention-adjusted instantaneous hazard for the particular interval of follow-up by multiplying the instantaneous hazard rate of the subject determined at (a)(i) with the time-averaged instantaneous log hazard ratio determined at (a)(ii),
thereby obtaining multiple intervention-adjusted instantaneous hazards for intervals of follow-up evaluated at (a);
(b) determining, using the multiple intervention-adjusted instantaneous hazards, intervention-adjusted cumulative hazard rates and cumulative event rates of cardiovascular events for intervals of follow-up for the subject from the subject's current age to the upper threshold age;
(c) determining predicted proportional reductions in the risk of experiencing a cardiovascular event as ratios of the intervention-adjusted cumulated hazard rates and the cumulative hazard rates that are not adjusted for the particular therapeutic intervention sequence; and
(d) determining predicted absolute reductions in the risk of experience of experiencing a cardiovascular event as absolute differences between the intervention-adjusted cumulative event rates and the cumulative event rates that are not adjusted for the particular therapeutic intervention sequence.
284. The method of paragraph 283, wherein the multiple measures of risk not adjusted for the particular therapeutic intervention have been obtained using the method of any of paragraphs 280-282; and/or wherein the method comprises determining the multiple measures of risk of cardiovascular events for the subject not adjusted for the particular therapeutic intervention using the method of any of paragraphs 280-282.
285. The method of any one of paragraphs 283-284, wherein the benefit prediction machine learning model comprises a causal DNN for Ordinary Differential Equations (c-DNN-ODE) model.
286. A system, comprising:
at least one computer hardware processor; and
at least one non-transitory computer readable storage medium storing processor-executable instructions that, when executed by the at least one processor, cause the at least one processor to perform the method of any one of the foregoing method paragraphs.
287. At least one non-transitory computer readable storage medium storing processor-executable instructions that, when executed by at least one processor, cause the at least one processor to perform the method of any one of the foregoing method paragraphs.
288. A method for estimating remaining lifetime risk of cardiovascular events, comprising: (a) determining previous and expected future cumulative exposure to modifiable causes of disease; (b) estimating remaining lifetime risk of cardiovascular events beginning at any age based on the cumulative exposure determined in step (a); (c) conditioning the estimation on biological sex, family history, and polygenic predisposition; and (d) conditioning the estimation on other exposures that reduce the capacity of an artery to tolerate accumulated plaque burden or increase the propensity for plaque disruption.
289. The method of paragraph 288, wherein the estimation remains valid on or off treatment based on reduction in cumulative exposure to the modifiable causes of disease.
290. A method for estimating an increasing benefit of lowering LDL over time, comprising: (a) determining a starting age and duration for LDL lowering; (b) assessing disease burden that accumulates prior to LDL lowering; and (c) estimating the increasing benefit of lowering LDL and other apoB-containing lipoproteins beginning at the starting age and extending for the duration, conditional on the disease burden assessed in step (b).
291. The method of paragraph 290, wherein the estimating comprises estimating the benefit of lowering LDL using an intervention.
292. A method for estimating an increasing benefit of lowering SBP over time, comprising: (a) determining a starting age and duration for SBP lowering; (b) assessing disease burden that accumulates prior to initiation of blood pressure lowering; and (c) estimating the increasing benefit of lowering SBP beginning at the starting age and extending for the duration, conditional on the disease burden assessed in step (b).
293. The method of paragraph 292, wherein the estimating comprises estimating the benefit of lowering SBP using an intervention.
294. A method for estimating the increasing combined benefit of lowering both LDL and SBP over time, comprising: (a) determining a starting age and duration for lowering both LDL and SBP; (b) assessing disease burden that accumulates prior to initiation of blood pressure lowering; and (c) estimating the increasing combined benefit of lowering both LDL and SBP beginning at the starting age and extending for the duration, conditional on the disease burden assessed in step (b).
295. The method of paragraph 294, wherein the estimating comprises estimating the benefit of lowering both LDL and SBP using one or more interventions.
296. A method for iteratively providing real-time updates on cardiometabolic health status, comprising: (a) monitoring evolving cumulative exposure to modifiable causes of disease; (b) providing updated predictions of remaining lifetime risk of cardiovascular events over any time interval based on the monitoring in step (a); (c) conditioning the updated predictions on biological sex, family history, and polygenic predisposition; (d) conditioning the updated predictions on other exposures that reduce the capacity of an artery to tolerate accumulated plaque burden or increase the propensity for plaque disruption; and (e) conditioning the updated predictions on cumulative achieved reductions in modifiable causes of disease from previous interventions and evolving disease burden from other exposures over time.
297. The method of paragraph 296, wherein the method extracts meaningful information from longitudinal measurements of clinical parameters to monitor cardiometabolic health for guiding interventions to prevent disease by slowing a trajectory of common diseases.
298. A method for determining optimal intervention parameters, comprising: (a) assessing cumulative achieved reductions in modifiable causes of disease from previous interventions; (b) assessing evolving disease burden from other exposures overtime; and (c) determining optimal intensity, duration, and timing of lowering LDL, SBP, and Lp(a) to achieve a desired clinical outcome, conditional on the assessments in steps (a) and (b).
299. The method of paragraph 298, wherein the determining comprises determining optimal magnitude, duration, and timing of lowering LDL, SBP, and Lp(a) using one or more interventions.
300. A method for designing and predicting results of randomized trials, comprising: (a) assessing disease burden that has accumulated prior to intervention for each participant; (b) designing randomized trials evaluating interventions to lower LDL, SBP, and Lp(a) alone or in combination; and (c) predicting both short-term and long-term benefit of the interventions, conditional on the disease burden assessed in step (a).
301. The method of paragraph 300, wherein the predicting captures both short-term and long-term benefit within a short-term trial.
302. A method for identifying optimal intervention candidates and timing, comprising: (a) identifying persons likely to benefit from early interventions to lower LDL, SBP, and Lp(a); (b) determining optimal timing for lowering LDL, SBP, and Lp(a) using one or more interventions; (c) longitudinally monitoring achieved cumulative reductions in targeted modifiable causes of disease; (d) monitoring corresponding reductions in short-term and long-term risks of cardiovascular events; and (e) conditioning the monitoring on evolving disease burden from other exposures over time.
303. The method of paragraph 302, further comprising informing reimbursement of siRNA therapeutics based on achieved reductions in modifiable causes of disease and expected reductions in risk of clinical events.
304. A method for dynamically pricing insurance instruments, comprising: (a) iteratively providing real-time updates on evolving cardiometabolic health status; (b) providing updated predictions of short-term and long-term risks of experiencing a cardiovascular event without intervention and in response to specific interventions to lower LDL, SBP, Lp(a) and other modifiable causes of disease; (c) conditioning the updated predictions on achieved cumulative reductions in modifiable causes of disease from previous interventions; and (d) conditioning the updated predictions on evolving disease burden from other exposures over time.
305. A method for creating a digital infrastructure for precision cardiometabolic health, comprising: (a) providing an autonomous-cloud database optimization architecture; and (b) combining the architecture with a stack of deep and machine learning algorithms that encode biological cause and effect to longitudinally monitor and proactively guide optimal timing and intensity of therapeutic interventions to extend healthy lifespan by personalizing prevention of cardiometabolic disease.
306. A method for estimating trajectory and cumulative exposure to LDL and other apoB-containing lipoproteins for one or more individual(s), comprising: (a) receiving input data comprising a plasma LDL level and a vector of contemporaneous exposures, wherein the vector of contemporaneous exposures is selected from age, biological sex, family history, HDL, total cholesterol, SBP, DBP, weight, waist circumference, BMI, HbA1c, history of diabetes, smoking history, polygenic predisposition, and inflammatory markers of each individual, and wherein the input data excludes individuals on lipid or blood pressure lowering therapies; (b) processing the input data using an LSTM neural network or a collaborative-filtering deep learning algorithm, optionally, when the collaborative-filtering deep learning algorithm is used, selecting a trajectory from the collaborative-filtering deep learning algorithm based on a similarity score of the vector; (d) estimating for the one or more individual(s) a trajectory of changing LDL levels over time based on the plasma LDL level; (e) calculating previous and expected future cumulative exposure to LDL based on the selected trajectory; and (f) outputting predicted plasma LDL levels at all ages, optionally from birth to age 80 years, and corresponding cumulative exposure to LDL at each age, wherein the cumulative exposure to LDL at each age comprises a sum of preceding predicted LDL levels.
307. The method of paragraph 306, wherein the cumulative exposure to LDL provides a dynamic biomarker for monitoring rate of plaque progression, size of accumulated plaque burden, and/or corresponding absolute risk of having an acute cardiovascular event at any time point.
308. A method for estimating risk of cardiovascular events for one or more individual(s) based on previous and expected future cumulative exposure to LDL, comprising: (a) receiving input data comprising predicted cumulative exposure to LDL at each age and other exposures, observed absolute risk of major cardiovascular events at each level of cumulative exposure to LDL observed in cumulative exposure to LDL driven naturally randomized trial, and a common vector of other contemporaneous exposures; (b) constructing a lifetable of predicted absolute risk of having a cardiovascular event at all levels of cumulative exposure to LDL and all corresponding ages, conditional on other exposures; (c) processing the input data using lifetable analysis of absolute risk of cardiovascular events at all levels of cumulative exposure to LDL adjusted by instantaneous hazards derived from proportional hazards models and time to event analyses measuring effect size of exposures in the cumulative exposure to LDL driven naturally randomized trial; (d) determining a personal plaque threshold comprising cumulative exposure to LDL above which cardiovascular events begin to occur for each individual conditional on accumulated plaque burden, capacity of an artery to tolerate the accumulated plaque burden, and propensity for plaque disruption; and (e) outputting lifetable derived cumulative event curves with predicted absolute risk of cardiovascular events at all levels of cumulative exposure to LDL and corresponding age based on individual trajectory of LDL, and an estimate of the personal plaque threshold for cumulative exposure to LDL above which cardiovascular events begin to occur for use as a therapeutic target.
309. The method of paragraph 308, wherein the propensity for plaque disruption includes both inherited and acquired factors.
310. The method of paragraph 308, wherein the personal plaque threshold serves as a therapeutic target for intervention planning.
311. A method for estimating increasing benefit of lowering LDL by magnitude and duration of LDL lowering for one or more individual(s), comprising: (a) receiving input data comprising absolute event rates and instantaneous hazard ratios during each time increment of follow-up adjusted for observed absolute difference in LDL between treatment groups during each month of follow-up in LDL lowering cardiovascular outcomes trials; (b) receiving additional input data comprising absolute event rates and instantaneous hazard ratios during each time increment of follow-up adjusted for observed absolute difference in LDL between treatment groups naturally randomized by nature to higher or lower lifetime exposure to LDL during each year of life in naturally randomized trials of genetic variants associated with lower LDL with concordant proportional changes in apoB, optionally from ages 30-80 years; (c) processing the input data using an ensemble of gradient-boosted machines or deep learning model, optionally a c-DNN-ODE model, to fit a non-linear curve to estimate increasing instantaneous hazard ratios from initiation of LDL lowering in the naturally randomized trials to up to 80 years of follow-up in the naturally randomized trials; (d) estimating increasing proportional reduction in major cardiovascular events for a standardized increment of LDL lowering by increasing duration of LDL lowering; and (e) outputting instantaneous and cumulative summary hazard ratio at each duration of LDL lowering standardized for a fixed increment of LDL lowering to provide expected proportional reduction in risk of cardiovascular events by magnitude and duration of LDL lowering.
312. The method of paragraph 311, wherein the naturally randomized trials comprise genetic variants associated with lower LDL with concordant proportional changes in apoB.
313. The method of paragraph 311, wherein the ages at which cardiovascular events begin to occur comprise ages 30-80 years.
314. The method of paragraph 311, wherein the standardized increment of LDL lowering provides a consistent basis for comparing therapeutic benefits across different intervention strategies.
315. A method for estimating increasing benefit of lowering LDL for each individual by magnitude, duration, and timing of LDL lowering, comprising: (a) receiving input data comprising magnitude of LDL lowering and age at which LDL lowering is started; (b) combining individual lifetable estimates of risk of major cardiovascular events at all levels of cumulative exposure to LDL and corresponding age with expected proportional reduction in risk from lowering LDL at each time increment; (c) processing the input data using lifetable analysis of absolute risk of cardiovascular events at all levels of cumulative exposure to LDL adjusted by instantaneous hazards derived from proportional hazards models and time to event analyses measuring effect size of exposures in cumulative exposure to LDL driven naturally randomized trial; (d) adjusting absolute event rates by predicted increasing instantaneous hazard ratio for magnitude of LDL reduction during each increasing time increment of LDL lowering; (e) estimating benefit of lowering LDL by magnitude, duration, and timing of LDL lowering conditional on accumulated plaque size of plaque burden and injury to arterial wall at time LDL lowering was initiated; and (f) outputting individual lifetable derived cumulative event curves with predicted absolute risk of cardiovascular events at all levels of cumulative exposure to LDL and corresponding age based on individual trajectory of LDL conditional on achieved magnitude, duration, and timing of LDL lowering.
316. The method of paragraph 315, wherein the method provides an estimate of expected individual benefit from lowering LDL by any amount, beginning at any age, and lasting for any duration.
317. The method of paragraph 315, wherein the timing of when LDL lowering is started reflects disease burden that accumulated prior to LDL lowering.
318. The method of paragraph 315, wherein the method integrates personalized risk profiles with treatment benefits for individual patients.
319. A method for estimating individual trajectory and cumulative exposure to SBP for one or more individual(s), comprising: (a) receiving input data comprising SBP level and a common vector of other contemporaneous exposures including age, biological sex, DBP, family history, plasma LDL, HDL, total cholesterol, weight, waist circumference, BMI, HbA1c, history of diabetes, and smoking history, wherein the input data excludes persons on lipid or blood pressure lowering therapies; (b) processing the input data using construction of vector embeddings for SBP readings contextualized by the contemporaneous common exposures using a fully connected deep learning algorithm and a temporal fusion transformer algorithm designed to positionally encode each SBP reading in sequence and pay attention to a longer window of previous SBP readings and evolving contextual information from other contemporaneous exposures; (c) estimating each person's unique trajectory of changing SBP levels over time based on a single SBP measurement to calculate their previous and expected future cumulative exposure to SBP; (d) providing a dynamic biomarker for monitoring accumulating pressure-induced injury to an artery wall and reduced capacity of the artery to tolerate accumulated plaque burden; and (e) outputting predicted SBP level at all ages from age 20 to age 80 years and corresponding cumulative exposure to SBP at each age comprising sum of preceding predicted SBP levels.
320. The method of paragraph 319, wherein the vector of contemporaneous exposures further includes polygenic predisposition when available.
321. The method of paragraph 319, wherein the vector of contemporaneous exposures further includes inflammation markers when available.
322. A method for estimating risk of cardiovascular events based on previous and expected future cumulative exposure to SBP for one or more individual(s), comprising: (a) receiving input data comprising predicted cumulative exposure to SBP at each level of cumulative exposure to LDL within a lifetable using age as a key to map accumulated SBP for each individual to their accumulated plaque burden; (b) estimating impact of each dynamically increasing increment of cumulative exposure to SBP on instantaneous risk of major cardiovascular events at all levels of accumulated plaque burden; (c) processing the input data using lifetable analysis of absolute risk of cardiovascular events at all levels of cumulative exposure to LDL adjusted by instantaneous hazard ratio from magnitude of cumulative exposure to SBP at each level of cumulative exposure to LDL; (d) deriving estimated effect size of an increment of increased cumulative exposure to SBP at all levels of cumulative exposure to LDL using a feature engineered deep learning algorithm; and (e) outputting lifetable derived cumulative event curves with predicted absolute risk of cardiovascular events at all levels of cumulative exposure to LDL incorporating individual trajectories and cumulative exposure to SBP to provide updated personal plaque thresholds based on effect of rising SBP over time.
323. The method of paragraph 322, wherein the personal plaque threshold is adjusted by reduced capacity of the artery to tolerate accumulated plaque burden due to accumulating pressure-induced irreversible structural injury to the artery wall.
324. A method for estimating increasing benefit of lowering SBP by magnitude and duration of SBP lowering for one or more individual(s), comprising: (a) receiving input data comprising absolute event rates and instantaneous hazard ratios during each time increment of follow-up adjusted for observed absolute difference in SBP between treatment groups during each month of follow-up in SBP lowering cardiovascular outcomes trials; (b) receiving additional input data comprising absolute event rates and instantaneous hazard ratios during each time increment of follow-up adjusted for observed absolute difference in SBP between treatment groups naturally randomized by nature to higher or lower lifetime exposure to SBP during each year of life in naturally randomized trials of genetic variants associated with lower SBP with concordant proportional changes in DBP from ages 30-80 years; (c) processing the input data using an ensemble of gradient-boosted machines or a deep learning model, optionally a c-DNN-ODE, to fit a non-linear curve to estimate increasing instantaneous hazard ratios from initiation of SBP lowering to up to 80 years of follow-up; and (d) outputting instantaneous and cumulative summary hazard ratio at each duration of SBP lowering standardized for a fixed increment of SBP lowering to estimate expected proportional reduction in risk of cardiovascular events by magnitude and duration of SBP lowering.
325. A method for estimating increasing benefit of lowering SBP for one or more individual(s) by magnitude, duration, and timing of SBP lowering, comprising: (a) receiving input data comprising magnitude of SBP lowering and age at which SBP lowering is started; (b) combining individual lifetable estimates of risk of major cardiovascular events at all levels of cumulative exposure to LDL and corresponding age and cumulative exposure to SBP with expected proportional reduction in risk from lowering SBP at each time increment; (c) processing the input data using lifetable analysis of absolute risk of cardiovascular events at all levels of cumulative exposure to LDL and cumulative exposure to SBP adjusted by increasing instantaneous hazard ratios; (d) estimating benefit of lowering SBP by magnitude, duration, and timing conditional on accumulated plaque burden and accumulated pressure-induced arterial wall injury at time SBP lowering was initiated; and (e) outputting individual lifetable derived cumulative event curves with predicted absolute risk of cardiovascular events at all levels of cumulative exposure to LDL conditional on achieved magnitude, duration, and timing of SBP lowering.
326. A method for estimating reduction in LDL, SBP, or Lp(a) for one or more individual(s) to achieve a desired clinical outcome, comprising: (a) receiving input data comprising cumulative exposure to LDL, cumulative exposure to SBP, common vector of contemporaneous exposures, and age at initiation of LDL or SBP lowering; (b) processing the input data using individual lifetable analyses using individual predicted cumulative exposure to LDL and SBP and estimated increasing proportional reductions in risk for major cardiovascular events by magnitude, duration, and timing of LDL and SBP lowering conditional on other exposures; (c) employing a hierarchical Markov search prioritizing LDL lowering conditional on rate of rise in SBP to select magnitude of LDL and SBP lowering required; (d) identifying age at which a once yearly intervention to lower LDL and a combined once-yearly intervention to lower LDL and SBP would no longer achieve desired outcome; and (e) outputting magnitude of LDL or LDL and SBP lowering used to achieve a desired outcome for each individual conditional on accumulated disease burden that develops before intervention is started.
327. A method for selecting optimal therapeutic intervention for one or more individual(s) to achieve a desired clinical outcome by magnitude, duration, and timing of lowering LDL, SBP, Lp(a), or any combination, comprising: (a) receiving input data comprising cumulative exposure to LDL, cumulative exposure to SBP, common vector of contemporaneous exposures, and age at initiation of LDL or SBP lowering; (b) employing reinforcement learning with Markov tree search where State is defined by current cardiometabolic health determined by a person's cumulative exposure to LDL and SBP, age, biological sex, family history, polygenic predisposition, and biomarkers; (c) passing parameters into an AI Agent that uses deep and machine learning algorithms that encode biological cause and effect to translate biomarkers into an estimate of current cardiometabolic health; (d) selecting optimal therapy to achieve selected desired outcome over any time horizon and recommending intervention as Action; (e) receiving achieved magnitude of change in targeted modifiable cause of disease and corresponding risk reduction as Reward; (f) creating new updated cardiometabolic State based on change in targeted modifiable cause of disease and change in other untargeted exposures; (g) feeding biological parameters back to AI Agent for updated estimate of expected risk; and (h) outputting real time longitudinally updated measure of cardiometabolic health, updated risk of cardiovascular events based on achieved cumulative reductions in modifiable causes of disease and accumulated disease burden, and updated guidance on optimal combination and intensity of LDL, SBP, and/or Lp(a) lowering.
328. The method of paragraph 327, wherein the AI Agent estimates current cardiometabolic health characterized by accumulated plaque burden, cumulative injury to artery wall, vulnerability to cardiometabolic diseases, expected rate of disease progression, corresponding personal plaque threshold for cardiovascular events conditional on other exposures, and expected absolute instantaneous and cumulative remaining lifetime risk of having a cardiovascular event.
329. The method of paragraph 327, wherein the method provides iteratively updated clinical guidance on maintaining cardiometabolic health based on evolving disease burden and revised estimates of risk and benefit.

### EXAMPLES

### Example 1

Aspects of the technology provide a sustainable competitive advantage by addressing three problems in cardiometabolic disease prevention. First, the technology quantifies how much the benefit of lowering LDL and SBP increases over time and for the first time quantifies the proportional reduction in risk from lowering LDL and SBP by both the magnitude and duration of exposure. This demonstrates that the clinical benefit of lowering LDL (and other apoB-containing lipoproteins) increases over time by slowing the progression of atherosclerosis to reduce the risk of atherosclerotic cardiovascular events including MI and stroke, and the clinical benefit of lowering SBP increases over time by slowing the progression of atherosclerosis and pressure-induced irreversible structural injury to the arterial wall. Quantification of the increasing clinical benefit over time from lowering LDL and SBP establishes the paradigm that cardiometabolic diseases can be effectively prevented by intervening early to slow the progression of disease. By contrast, if the proportional reduction in risk from lowering LDL and SBP does not increase over time but instead is constant, then there is no rationale for early intervention because lowering LDL and SBP would not slow the progression of disease. It can be demonstrated mathematically that if lowering LDL and SBP produces a constant proportional reduction in risk, then the optimal strategy to prevent clinical events would be to wait to lower LDL or SBP until enough underlying disease develops to increase risk above an absolute threshold.

Second, aspects of the technology address the prevention paradox by providing a method to identify the optimal timing, intensity, and combination of interventions useful to each person to prevent the development of cardiometabolic disease. This addresses the fundamental challenge of determining when to initiate LDL and SBP lowering interventions and the magnitude of reduction required, given that the benefit of lowering LDL and SBP increases over time. By quantifying the proportional reduction in risk from lowering LDL and SBP by the magnitude and duration of therapy, the model described herein can compute how much each person needs to lower their LDL and SBP and when to personalize the prevention of MI, stroke, and hypertension.

Third, aspects of the technology create deep and machine learning algorithms that can explain the reasoning and biological rationale used to inform estimates of risk and benefit, and recommendations for actions to prevent disease. Aspects of the technology introduce algorithms that are designed to encode biological cause and effect, including the first algorithms that are explicitly trained to learn the biology of how common diseases develop and how to use this information to estimate risk and benefit and discover how to optimally prevent cardiometabolic disease for each person. Creating Deep Causal AI algorithms that can explain biological reasoning provides transparency, builds trustworthiness, and provides a mechanism to empirically test and iteratively improve all outputs designed to extend the healthy lifespan by intervening early to slow the trajectory of how common diseases develop.

### Assessment of the Current State of Cardiometabolic Health

Assessment of the current state of cardiometabolic health begins with standardization and conversion of input features into a dense input feature vector. Raw measurements for the clinical, physical, and biochemical measurements used to characterize the current state of cardiometabolic health vary widely in magnitude and are measured on different scales. Therefore, the measured input values are first standardized to improve computational efficiency, stabilize gradient flow and prevent exploding or vanishing gradients, and prevent bias toward features with larger numeric ranges. This step is designed to improve computational efficiency and to preserve use of the same dense input feature vector for multiple different sequential deep and machine learning algorithms. Standardization after input of the raw measurements into the Deep Causal AI Agent also preserves the potential to employ an algorithm to recover an estimate of the magnitude of the effect size for each input feature.

Next, each input feature included in the vector undergoes positional encoding using age as the position function. Positional encoding by age injects a biological context into each included feature and is designed to permit the value of each feature to be interpreted within the context of the age at which it was measured. This formulation of positional encoding provides unique biological information about the likely trajectory of prior values for physiologic and biochemical features that may dynamically change value over time. The computed positionally encoded features are then appended to the dense input feature vector.

The positionally encoded dense feature vector is then passed in parallel to two different pre-trained bidirectional long short-term memory (Bi-LSTM) recurrent neural networks. The first Bi-LSTM is designed to predict the trajectory and cumulative exposure to LDL for an individual person (LDL Bi-LSTM). The forward pass (left-to-right) of this Bi-LSTM predicts the LDL level at all future ages (up to the age 80 years, or other pre-defined age limit) based on the measured LDL level at the current age and the combined effect of the other positionally encoded input features. The backward pass (right-to-left) predicts the LDL level at all previous ages from the current age going backward until birth using the same fully connected dense neural network. The predicted LDL level at all ages is then plotted to provide the predicted trajectory of LDL levels throughout life for the individual person being evaluated. The predicted LDL level at each age is then summed (or the area under the predicted curve for LDL trajectory is integrated) to iteratively compute the cumulative exposure to LDL at every age (measured as Plaque Years of LDL in mmol/L).

Cumulative exposure to LDL is a bio-engineered feature that represents a dynamic biomarker and provides unique biological information. It is a direct estimate of the number of LDL and other atherogenic apoB-containing lipoproteins that the arterial wall has been exposed to over time. Therefore, it is an indirect estimate of the number of LDL particles that have become trapped within the artery wall over time. As a result, cumulative exposure to LDL (measured in mmol/L of total Plaque Years of exposure to LDL) can be used to estimate the size of the accumulated atherosclerotic plaque burden at any point in time, the rate of plaque progression, and the expected size of the accumulated plaque burden at all future time points. The values for the predicted LDL level and corresponding cumulative exposure to LDL at all ages is then appended to the positionally encoded dense input feature vector.

The second Bi-LSTM is designed to predict the trajectory, rate of rise, and cumulative exposure to SBP for an individual person (SBP Bi-LSTM). The forward pass (left-to-right) of this Bi-LSTM predicts the SBP level at all future ages (up to the age 80 years, or other pre-defined age limit) based on the measured SBP level at the current age and the combined effect of the other input features. The backward pass (right-to-left) predicts the SBP level at all previous ages from the current age backwards to age 20 years using the same fully connected dense neural network. The SBP levels before age 20 are discounted because they evolve in response to the rapidly changing physiologic requirements during the rapid growth of childhood and adolescence, and because SBP levels high enough to cause accumulating irreversible structural injury to the arterial wall during this time is exceedingly rare.

The predicted SBP level at all ages is then plotted to describe the predicted trajectory of changes in SBP and the predicted rate of rise in SBP for the individual person being evaluated. The predicted rate of rise in SBP is a unique bio-engineered feature that can be combined with the measured SBP at the current age to predict whether a person is likely to develop hypertension, and to predict at what age a person is likely to develop hypertension based on how rapidly SBP is predicted to rise with age (e.g., based on comparison of predicted SBP levels and a specified threshold). This information informs selection of the optimal sequence of current and anticipated future actions useful achieving the combined goal of preventing MI, stroke, hypertension, and diabetes.

The predicted SBP level at each age is then summed (or the area under the predicted curve for SBP trajectory is integrated) to iteratively compute the cumulative exposure to SBP at every age (measured in mmHg years). Cumulative exposure to SBP is another bio-engineered feature that represents a dynamic biomarker that provides unique biological information. Like cumulative exposure to LDL, elevated SBP causes irreversible structural injury to the artery wall that accumulates over time. Specifically, elevated SBP causes non-laminar blood flow at vulnerable branch points within the arterial tree. The non-laminar flow, in turn, causes an increased flux of LDL and other atherogenic apoB-containing lipoproteins within the artery wall. In addition, the elevated SBP causes hypertrophy of vascular smooth muscle cells, which, in turn, cause the secretion of a greater concentration of the proteoglycans that trap LDL and other apoB-containing lipoproteins within the artery wall. This combination of increased flux of atherogenic lipoproteins into the artery wall and a greater concentration of proteoglycans that can trap the increased concentration of LDL within the artery wall results in progressively increasing focal SBP induced plaque accumulation over time at vulnerable branch points within the arteries. As a result, SBP has a cumulative effect on the development of atherosclerotic plaque at specific vulnerable points within the vascular tree. In addition, elevated SBP causes accumulating diffuse structural injury to the artery wall resulting in vascular stiffening and inflammation (arteriosclerosis), which reduces the capacity of the artery to tolerate the accumulated plaque burden by making it more likely that a thrombus overlying a disrupted atherosclerotic plaque will occlude the vessel at any level of accumulated plaque burden. Finally, elevated SBP also increases the risk of plaque disruption, thus increasing the risk of acute atherosclerotic cardiovascular events. For these reasons, LDL and SBP appear to have independent, additive, causal, and cumulative effects on the risk of acute atherosclerotic cardiovascular events. The bio-engineered cumulative exposure to SBP biomarker computed by the SBP Bi-LSTM provides unique information by quantifying these biological effects.

The values for the predicted SBP level, the instantaneous rate of rise in SBP, and the corresponding computed cumulative exposure to SBP at all ages is then appended to the positionally encoded dense input feature vector. The values for predicted SBP, predicted instantaneous rise in SBP, and cumulative exposure to SBP at each age are then positionally encoded by the corresponding age, and these values are also appended to the dense input feature vector.

The results of the two Bi-LSTM provide an assessment of the current state of cardiometabolic health, including an estimate of the size of the atherosclerotic plaque burden that has accumulated, and the rate at which the plaque burden is progressing; the current SBP and estimates of the rate at which SBP is rising over time, and how much structural injury caused by elevated SBP has accumulated. The state of cardiometabolic health may also include the current weight, waist circumference, and HbA1c level, and where available, estimates of how much HbA1c rises in response to weight gain due to excess energy balance, and how much structural injury caused by elevated glucose levels has accumulated.

### Predicting the Risk of Cardiometabolic Disease Over All Time Intervals

Predicting the risk of cardiometabolic disease over all time intervals begins with passing the positionally encoded dense feature vector (including the appended results of the two parallel Bi-LSTM) into a pre-trained Survival Deep Neural Network with Piecewise Exponential Models (Survival DNN with PEM), using cumulative exposure to LDL as the interval of follow-up. This modification of the Survival DNN with PEM is specifically designed to predict how much an individual person's unique combination of exposures impacts the risk of having an acute cardiovascular event at all levels of accumulated plaque burden. Cumulative exposure to LDL is used as the piecemeal interval of survival follow-up in this algorithm because it is an estimate of the size of the accumulated atherosclerotic plaque burden at any point in time, and because the size of the accumulated plaque burden, in turn, is the strongest determinant of the risk of having an acute atherosclerotic cardiovascular event.

However, the risk of having an acute cardiovascular event from a disrupted atherosclerotic plaque does not begin to increase in a measurable way until after the size of the accumulated plaque burden exceeds a specific threshold (measured in cumulative exposure to LDL). Furthermore, at all levels of plaque burden, the risk of having an acute cardiovascular event depends not only on the size of the accumulated plaque burden but also on how much other exposures combine to impact the capacity of the artery to tolerate the accumulated plaque burden, the propensity for plaque disruption within the artery, and the inherited vulnerability to trapping atherosclerotic particles. Therefore, the atherosclerotic plaque size threshold above which cardiovascular events begin to occur, and the risk of having an acute cardiovascular event at all levels of accumulated plaque burden, varies substantially between individuals depending on their unique combination of other exposures.

To learn this biological complexity, the Survival DNN with PEM is partitioned into increments of increasing cumulative exposure to LDL measured in Plaque Years of LDL (mmol/L), which is used as an estimate of the size of the incrementally increasing plaque burden. At each of these piecemeal increments of cumulative exposure to LDL, a fully connected dense deep neural network estimates the log hazard ratio of having an acute cardiovascular event at that size of accumulated plaque burden due to exposure to the combination of other features that impact the capacity of the artery to tolerate the accumulated plaque burden, conditional on surviving to that level of plaque burden without an event. The deep neural network outputs a predicted log hazard ratio for the risk of having an atherosclerotic cardiovascular event at each level of plaque burden and stores them in a vector. This vector of estimated log hazard ratios represent a direct estimate of the biological effect of how a person's combination of exposures impacts the risk of having an atherosclerotic cardiovascular event at every level of accumulated plaque burden size.

Next, the output of the Survival DNN with PEM is used to estimate the absolute risk of having an acute cardiovascular event at all levels of accumulated plaque burden depending on a person's combination of other exposures. This is accomplished by constructing a lifetable of the absolute instantaneous hazard of experiencing an atherosclerotic cardiovascular event at all levels of cumulative exposure to LDL (plaque burden) at the average levels of other covariates (e.g., all other exposures) in a reference population. This data is derived from naturally randomized experiments using genetic instrumental variable LDL scores. These studies demonstrate that persons randomized by nature to higher lifelong exposure to LDL have a higher measured LDL level, a higher corresponding cumulative exposure to LDL, and a higher absolute cumulative risk of having an atherosclerotic cardiovascular event at all ages as compared to persons randomized by nature to lower LDL. However, when using cumulative exposure to LDL as the increment of follow-up from the time of randomization, each of these groups has the same absolute cumulative risk of atherosclerotic cardiovascular events at the same cumulative exposure to LDL (accumulated plaque size) regardless of the age at which the cumulative exposure to LDL was achieved. This naturally randomized biological evidence demonstrates that the absolute risk of atherosclerotic cardiovascular events is determined by the size of the accumulated plaque burden (measured in cumulative exposure to LDL), regardless of how rapidly the plaque burden is accumulating when all other exposures are equal. The results of these naturally randomized trials also provides the motivation, evidence, and justification for using cumulative exposure to LDL as the increment of follow-up in the Survival DNN with PEM described above.

The absolute instantaneous hazard of having an atherosclerotic event for an individual person at each level of cumulative exposure to LDL (accumulated plaque burden) is then estimated by multiplying the instantaneous hazard rate in the reference group by the log hazard ratio derived from the DNN with PEM estimating how much a person's unique combination of exposures impacts the risk of having an atherosclerotic cardiovascular event at each level of cumulative exposure to LDL (accumulated plaque burden). The absolute instantaneous hazards are then summed to compute the cumulative hazard and absolute cumulative event rates at each level of cumulative exposure to LDL (plaque burden) for the person under evaluation conditional on surviving without an event up to that level of plaque burden.

Next, the personal plaque threshold at which atherosclerotic cardiovascular events are predicted to occur for the person under evaluation is computed by plotting the predicted absolute cumulative event rates by cumulative exposure to LDL. This plot identifies the cumulative exposure to LDL corresponding to the size of the accumulated plaque burden at which atherosclerotic cardiovascular events are predicted to begin to occur for the person under study. This level of cumulative exposure to LDL at which the size of the accumulated atherosclerotic plaque burden exceeds the threshold size at which atherosclerotic cardiovascular events begin to occur for a specific person is their ‘personal plaque threshold’. It follows that this plot can also be used to identify the personal plaque threshold for any level of cumulative lifetime risk of atherosclerotic cardiovascular events by identifying the cumulative exposure to LDL and the corresponding size of the accumulated plaque burden at the any specified level of cumulative lifetime risk. The cumulative exposure to LDL corresponding to a specific cumulative lifetime risk of cardiovascular events can be used as a therapeutic target to personalize the prevention of cardiovascular events by providing guidance about how much a person's LDL level must be reduced to slow their rate of plaque progression enough to keep their cumulative exposure to LDL and the corresponding size of their accumulated plaque burden below the threshold required to achieve the desired level of cumulative lifetime risk.

Next, the predicted absolute instantaneous hazard, cumulative hazard, and cumulative event rates at each age for the person under consideration are computed by transposing the lifetable of ‘risk by cumulative exposure to LDL’ into a lifetable of ‘risk by age’. This transposition is accomplished by mapping the age at which each corresponding level of cumulative exposure to LDL occurs for the person under evaluation using the output from the LDL Bi-LSTM predicting the person's trajectory of LDL levels over time. The lifetable of risk by age provides the predicted absolute instantaneous hazard, cumulative hazard, and cumulative event rates at each age for the person under consideration based on the size of the accumulated plaque burden (cumulative exposure to LDL), their rate of plaque progression, and their combination of other exposures. The creation of a lifetable of absolute hazards by age provides a mechanism to communicate risk in a clinically useful way, compute updated predictions of remaining lifetime risk over time as a person survives previous risk intervals without experiencing an event, compute the benefit of reducing exposure to the modifiable causes of disease by both magnitude, duration, and timing of intervention, and catalogue and quantify the legacy benefit derived from earlier interventions to reduce exposure to the modifiable causes of disease, thus preventing catastrophic forgetting of the benefit of earlier interventions, to accurately predict remaining lifetime risk and benefit, and to guide the appropriate selection of optimal sequence of interventions required to achieve the desired therapeutic goals, accounting for the accruing benefit of earlier interventions to reduce exposure to the modifiable causes of disease.

The results of the Survival DNN with PEM using cumulative exposure to LDL, combined with the lifetable analyses informed by the results of the naturally randomized trials of cumulative exposure to LDL, provide the predicted risk of developing an atherosclerotic cardiovascular event based on the size of a person's accumulated plaque burden, and their combination of other exposures that impact the capacity of the artery to tolerate the accumulated plaque burden, at all future time points up to any age. In addition, simple linear functions provide predictions for the risk of developing hypertension based on the current level of SBP and the predicted rate of rise in SBP over time, including the predicted age at which hypertension is likely to occur (e.g., by comparing predicted SBP to a predetermined threshold), and the risk of developing T2D based on the predicted increase in HbA1c in response to excess energy induced weight gain or increases in adiposity, including the personal weight threshold at which a person's HbA1c level is likely to exceed the threshold for pre-diabetes or T2D.

### Predicting the Benefit of Interventions Designed to Reduce the Risk of Cardiometabolic Disease

Predicting the benefit of interventions designed to reduce the risk of cardiometabolic disease involves predicting the expected proportional and absolute reductions in the risk of atherosclerotic cardiovascular events in response to interventions designed to lower LDL, SBP, or both, which involves two distinct steps. First, a Causal Deep Neural Networks for Ordinary Differential Equations (c-DNN-ODE) is pre-trained exclusively on causal data from randomized trials of LDL and SBP lowering therapies, respectively, and Mendelian randomization studies with individual participant follow-up data evaluating genetic variants associated with lower LDL (apoB) or SBP, respectively. A c-DNN-ODE combines a deep neural network to estimate a function describing a smooth curve that can be used to compute the instantaneous hazard ratio for a standard increment of lower LDL (1 mg/dL) or lower SBP (1 mmHg) during each interval of treatment over time (without the need to specify the closed form of the function). The DNN is combined with an ordinary differential equation solver to estimate how much the benefit of lowering LDL or SBP changes during infinitesimally small increments of increasing duration of treatment. The c-DNN-ODE thus transforms the idea of passing data through a fixed sequence of layers in a neural network into a continuous process of transformations between layers where data evolves smoothly over time (just like a system governed by a differential equation), and therefore can used to be efficiently quantify how much the magnitude of the instantaneous hazard ratio for an increment of lower LDL or SBP changes over time, where the hazard ratio is a quantitative estimate of the expected proportional clinical benefit of lowering LDL or SBP.

This solution demonstrates that the results of randomized trials can be reanalyzed by constructing a lifetable to recover the instantaneous hazards for the desired outcome event (or composite event) in either treatment arm at every interval of follow-up. Recovery of the instantaneous hazards permits the computation of the corresponding instantaneous hazard ratio within each increment of follow-up. Combining the observed instantaneous hazard ratios during each interval of follow-up with the corresponding absolute difference in LDL or SBP between the randomized treatment arms observed during the same increment of follow-up permits computation of the instantaneous hazard ratio per unit lower LDL, or per unit lower SBP, during each increment of follow-up during the trial. Combining the estimated hazard ratio per unit lower LDL, or per unit lower SBP, during each increment of follow-up from multiple randomized trials in an inverse variance-weighted meta-analysis provides a robust estimate of the magnitude of the instantaneous hazard ratio per unit lower LDL, or per unit lower SBP, during each treatment interval. Plotting the magnitude of the summary hazard ratio during each interval of follow-up provides a visual and quantitative assessment of how the hazard ratio changes over time, thus providing a direct estimate of how much the benefit of lowering LDL or SBP increases over time.

Extending this same intuition to the analysis of nature's randomized trials using individual participant data from Mendelian randomization studies evaluating genetic variants associated with lower LDL (apoB) or SBP, respectively, permits the construction of lifetables containing the instantaneous hazards and instantaneous hazard ratios during each year of life (or other interval of follow-up) among participants randomized by nature to lifelong exposure to lower LDL or lower SBP, respectively, beginning at birth until age 80 years of age. Combining the observed instantaneous hazard ratios during each interval of follow-up with the corresponding absolute difference in LDL or SBP observed between the groups randomized by nature to higher or lower LDL or SBP, respectively, during the same increment of follow-up, permits computation of the instantaneous hazard ratio per unit lower LDL, or per unit lower SBP, during each year of life. Combining the results of numerous different Mendelian randomization studies evaluating hundreds of different genetic variants associated with lower LDL or lower SBP, respectively, in an inverse variance-weighted meta-analysis provides a more robust estimate of the magnitude of the hazard ratio per unit lower LDL, or per unit lower SBP, during each year of life (or other interval of follow-up) among participants randomized by nature to lifelong exposure to lower LDL or lower SBP, respectively.

The c-DNN-ODE takes as input the estimated instantaneous hazard ratios ordered by duration of follow-up from the analyses of both the randomized trials and Mendelian randomization studies and processes this data to differentiate a continuous series of infinitesimally small increments of increasing follow-up time to provide a continuous estimate of the magnitude of the instantaneous hazard ratio for a one-unit increment of lower LDL, or lower SBP, for any duration of intervention. Finally, the time-averaged hazard ratio for a one-unit lower LDL, or SBP, for all durations of sustained intervention of follow-up is computed by iteratively calculating the weighted average of the c-DNN-ODE estimated instantaneous hazards in sequence from the start of therapy.

Second, the expected benefit of lowering LDL or SBP over any time interval for the individual person under consideration is computed as follows. The predicted instantaneous hazard of experiencing an atherosclerotic event during each interval of follow-up from the current age to age 80 years in the lifetable of risk by age constructed for that person using the outputs from the Bi-LSTM and Survival DNN with PEM using cumulative LDL as the increment of analysis is multiplied by the c-DNN-ODE estimated time-averaged instantaneous log hazard ratio for a one-unit lower LDL or SBP corresponding to the duration of treatment at that interval of follow-up (or age), and adjusted for the expected absolute magnitude of the reduction in LDL or SBP in response to the recommended intervention using the usual Wald ratio of effect estimates method. The estimated cumulative hazard of experiencing an atherosclerotic cardiovascular event, and the corresponding cumulative event rate, at each age of follow-up, conditional on surviving to an interval without experiencing an event, is then computed using standard lifetable analysis of the treatment adjusted predictions. Finally, the expected clinical benefit of lowering LDL or SBP for the individual person under consideration is computed as follows. The predicted proportional reduction in the risk of experiencing an atherosclerotic cardiovascular event at any duration of follow-up is computed as the ratio of the predicted cumulative hazard at that duration of follow-up (age) adjusted for the magnitude and duration of LDL or SBP lowering, to the expected cumulative hazard without intervention to lower LDL or SBP. And the predicted absolute reduction in the risk of atherosclerotic events is computed as the absolute difference between the predicted absolute cumulative event rate at a specific duration of follow-up (age) adjusted for the magnitude and duration of LDL or SBP lowering, to the expected absolute cumulative event rate at the same duration of follow-up without intervention to lower LDL or SBP.

Next, the expected benefit from all possible combinations and sequences of interventions to lower LDL, SBP, or both for the individual person under consideration is computed. This step informs selection of the optimal timing and sequence of interventions used to personalize the prevention of atherosclerotic cardiovascular events because both the proportional and absolute reductions in cardiovascular events expected in response to any specific intervention or combination of interventions is determined by the magnitude, duration, and timing of lowering LDL and SBP. The expected benefit for all possible sequences of interventions to lower LDL, SBP, or both is computed as follows. First, the lifetable predicting the instantaneous hazard, cumulative hazard, and cumulative rate of atherosclerotic cardiovascular events from birth to age 80 years for the person under consideration is imported from the previous steps. Second, seven additional columns are added to this lifetable: a column for the expected plasma LDL level at each age from birth until age 80 based on the predicted trajectory of LDL using the forward and backward passes of the LDL Bi-LSTM, which is adjusted for the intensity of LDL lowering when an LDL lowering intervention is being evaluated; a column for the predicted cumulative exposure to LDL to estimate the size of the accumulated plaque burden at each age from birth until age 80 computed by summing the predicted LDL level at each age for the person under study, which provides a running assessment of how close a person is to their personal plaque threshold above which atherosclerotic cardiovascular events begin to occur and is adjusted for the intensity of LDL lowering when predicting the benefit of adding an LDL lowering therapy; a column for the measured or predicted SBP at each age from the current age until age 80 derived from the forward pass of the SBP Bi-LSTM for the person under consideration, which provides a running assessment of the age at which the predicted SBP is likely to exceed 130 mmHg, and to exceed 140 mmHg (or the therapeutic threshold for hypertension); a column for the measured or predicted weight at each age from the current age until age 80 for the person under consideration computed using the simplifying assumption that the person's current age and sex adjusted weight percentile remains constant throughout life, which is replaced by more precise predictions when the module for preventing obesity and T2D diabetes is added; a column for the measured or predicted plasma HbA1c level at each age from the current age until age 80 for the person under consideration computed using the simplifying assumption that the person's current age and sex adjusted HbA1c percentile remains constant throughout life, which by combining the two simplifying assumptions regarding the trajectory of weight and HbA1c for each person, provides a running assessment of the predicted weight at which the HbA1c level is likely to exceed 5.7% (pre-diabetes) and 6.5% (diabetes) thus providing a rough estimate of the personal weight threshold for developing diabetes, with more precise predictions for the individual trajectory of weight gain, the predicted sensitivity to weight gain (excess energy) induced increases in HbA1c, and the personal weight threshold for developing diabetes provided when the module for preventing obesity and T2D diabetes is added; a column for treatment to lower LDL, quantified by the expected proportional reduction in LDL from the intensity of the selected intervention, where the predicted proportional reduction in LDL from the selected treatment is also multiplied by the predicted or measured LDL level at each age that the intervention is administered to adjust the values in the columns for the predicted LDL and cumulative LDL during LDL lowering therapy; and a column for treatment to lower SBP, quantified by the expected proportional reduction in SBP from the intensity of the selected intervention, where the predicted proportional reduction in SBP from the selected treatment is also multiplied by the predicted or measured SBP level at each age that the intervention is administered to the adjust the values in the column for the predicted SBP during SBP lowering therapy.

Third, to limit the number of potential combinations and sequences of LDL and SBP lowering therapies to be evaluated, a domain expert clinical translation heuristic is invoked. Only sequences that continue the current intervention, intensify the current intervention, or add another intervention are considered. This domain expert clinical translation heuristic eliminates consideration of discontinuous intervention sequences that stop and start interventions in random order, which are not clinically plausible.

Fourth, the predicted proportional and absolute clinical benefit for all clinically plausible combinations and sequences of interventions at all ages extending from the current until age 80 years are then systematically computed as follows. An intervention to lower LDL is selected, and the expected proportional reduction in the causal exposure is noted (e.g., a 36% time-averaged reduction in LDL expected in response to the intervention). All sequences begin with an intervention to lower LDL because the initial therapeutic choice always includes lowering LDL to slow the progression of atherosclerosis because cumulative exposure to LDL determines the size of the accumulated plaque burden, and the size of the accumulated plaque burden is the strongest cause of atherosclerotic cardiovascular events, and because the decision to lower SBP is intended to lower the risk of atherosclerotic cardiovascular events, and therefore reduction in risk achieved by lowering SBP can always be enhanced by also lowering LDL at the same time. The age at which the intervention is to be started is selected (e.g., the current age). Once the intervention is started, it is assumed that it will continue uninterrupted until age 80 years. At each age that the selected intervention is administered, the predicted instantaneous hazard for experiencing an atherosclerotic cardiovascular event during that age interval for the person under consideration is multiplied by the time-averaged log hazard ratio for a one unit reduction in the target of the intervention based on the duration of treatment derived from the c-DNN-ODE, adjusted for the expected absolute reduction in the targeted modifiable cause of disease in response to the intensity of the selected intervention. The intervention adjusted instantaneous hazards during each year of life are then combined to compute the cumulative hazard and cumulative rate of atherosclerotic events at each age from the current age until age 80 years for the person under consideration. The result of these computations provide the predicted absolute cumulative event rate at every age, extending from the current age until age 80, with and without the selected treatment initiated at the selected age. The corresponding predicted proportional and absolute reductions in the rate of atherosclerotic cardiovascular events at every age and corresponding duration of intervention is then computed by comparing the treatment adjusted and unadjusted predicted cumulative hazard rates at each age. Next, the computations are repeated, but starting the same intervention one year later. This process continues iteratively to compute the expected clinical benefit of starting the selected treatment at every age extending from the current age until age 80 years. Once the evaluation of the selected intervention beginning at each year of life from the current age until age 80 years is exhausted, the predicted benefit of intensifying the current treatment (e.g., intensifying treatment to produce an expected 50% time-averaged reduction in LDL) beginning at each age is computed following the same protocol. Finally, the clinical benefit of adding a SBP lowering therapy (e.g., to produce a time-averaged 10% reduction in SBP) beginning at different ages for each of the sequences of LDL lowering is computed. The combined effect of lowering LDL and SBP during an increment of time is computed as follows. The predicted instantaneous hazard for experiencing an atherosclerotic cardiovascular event during that age interval for the person under consideration is multiplied by the time-averaged log hazard ratio for a one-unit reduction in LDL based on the duration of treatment derived from the LDL-specific c-DNN-ODE, adjusted for the expected absolute reduction in LDL in response to the intensity of the selected intervention. This quantity is then multiplied by the time-averaged log hazard ratio for a one-unit reduction in SBP based on the duration of treatment derived from the SBP-specific c-DNN-ODE, adjusted for the expected absolute reduction in SBP in response to the intensity of the selected intervention. This produces an estimate of the predicted instantaneous hazard of experiencing an atherosclerotic cardiovascular during the age interval, conditional on the magnitude and duration of LDL and SBP reduction experienced during that age interval. The combined LDL and SBP lowering intervention adjusted instantaneous hazard during each year of life are then combined to compute the cumulative hazard and cumulative rate of atherosclerotic events at each age from the current age until age 80 years for the person under consideration. The result of these computations provides the predicted absolute cumulative event rate at every age, extending from the current age until age 80, with and without the selected combination and sequence of interventions initiated at the selected age(s). The corresponding predicted proportional and absolute reductions in the rate of atherosclerotic cardiovascular events at every age for the timing and sequence of interventions is then computed by comparing the treatment adjusted and unadjusted predicted cumulative hazard rates at each age.

Next, the predicted absolute cumulative rate of atherosclerotic cardiovascular events at each age in response to a sequence of interventions to lower LDL, SBP, or both, for the person under consideration is stored in a vector. These predicted event rate vectors are then iteratively combined to construct a Markov Decision Process (MDP) matrix. This MDP matrix represents the potential solution space for selecting the optimal timing and sequence of interventions for each person to personalize the prevention of cardiometabolic disease, conditional on using a sequence of interventions to lower LDL, SBP, (weight, and HbA1c) that satisfies the domain expert heuristic for clinical translation.

The expected proportional reduction in the risk of atherosclerotic cardiovascular events during each year of a therapy to lower LDL, SBP, or both is estimated from a c-DNN-ODE trained exclusively on causal evidence from randomized trials and Mendelian randomization studies divided into discrete time-units of follow-up. The predicted benefit of lowering LDL, SBP, or both beginning at any age and extending for any duration for the person under consideration is then computed by combining these time-dependent estimates of proportional benefit with the predicted hazard of experiencing an atherosclerotic cardiovascular event during each increment of follow-up without intervention. The predicted benefits for all possible sequences of lowering LDL, SBP or both depending on the magnitude, duration, and timing of those interventions are then stored in an MDP solution state matrix to facilitate identification of the optimal timing and sequence of interventions to personalize the prevention of cardiometabolic disease for the person under consideration.

### Discovering the Optimal Sequence of Interventions to Personalize the Prevention of Cardiometabolic Disease

Discovering the optimal sequence of interventions to personalize the prevention of cardiometabolic disease involves passing the MDP solution state matrix (composed of vectors containing the predicted rate of atherosclerotic cardiovascular events at every age for all possible sequences of interventions to lower LDL, SBP, or both) as an input into a Reinforcement Learning (RL) Model Dependent Proximal Policy Optimization (MD-PPO) algorithm with Monte Carlo Tree Search (MCTS) constrained by a domain expert defined Optimal Global Policy Guideline. The reinforcement learning algorithm is designed to discover the optimal sequence of interventions for each person by using Model Dependence, where the RL algorithm relies on the Deep Causal AI Agentic series of deep and machine learning algorithms to quantify the predicted absolute event rates and corresponding predicted clinical benefit at all future ages and for all possible sequences of interventions to lower LDL, SBP, or both. These algorithms are designed to encode biological cause and effect and have been trained on randomized evidence to learn how cardiometabolic diseases develop over time, and how to use this information to predict the expected clinical benefit of interventions based on the magnitude, duration, and timing of lowering LDL, SBP, or both.

The algorithm employs Proximal Policy Optimization, where the first action in the optimal sequence of actions that achieves the desired goal of personalizing the prevention of cardiometabolic disease for the person under consideration is the optimal proximal policy (or optimal clinical strategy). The objective of the RL algorithm is to discover first step in the optimal sequence of actions to personalize the prevention of cardiometabolic disease, and then use the observed reductions in the targeted cause of disease achieved in response to the recommended action in combination with the biological changes in other exposures not targeted by the recommended action to learn how the person's cardiometabolic health is evolving over time, and then use this information adjust the estimates of risk and benefit to either reinforce the recommended next action in the previously identified optimal sequence of interventions or adjust the recommended next action in the updated optimal sequence of interventions used to achieve the desired goal.

Markov Chain Tree Search is employed because the number of possible sequences of interventions to lower LDL, SBP, or both that must be evaluated depends on the number and type of interventions being considered for recommendation, the possible dosages or intensities of the interventions being considered, and the age at which the first intervention is started. The search for the optimal sequence of interventions in the total solution space can proceed either comprehensively by brute force if the total number of possible sequences of interventions is limited, or randomly using a Monte Carlo Tree Search algorithm if the number of potential possible sequences of interventions creates a solution space that is too vast for an exhaustive search. To create a more computationally feasible solution space, a MCTS algorithm can be used to randomly generate a large number of possible sequences of interventions to lower LDL, SBP, or both for consideration (constrained by the same domain expert clinical translation heuristic as in previous steps).

The Domain Expert Optimal Policy Guideline serves as the overall objective of the RL MD-PPO algorithm to discover the optimal sequence of interventions to personalize the prevention of cardiometabolic disease for the person under consideration. However, the explicit parameters defining this goal are customizable. The Domain Expert Optimal Policy Guideline introduces a mechanism to ‘inject clinical expertise’ into the RL algorithm to ensure that the recommended sequence of actions achieves an explicitly defined overall goal that is consistent with personal preferences, local clinical practice guidelines, or other clinical or policy objective. The default parameters of the Domain Expert Optimal Policy Guideline are set as follows (but can be fully customized). Defining the Prevention of Cardiometabolic Disease includes maintaining a cumulative lifetime risk of experiencing an atherosclerotic cardiovascular event of less than 5% at all ages up to age 80 years (both the desired cumulative event rate threshold and the age or duration of follow-up are fully customizable to permit consideration of both short-term and long-term benefits and different intensities of short-term or long-term therapeutic goals), preventing the development of hypertension (with a customizable SBP threshold for the diagnosis of hypertension), preventing the development of T2D (with a customizable HbA1c threshold to initiate interventions to either lower, or prevent further rises, in HbA1c), and lowering LDL, SBP, weight, and HbA1c by just the amount each person needs when they need it to personalize the prevention of atherosclerotic cardiovascular events (including MI and stroke), hypertension, and T2D using the fewest total intervention units possible (to maximize clinical and economic return on investment).

Prioritization of initial intervention to achieve desired goal involves lowering LDL by the amount used to slow the rate of plaque progression enough to keep the predicted size of the accumulated plaque burden at age 80 years below the selected personal plaque threshold (i.e. the cumulative exposure to LDL at which the cumulative lifetime risk of atherosclerotic cardiovascular events is predicted to reach 5% for the person under consideration). This personal plaque threshold, measured in cumulative exposure to LDL (Plaque Years of LDL in mmol/L) used to keep the cumulative lifetime risk of atherosclerotic cardiovascular events below 5%, is conditional on the person's current LDL level, the existing size of their accumulated plaque burden (defined by their current cumulative exposure to LDL), their predicted rate of plaque progression, and their exposure to other features that reduce the capacity of the artery wall to tolerate the accumulated plaque burden.

Prioritization of subsequent intervention(s) used to achieve a desired goal includes adding a SBP lowering intervention when useful for maintaining the remaining lifetime risk of atherosclerotic events below the selected threshold of 5% at all ages up to age 80 years, by protecting the artery wall from additional accumulating irreversible structural injury caused by elevated SBP, or when SBP exceeds 130 mmHg to prevent further rises in SBP and thus prevent the development of hypertension, whichever comes first, thus ensuring the prevention of both atherosclerotic cardiovascular events and hypertension. It also includes adding a Nutrient Stimulated Hormone (NuSH), or other intervention, to prevent further weight gain, or increased adiposity, by preventing further excess energy balance to thus prevent further rises in HbA1c when useful for maintaining the remaining lifetime risk of atherosclerotic events below the selected threshold of 5% at all ages up to age 80 years, by protecting the artery wall from additional accumulating irreversible structural injury caused by elevated circulating glucose levels, or when HbA1c levels exceeds 5.7% to 6.0% (depending on age) to prevent further rises in HbA1c and thus prevent the development of T2D, whichever comes first, thus ensuring the prevention of both atherosclerotic cardiovascular events and T2D. Although the default settings of the Domain Expert Optimal Policy Guideline focus on lowering LDL, SBP, weight, and HbA1c, it can be easily extended to include additional interventions that reduce Lp(a), including siRNA directed against LPA, and other interventions (including therapies directed against triglyceride-rich apoB-containing lipoproteins, as well as the effects of diet, and exercise).

The Value Function for Selecting Optimal Sequence of Interventions ensures that each sequence of interventions that achieves the overall objective of personalizing the prevention cardiometabolic disease for the person under consideration as defined by the Domain Expert Optimal Policy Guideline (i.e. maintaining a cumulative lifetime risk of atherosclerotic events less 5% by age 80 years while also preventing the development of hypertension and T2D) is then quantitatively scored and ranked using a Value Function. The sequence of actions with the highest ranked Penalized and Discounted Value Function is selected as the recommended optimal sequence of actions to personalize the prevention of cardiometabolic disease for the person under consideration. The Value Function is computed as follows. The predicted cumulative hazard of experiencing an atherosclerotic cardiovascular event at all ages between the current age and 80 years are summed (or integrated to produce the area under the cumulative event curve). The objective is to minimize this value and therefore minimize the cumulative lifetime risk of having an atherosclerotic event. A Penalty is added for each intervention that is used during every year that the intervention is used. The penalty is designed to achieve the goal of lowering LDL, SBP, weight, and HbA1c by only the amount that each person needs, when they it, to achieve the desired goal using the fewest number of treatments. The penalty is also designed to prioritize the use of combination therapies (which are penalized as a single unit) and therapies that ensure compliance (e.g., ultra-long acting siRNA) and thus produce the maximum absolute reduction in the targeted modifiable cause of disease and greatest corresponding reductions in clinical events. In addition, the penalty is designed to prevent the MD-PPO from learning to select the sequence of interventions defined by lowering LDL, SBP, weight, and HbA1c by the maximum amount beginning at the current age and extending until age 80 years. Although this sequence of interventions will produce the greatest proportional and absolute reductions in the risk of atherosclerotic cardiovascular events while preventing hypertension and T2D, it would not achieve the goal of lowering LDL, SBP, weight, and HbA1c by only the amount needed and only when needed to personalize prevention for the person under consideration, and it would represent the sequence of actions that consumes the maximum amount of resources, thus reducing the economic return on investment. A Discounting parameter is also added to the Value Function, and is designed to prioritize preventing the development of cardiometabolic disease early in life because early onset of disease has the greatest clinical and economic costs. In addition, the discounting parameter is designed to prioritize early interventions that produce modest, sustained reductions in LDL over time to slow the progression of atherosclerosis and thus minimize the residual risk of cardiovascular events caused by the accumulated plaque burden present at any point in time, rather than more aggressive LDL lowering, and combinations of LDL and SBP interventions, started later in life, because this strategy allows atherosclerotic plaque and arterial wall injury to accumulate early in life, leading to a larger plaque burden, and more irreversible structural injury to the artery wall accumulating early in life before therapy is initiated thus leading to a higher residual risk and a corresponding higher absolute rate of events that occur earlier in life, even though the total cumulative event rate by age 80 years may be very similar (because more aggressive later treatment prevents fewer early events but a greater number of later events, as compared to modest early sustained reductions in LDL).

The MD-PPO with MCTS reinforcement learning algorithm constrained by a domain expert defined Optimal Global Policy Guideline to discover the optimal sequence of interventions for each person to personalize the prevention of cardiometabolic disease that focuses on reducing LDL, SBP, weight, and HbA1c by the amount needed when needed to personalize the prevention of heart attack, stroke, hypertension, and diabetes using the fewest number of interventions possible.

### Recommended Action, Reward, Updated State, Updated Analysis, and Recommended Next Action

The output of the Deep Causal AI Agent is twofold: a narrative explaining the reasoning and biological rationale explaining why a person is at risk cardiometabolic disease, how they can minimize their risk, how much they will benefit from specific actions to minimize risk, and the recommended optimal sequence of actions used to prevent cardiometabolic disease; and recommended immediate action (which can include observation) to personalize the prevention of cardiometabolic disease, with a description of the expected subsequent actions needed and when they are likely to be needed based on the person's predicted current cardiometabolic health trajectory.

The realized reward of the recommended action is determined by the actual achieved absolute reduction in exposure to the modifiable cause of disease targeted by the recommended intervention, and the corresponding predicted reduction in the risk of cardiometabolic disease determined by the achieved absolute reduction in the modifiable cause of disease over each subsequent time interval, including the expected reduction in the risk of cardiometabolic disease over the next 1 year, 2 years, 5 years, 10 years, 20 years, remaining lifetime, or any other interval of interest. Because the magnitude of the reward or expected clinical benefit is quantified by the absolute reduction in the modifiable cause of disease achieved in response to the recommended intervention(s) or sequence of interventions, the realized reward or clinical benefit depends on compliance with the recommended intervention(s) and how well the intervention(s) are implemented. This objective quantification of the reward function distinguishes the expected absolute reduction in the modifiable cause of disease and corresponding expected proportional reduction in the risk of cardiometabolic disease that was used to inform selection of the recommended action, with the achieved absolute reduction and corresponding expected proportional reduction from the achieved reduction, which is used as the metric of success for the recommended action.

The updated state of cardiometabolic health is determined by two dynamic processes: the achieved absolute reduction in the modifiable cause of disease targeted by the recommended intervention, and the absolute changes in other exposures not targeted by the recommended interventions(s) due to aging and the evolving biology of how common diseases develop during the same interval of longitudinal follow-up. The updated values of a person's clinical characteristics, physical biometrics, and biochemical measurements are then passed back into the Deep Causal AI Agent to provide an updated assessment of their current state of cardiometabolic health; an updated prediction of the risk of cardiometabolic disease over all subsequent time intervals based on the updated state of cardiometabolic health and the reductions in the modifiable causes of disease achieved in response to prior interventions; an updated prediction of the expected benefit of interventions designed to reduce exposure to the modifiable causes of disease based on the updated state of cardiometabolic health and the reductions in the modifiable causes of disease achieved in response to prior interventions, including consideration of the legacy benefit from the achieved absolute reductions in the modifiable causes of disease in response to earlier interventions; and updated recommendations about the optimal timing, type, intensity, combination, and sequence of actions used to prevent MI, stroke, hypertension, and T2D.

The output of the Deep Causal AI Agent includes an updated narrative explaining the reasoning and biological rationale for why a person is at risk, how their cardiometabolic health is evolving, how to reduce their risk of cardiometabolic disease, how much they have benefited from previous actions, how much they would benefit from subsequent actions to prevent disease, and updated recommendations for the optimal sequence of actions used to prevent cardiometabolic disease. The updated recommendation for the optimal next immediate action could include continuing the current intervention, intensifying the current intervention, or adding another intervention. This process repeats iteratively at regular follow-up intervals to monitor cardiometabolic health, and adjust guidance about the optimal recommended actions and sequence of actions used to prevent MI, stroke, hypertension, and T2D based on the person's evolving cardiometabolic health and achieved reductions in the modifiable causes of disease in response to the recommended actions.

### Example 2

This example provides examples of various algorithms part of the techniques described herein.

### 1. Standardization of Measurement Values Included in the Dense Input Feature Vector

Raw measurements for the clinical, physical, and biochemical measurements used to characterize the current state of cardiometabolic health vary widely in magnitude and are measured on different scales.
Therefore, the measured input values are first standardized to:
   Improve computational efficiency;
   Stabilize gradient flow, and prevent exploding or vanishing gradients;
   Prevent bias toward features with larger numeric ranges;
Numerous standardization methods are available. One method that is particularly useful when input features are naturally bounded (such as biological data) is Min-Max Scaling (Normalization) calculated using the formula

$${x}^{′} = \frac{x - {x}_{min}}{{x}_{max} - {x}_{min}}$$

where: x is the original feature value, and xₘᵢₙ and xₘₐₓ are the minimum and maximum values of the feature.

### 2. Positional Encoding of Dense Input Feature Values by Age.

The interpretation of the absolute value of a measured biological parameter can vary substantially depending on biological context, including age, at which the parameter was measured.
   For example, a SBP of 130 mmHg in a 25 year old man is 10 mmHg above the age and sex adjusted population median SBP level of 115 mmHg.
   By contrast, the same SBP level of 130 mmHg in a 65 year old man is 15 mmHg below the age and sex adjusted population median SBP level of 145 mmHg.
   Thus, the same SBP reading can have very different biological and physiological implications depending on the age and sex of the person being evaluated.
Positional encoding can be added to the input features to provide a sense of the timing within the a person's evolving health, biological context, or age at which the feature was measured.
Therefore, to provide biological context for interpreting the value of all the clinical, physical, and biochemical measurements used to characterize the current state of cardiometabolic health, the standardized values are then positionally encoded by age.
   And the positionally encoded standardized values are then appended to the dense input feature vector.
   The original standardized measurements are retained to preserve any information that may be contained in a feature interpreted without regard to the timing during the person's cardiometabolic health trajectory at which the parameter is measured.
Numerous positional encoding methods are available. One method that is particularly useful for positionally encoding by a naturally ordered, evenly spaced, biological variable such as age is positional encoding using sine and cosine calculated as follows:

$$P {E}_{( pos , 2 \text{?} )} = sin ( \frac{pos}{{10000}^{\frac{\text{?}}{d}}} )$$

$$P {E}_{( pos , 2 \text{?} + 1 )} = cos ( \frac{pos}{{10000}^{\frac{\text{?}}{d}}} )$$

$$\text{?} \text{indicates text missing or illegible when filed}$$

where:
pos is the position in the sequence (age at measurement),
d is the dimension of the positional encoding vector (matching the dimension of the input embeddings),
i is the index of the feature within the positional encoding vector,
for even indices (2ᵢ): Use the sine function to encode the position,
for odd indices (2ᵢ+1): Use the cosine function to encode the position, and
the term 10000^{(2i/t)} defines a different frequency for each dimension.
Once the positional encoding is computed for each position in the input sequence, it is appended to the input feature vectors as follows:

$${𝒳}_{input} - {𝒳}_{embedding} + P E$$

The positional encodings are then added element-wise to the input embeddings.

### 3. Bidirectional Long Short-Term Memory (Bi-LSTM) Algorithms

A Long Short-Term Memory (LSTM) is a type of Recurrent Neural Network (RNN) designed to effectively learn and capture long-term dependencies in sequential data, such as predicting the trajectory of how levels of LDL and SBP change throughout life.
A Bidirectional Long Short-Term Memory (Bi-LSTM) processes input sequences in both forward and backward directions, providing a richer context for considering both past and future information:
   This makes it well-suited for tasks where understanding the full context of a sequence is crucial;
   Such as predicting the future and past levels of LDL or SBP from a single measured value to provide an estimate of total cumulative exposure LDL and SBP, which are engineered features that have crucial biological meaning;

### Training Data:

Individual participant data from 16,235 participants enrolled in one of three (3) long-term prospective cohort studies for whom repeated longitudinal measures of LDL, HDL, SBP, weight, waist circumference, BMI, and HbA1c (or fasting plasma glucose) are available over 25-59 years of follow-up (participants censored at time lost to follow-up, death, or first atherosclerotic cardiovascular events, or initiation of lipid lowering therapy);
Loss function: the customary loss function for a Bi-LSTM is the mean squared error (MSE)

### Calculation of MSE Loss for a Bidirectional LSTM:

### 1. Passing Data Through the Bi-LSTM:

The input sequence data are processed in both directions (forward and backward):
Forward LSTM: Processes the input sequence from the left to right.
Backward LSTM: Processes the input sequence from the right to left.

At each time step t, the forward and backward hidden states [hₜ→ and hₜ←] are concatenated (or summed, depending on implementation) to form the full hidden state hₜ. The combined hidden state is then passed to the next layer or used for prediction during inference.

### 2. Predictions:

After processing the input sequence through the Bi-LSTM layers, the output at each time step is typically passed through a dense (fully connected) layer or a series of layers to produce the final predictions ŷₜ at each time step t

The model outputs a sequence of predictions, one for each time step in the input sequence.

### 3. Calculation of the Squared Differences for Each Time Step:

For each time step t in the sequence, the squared difference between the predicted and measured values is computed as:

$${( {y}_{t} - {\hat{y}}_{t} )}^{2}$$

Where: yₜ is the true value at time step t and ŷₜ is the predicted value at time step t

### 4. Calculation of the Average the Squared Differences Over All Time Steps and All Sequences:

The MSE loss over the entire sequence is calculated by summing the squared errors across all time steps and taking the average:

$$M S E = \frac{1}{T} {\sum }_{r = 1}^{r} {( {y}_{t} - {\hat{y}}_{t} )}^{2}$$

where Tis the total number of time steps in the sequence.

### 5. Backpropagation and Gradient Descent:

Once the MSE loss is computed, backpropagation is used to calculate the gradients of the loss with respect to the model parameters (LSTM weights, biases, etc.). These gradients are then used to update the model parameters using an optimization algorithm such as stochastic gradient descent (SGD) or the Adam Optimizer.
The LSTM models can be implemented using PyTorch's native libraries, which provide direct support with well-optimized functions for LSTM, including:
   torch.nn—The module includes nn.LSTM for implementing the LSTM layer, as well as nn.LSTMCell for finer control over LSTM cell computations. Additional modules like nn.Linear are used to map the LSTM outputs to desired output dimensions.
   torch.optim—For defining optimization algorithms, such as Adam or SGD, to train the model.
   torch.utils.data—This is useful for managing time-series datasets and implementing custom data loaders or transformers.
      4. Survival Deep Neural Network with Piecemeal Exponential Modeling
A Survival Deep Neural Network with a Piecewise Exponential Model (Survival DNN with PEM) is a neural network designed for survival analysis, which involves predicting the time until an event occurs (in this case, an atherosclerotic cardiovascular event including MI or stroke). Unlike standard regression, survival analysis must account for censored data, where the event of interest has not occurred for some individuals by the end of the observation period.
In a PEM, the follow-up is divided into intervals, and within each interval, the hazard rate (the rate at which the event occurs during that interval) is assumed to be constant (the exponential assumption).
For this technology, the follow-up is divided into intervals of cumulative exposure to LDL to estimate the size of the accumulated plaque burden.
The survival DNN model is designed to predict the log hazard ratio (Log HR) during each follow-up interval, i.e., at each level of size of the accumulated plaque burden measured in Plaque Years (mml/L) of cumulative exposure to LDL.
The piecewise exponential model discretizes the time-to-event problem into intervals, allowing for flexible modeling of the hazard ratios over time, assuming only that the hazard rate is constant during each growing interval of plaque burden (interval of cumulative exposure to LDL), but may vary at different levels of plaque burden. This reflects biology of atherosclerosis, where events begin to occur only after a specific accumulated plaque burden size accrues.
Training data: Individual participant data from 1,623,491 participants enrolled in one of three (3) long-term prospective biobank cohort studies for whom at least one LDL or SBP measurement was available, and for whom medical records were available recording age (date) at which the first episode of a fatal or non-fatal MI, fatal or non-fatal ischemic stroke, or coronary revascularization occurred. Participants were censored at time of last follow-up, death, or first atherosclerotic cardiovascular event. In addition, summary data from 2.6 million participants with LDL measurements and age (dates) of first atherosclerotic cardiovascular event were used for additional external validation experiments.
Loss function: the customary loss function for a Survival DNN with PEM estimating the log HR during is the negative log-likelihood, as described below.
   Method for Calculating the Negative Log-Likelihood when Predicting Log HR:

In this implementation, the Survival DNN with PEM predicts the log hazard ratio (Log HRᵢ) for individual i based on their covariates; during each of K intervals (of cumulative exposure to LDL or equivalently at each measured size of the accumulated plaque burden).

The hazard rate λ_{k,i} for the i-th participant in the K-th follow-up interval is:

$${λ}_{k , i} = {λ}_{k} \cdot exp ( log ( H {R}_{i} ) ) = {λ}_{k} \cdot H {R}_{i}$$

where:
λₖ is the baseline hazard rate for the K-th time interval,
log(HRᵢ) is the predicted log hazard ratio for individual i, and
HRᵢ is the hazard ratio obtained by exponentiating the predicted log HR.

### 1. For Uncensored Data (Event Occurs):

For an individual i who experiences the event at time tᵢ (in interval kᵢ), the log-likelihood contribution is based on:
   The predicted hazard ratio HRᵢ multiplied by the baseline hazard λₖ
   The cumulative hazard up to that time
The log-likelihood for uncensored data is:

$$log {L}_{i} = log ( {λ}_{{k}_{i}} \cdot H {R}_{i} ) - {\sum }_{k = 1}^{{k}_{i}} {λ}_{k} \cdot H {R}_{i} \cdot Δ {t}_{k}$$

where:
HRᵢ=exp(log(HRᵢ)) is the predicted hazard ratio for individual i
λ_{k,i} is the baseline hazard in interval Kᵢ
Δtₖ is the length of interval K
The first term represents the likelihood of the event occurring at time tᵢ,
The second term is the cumulative hazard up to tᵢ, derived from the hazard in each interval

### 2. For Censored Data (Event Does Not Occur):

For censored individuals, the event has not occurred, so the likelihood of surviving beyond the censoring time is calculated. The log-likelihood contribution is based on the survival probability, which is related to the cumulative hazard:

$$log {L}_{i} = {\sum }_{k = 1}^{{k}_{i}} {λ}_{k} \cdot H {R}_{i} \cdot Δ {t}_{k}$$

This represents the survival probability up to the censoring time, calculated using the cumulative hazard.

### 3. Overall Log-Likelihood:

The total log-likelihood for the entire dataset is the sum of the log-likelihood contributions from all individuals, combining both uncensored and censored cases:

$$Log - Likelihood = {\sum }_{i = 1}^{N} log {L}_{i}$$

where N is the number of participants. The loss function is the negative log-likelihood

$$Loss := - {\sum }_{i = 1}^{N} log {L}_{i}$$

Once the negative log-likelihood loss is computed, backpropagation is used to calculate the gradients of the loss with respect to the model parameters (weights, biases, etc.). These gradients are then used to update the model parameters using an optimization algorithm such as stochastic gradient descent (SGD) or Adam.

A survival deep neural network with PEM can be implemented using several libraries in PyTorch that support time-to-event data modelling, including:
torch.nn—Core PyTorch library for neural network layers like nn.Linear, nn.ReLU, and other activation layers for building the model architecture.
pycox—A package built on PyTorch specifically for survival analysis. It provides implementations of common survival loss functions (e.g., partial log-likelihood) and methods like PEM, enabling efficient training on time-to-event data. Implement of PEM is done by using Pycox's utilities.
lifelines (optional)—A survival analysis library (not based on PyTorch) that provides some additional methods for data handling, which is useful for preprocessing survival data.
   5. Causal Deep Neural Network for Ordinary Differential Equations (c-DNN-ODE)
A Causal Deep Neural Network for Ordinary Differential Equations is designed to find a function that describes the change in one variable (Y) over time caused by another variable (X) by modelling continuous changes in the data as it passes between layers of the neural network to capture the non-linear dynamics of how the causal effect of X on Y changes over time.
For this technology, biological cause and effect is modelled using the observed log HR during every month of follow-up in randomized trials evaluating LDL or SBP lowering therapies, respectively, standardized for a 1 unit absolute observed difference in LDL or SBP during the corresponding time interval; and the observed log HR during every year of life among participants randomized by nature to higher or lower LDL or SBP, respectively, standardized for a 1 unit absolute observed difference in LDL or SBP during the corresponding time interval.
The c-DNN-ODE algorithm is designed to quantify how much the proportional reduction in atherosclerotic cardiovascular events caused by lowering LDL or SBP changes over time.
By using a DNN for ODE, we model both the relationship between x and y; and the dynamics of the rate of change (the derivative,

$$\frac{dy}{dx} ) .$$

This differential information allows the network to infer a likely “true curve” representing the relationship governed by the biological impact of slowing the trajectory of atherosclerosis by reducing the number of atherogenic lipoproteins that become trapped within the artery wall over time, incorporating the inherent variability of data points.
   It is a ‘causal’ model because the algorithm is trained exclusively on randomized data from: randomized trials of LDL and SBP lowering therapies, and
   Mendelian randomization studies evaluating genetic variants associated LDL and SBP designed as naturally randomized trials.
The model not only matches the predicted values with experimental results, but also learns the trajectory of y over x, constrained by a smooth, plausible curve that reflects a governing law (biological effect);
By simultaneously learning the derivative (or instantaneous slope) of the change in y at each value of x along with predictions of the output, the network constrains itself to a more consistent and smooth shape that aligns with the expected non-linear biological effect of reducing LDL or SBP on the underlying biological processes of atherosclerotic plaque progression and the propensity of the accumulated plaque to physically disrupt.
Within an ODE approach, the network is regularized by focusing not only on fitting y values, but also on matching the rate of change of y over x.
This focus on differential information helps smooth out experimental variability, leading the model to converge toward the most likely true, smooth curve that represents the biological relationship.
This robustness to variability enhances the model's capacity to generalize, providing a clearer picture of the actual underlying curve.
In addition, when leaning a function by fitting randomized causal experimental data, using an ODE solver eliminates the need for regularization techniques (including L1 and L2 regularization methods).
When learning with causal data from randomized experiments, a typical regularization method shrinks all estimates to reduce sensitivity to outliers, thus biasing the predicted true causal effects toward the null; and therefore systematically underestimating both the proportional and absolute magnitude of the benefit of reducing exposure to a modifiable cause of disease; and systematically underestimating how much relative or proportional benefit increases over time. Thus, combining an ODE with a DNN eliminates the potential for learning estimates of the true causal effects that are systematically biased toward the null.
Training Data: Individual participant data from 1,623,491 participants enrolled in one of three (3) long-term prospective biobank cohort studies for whom at least one LDL or SBP measurements was available, and for whom medical records were available recording age (date) at which the first episode of a fatal or non-fatal MI, fatal or non-fatal ischemic stroke, or coronary revascularization occurred. Participants were censored at time of last follow-up, death, or first atherosclerotic cardiovascular event. Data from 527,512 participants enrolled in 76 randomized trials evaluating LDL or BP lowering therapies that provided time-to-event curves, and measurements of the absolute difference in LDL or SBP between the randomized groups. In addition, summary data from 2.6 million participants with the age (dates) at which a first atherosclerotic cardiovascular event was recorded, was used for additional external validation experiments.
An illustrative DNN for ODE and associated simultaneous calculations and derivatives are shown in FIGS. 10A and 10B.
The overall derivative of

$$\frac{dv}{dx}$$

for the change in Y for a given X can be calculated from the partial derivatives at each node using the chain rule:

$$\frac{dy}{dx} = \frac{dy}{{dh}_{3}} \cdot \frac{{dh}_{3}}{{dh}_{1}} \cdot \frac{{dh}_{1}}{dx} + \frac{dy}{{dh}_{3}} \cdot \frac{{dh}_{3}}{{dh}_{2}} \cdot \frac{{dh}_{2}}{dx} + \frac{dy}{{dh}_{4}} \cdot \frac{{dh}_{4}}{{dh}_{1}} \cdot \frac{{dh}_{1}}{dx} + \frac{dy}{{dh}_{4}} \cdot \frac{{dh}_{4}}{{dh}_{2}} \cdot \frac{{dh}_{2}}{dx}$$

The partial derivatives at each node are weighted by the same learned weights as those used to predict the value of Y at each node; and f( ) represents the activation function of the node output (e.g., ReLU, sigmoid, etc.), so that all derivatives are computed on the activated node output.
Loss function: The loss function for a DNN-ODE is typically the sum of the squared differences between the predicted and actual values. Because the model simultaneously calculates both the predicted output y, and the derivative

$$\frac{dy}{dx}$$

or the rate of change in y with respect to x, the loss function seeks to minimize the mean squared error from the absolute difference between the predicted and observed data for both of these parameters.
Specifically the loss function is calculated as follows:
   the neural network takes X as input and outputs Y and the rate of change of Y to fit a function:

$$\frac{dy}{dt} = f ( t , y )$$

During each epoch, a numerical integration method (like the Euler method or Runge-Kutta) is used to reconstruct the value of Y at different points along X

$$y ( {t}_{1} ) = ODE - Solver ( f , {x}_{0} , {t}_{0} , {t}_{1} )$$

The predicted and observed values of Y, and the predicted and observed rate of change in Y at each value of X, are then compared to calculate the mean squared error as:

$$Loss = \sum {( {( {y}_{pred} - {y}_{true} )}^{2} + α \cdot ( \frac{{dy}_{pred}}{dx} - \frac{{dy}_{true}}{dx} ) )}^{2}$$

where: y_{pred} and yₜᵣᵤₑ and are the predicted and actual y values

$$\frac{{dy}_{pred}}{dx} and \frac{{dy}_{true}}{dx}$$

are the predicted and actual slopes a is a weighting factor to balance the contribution of the two terms
Using backpropagation: the network updates its weights and biases to simultaneously minimize the combined squared errors for both the predicted values of Y and the instantaneous rate of change in Y.
A Causal Deep Neural Network designed to solve or approximate ODEs can be implemented using a combination of standard PyTorch modules along with specialized libraries for solving differential equation, including:
   torch.nn—For building the base layers of the neural network, e.g., nn.Linear, nn.Sequential, etc.
   torchdiffeq—An external library specifically for solving differential equations with PyTorch. This library provides solvers like torchdiffeq.odeint that can handle neural ODEs. The ODE function can be defined as a neural network and passed to odeint to integrate over time.
   Autograd (automatic differentiation)—PyTorch's autograd functionality computes gradients in a neural ODE. It enables the computation of the derivatives of loss with respect to model parameters.

### Example 3

This is an illustrative example of lifetable methodology.

If the baseline risk of an ASCVD event in an interval, t, is assessed to be hₜ, then the risk for the specific individual is hₜ*e^{ln(HR)}—where the ln(HR) is derived from the Survival DNN with PEM for estimating risk; and derived from the Causal DNN for ODE for estimating time-dependent benefit by magnitude and duration of intervention. Within each interval, t, two crucial quantities can be derived:
aₜ: the risk of an ASCVD event in interval t, given ASCVD-free survival up to the start of interval t
bₜ: the risk of a non-CVD death in interval t, given ASCVD-free survival up to the start of interval t From these two quantities all else follows.

The example lifetable begins at the current age of the individual. Equivalently, the lifetable could begin at the current cumulative exposure to LDL (plaque size) of the individual.

| | Interval | | |
|---|---|---|---|
| | 0 | | |
| | Starting | | |
| | at | | |
| | current | Interval | Interval |
| | age | t -1 | t |
| Risk of ASCVD event | 0 | aₜ₋₁ | aₜ |
| in age interval | | | |
| Risk of non - - - CVD | 0 | bₜ₋₁ | bₜ |
| death in age interval | | | |
| Proportion of original | 0 | cₜ₋₁ | cₜ = eₜ₋₁* |
| cohort having non-CVD | | | bₜ |
| death in interval | | | |
| Proportion of original | 0 | dₜ₋₁ | dₜ = eₜ₋₁* |
| cohort having ASCVD | | | aₜ |
| event in interval | | | |
| Proportion surviving | 1 | eₜ₋₁ | eₜ₋ = eₜ₋₁ − |
| free of ASCVD at | | | cₜ- dₜ |
| end of Interval | | | |
| Cumulative proportion | 0 | fₜ₋₁ | fₜ₋ = eₜ₋₁ + |
| with ASCVD events | | | dₜ |
| by end of interval | | | |
| Cumulative proportion | 0 | mₜ₋₁ | mₜ₋ = Mₜ₋₁ + |
| dying from non-CVD | | | cₜ |
| causes by end of interval | | | |

Note that eₜ + fₜ + mₜ = 1 for all t.

Changing the combination of exposures changes the hazard terms aₜ and bₜ to aₜ*and bₜ*: repeating the lifetable calculations leads to a revised ASCVD-free surviving proportion eₜ*and revised cumulative ASCVD proportion fₜ*

From these, the following quantities are calculated:
ASCVD-survival curve: eₜ: Probability of surviving free of ASCVD at end of (age or cumulative LDL exposure) interval t
Cumulative ASCVD risk curve: fₜ: Cumulative proportion with ASCVD events by end of (age or cumulative LDL exposure) interval t
Expected age or cumulative exposure to LDL (size of plaque burden) at first event: Σₜ eₜ*(width of interval t)

### Example 4

This is an illustrative example of the reinforcement learning value function. In particular, this example illustrates the Reinforcement Learning algorithm using Model Dependent Proximal Policy Optimization (MD-PPO) with Markov Chain Tree Search (MCTS) constrained by Domain Expert Global Policy Guidelines.

### Calculating the Value Function for the Model-Based Reinforcement Learning Problem

The value function V(s) represents the expected cumulative reward an agent can obtain from a given state s by following a certain policy z The value function for a state s can be calculated as:

$$V ( s ) = {max}_{a} ( R ( s , a ) + γ {\sum }_{{s}^{′}} P ( {s}^{′} \vert s , a ) V ( {s}^{′} ) )$$

where:
   R(s, a) is the immediate reward for taking action a in state s
   P(s′|s,a) is the probability of transitioning to state s′ after taking action a in state s
   γ is the discount factor, which reduces the value of future rewards
   V(s′) is the value of the next state s′
   maxₐ ensures the agent takes the action that maximizes the expected return

### Penalized Value Function

A penalized value function introduces penalties for certain actions or states. In the framework of this technology, a penalty is assigned for using each intervention during each interval of follow-up;

The penalized value function incorporates a cost C(s,a) for taking action a in state s, which is a negative reward or penalty (in this case for adding additional interventions).

The value function with penalties can be written as:

$$V ( s ) = {max}_{a} ( R ( s , a ) - C ( s , a ) + γ {\sum }_{{s}^{′}} P ( {s}^{′} \vert s , a ) V ( {s}^{′} ) )$$

### Where:

C(s, a) is the penalty or cost for taking action a in state s
All other terms are the same as in the standard value function.

### Discounted Value Function

(1) The discount factor γ (with 0≤γ≤1) is used to reduce the value of future rewards, with larger y values placing more importance on long-term rewards and smaller y values emphasizing short-term rewards.

The discounted value function is given by:

$$V ( s ) = {max}_{a} ( R ( s , a ) + γ {\sum }_{{s}^{′}} P ( {s}^{′} \vert s , a ) V ( {s}^{′} ) )$$

When combining penalization and discounting, the overall value function becomes:

$$V ( s ) = {max}_{a} ( R ( s , a ) - C ( s , a ) + γ {\sum }_{{s}^{′}} P ( {s}^{′} \vert s , a ) V ( {s}^{′} ) )$$

Implementation of the model-based PPO reinforcement learning system combined with MCTS requires a mix of libraries for neural network implementation, reinforcement learning frameworks, and custom MCTS structures, including:

### 1. Core Libraries for PPO and Neural Network Models

PyTorch (torch, torch.nn, torch.optim): For implementing and training the neural network models that act as function approximators within PPO. PyTorch will be used to create both policy and value networks for PPO, and torch.optim can be used for managing the optimizer (e.g., Adam) used in training.

### 2. Libraries for Monte Carlo Tree Search (MCTS)

Since MCTS isn't built into most standard RL libraries, a custom MCTS module can be created or available libraries can be used, including:
anytree: A Python library for creating and managing tree structures, which can be useful for implementing MCTS by creating nodes, parents, and children relationships. It's a general-purpose library, so you'd still implement MCTS logic manually.
AlphaZero General (AZG): This GitHub repository is an implementation of AlphaZero's neural MCTS, which can be adapted and modified. It includes a neural network with MCTS and can be tailored to work within a model-based PPO framework.

### Repository: AlphaZero General (AZG)

custom MCTS implementations: MCTS is commonly implemented from scratch in Python, as it requires specific tuning based on the problem context. A basic MCTS implementation involves defining selection, expansion, simulation, and backpropagation phases, each of which could be tailored to PPO's needs.

### 3. Model-Based RL-Specific Libraries

For model-based reinforcement learning, the Deep Causal AI learned model of the environment can be implemented using:
DynRL: A model-based reinforcement learning library compatible with PyTorch that offers model-learning modules or allows integration of custom models;
torchdyn: A library focused on dynamical systems in PyTorch, which can help build the transition models for the model-based learned environment.
   4. Data and Logging Tools for Model-Based PPO with MCTS

For managing complex experiment data, logging, visualization, the following libraries are used:
Weights & Biases (wandb): An experiment tracking tool that is useful for tracking the model training process, PPO metrics, and MCTS tree statistics over time.
Gymnasium: The updated version of OpenAI Gym, which provides a standard interface for environments used in reinforcement learning. It can be integrated to test a PPO+MCTS agents on a variety of RL environments.

### Example 5

This example illustrates optimal intervention policy selection using reinforcement learning proximal policy optimization, as well as various alternative methods for determining sequence scores for respective therapeutic intervention sequences.

### Intervention Policy Selection Using RL-PPO

In this example, a set of vectors containing the predicted cumulative event rates at all ages for each possible combination & sequence of therapies are then combined into a Markov Decision Process Matrix and passed as input into the Deep Causal AI Model-Dependent Reinforcement Learning Proximal Policy Optimization with Monte Carlo Tree Search (MD_RL PPO with MCTS) constrained by domain expert Global Policy Guidelines (GPG). This Guideline (which can be modified to suit local preferences or to DESIGN RCTs) states that the optimal policy (clinical strategy) to personalize the prevention of cardiometabolic disease is the one that lowers LDL, SBP, Lp(a), HbA1c, and Weight by just the amount each person needs, when they need it, to keep the personal below their personal Plaque Threshold for ASCVD events while also protecting the artery wall by preventing the development of HTN & T2D using the fewest interventions possible. The optimal policy is selected, in this example, by calculating a Value function given by:

$${V ( s )}_{i} = {\sum }_{a ge = i}^{80} {( ( ( - 100 ) * cumHaz ) * ( discount ) )}_{i} - {\sum }_{age = i}^{80} ( {( no . Rx )}_{i} )$$

TABLE 1
Calculation of Cumulative Hazard Input to the Value Function; Columns 1-10

| | | | | | | | 1-year | | |
|---|---|---|---|---|---|---|---|---|---|
| | | | | | | 1-year | survival | Sum betas | Sum betas |
| | | | | | Age at | survival | free of | for CV | for nonCV |
| | | | | | start of | free of | nonCV | event at | death at |
| | | | | | interval | CV event | event | that age | that age |
| Age_int | Cum_fail | Int_hazard | Cum_haz | Instant_surv | age | Ha | Hb | InRR_a | InRR_b |
| 30 | 0 | 0 | 0 | 1 | 30 | 1 | 1 | 0 | 0 |
| 30.25 | 0.0001 | 0.0001 | 0.0001 | 0.9999 | 30.25 | 0.9999 | 1 | 0 | 0 |
| 30.5 | 0.0001 | 0 | 0.0001 | 1 | 30.5 | 1 | 1 | 0 | 0 |
| 31 | 0.0001 | 0 | 0.0001 | 1 | 31 | 1 | 1 | 0 | 0 |
| 31.25 | 0.0001 | 0.0001 | 0.0002 | 0.9999 | 31.25 | 0.9999 | 1 | 0 | 0 |
| 31.5 | 0.0002 | 0 | 0.0002 | 1 | 31.5 | 1 | 1 | 0 | 0 |
| 31.75 | 0.0002 | 0 | 0.0002 | 1 | 31.75 | 1 | 1 | 0 | 0 |
| 32 | 0.0002 | 0 | 0.0002 | 1 | 32 | 1 | 1 | 0 | 0 |
| 32.25 | 0.0003 | 0.0002 | 0.0004 | 0.9999 | 32.25 | 0.9999 | 1 | 0 | 0 |
| 32.5 | 0.0003 | 0 | 0.0004 | 1 | 32.5 | 1 | 1 | 0 | 0 |
| 33 | 0.0004 | 0 | 0.0004 | 1 | 33 | 1 | 1 | 0 | 0 |
| 33.25 | 0.0005 | 0.0001 | 0.0005 | 0.9999 | 33.25 | 0.9999 | 1 | 0 | 0 |
| 33.5 | 0.0005 | 0 | 0.0005 | 1 | 33.5 | 1 | 1 | 0 | 0 |
| 34 | 0.0005 | 0 | 0.0005 | 1 | 34 | 1 | 1 | 0 | 0 |
| 34.25 | 0.0006 | 0.0001 | 0.0006 | 0.9999 | 34.25 | 0.9999 | 1 | 0 | 0 |
| 34.5 | 0.0007 | 0.0001 | 0.0007 | 0.9999 | 34.5 | 0.9999 | 1 | 0 | 0 |
| 35 | 0.0007 | 0 | 0.0007 | 1 | 35 | 1 | 1 | 0 | 0 |
| 70 | 0.1114 | 0.0018 | 0.1183 | 0.9982 | 70 | 0.9982 | 1 | 0 | 0 |
| 70.25 | 0.113 | 0.0018 | 0.1201 | 0.9982 | 70.25 | 0.9982 | 1 | 0 | 0 |
| 70.5 | 0.1143 | 0.0015 | 0.1216 | 0.9985 | 70.5 | 0.9985 | 1 | 0 | 0 |
| 70.75 | 0.116 | 0.0019 | 0.1235 | 0.9981 | 70.75 | 0.9981 | 1 | 0 | 0 |
| 71 | 0.1176 | 0.0018 | 0.1253 | 0.9982 | 71 | 0.9982 | 1 | 0 | 0 |
| 71.25 | 0.1194 | 0.002 | 0.1273 | 0.998 | 71.25 | 0.998 | 1 | 0 | 0 |
| 71.5 | 0.1211 | 0.0019 | 0.1292 | 0.9981 | 71.5 | 0.9981 | 1 | 0 | 0 |
| 71.75 | 0.1226 | 0.0018 | 0.131 | 0.9982 | 71.75 | 0.9982 | 1 | 0 | 0 |
| 72 | 0.1242 | 0.0018 | 0.1328 | 0.9982 | 72 | 0.9982 | 1 | 0 | 0 |
| 72.25 | 0.1258 | 0.0019 | 0.1347 | 0.9981 | 72.25 | 0.9981 | 1 | 0 | 0 |
| 72.5 | 0.1275 | 0.002 | 0.1367 | 0.998 | 72.5 | 0.998 | 1 | 0 | 0 |
| 72.75 | 0.1292 | 0.0019 | 0.1386 | 0.9981 | 72.75 | 0.9981 | 1 | 0 | 0 |
| 73 | 0.1309 | 0.0019 | 0.1405 | 0.9981 | 73 | 0.9981 | 1 | 0 | 0 |
| 73.25 | 0.1324 | 0.0018 | 0.1423 | 0.9982 | 73.25 | 0.9982 | 1 | 0 | 0 |
| 73.5 | 0.1339 | 0.0017 | 0.144 | 0.9983 | 73.5 | 0.9983 | 1 | 0 | 0 |
| 73.75 | 0.1355 | 0.0019 | 0.1459 | 0.9981 | 73.75 | 0.9981 | 1 | 0 | 0 |
| 74 | 0.1368 | 0.0015 | 0.1474 | 0.9985 | 74 | 0.9985 | 1 | 0 | 0 |
| 74.25 | 0.1388 | 0.0023 | 0.1497 | 0.9977 | 74.25 | 0.9977 | 1 | 0 | 0 |
| 74.5 | 0.1404 | 0.0018 | 0.1515 | 0.9982 | 74.5 | 0.9982 | 1 | 0 | 0 |
| 74.75 | 0.1421 | 0.0019 | 0.1534 | 0.9981 | 74.75 | 0.9981 | 1 | 0 | 0 |
| 75 | 0.1438 | 0.002 | 0.1554 | 0.998 | 75 | 0.998 | 1 | 0 | 0 |
| 75.25 | 0.1456 | 0.0021 | 0.1575 | 0.9979 | 75.25 | 0.9979 | 1 | 0 | 0 |
| 75.5 | 0.1476 | 0.0023 | 0.1598 | 0.9977 | 75.5 | 0.9977 | 1 | 0 | 0 |
| 75.75 | 0.1493 | 0.002 | 0.1618 | 0.998 | 75.75 | 0.998 | 1 | 0 | 0 |
| 76 | 0.1512 | 0.0022 | 0.164 | 0.9978 | 76 | 0.9978 | 1 | 0 | 0 |
| 76.25 | 0.1529 | 0.002 | 0.166 | 0.998 | 76.25 | 0.998 | 1 | 0 | 0 |
| 76.5 | 0.1549 | 0.0023 | 0.1683 | 0.9977 | 76.5 | 0.9977 | 1 | 0 | 0 |
| 76.75 | 0.1563 | 0.0017 | 0.17 | 0.9983 | 76.75 | 0.9983 | 1 | 0 | 0 |
| 77 | 0.1579 | 0.002 | 0.172 | 0.998 | 77 | 0.998 | 1 | 0 | 0 |
| 77.25 | 0.1599 | 0.0023 | 0.1743 | 0.9977 | 77.25 | 0.9977 | 1 | 0 | 0 |
| 77.5 | 0.1616 | 0.0021 | 0.1764 | 0.9979 | 77.5 | 0.9979 | 1 | 0 | 0 |
| 77.75 | 0.1637 | 0.0025 | 0.1789 | 0.9975 | 77.75 | 0.9975 | 1 | 0 | 0 |
| 78 | 0.1661 | 0.0029 | 0.1818 | 0.9971 | 78 | 0.9971 | 1 | 0 | 0 |
| 78.25 | 0.1679 | 0.0022 | 0.184 | 0.9978 | 78.25 | 0.9978 | 1 | 0 | 0 |
| 78.5 | 0.1697 | 0.0022 | 0.1862 | 0.9978 | 78.5 | 0.9978 | 1 | 0 | 0 |
| 78.75 | 0.1712 | 0.0018 | 0.188 | 0.9982 | 78.75 | 0.9982 | 1 | 0 | 0 |
| 79 | 0.1727 | 0.0018 | 0.1898 | 0.9982 | 79 | 0.9982 | 1 | 0 | 0 |
| 79.25 | 0.1749 | 0.0027 | 0.1925 | 0.9973 | 79.25 | 0.9973 | 1 | 0 | 0 |
| 79.5 | 0.1761 | 0.0015 | 0.194 | 0.9985 | 79.5 | 0.9985 | 1 | 0 | 0 |
| 79.75 | 0.1781 | 0.0024 | 0.1964 | 0.9976 | 79.75 | 0.9976 | 1 | 0 | 0 |
| 80 | 0.1801 | 0.0024 | 0.1988 | 0.9976 | 80 | 0.9976 | 1 | 0 | 0 |
| 80.25 | 0.1825 | 0.0029 | 0.2017 | 0.9971 | 80.25 | 0.9971 | 1 | 0 | 0 |
| | 0.1825 | | | | | | | | |

Calculation of Cumulative Hazard Input to the Value Function; Columns 11-20

| | | | | Survival | | | | | |
|---|---|---|---|---|---|---|---|---|---|
| | | Proportion | | free of CV | | | | Cumulative | |
| | | of original | Proportion | event | | | | proportion | Internal |
| Risk of | Risk of | cohort | of original | AND | Cumulative | Cumulative | | with | test: sum |
| CV | nonCV | having | cohort | nonCV | proportion | proportion | | EITHER | cumulative |
| event | event | nonCV | having CV | death at | with CV | with nonCV | | CV event | probability |
| during | during | death | death | the end of | event by | event by | Internal | OR nonCV | of VC |
| age | age | during age | during age | age | end of age | end of age | test: sum | death by | event OR |
| interval | interval | interval | interval | interval | interval | interval | equal 1 | end of age | nonCV |
| a_t | b_t | c_t | d_t | e_t | f_t | m_t | e + f + m | interval | death |
| 0 | 0 | 0 | 0 | 1 | 0 | 0 | 1 | 0 | 0 |
| 1E−04 | 0 | 0 | 1E−04 | 0.9999 | 1E−04 | 0 | 1 | 1E−04 | 1E−04 |
| 0 | 0 | 0 | 0 | 0.9999 | 1E−04 | 0 | 1 | 1E−04 | 1E−04 |
| 0 | 0 | 0 | 0 | 0.9999 | 1E−04 | 0 | 1 | 1E−04 | 1E−04 |
| 1E−04 | 0 | 0 | 1E−04 | 0.9998 | 0.0002 | 0 | 1 | 0.0002 | 0.0002 |
| 0 | 0 | 0 | 0 | 0.9998 | 0.0002 | 0 | 1 | 0.0002 | 0.0002 |
| 0 | 0 | 0 | 0 | 0.9998 | 0.0002 | 0 | 1 | 0.0002 | 0.0002 |
| 0 | 0 | 0 | 0 | 0.9998 | 0.0002 | 0 | 1 | 0.0002 | 0.0002 |
| 0.0002 | 0 | 0 | 0.0002 | 0.9996 | 0.0004 | 0 | 1 | 0.0004 | 0.0004 |
| 0 | 0 | 0 | 0 | 0.9996 | 0.0004 | 0 | 1 | 0.0004 | 0.0004 |
| 0 | 0 | 0 | 0 | 0.9996 | 0.0004 | 0 | 1 | 0.0004 | 0.0004 |
| 1E−04 | 0 | 0 | 1E−04 | 0.9995 | 0.0005 | 0 | 1 | 0.0005 | 0.0005 |
| 0 | 0 | 0 | 0 | 0.9995 | 0.0005 | 0 | 1 | 0.0005 | 0.0005 |
| 0 | 0 | 0 | 0 | 0.9995 | 0.0005 | 0 | 1 | 0.0005 | 0.0005 |
| 1E−04 | 0 | 0 | 1E−04 | 0.9994 | 0.0006 | 0 | 1 | 0.0006 | 0.0006 |
| 1E−04 | 0 | 0 | 9.99E−05 | 0.9993 | 0.0007 | 0 | 1 | 0.0007 | 0.0007 |
| 0 | 0 | 0 | 0 | 0.9993 | 0.0007 | 0 | 1 | 0.0007 | 0.0007 |
| 0.0018 | 0 | 0 | 0.001602 | 0.888357 | 0.111643 | 0 | 1 | 0.111643 | 0.111643 |
| 0.0018 | 0 | 0 | 0.001599 | 0.886758 | 0.113242 | 0 | 1 | 0.113242 | 0.113242 |
| 0.0015 | 0 | 0 | 0.00133 | 0.885428 | 0.114572 | 0 | 1 | 0.114572 | 0.114572 |
| 0.0019 | 0 | 0 | 0.001682 | 0.883746 | 0.116254 | 0 | 1 | 0.116254 | 0.116254 |
| 0.0018 | 0 | 0 | 0.001591 | 0.882155 | 0.117845 | 0 | 1 | 0.117845 | 0.117845 |
| 0.002 | 0 | 0 | 0.001764 | 0.880391 | 0.119609 | 0 | 1 | 0.119609 | 0.119609 |
| 0.0019 | 0 | 0 | 0.001673 | 0.878718 | 0.121282 | 0 | 1 | 0.121282 | 0.121282 |
| 0.0018 | 0 | 0 | 0.001582 | 0.877136 | 0.122864 | 0 | 1 | 0.122864 | 0.122864 |
| 0.0018 | 0 | 0 | 0.001579 | 0.875557 | 0.124443 | 0 | 1 | 0.124443 | 0.124443 |
| 0.0019 | 0 | 0 | 0.001664 | 0.873894 | 0.126106 | 0 | 1 | 0.126106 | 0.126106 |
| 0.002 | 0 | 0 | 0.001748 | 0.872146 | 0.127854 | 0 | 1 | 0.127854 | 0.127854 |
| 0.0019 | 0 | 0 | 0.001657 | 0.870489 | 0.129511 | 0 | 1 | 0.129511 | 0.129511 |
| 0.0019 | 0 | 0 | 0.001654 | 0.868835 | 0.131165 | 0 | 1 | 0.131165 | 0.131165 |
| 0.0018 | 0 | 0 | 0.001564 | 0.867271 | 0.132729 | 0 | 1 | 0.132729 | 0.132729 |
| 0.0017 | 0 | 0 | 0.001474 | 0.865797 | 0.134203 | 0 | 1 | 0.134203 | 0.134203 |
| 0.0019 | 0 | 0 | 0.001645 | 0.864152 | 0.135848 | 0 | 1 | 0.135848 | 0.135848 |
| 0.0015 | 0 | 0 | 0.001296 | 0.862856 | 0.137144 | 0 | 1 | 0.137144 | 0.137144 |
| 0.0023 | 0 | 0 | 0.001985 | 0.860871 | 0.139129 | 0 | 1 | 0.139129 | 0.139129 |
| 0.0018 | 0 | 0 | 0.00155 | 0.859321 | 0.140679 | 0 | 1 | 0.140679 | 0.140679 |
| 0.0019 | 0 | 0 | 0.001633 | 0.857689 | 0.142311 | 0 | 1 | 0.142311 | 0.142311 |
| 0.002 | 0 | 0 | 0.001715 | 0.855973 | 0.144027 | 0 | 1 | 0.144027 | 0.144027 |
| 0.0021 | 0 | 0 | 0.001798 | 0.854176 | 0.145824 | 0 | 1 | 0.145824 | 0.145824 |
| 0.0023 | 0 | 0 | 0.001965 | 0.852211 | 0.147789 | 0 | 1 | 0.147789 | 0.147789 |
| 0.002 | 0 | 0 | 0.001704 | 0.850507 | 0.149493 | 0 | 1 | 0.149493 | 0.149493 |
| 0.0022 | 0 | 0 | 0.001871 | 0.848636 | 0.151364 | 0 | 1 | 0.151364 | 0.151364 |
| 0.002 | 0 | 0 | 0.001697 | 0.846938 | 0.153062 | 0 | 1 | 0.153062 | 0.153062 |
| 0.0023 | 0 | 0 | 0.001948 | 0.84499 | 0.15501 | 0 | 1 | 0.15501 | 0.15501 |
| 0.0017 | 0 | 0 | 0.001436 | 0.843554 | 0.156446 | 0 | 1 | 0.156446 | 0.156446 |
| 0.002 | 0 | 0 | 0.001687 | 0.841867 | 0.158133 | 0 | 1 | 0.158133 | 0.158133 |
| 0.0023 | 0 | 0 | 0.001936 | 0.83993 | 0.16007 | 0 | 1 | 0.16007 | 0.16007 |
| 0.0021 | 0 | 0 | 0.001764 | 0.838167 | 0.161833 | 0 | 1 | 0.161833 | 0.161833 |
| 0.0025 | 0 | 0 | 0.002095 | 0.836071 | 0.163929 | 0 | 1 | 0.163929 | 0.163929 |
| 0.0029 | 0 | 0 | 0.002425 | 0.833647 | 0.166353 | 0 | 1 | 0.166353 | 0.166353 |
| 0.0022 | 0 | 0 | 0.001834 | 0.831813 | 0.168187 | 0 | 1 | 0.168187 | 0.168187 |
| 0.0022 | 0 | 0 | 0.00183 | 0.829983 | 0.170017 | 0 | 1 | 0.170017 | 0.170017 |
| 0.0018 | 0 | 0 | 0.001494 | 0.828489 | 0.171511 | 0 | 1 | 0.171511 | 0.171511 |
| 0.0018 | 0 | 0 | 0.001491 | 0.826997 | 0.173003 | 0 | 1 | 0.173003 | 0.173003 |
| 0.0027 | 0 | 0 | 0.002233 | 0.824764 | 0.175236 | 0 | 1 | 0.175236 | 0.175236 |
| 0.0015 | 0 | 0 | 0.001237 | 0.823527 | 0.176473 | 0 | 1 | 0.176473 | 0.176473 |
| 0.0024 | 0 | 0 | 0.001976 | 0.821551 | 0.178449 | 0 | 1 | 0.178449 | 0.178449 |
| 0.0024 | 0 | 0 | 0.001972 | 0.819579 | 0.180421 | 0 | 1 | 0.180421 | 0.180421 |
| 0.0029 | 0 | 0 | 0.002377 | 0.817202 | 0.182798 | 0 | 1 | 0.182798 | 0.182798 |
| 0.2017 | | | | | | | | 0.182798 | |

Column headings, left to right: Age at start of interval; 1-year survival free of CV event; 1-year survival free of nonCV death; Sum betas for CV event at that age; Sum betas for nonCV death at that age; Risk of CV event during age interval; Risk of nonCV event during age interval; Proportion of original cohort having nonCV death during age interval; Proportion of original cohort having CV death during age interval; Survival free of CV event AND nonCV death at the end of age interval; Cumulative proportion with CV event by end of age interval; Cumulative proportion with nonCV event by end of age interval; Internal test: sum equal 1; Cumulative proportion with EITHER CV event OR nonCV death by end of age interval; Internal test: sum cumulative probability of VC event OR nonCV death

Next an example step-by-step calculation of Value Function for alternative clinical strategies (proximal policies) is provided. The objective is to select the optimal proximal policy (initial treatment strategy & subsequent sequence of interventions) that maximizes the Cumulative Value function: this is equivalent to minimizing the area under the cumulative hazard curve for lifetime risk of ASCVD events using the fewest possible interventions by introducing a penalty for each intervention unit used. For example, we can compare the strategies of lowering LDL by 33% beginning at age 40 with lowering LDL by 50% beginning at age 55. The first example is for a policy with yearly administration of siRNA directed against PCSK9 as shown in Table 2.

TABLE 2
Calculation of Cumulative Value for once yearly genetic intervention to lower LDL begining at age 40
Policy (clinical srategy): Lower LDL by 33% beginning at current age of 40 years with once yearly
siRNA directed against PCSK9

| | | | | | Discounted | | | |
|---|---|---|---|---|---|---|---|---|
| | | | Sum of the | | Sum of | | Discounted | Penalty |
| | Instantaneous | Cumulative | Cumulative | | Cumulative | | Interval | (No. of |
| Age | Hazard | Hazard | Hazard | Discount | Hazard | Scaler | Value | Treatments) |
| 40 | 0.0009 | 0.0009 | 0.0009 | 0.9980 | 0.0009 | −100 | −0.0855 | 1 |
| 41 | 0.0006 | 0.0011 | 0.0011 | 0.9980 | 0.0022 | −100 | −0.2228 | 1 |
| 42 | 0.0008 | 0.0021 | 0.0044 | 0.9980 | 0.0044 | −100 | −0.4359 | 1 |
| 43 | 0.0006 | 0.0027 | 0.0070 | 0.9980 | 0.0070 | −100 | −0.7033 | 1 |
| 44 | 0.0007 | 0.0033 | 0.0103 | 0.9980 | 0.0104 | −100 | −1.0356 | 1 |
| 45 | 0.0010 | 0.0032 | 0.0145 | 0.9980 | 0.0146 | −100 | −1.1610 | 1 |
| 46 | 0.0009 | 0.0051 | 0.0187 | 0.9980 | 0.0187 | −100 | −1.9698 | 1 |
| 47 | 0.0009 | 0.0059 | 0.0256 | 0.9980 | 0.0258 | −100 | −3.5648 | 1 |
| 48 | 0.0011 | 0.0170 | 0.0326 | 0.9980 | 0.0327 | −100 | −3.2651 | 1 |
| 49 | 0.0011 | 0.0182 | 0.0408 | 0.9980 | 0.0409 | −100 | −4.0891 | 1 |
| 50 | 0.0015 | 0.0096 | 0.0504 | 0.9980 | 0.0505 | −100 | −5.0537 | 1 |
| 51 | 0.0013 | 0.0108 | 0.0613 | 0.9980 | 0.0614 | −100 | −6.1398 | 1 |
| 52 | 0.0016 | 0.0124 | 0.0736 | 0.9980 | 0.0738 | −100 | −7.3789 | 1 |
| 53 | 0.0015 | 0.0138 | 0.0824 | 0.9980 | 0.0826 | −100 | −8.7573 | 1 |
| 54 | 0.0012 | 0.0154 | 0.1028 | 0.9980 | 0.1030 | −100 | −10.3001 | 1 |
| 55 | 0.0021 | 0.0173 | 0.1201 | 0.9980 | 0.1204 | −100 | −12.0354 | 1 |
| 56 | 0.0020 | 0.0192 | 0.1393 | 0.9980 | 0.1395 | −100 | −13.9548 | 1 |
| 57 | 0.0020 | 0.0210 | 0.1601 | 0.9980 | 0.1606 | −100 | −16.0632 | 1 |
| 58 | 0.0023 | 0.0231 | 0.1835 | 0.9980 | 0.1838 | −100 | −18.3827 | 1 |
| 59 | 0.0022 | 0.0252 | 0.2086 | 0.9980 | 0.2091 | −100 | −20.9061 | 1 |
| 60 | 0.0026 | 0.0278 | 0.2363 | 0.9980 | 0.2367 | −100 | −23.6778 | 1 |
| 61 | 0.0025 | 0.0299 | 0.2662 | 0.9980 | 0.2667 | −100 | −26.6693 | 1 |
| 62 | 0.0027 | 0.0324 | 0.2986 | 0.9980 | 0.2992 | −100 | −29.9152 | 1 |
| 63 | 0.0024 | 0.0366 | 0.3332 | 0.9980 | 0.3393 | −100 | −33.9870 | 1 |
| 64 | 0.0022 | 0.0371 | 0.3703 | 0.9980 | 0.3211 | −100 | −33.1088 | 1 |
| 65 | 0.0026 | 0.0395 | 0.4099 | 0.9980 | 0.4107 | −100 | −31.0695 | 1 |
| 66 | 0.0028 | 0.0420 | 0.4518 | 0.9980 | 0.4527 | −100 | −45.2736 | 1 |
| 67 | 0.0029 | 0.0445 | 0.4985 | 0.9980 | 0.4974 | −100 | −48.7445 | 1 |
| 68 | 0.0027 | 0.0471 | 0.5135 | 0.9980 | 0.5446 | −100 | −54.4605 | 1 |
| 69 | 0.0026 | 0.0495 | 0.5990 | 0.9980 | 0.5982 | −100 | −58.4187 | 1 |
| 70 | 0.0027 | 0.0520 | 0.6450 | 0.9980 | 0.6463 | −100 | −64.8277 | 1 |
| 71 | 0.0029 | 0.0547 | 0.6997 | 0.9980 | 0.7011 | −100 | −70.3065 | 1 |
| 72 | 0.0029 | 0.0573 | 0.7570 | 0.9980 | 0.7585 | −100 | −75.8492 | 1 |
| 73 | 0.0028 | 0.0599 | 0.8169 | 0.9980 | 0.8185 | −100 | −81.8499 | 1 |
| 74 | 0.0029 | 0.0626 | 0.8794 | 0.9980 | 0.8812 | −100 | −88.1206 | 1 |
| 75 | 0.0032 | 0.0655 | 0.9450 | 0.9980 | 0.9469 | −100 | −98.6872 | 1 |
| 76 | 0.0031 | 0.0684 | 1.0133 | 0.9980 | 1.0154 | −100 | −101.5372 | 1 |
| 77 | 0.0032 | 0.0713 | 1.0827 | 0.9980 | 1.0868 | −100 | −108.8827 | 1 |
| 78 | 0.0033 | 0.0743 | 1.1990 | 0.9980 | 1.1613 | −100 | −116.1123 | 1 |
| 79 | 0.0031 | 0.0772 | 1.2362 | 0.9980 | 1.2386 | −100 | −123.8637 | 1 |
| 80 | 0.0032 | 0.0801 | 1.3163 | 0.9980 | 1.3189 | −100 | −131.8894 | 1 |
| | | | | | | | | Sum: 40 |

| | | |
|---|---|---|
| Cumulative Discounted Value | $\text{?}$ | −140.83 |
| Cumulative Penalty | $\text{?}$ | 40 |
| Cumulative Discounted Penalized Value | $\text{?}$ | −180.83 |

[symbol]

[symbol]

indicates data missing or illegible when filed

TABLE 2
Calculation of Cumulative Value for once yearly genetic intervention to lower LDL begining at age 40
Policy (clinical srategy): Lower LDL by 33% beginning at current age of 40 years with once yearly
siRNA directed against PCSK9

| | | | | | Discounted | | | |
|---|---|---|---|---|---|---|---|---|
| | | | Sum of the | | Sum of | | Discounted | Penalty |
| | Instantaneous | Cumulative | Cumulative | | Cumulative | | Interval | (No. of |
| Age | Hazard | Hazard | Hazard | Discount | Hazard | Scaler | Value | Treatments) |
| 40 | 0.0010 | 0.0010 | 0.0010 | 0.9980 | 0.0010 | −100 | −0.1014 | 0 |
| 41 | 0.0006 | 0.0016 | 0.0026 | 0.9980 | 0.0026 | −100 | −0.2605 | 0 |
| 42 | 0.0011 | 0.0027 | 0.0053 | 0.9980 | 0.0053 | −100 | −0.5311 | 0 |
| 43 | 0.0008 | 0.0035 | 0.0088 | 0.9980 | 0.0088 | −100 | −0.8818 | 0 |
| 44 | 0.0010 | 0.0045 | 0.0133 | 0.9980 | 0.0133 | −100 | −1.3327 | 0 |
| 45 | 0.0015 | 0.0060 | 0.0193 | 0.9980 | 0.0183 | −100 | −1.9339 | 0 |
| 46 | 0.0014 | 0.0074 | 0.0267 | 0.9980 | 0.0268 | −100 | −2.6754 | 0 |
| 47 | 0.0015 | 0.0089 | 0.0356 | 0.9980 | 0.0357 | −100 | −3.5671 | 0 |
| 48 | 0.0019 | 0.0108 | 0.0464 | 0.9980 | 0.0465 | −100 | −4.6493 | 0 |
| 49 | 0.0023 | 0.0131 | 0.595 | 0.9980 | 0.0696 | −100 | −5.5619 | 0 |
| 50 | 0.0027 | 0.0188 | 0.0753 | 0.9980 | 0.0755 | −100 | −7.5451 | 0 |
| 51 | 0.0024 | 0.0162 | 0.0935 | 0.9980 | 0.0937 | −100 | −9.3687 | 0 |
| 52 | 0.0031 | 0.0313 | 0.1146 | 0.9980 | 0.1180 | −100 | −11.5030 | 0 |
| 53 | 0.0029 | 0.0242 | 0.1390 | 0.9980 | 0.1393 | −100 | −13.9279 | 0 |
| 54 | 0.0035 | 0.0277 | 0.1667 | 0.9980 | 0.1670 | −100 | −16.7034 | 0 |
| 55 | 0.0035 | 0.0312 | 0.1979 | 0.9980 | 0.1988 | −100 | −19.8307 | 2 |
| 56 | 0.0028 | 0.0340 | 0.2329 | 0.9980 | 0.2323 | −100 | −23.2346 | 2 |
| 57 | 0.0026 | 0.0366 | 0.2685 | 0.9980 | 0.2690 | −100 | −26.9040 | 2 |
| 58 | 0.0028 | 0.0394 | 0.3079 | 0.9980 | 0.3085 | −100 | −30.8536 | 2 |
| 59 | 0.0026 | 0.0420 | 0.3499 | 0.9980 | 0.3506 | −100 | −35.0608 | 2 |
| 60 | 0.0030 | 0.0460 | 0.3949 | 0.9980 | 0.3957 | −100 | −39.5554 | 2 |
| 61 | 0.0027 | 0.0476 | 0.4475 | 0.9980 | 0.4434 | −100 | −44.3376 | 2 |
| 62 | 0.0028 | 0.0504 | 0.4928 | 0.9980 | 0.4939 | −100 | −49.3915 | 2 |
| 63 | 0.0029 | 0.0529 | 0.5458 | 0.9980 | 0.5469 | −100 | −54.6928 | 2 |
| 64 | 0.0027 | 0.0566 | 0.6014 | 0.9980 | 0.6026 | −100 | −60.2998 | 2 |
| 65 | 0.0026 | 0.0580 | 0.6594 | 0.9980 | 0.6607 | −100 | −66.0738 | 2 |
| 66 | 0.0024 | 0.0605 | 0.7199 | 0.9980 | 0.7213 | −100 | −72.3330 | 2 |
| 67 | 0.0026 | 0.0631 | 0.7830 | 0.9980 | 0.7645 | −100 | −78.4546 | 2 |
| 68 | 0.0023 | 0.0654 | 0.8484 | 0.9980 | 0.8501 | −100 | −85.0116 | 2 |
| 69 | 0.0026 | 0.0677 | 0.9161 | 0.9980 | 0.9180 | −100 | −91.7965 | 2 |
| 70 | 0.0029 | 0.0700 | 0.9881 | 0.9980 | 0.9881 | −100 | −98.8223 | 2 |
| 71 | 0.0024 | 0.0725 | 1.0586 | 0.9980 | 1.0607 | −100 | −106.0220 | 2 |
| 72 | 0.0026 | 0.0746 | 1.1334 | 0.9980 | 1.1357 | −100 | −113.5656 | 2 |
| 73 | 0.0022 | 0.0778 | 1.2104 | 0.9980 | 1.2128 | −100 | −121.2845 | 2 |
| 74 | 0.0023 | 0.0799 | 1.2898 | 0.9980 | 1.2911 | −100 | −129.2347 | 2 |
| 75 | 0.0025 | 0.0818 | 1.3716 | 0.9980 | 1.3743 | −100 | −137.4347 | 2 |
| 76 | 0.0024 | 0.0842 | 1.4558 | 0.9980 | 1.4587 | −100 | −145.8704 | 2 |
| 77 | 0.0024 | 0.0866 | 1.5424 | 0.9980 | 1.5455 | −100 | −154.5482 | 2 |
| 78 | 0.0025 | 0.0891 | 1.6314 | 0.9980 | 1.6347 | −100 | −163.4718 | 2 |
| 79 | 0.0022 | 0.0913 | 1.7228 | 0.9980 | 1.7252 | −100 | −172.6204 | 2 |
| 80 | 0.0023 | 0.0996 | 1.8164 | 0.9980 | 1.8200 | −100 | −182.0009 | 2 |
| | | | | | | | | Sum: 50 |

| | | |
|---|---|---|
| Cumulative Discounted Value | $\text{?}$ | −180.00 |
| Cumulative Penalty | $\text{?}$ | 50 |
| Cumulative Discounted Penalized Value | $\text{?}$ | −232.00 |

[symbol]

[symbol]

indicates data missing or illegible when filed

Another example of calculating a Value function is shown in Table 3. In this case, for a clinical strategy that involves twice yearly genetic intervention siRNA directed against PCSK9.

Comparing these two ‘policies’ or treatment strategies: a once yearly intervention to lower LDL by a time-averaged 33%; and twice yearly intervention to lower LDL by 50%. Both policies achieve the goal of maintaining lifetime risk of having an ASCVD event below a hypothetical of 10% by age 80 years. However, the once yearly siRNA policy achieves a lower area under the curve for cumulative lifetime risk (140.8 v. 189.0) and a lower penalty for the total number of treatment units required to achieve the goal (40 v. 50). Therefore, the once yearly siRNA treatment strategy has a greater total cumulative Value (−180.8 v. −232.0) and is thus the preferred clinical strategy (proximal policy) to achieve the stated remaining lifetime risk goal.

This example illustrates that modest, sustained early reductions in LDL (33%) have a greater overall cumulative clinical benefit as compared to more aggressive LDL lowering (LDL) started later in life—thus illustrating the benefit of early intervention to lower LDL to reduce the lifetime risk of ASCVD events by slowing the progression of atherosclerosis, and quantifying increasing benefit over time.

### Alternative Techniques for Scoring Therapeutic Intervention Sequences

One alternative approach to scoring a therapeutic intervention sequence for a subject is by quantifying its impact on the subject's remaining lifetime risk of having a MACE. In this approach, an intervention or a sequence of multiple interventions that produces the lowest remaining lifetime risk of MACE scores ‘highest’ and therefore wins. This cumulative lifetime risk can be calculated using the techniques described herein. However, scoring sequences by having he score quantified as remaining lifetime risk of MACE (in which case, the lowest score ‘wins’; or the corresponding absolute or proportional reduction in risk, in which case the highest ‘score’ wins) leads the algorithms to learn how to ‘win’ this chess match with nature by simply determining that all interventions should begin immediately to achieve the maximum benefit.

However, simply treating everyone with everything beginning immediately is not the optimal use of resources substantially undermining the benefit-to-cost ratio increase from early intervention to prevent events by slowing disease progression, exposures everyone to same level of potential risk for adverse side effects while offering widely variable levels of benefit to each person, and does not permit any individualization of interventions to personalize the prevention of cardiovascular (or cardiometabolic) disease or any flexibility to incorporate local expertise or policy constraints.

Another alternative approach is using the benefit-cost-ratio as a score for each sequence of therapeutic interventions. The selection of the targets, timing, and sequence of interventions for each person can be selected as the one that achieves the highest benefit-to-cost ratio (from the perspective of society, a payer, or an individual). Alternatively, one use the benefit-to-risk ratio as the ‘score’, where the winner is the target(s), intensity, timing, and sequence of interventions that produces the greatest benefit while minimizing risk (however, those risk would have to be defined and quantified; which is very difficult because most adverse drug reactions tend to be rare and therefore difficult to precisely quantify).

In addition, one could easily simplify the potential solution space (of therapeutic intervention sequences) by limiting the interventions to specific therapies whose impact on LDL, SBP, Lp(a), or any combination of them can be quantified.

For example, consider limiting possible interventions to an siRNA therapy directed against PCSK9 anticipated to produce a 40% time-averaged yearly reduction in LDL, and an siRNA therapy against AGT anticipated to produce a 5% time-averaged yearly reduction in SBP. When given as a single once-yearly combination therapy, the combined PCSK9-AGT siRNA is anticipated to reduce cumulative exposure to LDL and BP by 40% and 5%, respectively each year, when taking into consideration the area-under the curve of the pharmacodynamic effects of the yearly siRNA that rapidly reduces LDL and SBP, reaches a nadir, remains at that plateau for a variable duration of time, and then begins to increase back to baseline before the next annual siRNA (with different time-dependent pharmacodynamic trajectories for the LDL and SBP lowering effects of the siRNA).

In this illustrative example, when we simply try to identify the optimal timing for a single intervention, the solution space becomes very limited. We simply want to know the optimal timing of when to start the annual combination siRNA therapy to achieve the therapeutic goal (e.g., keeping remining lifetime risk less than 1 in 20, or less than 5%).

To select the option that has the best ‘score’ and therefore wins, we can proceed as follows. First, we simply calculate the benefit of a 40% sustained reduction in LDL and a 5% sustained reduction in SBP to estimate the corresponding absolute reductions in LDL and SBP for a given person (which is simply the product of their age- and sex-specific untreated LDL multiplied by 0.40; and their untreated age-and-sex-specific SBP by 0.05) to determine the expected proportional risk reduction at every year from the current age until age 80 (where the proportional risk reduction increases over time depending on the absolute magnitude of LDL and SBP reduction and the duration of intervention). The corresponding absolute reduction in risk of MACE is then simply the sum of the instantaneous hazards during each remaining year of life—both those without treatment and those with treatment. To identify the best option for when to start, we just calculate the remaining lifetime risk conditional on starting the siRNA therapy now, as compared to starting the siRNA next year, the year after, and at all subsequent ages.

For each candidate ‘optimal timing’ of when to begin the siRNA therapy, to calculate the remaining cumulative lifetime risk of a MACE, we create a lifetable calculating the instantaneous hazard of MACE during each year of life before treatment starts and the corresponding reduced instantaneous hazard of MACE during each subsequent year of life beginning with the age at which treatment was started, and then simply sum these hazards to get the cumulative hazard of MACE conditional on when treatment with the siRNA therapy was started. Note here that for the most extreme case of starting treatment immediately, there are no ages or other risk intervals off of treatment.

In this example, then, the ‘score’ for starting an annual siRNA at each age can be formulated in numerous ways: the lowest remaining absolute cumulative life risk, or the latest starting age that can keep remaining lifetime below the target (e.g. below 5%).

Note that the MD-PPO approach described herein may become useful in extreme cases, where the number of possible targets, intensities of LDL and SBP lowering in response to the possible interventions, sequences of interventions, and order of interventions is large in which case the solution space becomes far too large to catalogue by brute force. As a result, a MDTS or other method to randomly sample the solution space, which can be somewhat constrained by a ‘policy’ of only adding or intensifying therapies (avoiding any solution that does not continue a therapy directed against an initially targeted exposure) may be employed, as described herein.

In such embodiments, the RL-PPO helps us to restrict possible solution space to those that are clinically reasonable or consistent with local resource constraints, clinical guidance, or other policy. The MD (causal world model of how atherosclerotic cardiovascular disease develops) further restricts the computational burden and improves the biological plausibility and clinical rationale of any selected policy by preventing the model to try to ‘learn’ the solution without being grounded in biology (to avoid confounding and other biases of non-randomized data) using the techniques described herein to estimate all instantaneous hazards, cumulative hazards, and reduced instantaneous and cumulative hazards due to the timing, intensity, sequence, and order of interventions used to reduce cumulative exposure to the modifiable causes of disease in quantitatively precise way that is directly informed exclusively by unconfounded causal randomized evidence to train the ‘causal AI model’ to learn the biology of how atherosclerosis develops. Indeed, one cannot perform the RL without or without a PPO without the techniques described herein because randomized longitudinal data under different treatment scenarios do not exist and can be approximated with causal methods described herein, in order to conduct the RL or ‘play the game against nature’ to determine the optimal strategy for personalizing the prevention of ASCVD for each person.

### Example 6

This example illustrates aspects of incorporating updates in the state of the patient's cardiometabolic health (e.g., for example at a later point in time, such as a year later.

The updated state of cardiometabolic health is determined by two dynamic processes: the achieved absolute reduction in the modifiable cause(s) of disease targeted by the recommended intervention(s); and the absolute changes in other exposures not targeted by the recommended interventions(s) due to aging and the evolving biology of how common diseases develop during the same interval of longitudinal follow-up. FIG. 9A illustrates measurements defining the updated state of cardiometabolic health of the subject.

The changes in the measured clinical, biometric, and biochemical features over the previous time interval are then passed as input into the Deep Causal AI Agent to perform updated predictive and prescriptive analyses. These updated measurements help the Deep Causal AI Agent ‘learn’ the individual trajectories of each person more precisely passed on the observed changes over time thus creating more precise individualized estimates of each person's projected trajectories of non-targeted levels of LDL, apoB, Lp(a), SBP, weight, waist circumference, and HbA1c. These more precise estimates of how each person's cardiometabolic health is evolving over time, in turn, inform more precise predictions of risk and the expected benefit of specific interventions that are used to longitudinally monitor cardiometabolic health over time and make updated recommendations to ensure that cardiometabolic health is being preserved.

The entire rationale for early intervention to lower LDL and other apoB-containing lipoproteins including Lp(a), SBP, and other modifiable exposures that cause irreversible structural injury that accumulates over time is to prevent this accumulating injury and thereby slow the progression of common disease. The only way to understand when to intervene and by how much is by showing that the benefit of reducing the modifiable causes of disease increases over time and precisely quantifying the magnitude of increasing benefit over time. Therefore, having a method for recording and quantifying the legacy benefit from earlier interventions prevents catastrophic forgetting of this benefit which would otherwise systematically & progressively underestimate the benefit of early intervention & thus underestimate both the clinical and economic value of early intervention to slow the progression of common cardiometabolic diseases, as illustrated in FIG. 9B.

This problem can be solved by quantifying the legacy benefit of earlier interventions among persons who survive to a specific age without an ASCVD event (and therefore survived these risk intervals which no longer contribute to the cumulative lifetime risk of ASCVD events) as shown in Table 4.

This example reveals how failure to incorporate the legacy benefit from earlier intervention leads to a higher predicted cumulative exposure to LDL at the current age, and a higher predicted remaining absolute cumulative lifetime risk of ASCVD events as compared to the lower predicted accumulated plaque burden size (cumulative exposure to LDL) and corresponding lower remaining cumulative lifetime risk of ACSVD due to the benefit from earlier LDL lowering. Failure to account for the legacy benefit can falsely lead to an updated recommendation to intensify the optimal intervention or combination of interventions(s) used to achieve the desired goal.

We now provide additional motivation and detail for why and how to quantify the legacy benefit. As a person ages, the amount of atherosclerosis that has developed and the amount of injury to the arterial wall that has accumulated increases. As a result, a person's short-term risk (e.g. 10-year risk) of experiencing a major atherosclerotic cardiovascular event (MACE) increases. This can be explained by the fact that there is a certain instantaneous risk of having a MACE during each year of life. When a person is younger, very little plaque and arterial wall injury have accumulated within their arteries. As a result, their short-term risk of MACE is very low. As the person ages, more atherosclerotic plaque and arterial wall injury accumulate, and therefore both their instantaneous and short-term risk increases over time.

By contrast, and somewhat counterintuitively, a person's remaining cumulative lifetime risk of an outcome decreases with age. This can be explained by the fact that a person's remaining lifetime risk of MACE is simply the cumulative sum of their instantaneous hazards during each year of life. As a person ages and does not experience a MACE, they have survived all previous risk intervals up their current age. Their ‘updated’ remaining lifetime risk of MACE is now less because it is simply the cumulative sum of all ‘remaining’ risk intervals (e.g., the instantaneous hazard of experiencing a MACE during the ‘remaining’ years of their life up to age 80 or other arbitrarily selected limit).

In effect, a person's ‘remaining’ lifetime risks to age 80 (for example) is merely the cumulative risk of MACE by age 80 less the cumulative risk up to their current age. Thus, a person's lifetime risk of MACE is at a maximum when young, because they have not yet survived any risk intervals. As that person ages, if they survive to a certain age without a MACE they have already survived all risk intervals at earlier ages and therefore their ‘remaining’ lifetime risk of MACE decreases.

This helps to understand how the ‘legacy benefit’ of an earlier intervention is calculated. When estimating remaining lifetime risk of a person who has not experienced a MACE using lifetable analysis (or any other similar iterative mathematical process), we reset ‘cumulative risk’ up to the current age as zero (0), as can be seen in the lifetable analysis shown in Table 4, because the person has survived all earlier risk intervals and they are no longer at risk from prior hazards that they already survived. By contrast, their cumulative exposure to LDL (and SBP) continues to increase because, although they have not experienced a MACE, LDL has continued to accumulative in their arterial walls to form a progressively increasing plaque burden, and irreversible structural arterial wall injury from blood pressure and/or other causes of injury has accumulated decreasing their capacity to tolerate the accumulated plaque burden. As a result, the instantaneous hazard of MACE increases during each year proportional to the amount of atherosclerotic plaque and arterial wall injury that has accumulated.

Without prior intervention to lower LDL or SBP, we must include the sum of those previous exposures to LDL and SBP (and the projected sum of all future exposures to LDL and SBP) to calculate the cumulative exposure to LDL and SBP up to that point and at all future points in time order to calculate the instantaneous hazard of MACE during each year of life going forward because those risks are proportional to the cumulative exposure to LDL and SBP at each age or other time point.

To capture the legacy benefit, we record the reduction in LDL or SBP during all prior years of any interventions to lower LDL and SBP beginning with the age at which either or both of those interventions were started. We then calculate cumulative exposure to LDL and SBP as the sum of the untreated values of LDL and SBP during each year of life up to the age at which the intervention was started, and the reduced values of LDL or SBP during each subsequent year of life that the intervention was used up to age 80 (or other age limit). The reduced LDL, SBP, or both at each prior age interval and all future age intervals reduces the predicted instantaneous hazard during all previous risk and all future risk intervals during which the person was receiving an intervention by lowering LDL and SBP—and this benefit increases over time because the instantaneous hazard of having a MACE (and therefore the sum of those instantaneous hazards or cumulative remaining lifetime risk) is proportional to the magnitude and duration of LDL and SBP lower, i.e. proportional to the cumulative reduction in LDL and SBP. As a result, the lifetable for a person who started intervention in the past reflects the reduced instantaneous hazard of MACE during all past with and without interventions to lower LDL or SBP, and all future time intervals under the assumption that treatment will continue. The practical effect is the construction of a lifetime reduced instantaneous hazards for MACE for all ages intervals during which the person was receiving an intervention to lower LDL, SBP, or both relative to what the instantaneous hazards would have been without intervention. These reduced instantaneous hazards are then summed to calculate the revised cumulative lifetime risk of having a MACE incorporating all prior interventions and assuming those interventions will continue.

Therefore, when a person survives to a certain age without MACE, their cumulative lifetime risk of MACE up to that age is reset at zero (because they have not had an event), but their cumulative exposures to LDL and SBP have are not reset to zero but they continue to determine the reduced instantaneous hazard during each year of life that interventions were used in the past and the instantaneous hazard of all future time intervals.

The practical effect is that the new revised instantaneous hazard of experiencing a MACE during the current year of life begins at zero (because the person survived all current risk intervals), but the instantaneous hazard during this year of life is lower than it would have been if the person had not started interventions to lower LDL, SBP, or both in the past—and the magnitude of reduced risk is proportional to the magnitude and duration (cumulative exposure) to LDL or SBP or both up to that point in time. In addition, all future instantaneous hazards will all be lower by an amount that is proportional to the cumulative reductions in LDL or SBP up to that point. This is the ‘legacy benefit’ from early intervention, and the foregoing discussion and Table 4 explain how to quantify it.

Therefore, the lifetable approach provides a convenient method to record and quantify the benefit of lowering LDL and SBP during each prior year of life (the ‘legacy benefit’) and all future years of intervention proportional to the magnitude and duration (reduction in cumulative exposure) to LDL, SBP, or both), while also allowing up to ‘discount’ the intervals that a person survived in the past to recalculate their remaining cumulative lifetime risk of MACE from their current age until age 80.

By contrast, if we did not record and quantify the ‘legacy benefit’ in this way, we would reset both the instantaneous hazard of MACE to zero, and the benefit of lowering LDL and SBP back to the benefit observed during the first year of intervention to lower LDL, SBP, or both. Because the benefit of lowering LDL and SBP is proportional to the magnitude and duration of LDL and SBP, failure to account for prior interventions to lower LDL and SBP would result in higher instantaneous hazards during the current year of life and all future years of life resulting in a higher remaining cumulative lifetime risk of MACE by failing to take into account the ‘legacy benefit’ from earlier interventions to lower LDL and SBP to slow the progression of accumulative atherosclerosis and arterial wall injury.

Therefore, failure to account for the ‘legacy benefit’ of earlier interventions to slow disease progression would overestimate the ‘residual risk’ of MACE during all future years of life. Indeed the ‘legacy benefit’ may be defined as the difference in residual risk between earlier and later interventions to lower LDL, SBP, or both. This not only provides a metric to quantify the legacy benefit—it also redefines ‘legacy benefit’ as the reduced residual risk during treatment to lower LDL or SBP due to prior interventions to slow disease progression.

TABLE 4
Illustration of quantifying legacy benefit
A Method to Record & Quantify the Legacy Benefit of Earlier Treaments

| | | | | IGNORING Legacy Benefit of | | | INCLUDING Legacy Benefit of | | |
|---|---|---|---|---|---|---|---|---|---|
| No treatment | | | | Earlier Treatment Starting age 40 | | | Earlier Treatment Starting age 40 | | |
| | Cumulative | | Cum | Cumulative | | Cum | Cumulative | | Cum |
| age | Exp LDL | Ln(HR) | Haz | Exp LDL | Ln(HR) | Haz | Exp LDL | Ln(HR) | Haz |
| 40 | 104.8 | | | 104.8 | | | 104.8 | [symbol] 0.100 | |
| 41 | 10 [symbol] . [symbol] | | | 10 [symbol] . [symbol] | | | 107.1 | [symbol] 0. [symbol] | |
| 42 | 111.7 | | | 111.7 | | | 10 [symbol] .4 | [symbol] .2 [symbol] | |
| 43 | 11 [symbol] .2 | | | 11 [symbol] .2 | | | 111. [symbol] | [symbol] 0. [symbol] 1 | |
| 44 | 11 [symbol] .7 | | | 11 [symbol] .7 | | | 114.1 | [symbol] 0. [symbol] | |
| 45 | 122. [symbol] | Survive to Age [symbol] | | 122. [symbol] | Survive to Age [symbol] | | 11 [symbol] | [symbol] 0. [symbol] | S [symbol] to |
| 46 | 12 [symbol] .8 | without [symbol] | | 12 [symbol] .8 | without [symbol] ASCVD | | 11 [symbol] . [symbol] | [symbol] 0.412 | age [symbol] |
| 47 | 12 [symbol] .3 | ASCVD Event | | 12 [symbol] | Event - IGNORING | | 121.2 | [symbol] 0.441 | without |
| 48 | 1 [symbol] 2.9 | | | 1 [symbol] 2.9 | earlier LDL | | 12 [symbol] . [symbol] | [symbol] 0.4 [symbol] | event [symbol] |
| 49 | 1 [symbol] 6. [symbol] | | | 1 [symbol] . [symbol] | lowering [symbol] at | | 12 [symbol] .0 | [symbol] 0. [symbol] 2 | INCLUDING |
| 50 | 140.1 | | | 140.1 | age 40 | | 128.4 | [symbol] 0. [symbol] | LDL |
| 51 | 1 [symbol] . [symbol] | | | 143.6 | | | 1 [symbol] 0. [symbol] | [symbol] 0. [symbol] | lowering |
| 52 | 147.2 | | | 147.2 | | | 133.2 | [symbol] 0. [symbol] | [symbol] ffect |
| 53 | 1 [symbol] 0 [symbol] | | | 1 [symbol] 0. [symbol] | | | 13 [symbol] . [symbol] | [symbol] 0. [symbol] | beginning |
| 54 | 154.4 | | | 154.4 | | | 1 [symbol] .0 | [symbol] 0. [symbol] | at age 40 |
| 55 | 158.0 | 0 | 0.004 | 158.0 | [symbol] 0.100 | 0.004 | 140.4 | [symbol] 0. [symbol] | 0.002 |
| 56 | 1 [symbol] 1. [symbol] | 0 | 0.008 | 1 [symbol] . [symbol] | [symbol] 0.220 | 0.007 | 14 [symbol] . [symbol] | [symbol] 0. [symbol] 0 | 0.004 |
| 57 | 165.2 | 0 | 0.013 | 1 [symbol] 2.8 | [symbol] 0.269 | 0.010 | 145.2 | [symbol] 0. [symbol] 45 | 0.006 |
| 58 | 168.8 | 0 | 0.018 | 165.2 | [symbol] 0.311 | 0.013 | 147.7 | [symbol] 0.660 | 0.008 |
| 59 | 17 [symbol] .4 | 0 | 0.022 | 167. [symbol] | [symbol] 0. [symbol] | 0.017 | 150.1 | [symbol] 0. [symbol] | 0.010 |
| 60 | 17 [symbol] .0 | 0 | 0.028 | 170.0 | [symbol] 0. [symbol] 2 | 0.020 | 152.5 | [symbol] 0. [symbol] 88 | 0.013 |
| 61 | 179. [symbol] | 0 | 0.034 | 172.4 | [symbol] 0.412 | 0.024 | 1 [symbol] . [symbol] | [symbol] 0.701 | 0.016 |
| 62 | 183.2 | 0 | 0.040 | 174.8 | [symbol] 0.441 | 0.027 | 157. [symbol] | [symbol] 0.71 [symbol] | 0.018 |
| 63 | 186.7 | 0 | 0.046 | 177.2 | [symbol] 0.467 | 0.031 | 15 [symbol] .7 | [symbol] 0.725 | 0.021 |
| 64 | 190. [symbol] | 0 | 0.052 | 17 [symbol] . [symbol] | [symbol] 0.492 | 0.034 | 162.1 | [symbol] 0. [symbol] 6 | 0.023 |
| 65 | 193. [symbol] | 0 | 0.058 | 182.0 | [symbol] 0.515 | 0.038 | 164.5 | [symbol] 0.747 | 0.026 |
| 66 | 197.4 | 0 | 0.065 | 184.4 | [symbol] 0.537 | 0.041 | 1 [symbol] . [symbol] | [symbol] 0. [symbol] | 0.02 [symbol] |
| 67 | 201. [symbol] | 0 | 0.072 | 1 [symbol] .8 | [symbol] 0. [symbol] | 0.045 | 1 [symbol] .3 | [symbol] 0. [symbol] 66 | 0.031 |
| 68 | 20 [symbol] . [symbol] | 0 | 0.078 | 1 [symbol] . [symbol] | [symbol] 0. [symbol] 7 | 0.04 [symbol] | 171.7 | [symbol] 0.77 [symbol] | 0.034 |
| 69 | 208.2 | 0 | 0.08 [symbol] | 191.6 | [symbol] 0. [symbol] 95 | 0.051 | 174.1 | 0.784 | 0.037 |
| 70 | 211.8 | 0 | 0.092 | 19 [symbol] .0 | [symbol] 0.61 [symbol] | 0.0 [symbol] | 17 [symbol] .5 | [symbol] 0.792 | 0.040 |
| 71 | 21 [symbol] .3 | 0 | 0.099 | 19 [symbol] .4 | [symbol] 0.630 | 0.058 | 178.9 | [symbol] 0. [symbol] 00 | 0.042 |
| 72 | 218.9 | 0 | 0.107 | 19 [symbol] . [symbol] | [symbol] 0. [symbol] 45 | 0.062 | 18 [symbol] . [symbol] | [symbol] 0. [symbol] | 0.045 |
| 73 | 222.4 | 0 | 0.114 | 201.2 | [symbol] 0.660 | 0.065 | 18 [symbol] . [symbol] | [symbol] 0. [symbol] 14 | 0.048 |
| 74 | 226.0 | 0 | 0. [symbol] 22 | [symbol] . [symbol] | [symbol] 0.6 [symbol] | 0.069 | 18 [symbol] .0 | [symbol] 0. [symbol] 21 | 0.0 [symbol] 1 |
| 75 | 229.5 | 0 | 0.01 [symbol] 0 | [symbol] .9 | [symbol] 0.688 | 0.072 | 188. [symbol] | [symbol] 0. [symbol] | 0.054 |
| 76 | 2 [symbol] .0 | 0 | 0.139 | 208. [symbol] | [symbol] 0. [symbol] 01 | 0.076 | 190. [symbol] | [symbol] 0.833 | 0.057 |
| 77 | 236. [symbol] | 0 | 0.147 | 210.6 | [symbol] 0.713 | 0.080 | 1 [symbol] .1 | [symbol] 0. [symbol] | 0.061 |
| 78 | 240.0 | 0 | 0.156 | 212. [symbol] | [symbol] 0.7 [symbol] | 0.083 | 1 [symbol] .4 | [symbol] 0. [symbol] | 0.064 |
| 79 | 24 [symbol] .5 | 0 | 0.164 | 21 [symbol] . [symbol] | [symbol] 0. [symbol] | 0.0 [symbol] | 19 [symbol] . [symbol] | [symbol] 0. [symbol] | 0.0 [symbol] |
| 80 | 247.0 | 0 | 0.173 | 21 [symbol] . [symbol] | [symbol] 0.747 | 0.091 | 200.1 | [symbol] 0. [symbol] 2 | 0.070 |
| | Cum [symbol] LDL | | Cum [symbol] Haz | Cum [symbol] LDL | | Cum [symbol] Haz | Cum [symbol] LDL | | Cum [symbol] Haz |

[symbol]

indicates data missing or illegible when filed

### Example 7

With respect to the Wald ratio, this refers to a commonly used method in statistics which is sometimes alternatively referred to as the “ratio of effect estimates” method. This method may be used in the context of the technology described herein (e.g., to determine the expected benefit of lowering LDL or SBP over any time interval for a subject under consideration) as follows.

First, from a randomized study of an intervention (including a LDL or SBP lowering therapy, or a genetic variant that lowers LDL or SBP, regardless of whether the variant is in the gene that encodes the specific target of a therapy in, e.g., PCSK9, in which case it can serve as an instrument to directly estimate the causal effect of lifelong treatment with a therapy), two ‘causal’ estimates of effect may be obtained.

These causal estimates of effect include: (1) the causal effect of the intervention on the biomarker of interest (and the associated standard error, SE); and (2) the causal effect of the intervention on the outcome of interest (and associated SE).

The magnitude of the intervention's causal effect on the outcome will depend on the magnitude of that intervention's effect on the biomarker change. For example, if an intervention causes a large reduction in LDL, it will cause a large reduction in MACE. On the other hand, if an intervention causes a small reduction in LDL, it will cause a small reduction in MACE.

In this example, by randomization, where all other exposures are equally distributed, we assume the magnitude of causal effect on outcome is proportional to the magnitude of effect on the biomarker (but only for those exposures where we know from randomized study experience and data that the relationship between the causal biomarker and disease is log-linear).

Therefore, to fairly compare the effect of different interventions, we standardize the estimated benefit per unit change in the causal biomarker. Effectively, in this example, we want a standard estimate of how much an intervention reduces the risk of MACE per mmol/L (38.67 mg/dl) reduction in LDL, and per 10 mmHg (or 1 mmHg) reduction in SBP.

This may be estimated by using the Wald ratio of the causal effect estimates. For example, if an intervention: (1) reduces LDL by 0.5 mmol/L; and (2) reduces the risk of MACE by 10% (HR 0.90; lnHR −0.10536), then the effect of this intervention on the risk of MACE per mmol/L reduction in LDL may be determined as the simple ratio of these two effect estimates: −0.10536/0.5=−0.2071. Exponentiating this (−0.2071=0.81). Thus, this intervention reduces risk of MACE by 19% per 1 mmol/L reduction in LDL.

Importantly, we also divide the SE of the lnHR estimating the causal effect of the intervention on MACE by the causal effect of that intervention on LDL. This step is performed when combining the standardized or adjusted causal effect sizes on MACE per mmol/L reduction in LDL to obtain an overall average summary estimate of causal effect (the inverse variance weighted average summary estimate of effect per mmol/L reduction in LDL).

This same process of adjusting the predicted benefit for the achieved reduction in a biomarker can be applied under any circumstance, including in the scenario described in the lifetables described herein.

At each time point, to estimate the reduced risk from an intervention, the predicted instantaneous hazard of having a MACE within that time interval is multiplied by the lnHR for the intervention based on the duration of intervention for a one unit reduction in the targeted biomarker (e.g. a 1 mmol/L reduction in LDL during the 10th year of sustained treatment). However, this only gives us the predicted benefit for a 1 mmol/L reduction in sustained LDL during that interval. If the actual reduction achieved from the intervention is 2 mmol/L, we then multiply (not divide) the lnHR by 2 (i.e., by the absolute change in in the biomarker; 0.2, 0.5, 1.5, 2, etc.) to get the effect of 2 mmol/L reduction in LDL during the 10th year of sustained treatment. We multiply in this case, because the effect estimates (lnHR) have already been standardized for a one unit change in biomarker.

### Example 8

As described herein, act 223 of process 200 involves determining absolute instantaneous hazard rates of experiencing a cardiovascular event at the respective levels of cumulative LDL exposure, at average levels of all other exposures, in the reference population. This example describes aspects of this determination.

There are two ways to establish the baseline absolute hazard of experiencing a MACE during each increment of increasing plaque burden: a) based on randomized experiments using the observed absolute event rates among all participants in the reference population; or b) based on randomized experiments using the observed absolute event rates among those participants in the reference population without a family history of ASCVD, who did not smoke, and who did not develop hypertension or diabetes and who had low cumulative exposure to SBP. Either approach may be used.

First, if we conduct a naturally randomized time-to-event trial by having nature randomize participants to different levels of higher or lower LDL, it is clear that those persons randomized by nature to lifelong higher LDL have a higher LDL level at all ages (and therefore a higher cumulative exposure to LDL at all ages) and a higher corresponding cumulative risk of MACE at all ages—when age is used as the increment of follow-up (x-axis) since the time of randomization (birth or conception, which are equivalent for our purposes). However, when we use cumulative exposure to LDL as the increment of follow-up (x-axis), participants in all groups have exactly the same cumulative lifetime risk of MACE at all levels of cumulative exposure regardless of age or the age at which they achieved any level of cumulative exposure.

This important naturally randomized experiment establishes that age is not the main determinant of MACE, but is merely an omnibus metric of cumulative exposure to LDL (and other exposures that cause accumulating irreversible structural injury). Rather, the risk of MACE is determined almost exclusively by the size of a person's accumulated plaque burden measured in plaque-years of cumulative exposure to LDL—when all other exposures are equally distributed in a randomized experiment.

Therefore, we can use this naturally randomized evidence as the ground truth to provide an estimate of the baseline instantaneous and cumulative hazards of MACE during every increment of increasing plaque burden measured as cumulative exposure to LDL. If we use all participants, this means that our ‘baseline’ or reference group reflects the instantaneous and cumulative absolute risk of MACE at all levels of plaque burden (cumulative LDL) for the average person in the population (separately for biological males and females because they have different biological vulnerability to LDL and SBP). The ‘average person’ in the population actually means at the average levels of all other exposures because the reference group will have an average level of SBP, weight, waist circumference, HbA1c, smoking history; and proportion of persons with type 2 diabetes, hypertension, family history, Lp(a), polygenic score, and all other exposures.

Thus the reference ‘person’ in this analysis is a composite of the average person in the population who has the population average levels of all exposures. Conceptually, we describe this as the risk of experiencing a MACE at all levels of accumulating plaque burden for persons with “average levels of all other exposures” in the reference population.

Second, as an alternative, we can take the naturally randomized time-to-event trial and define the reference group as the subgroup of persons who have lower cumulative exposure to SBP and have never smoked; have average Lp(a), polygenic scores, weight, waist circumference, HbA1c, and all other biomarkers; and who do not have a family history of ASCVD and have not developed T2D, HTN, or other cardiometabolic conditions that can injure the artery wall. This establishes a reference group that reflects just the effect of cumulative exposure to LDL and therefore gives us an estimate of the magnitude of the instantaneous and cumulative hazards of MACE determined by the size of the accumulated plaque burden, without other forms of accumulated injury to the artery wall.

In this alternative formulation, the reference level personal plaque threshold for developing MACE is for persons without other forms of arterial wall injury, and therefore have the maximum capacity to tolerate the accumulated plaque burden. As a result, exposure to each additional cause of accumulating arterial wall injury, propensity for increased plaque rupture, or inherited vulnerability to accumulating plaque or plaque disruption increases the contemporaneous instantaneous risk of MACE and the resulting cumulative risk of MACE at all levels of accumulate plaque burden—and thus showing how each of these exposures reduces the ‘personal plaque threshold’ by injuring the arterial wall and reducing the capacity of their arteries to tolerate the accumulated plaque burden thus increasing the instantaneous and cumulative lifetime risk of MACE. These two formulations are computationally and biologically equivalent, but the latter is perhaps more intuitive.

### Example Computer System

FIG. 11 illustrates an example of a suitable computing system environment 1100 on which aspects of the technology described herein may be implemented. The computing system environment 1100 is only one example of a suitable computing environment and is not intended to suggest any limitation as to the scope of use or functionality of the technology described herein. Neither should the computing environment 1100 be interpreted as having any dependency or requirement relating to any one or combination of components illustrated in the exemplary operating environment 1100.

The technology described herein is operational with numerous other general purpose or special purpose computing system environments or configurations. Examples of well-known computing systems, environments, and/or configurations that may be suitable for use with the technology described herein include, but are not limited to, personal computers, server computers, hand-held or laptop devices, multiprocessor systems, microprocessor-based systems, set top boxes, programmable consumer electronics, network PCs, minicomputers, mainframe computers, distributed computing environments that include any of the above systems or devices, and the like.

The computing environment may execute computer-executable instructions, such as program modules. Generally, program modules include routines, programs, objects, components, data structures, etc. that perform particular tasks or implement particular abstract data types. The technology described herein may also be practiced in distributed computing environments where tasks are performed by remote processing devices that are linked through a communications network. In a distributed computing environment, program modules may be located in both local and remote computer storage media including memory storage devices.

With reference to FIG. 11, an exemplary system for implementing the technology described herein includes a general purpose computing device in the form of a computer 1100. Components of computer 1110 may include, but are not limited to, a processing unit 1120, a system memory 1130, and a system bus 1121 that couples various system components including the system memory to the processing unit 1120. The system bus 1121 may be any of several types of bus structures including a memory bus or memory controller, a peripheral bus, and a local bus using any of a variety of bus architectures. By way of example, and not limitation, such architectures include Industry Standard Architecture (ISA) bus, Micro Channel Architecture (MCA) bus, Enhanced ISA (ELISA) bus, Video Electronics Standards Association (VESA) local bus, and Peripheral Component Interconnect (PCI) bus also known as Mezzanine bus.

Computer 1110 typically includes a variety of computer readable media. Computer readable media can be any available media that can be accessed by computer 1110 and includes both volatile and nonvolatile media, removable and non-removable media. By way of example, and not limitation, computer readable media may comprise computer storage media and communication media. Computer storage media includes volatile and nonvolatile, removable, and non-removable media implemented in any method or technology for storage of information such as computer readable instructions, data structures, program modules or other data. Computer storage media includes, but is not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium which can be used to store the desired information, and which can be accessed by computer 1110.

Communication media typically embodies computer readable instructions, data structures, program modules, or other data in a modulated data signal such as a carrier wave or other transport mechanism and includes any information delivery media. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, RF, infrared, and other wireless media. Combinations of any of the above should also be included within the scope of computer readable media.

The system memory 1130 includes computer storage media in the form of volatile and/or nonvolatile memory such as read only memory (ROM) 1131 and random access memory (RAM) 1132. A basic input/output system 1133 (BIOS), containing the basic routines that help to transfer information between elements within computer 1110, such as during start-up, is typically stored in ROM 1131. RAM 1132 typically contains data and/or program modules that are immediately accessible to and/or presently being operated on by processing unit 1120. By way of example, and not limitation, FIG. 11 illustrates operating system 1134, application programs 1135, other program modules 1136, and program data 1137.

The computer 1110 may also include other removable/non-removable, volatile/nonvolatile computer storage media. By way of example only, FIG. 11 illustrates a hard disk drive 1141 that reads from or writes to non-removable, nonvolatile magnetic media, a flash drive 1151 that reads from or writes to a removable, nonvolatile memory 1152 such as flash memory, and an optical disk drive 1155 that reads from or writes to a removable, nonvolatile optical disk 1156 such as a CD ROM or other optical media. Other removable/non-removable, volatile/nonvolatile computer storage media that can be used in the exemplary operating environment include, but are not limited to, magnetic tape cassettes, flash memory cards, digital versatile disks, digital video tape, solid state RAM, solid state ROM, and the like. The hard disk drive 1141 is typically connected to the system bus 1121 through a non-removable memory interface such as interface 1140, and magnetic disk drive 1151 and optical disk drive 1155 are typically connected to the system bus 1121 by a removable memory interface, such as interface 1150.

The drives and their associated computer storage media described above and illustrated in FIG. 11, provide storage of computer readable instructions, data structures, program modules, and other data for the computer 1110. In FIG. 11, for example, hard disk drive 1141 is illustrated as storing operating system 1144, application programs 1145, other program modules 1146, and program data 1147. Note that these components can either be the same as or different from operating system 1134, application programs 1135, other program modules 1136, and program data 1137. Operating system 1144, application programs 1145, other program modules 1146, and program data 1147 are given different numbers here to illustrate that, at a minimum, they are different copies. An actor may enter commands and information into the computer 1110 through input devices such as a keyboard 1162 and pointing device 1161, commonly referred to as a mouse, trackball, or touch pad. Other input devices (not shown) may include a microphone, joystick, game pad, satellite dish, scanner, or the like. These and other input devices are often connected to the processing unit 1120 through a user input interface 1160 that is coupled to the system bus, but may be connected by other interface and bus structures, such as a parallel port, game port, or a universal serial bus (USB). A monitor 1191 or other type of display device is also connected to the system bus 1121 via an interface, such as a video interface 1190. In addition to the monitor, computers may also include other peripheral output devices such as speakers 1197 and printer 1196, which may be connected through an output peripheral interface 1195.

The computer 1110 may operate in a networked environment using logical connections to one or more remote computers, such as a remote computer 1180. The remote computer 1180 may be a personal computer, a server, a router, a network PC, a peer device, or other common network node, and typically includes many or all of the elements described above relative to the computer 1110, although only a memory storage device 1181 has been illustrated in FIG. 11. The logical connections depicted in FIG. 11 include a local area network (LAN) 1181 and a wide area network (WAN) 1183, but may also include other networks. Such networking environments are commonplace in offices, enterprise-wide computer networks, intranets and the Internet.

When used in a LAN networking environment, the computer 1110 is connected to the LAN 1181 through a network interface or adapter 1180. When used in a WAN networking environment, the computer 1110 typically includes a modem 1182 or other means for establishing communications over the WAN 1183, such as the Internet. The modem 1182, which may be internal or external, may be connected to the system bus 1121 via the actor input interface 1160, or other appropriate mechanism. In a networked environment, program modules depicted relative to the computer 1110, or portions thereof, may be stored in the remote memory storage device. By way of example, and not limitation, FIG. 11 illustrates remote application programs 1185 as residing on memory device 1181. It will be appreciated that the network connections shown are exemplary and other means of establishing a communications link between the computers may be used.

Having thus described several aspects of at least one embodiment of the technology described herein, it is to be appreciated that various alterations, modifications, and improvements will readily occur to those skilled in the art.

Such alterations, modifications, and improvements are intended to be part of this disclosure, and are intended to be within the spirit and scope of disclosure. Further, though advantages of the technology described herein are indicated, it should be appreciated that not every embodiment of the technology described herein will include every described advantage. Some embodiments may not implement any features described as advantageous herein and in some instances one or more of the described features may be implemented to achieve further embodiments. Accordingly, the foregoing description and drawings are by way of example only.

The above-described embodiments of the technology described herein can be implemented in any of numerous ways. For example, the embodiments may be implemented using hardware, software, or a combination thereof. When implemented in software, the software code can be executed on any suitable processor or collection of processors, whether provided in a single computer or distributed among multiple computers. The software code may be implemented in any suitable computing environment including, for example, a cloud computing environment. Such processors may be implemented as integrated circuits, with one or more processors in an integrated circuit component, including commercially available integrated circuit components known in the art by names such as CPU chips, GPU chips, microprocessor, microcontroller, or co-processor. Alternatively, a processor may be implemented in custom circuitry, such as an ASIC, or semicustom circuitry resulting from configuring a programmable logic device. As yet a further alternative, a processor may be a portion of a larger circuit or semiconductor device, whether commercially available, semi-custom or custom. As a specific example, some commercially available microprocessors have multiple cores such that one or a subset of those cores may constitute a processor. However, a processor may be implemented using circuitry in any suitable format.

Further, it should be appreciated that a computer may be embodied in any of a number of forms, such as a rack-mounted computer, a desktop computer, a laptop computer, or a tablet computer. Additionally, a computer may be embedded in a device not generally regarded as a computer but with suitable processing capabilities, including a Personal Digital Assistant (PDA), a smart phone or any other suitable portable or fixed electronic device.

Also, a computer may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computer may receive input information through speech recognition or in other audible format.

Such computers may be interconnected by one or more networks in any suitable form, including as a local area network or a wide area network, such as an enterprise network or the Internet. Such networks may be based on any suitable technology and may operate according to any suitable protocol and may include wireless networks, wired networks or fiber optic networks.

Also, the various methods or processes outlined (e.g., the processes described herein in connection with FIGS. 2A-2E) herein may be coded as software that is executable on one or more processors that employ any one of a variety of operating systems or platforms. Additionally, such software may be written using any of a number of suitable programming languages and/or programming or scripting tools, and also may be compiled as executable machine language code or intermediate code that is executed on a framework or virtual machine.

In this respect, aspects of the technology described herein may be embodied as a computer readable storage medium (or multiple computer readable media) (e.g., a computer memory, one or more floppy discs, compact discs (CD), optical discs, digital video disks (DVD), magnetic tapes, flash memories, circuit configurations in Field Programmable Gate Arrays or other semiconductor devices, or other tangible computer storage medium) encoded with one or more programs that, when executed on one or more computers or other processors, perform methods that implement the various embodiments described above.

As is apparent from the foregoing examples, a computer readable storage medium may retain information for a sufficient time to provide computer-executable instructions in a non-transitory form. Such a computer readable storage medium or media can be transportable, such that the program or programs stored thereon can be loaded onto one or more different computers or other processors to implement various aspects of the technology as described above. As used herein, the term “computer-readable storage medium” encompasses only a non-transitory computer-readable medium that can be considered to be a manufacture (i.e., article of manufacture) or a machine. Alternatively or additionally, aspects of the technology described herein may be embodied as a computer readable medium other than a computer-readable storage medium, such as a propagating signal.

The terms “program” or “software” are used herein in a generic sense to refer to any type of computer code or set of computer- and/or processor-executable instructions that can be employed to program a computer or other processor to implement various aspects of the technology as described above. Additionally, it should be appreciated that according to one aspect of this embodiment, one or more computer programs that when executed perform methods of the technology described herein need not reside on a single computer or processor, but may be distributed in a modular fashion amongst a number of different computers or processors to implement various aspects of the technology described herein.

Computer-executable instructions may be in many forms, such as program modules, executed by one or more computers or other devices. Generally, program modules include routines, programs, objects, components, data structures, etc. that perform particular tasks or implement particular abstract data types. Typically, the functionality of the program modules may be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the dataset fields with locations in a computer-readable medium that conveys relationship between the dataset fields. However, any suitable mechanism may be used to establish a relationship between information in fields of a data structure, including through the use of pointers, tags or other mechanisms that establish relationship between data elements.

Various aspects of the technology described herein may be used alone, in combination, or in a variety of arrangements not specifically described in the embodiments described in the foregoing and is therefore not limited in its application to the details and arrangement of components set forth in the foregoing description or illustrated in the drawings. For example, aspects described in one embodiment may be combined in any manner with aspects described in other embodiments.

Also, the technology described herein may be embodied as a method, of which examples are provided herein including with reference to FIGS. 2A-2E. The acts performed as part of any of the methods may be ordered in any suitable way. Accordingly, embodiments may be constructed in which acts are performed in an order different than illustrated, which may include performing some acts simultaneously, even though shown as sequential acts in illustrative embodiments.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

What is claimed is:

1. A method for estimating a cumulative LDL exposure trajectory for a subject, the method comprising:
using at least one computer hardware processor to perform:
obtaining cardiometabolic health data for a subject comprising: one or more values for one or more clinical characteristics of the subject, and/or one or more values for one or more physical measurements of the subject, and/or one or more values for one or more biochemical measurements of the subject;
encoding the cardiometabolic health data for the subject into a feature vector;
estimating an LDL level trajectory for the subject by processing the feature vector using an LDL trajectory prediction machine learning (ML) model that has been trained to estimate an LDL level for a subject at each of multiple prior ages and each of multiple future ages using training data comprising for each of a plurality of participants, repeated longitudinal measures of LDL levels and values for the one or more clinical characteristics and/or the one or more physical measurements and/or the one or more biochemical measurements over at least 10, at least 20, at least 30, or at least 50 years of follow-up, wherein the LDL level trajectory for the subject comprises estimated LDL levels for the subject including an estimated LDL level for each of multiple prior ages of the subject and multiple future ages of the subject;
estimating, using the LDL level trajectory, a cumulative LDL exposure trajectory for the subject with respect to a set of ages, wherein the cumulative LDL exposure trajectory comprises an estimated cumulative LDL exposure level for the subject at each age in the set of ages; and
outputting the estimate cumulative LDL exposure trajectory for the subject.

2. The method of claim 1, wherein the one or more values for one or more clinical characteristics comprise:
(i) values for one or more demographic characteristics of the subject, one or more genetic characteristics of the subject, one or more family history characteristics of the subjects, one or more comorbidities, and/or risk factors;
(ii) the one or more values for one or more physical measurements of the subject comprise one or more values for physical measurements of quantities that are risk factors for cardiometabolic disease and/or physiologic al measurements selected from blood pressure measurements and measurements indicative of adiposity; and/or
(iii) the one or more values for one or more biochemical measurements of the subject comprise one or more values for biochemical measurements of quantities that are risk factors for cardiovascular disease and/or biochemical measurements selected from measurements of one or more biochemical markers.

3. The method of claim 1, wherein the cardiometabolic health data includes a value indicating the subject's age, a value indicating the subject's biological sex, a value indicating the subject's LDL level, a value indicating the subject's SBP, and a value indicating the subject's DBP.

4. The method of claim 1, wherein encoding the cardiometabolic health data for the subject into a feature vector comprises:
standardizing at least some of values in the cardiometabolic health data to obtain an initial feature vector; and
positionally encoding at least some feature values in the initial feature vector by the subject's age or ages at which the at least some of feature values were measured to obtain the feature vector.

5. The method of claim 4, wherein performing the standardizing comprises:
performing min-max standardizing the at least some of the values, for continuous variables, in the subject characteristic and/or measurement data, and/or
numerically encoding any dichotomous or ordinal values in the subject characteristic and/or measurement data.

6. The method of claim 4, wherein the positionally encoding comprises:
generating a positional encoding of the initial feature vector using sinusoidal encoding of at least some elements of the initial feature vector, wherein the sinusoidal encoding using age in years as position; and
generating the feature vector by appending the positional encoding of the initial feature vector to the initial feature vector.

7. The method of claim 1, wherein the LDL trajectory prediction ML model is a recurrent neural network model or a neural network model having a transformer architecture.

8. The method of claim 7, wherein the LDL trajectory prediction ML model is a recurrent neural network model, wherein the recurrent neural network model is a bi-directional LSTM (bi-LSTM) model, a bi-directional GRU model, or an ODE-RNN model.

9. The method of claim 8, wherein:
the LDL trajectory prediction ML model is the bi-LSTM model, and
processing the feature vector using the LDL trajectory prediction ML model comprises:
estimating an LDL level for the subject at each of multiple future ages using a forward pass of the bi-LSTM model; and
estimating an LDL level for the subject at each of multiple prior ages using a backward pass of the bi-LSTM model.

10. The method of claim 9, wherein the bi-LSTM model has at least 5,000 parameters.

11. The method of claim 1, further comprising:
encoding the cardiometabolic health data for the subject into a second feature vector;
estimating an SBP level trajectory for the subject by processing the second feature vector using an SBP trajectory prediction ML model that has been trained to estimate an SBP level for a subject at each of multiple prior ages and each of multiple future ages using training data comprising, for each of a plurality of participants, repeated longitudinal measures of SBP levels and values for the one or more clinical characteristics and/or the one or more physical measurements and/or the one or more biochemical measurements over at least 10, at least 20, at least 30, or at least 50 years of follow-up,
wherein the SBP level trajectory for the subject comprises an estimated SBP level for the subject for each of multiple prior ages of the subject and multiple future ages of the subject.

12. The method of claim 11, further comprising:
encoding the cardiometabolic health data for the subject into a third feature vector;
estimating an Lp(a) level trajectory for the subject by processing the third feature vector using an Lp(a) trajectory prediction ML model that has been trained to estimate Lp(a) levels for the subject at each of the multiple prior ages and each of the multiple future ages using training data comprising, for each of a plurality of participants, repeated longitudinal measures of Lp(a) levels and values for the one or more clinical characteristics and/or the one or more physical measurements and/or the one or more biochemical measurements over multiple years of follow-up,
wherein the Lp(a) level trajectory for the subject comprises an estimated L(p) level for the subject for each of multiple prior ages of the subject and multiple future ages of the subject.

13. The method of claim 1, further comprising:
determining, using at least some of the cardiometabolic health data including the cumulative LDL
exposure trajectory, a first trained machine learning (ML) model and for each of multiple time intervals, one or more measures of risk that the subject develops cardiovascular disease to obtain multiple measures of risk corresponding to the multiple time intervals;
determining, using the multiple measures of risk that the subject develops cardiovascular disease and at least one second ML model, benefit of administering to the subject one or more therapeutic interventions designed to reduce risk of cardiovascular disease by targeting one or more modifiable causes of the cardiovascular disease; and
identifying, using the determined benefit of administering the one or more therapeutic interventions, at least one therapeutic intervention to recommend being administered to the subject.

14. The method of claim 13, wherein determining the one or more measures of risk that the subject develops cardiovascular disease, for each of the multiple time intervals, to obtain the multiple measures of risk corresponding to the multiple time intervals, comprises:
(a) estimating, using the first trained ML model and the at least some of the cardiometabolic health data including the cumulative LDL exposure trajectory for the subject, values indicative of log hazard ratios for risk of the subject having a cardiovascular event at respective levels of cumulative LDL exposure; and
(b) estimating, using the values indicative of the log hazard ratios,
(i) absolute instantaneous hazard rates of the subject having a cardiovascular event at the respective levels of cumulative LDL exposure, and
(ii) cumulative lifetime risks of the subject having a cardiovascular event at the respective levels of cumulative LDL exposure;
(c) estimating, using the cumulative LDL exposure trajectory for the subject, the multiple measures of risk to include:
(i) absolute instantaneous hazard rates of the subject having a cardiovascular event at respective ones of the multiple time intervals, and
(ii) cumulative lifetime hazard and event rates of the subject having a cardiovascular event at the respective ones of the multiple time intervals.

15. The method of claim 14, wherein the first trained ML model is a trained survival model using cumulative LDL exposure as an interval of follow-up.

16. The method of claim 15, wherein the first trained ML model is a trained survival DNN model with piecewise exponential models (PEM) using cumulative LDL exposure as the interval of follow-up, wherein the trained survival DNN model comprises a different set of trained weights for each interval of follow-up, wherein each interval of follow-up is an interval of cumulative LDL exposure.

17. The method of claim 1, further comprising:
administering and/or commencing administering to the subject the at least one identified therapeutic intervention,
wherein the at least one identified therapeutic intervention is a DNA-based therapeutic intervention, an RNA-based therapeutic intervention, a protein-based therapeutic intervention, or a pharmacological therapeutic intervention.

18. The method of claim 17, wherein the at least one identified therapeutic intervention is LDL-lowering siRNA, AGT-lowering siRNA, or a combination therapy of LDL-lowering siRNA and AGT-lowering siRNA.

19. A system, comprising:
at least one computer hardware processor; and
at least one non-transitory computer-readable storage medium storing processor executable instructions that, when executed by the at least one computer hardware processor, cause the at least one computer hardware processor to perform a method for estimating a cumulative LDL exposure trajectory for a subject, the method comprising:
obtaining cardiometabolic health data for a subject comprising: one or more values for one or more clinical characteristics of the subject, and/or one or more values for one or more physical measurements of the subject, and/or one or more values for one or more biochemical measurements of the subject;
encoding the cardiometabolic health data for the subject into a feature vector;
estimating an LDL level trajectory for the subject by processing the feature vector using an LDL trajectory prediction machine learning (ML) model that has been trained to estimate am LDL level for a subject at each of multiple prior ages and each of multiple future ages using training data comprising for each of a plurality of participants, repeated longitudinal measures of LDL levels and values for the one or more clinical characteristics and/or the one or more physical measurements and/or the one or more biochemical measurements over at least 10, at least 20, at least 30, or at least 50 years of follow-up, wherein the LDL level trajectory for the subject comprises estimated LDL levels for the subject including an estimated LDL level for each of multiple prior ages of the subject and multiple future ages of the subject;
estimating, using the LDL level trajectory, a cumulative LDL exposure trajectory for the subject with respect to a set of ages, wherein the cumulative LDL exposure trajectory comprises an estimated cumulative LDL exposure level for the subject at each age in the set of ages; and
outputting the estimate cumulative LDL exposure trajectory for the subject.

20. At least one non-transitory computer-readable storage medium storing processor executable instructions that, when executed by at least one computer hardware processor, cause the at least one computer hardware processor to perform a method for estimating a cumulative LDL exposure trajectory for a subject, the method comprising:
obtaining cardiometabolic health data for a subject comprising: one or more values for one or more clinical characteristics of the subject, and/or one or more values for one or more physical measurements of the subject, and/or one or more values for one or more biochemical measurements of the subject;
encoding the cardiometabolic health data for the subject into a feature vector;
estimating an LDL level trajectory for the subject by processing the feature vector using an LDL trajectory prediction machine learning (ML) model that has been trained to estimate am LDL level for a subject at each of multiple prior ages and each of multiple future ages using training data comprising for each of a plurality of participants, repeated longitudinal measures of LDL levels and values for the one or more clinical characteristics and/or the one or more physical measurements and/or the one or more biochemical measurements over at least 10, at least 20, at least 30, or at least 50 years of follow-up, wherein the LDL level trajectory for the subject comprises estimated LDL levels for the subject including an estimated LDL level for each of multiple prior ages of the subject and multiple future ages of the subject;
estimating, using the LDL level trajectory, a cumulative LDL exposure trajectory for the subject with respect to a set of ages, wherein the cumulative LDL exposure trajectory comprises an estimated cumulative LDL exposure level for the subject at each age in the set of ages; and
outputting the estimate cumulative LDL exposure trajectory for the subject.
